(19) United States
(12) Patent Application Publication
(10) Pub. No.: US 2006/0088825 A1
(43) Pub. Date: Apr. 27, 2006

(54) Human delta-n p73 molecules and uses thereof

(76) Inventors: Ian Hayes, Cork (IE); Gennaro Melino, Roma (IT); Vincenzo De Laurenzi, Castel Gandolfo (IT); Daniela Barcaroli, Pescara (IT); Eleonora Candi, Morena (IT); Francesca Bernassola, Roma (IT); Andreas Tobler, Bern (CH); Urban Novak, Bern (CH)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 10/489,730

(22) PCT Filed: Sep. 17, 2002
(86) PCT No.: PCT/GB02/04238

§ 371 Date: Oct. 26, 2004

Related U.S. Application Data
(60) Provisional application No. 60/322,436, filed on Sep. 17, 2001.

(51) Int. Cl. C12Q 1/68; G01N 33/53; C07H 21/04; C12P 21/06; C12N 9/02

(52) U.S. Cl. 435/6; 435/7.1; 435/69.1; 435/189; 435/320.1; 435/325; 530/388.26; 536/23.2

(57) ABSTRACT

The present invention is in the field of molecular biology and genetics. More specifically, the invention relates to human ΔN p73, a novel gene associated with apoptosis regulation. The present invention provides and includes nucleic acid molecules, proteins and antibodies associated with ΔN p73 and methods utilizing such agents, for example in gene isolation, gene analysis, the production of transformed cell lines, and transgenic cells modified to over- or under-express ΔN p73. Moreover, the present invention includes use of the agents of the invention for the diagnosis, prevention and treatment of diseases associated with decreases or increased apoptosis.

### FIELD OF THE INVENTION

The present invention is in the field of molecular biology and genetics. More specifically, the invention relates to nucleic acid and amino acid sequences of a novel inhibitor of apoptosis, the human protein ΔN p73. The present invention provides and includes nucleic acid molecules, proteins, and antibodies associated with ΔN p73 and also provides methods utilizing such agents, for example in gene isolation, gene analysis, the production of transformed cell lines, and transfected and transformed cells and organisms modified to over- or under-express ΔN p73. Moreover, the present invention includes use of the agents of the invention for the diagnosis, prevention and treatment of diseases associated with decreased or increased apoptosis.

### BACKGROUND OF THE INVENTION

Normal development, growth, and homeostasis in multi-cellular organisms require a careful balance between the production and destruction of cells in tissues throughout the body. Cell division is a carefully coordinated process with numerous checkpoints and control mechanisms. These mechanisms are designed to regulate DNA replication and to prevent inappropriate or excessive proliferation. In contrast, apoptosis is the genetically controlled process by which cells die under both physiological conditions, when unneeded or damaged cells are eliminated without causing the tissue destruction and inflammatory responses that are often associated with acute injury and necrosis, and a variety of pathological conditions.

The term “apoptosis” was first used to describe the morphological changes that characterize cells undergoing programmed cell death. Apoptotic cells have a shrunken appearance with an altered membrane lipid content and highly condensed nuclei. DNA fragmentation caused by the activation of endogenous endonucleases results in a DNA ladder pattern which is readily visualized in agarose cells. Phosphatidylserine, a phospholipid normally located on the inner side of the membrane lipid bilayer, is “flipped” to the outside surface of the plasma membrane, where it serves as a signal for the recognition and phagocytosis of the apoptotic cell. Apoptotic cells are rapidly phagocytosed by neighboring cells or macrophages without leaking their potentially damaging contents into the surrounding tissue or triggering an inflammatory response.

The processes and mechanisms regulating apoptosis are highly conserved throughout the phylogenetic tree, and much of the current knowledge about apoptosis is derived from studies of the nematode Caenorhabditis elegans and the fruit fly Drosophila melanogaster. Aberrations in apoptosis regulation have recently been recognized as significant factors in the pathogenesis of human disease. For example, inappropriate cell survival can cause or contribute to many diseases such as cancer, autoimmune diseases, and inflammatory diseases. In contrast, increased apoptosis can cause immunodeficiency diseases such as AIDS, neurodegenerative disorders, and myelodysplastic syndromes.

Many pathological conditions result, at least in part, from aberrant control of cell proliferation, differentiation and/or apoptosis. For example, neoplasia is characterized by a clonally derived cell population which has a diminished capacity for responding to normal cell proliferation control signals. Oncogenic transformation of cells leads to a number of changes in cellular metabolism, physiology, and morphology. One characteristic alteration of oncogenically transformed cells is a loss of responsiveness to constraints on cell proliferation and differentiation normally imposed by the appropriate expression of cell growth regulatory genes.

The tumor suppressor gene p53 induces cell cycle arrest and promotes apoptosis thereby preventing transformation of cells. Inactivation of the tumor suppressor gene p53 is the most common genetic defect in cancer affecting more than half of all human tumors. The p53 protein is stabilized in response to genotoxic stress, metabolic changes, and other potentially dangerous events which can result in transformation of cells. p53 executes its function mainly as a transcription factor inducing genes responsible for cell cycle regulation, like p21 or genes promoting apoptosis like the bc1-2 antagonist bax. p53 function is believed to be under complex control through several pathways. For example mdm2, a gene induced by p53, is directly involved in inhibition and degradation of p53 creating a regulatory feedback loop which is further modulated by p14arf.

A p73 gene was discovered as the first homologue of the tumor suppressor p53. Kaghad et al., Cell 90:809-819 (1997). The two proteins work as transcription factors and share significant structural similarity, being formed by three highly homologous domains, the N-terminal transactivation (TA) domain, a DNA-binding domain (DBD), and an oligomerization domain (OD). Due to the homology of p73 to p53, especially in the DNA binding domain, p73 is believed to bind to p53 responsive elements to activate the same genes involved in cell cycle regulation and apoptosis as does p53, although to a different extent. p73 maps to human chromosome 1p36, a region that is deleted in a variety of human cancers including colon cancer, breast cancer, and neuroblastoma.

However, despite the remarkable structural similarities of the genes, knockout mice for p53, p63 (a related homologue of p53), and p73 display no obvious overlapping features. Yang et al., Nat. Rev. Mol. Cell Biol. 1:199-207 (2000). In particular, p73−/− mice show abnormalities in fluid dynamics of the nervous and respiratory systems, defective neurogenesis, reproductive and social behaviour indicating a role for p73 in the development of the nervous and immune systems. Yang et al., Nature 404:99-103 (2000).

Nonetheless, several lines of evidence suggest the involvement of p73 in cancer. Exogenous expression of p73, similarly to p53, induces irreversible cell cycle and growth arrest and promotes apoptosis. See, e.g., De Laurenzi et al., J. Biol. Chem. 275:15226-15231 (2000); Jost et al., Nature 389:191-194 (1997); Ueda et al., Oncogene 18:4993-4998 (1999). Although p73 is not transcriptionally regulated by DNA damage, p73 protein is stabilized by phosphorylation through the c-Ab1 tyrosine kinase pathway in response to DNA damage. Gong et al., Nature 39:806-809 (1999); Agami et al., Nature 399:809-813 (1999); Yuan et al., Nature 399:814-817 (1999). These data support the existence of a rescue pathway mediated by MLH/c-Ab1/p73 which triggers apoptosis following DNA damage, independent from p53. However, unlike p53, p73 mutations are extremely rare in human cancers, see, e.g., Levrero et al., Cell Death Differ. 6:1146-1153 (1999), and p73 knockout mice do not develop spontaneous tumors. Yang et al., Nature 404:99-103 (2000). Still, several reports suggest that an altered expression of this gene rather than its mutation might be involved in cancer. See, e.g., id.; Kaelin, Oncogene 18:7701-7705 (1999); De Laurenzi et al., J. Exp. Med. 188:1763-1768 (1998).

At variance with p53, whose transcription is believed to generate a single species of mRNA, expression of p73 generates several alternatively spliced transcripts differing at the C-terminus that differ in vitro in their potential to activate p53-responsive genes, such as p21^{Waf1/Cip1} and bax, thus showing different functional properties. As shown in FIG. 1, there are six known C-terminal variant isoforms of p73: α (full length), β (missing exon 13), γ (missing exon 11), δ (missing exons 11-13), ε (missing exons 11 and 13) and ζ (missing exons 11 and 12), all of which contain an N-terminal TA domain homologous to the TA domain of p53. These isoforms are referred to herein collectively as “TA p73”. Similar splice variants occur in p63.

The DBD of TA p73 is capable of activating the promoters of p53 responsive genes such as bax, mdm2, p21, etc. in vitro. TA p73α and TA p73β transcripts have been detected in all human tissues, and endogenous TA p73α protein has been detected in cell extracts of HT-29, IMR-32, and SK-N-SH cells. It has been reported that overexpressed beta, gamma and delta splice variants of p63 and TA p73 mimic some functions of p53, such as oligomerization, activation of promoters containing p53 binding sites, and apoptosis induction.

Alpha splice variants of p63 and p73, including TA p63α, ΔN p63α, and TA p73α, show dramatically reduced p53-like function. These alpha splice variants possess a C-terminal Sterile Alpha Motif (SAM) domain, as shown in FIG. 2. SAM domains are found in many proteins involved in cell signaling, including polyhomeotic proteins, diacylglycerol kinases, liprins, serine/threonine kinases, adapter proteins, the Eph family of tyrosine kinase receptors, and the ETS family of transcription factors. SAM domains associate with other SAM domains to form homo-oligomers and hetero-oligomers, and also associate with other proteins such as AF6. Abnormal SAM-mediated oligomerization is causally linked with human leukemias.

In mice, two variants with a different N-terminus (ΔN p73 variants) were found and are thought to derive from the usage of two different promoters: one located upstream of exon 1 and one located in intron 3. Yang et al., Nature 404:99-103 (2000). More particularly, it was reported in mice that murine ΔN p73 variants inhibit the full length p73 variant (TA p73) yet do not activate transcription from p53-responsive promoters. Id. This dominant negative effect is thought to be mediated either by competition through its DBD and/or by hetero-oligomerization and sequestration through its oligomerization domain (“OD”). Further, ectopic expression of these variants in mice was reported to inhibit p53-induced apoptosis and to protect p73−/− neurons from death induced by NGF withdrawal. Pozniak et al., Science 289:304-306 (2000).

Thus, it is desirable to identify agents which can modify the activity of p53-related proteins so as to modulate apoptosis, cell proliferation, and differentiation for therapeutic or prophylactic benefit. Until the present invention, no human ΔN p73 variants had been cloned or characterized.

### SUMMARY OF THE INVENTION

The present invention includes and provides an isolated nucleic acid molecule comprising a nucleic acid sequence selected from the group consisting of SEQ ID NOs: 1, 3, and 5, and a nucleic acid sequence complementary to a nucleic acid sequence selected from the group consisting of SEQ ID NOs: 1, 3, and 5.

The present invention also provides and includes an isolated nucleic acid molecule comprising a first nucleic acid sequence selected from the group consisting of SEQ ID NO: 8 and a nucleic acid sequence complementary to SEQ ID NO: 8, wherein said first nucleic acid molecule does not include at least one of a second nucleic acid sequence selected from the group consisting of exon 1, exon 2, and exon 3 of the nucleic acid sequence encoding TA p73.

The present invention also provides and includes an isolated nucleic acid molecule comprising a nucleic acid sequence with an identity of at least 90% to a nucleic acid sequence selected from the group consisting of SEQ ID NOs: 1, 3, 5 and complements thereof.

The present invention also provides and includes an isolated nucleic acid molecule comprising a first nucleic acid sequence with an identity of at least 90% to a second nucleic acid sequence selected from the group consisting of SEQ ID NO: 8 and complement thereof, wherein said first nucleic acid molecule does not include at least one of a third nucleic acid sequence selected from the group consisting of exon 1, exon 2, and exon 3 of the nucleic acid sequence encoding TA p73.

The present invention also provides and includes an isolated nucleic acid molecule encoding an amino acid sequence selected from the group consisting of SEQ ID NOs: 2, 4, and 6.

The present invention also provides and includes an isolated nucleic acid molecule encoding an amino acid sequence of SEQ ID NO: 9, wherein said nucleic acid molecule does not include at least one of a nucleic acid sequence selected from the group consisting of exon 1, exon 2, and exon 3 of the nucleic acid sequence encoding TA p73.

The present invention also provides and includes an isolated nucleic acid molecule comprising at least 10 but not more than 1500 consecutive nucleotides of a complement of a nucleic acid sequence selected from the group consisting of SEQ ID NOS: 1, 3, and 5.

The present invention also provides and includes an isolated nucleic acid molecule comprising at least 10 but not more than 272 consecutive nucleotides of SEQ ID NO: 8.

The present invention also provides and includes a nucleic acid probe comprising a first nucleic acid sequence of SEQ ID NO: 32, but not at least one of a second nucleic acid sequence selected from the group consisting of exon 1, exon 2, or exon 3 of the nucleic acid sequence encoding TA p73.

The present invention also provides and includes a vector having a nucleic acid molecule comprising a nucleic acid sequence selected from the group consisting of SEQ ID NOs: 1, 3, and 5 and a nucleic acid sequence complementary to a nucleic acid sequence selected from the group consisting of SEQ ID NOs: 1, 3, and 5.

The present invention also provides and includes a vector having a nucleic acid molecule comprising a nucleic acid sequence of at least 10 consecutive nucleotides of the complement of SEQ ID NO: 8.

The present invention also provides and includes an isolated nucleic acid molecule comprising a promoter which comprises SEQ ID NO: 7 operably linked to a heterologous nucleic acid sequence.

The present invention also provides and includes an isolated polypeptide comprising an amino acid sequence of SEQ ID NO: 9.

The present invention also provides and includes an antibody that selectively binds to a polypeptide comprising SEQ ID NO: 9.

The present invention also provides and includes an antibody that selectively binds to a polypeptide consisting of SEQ ID NO: 9.

The present invention also provides and includes an method for at least partially inhibiting apoptosis in a cell comprising: providing an expression vector comprising a nucleic acid sequence encoding a polypeptide selected from the group consisting of SEQ ID NOs: 2, 4, and 6, operably linked to an expression control sequence; introducing the expression vector into the cell; and maintaining the cell under conditions permitting expression of the encoded polypeptide in the cell.

The present invention also provides and includes a method for at least partially inhibiting the expression of at least one of a p53 molecule, a p63 molecule, and a TA p73 molecule in a cell comprising: providing an expression vector comprising a nucleic acid sequence encoding a polypeptide selected from the group consisting of SEQ ID NOs: 2, 4, and 6, operably linked to an expression control sequence; introducing the expression vector into the cell; and maintaining the cell under conditions permitting expression of the encoded polypeptide in the cell.

The present invention also provides and includes a method for at least partially inhibiting the production of a ΔN p73 polypeptide in a cell comprising: providing an isolated nucleic acid molecule comprising at least 10 consecutive nucleotides of the complement of SEQ ID NO: 8; introducing the nucleic acid molecule into the cell; and maintaining the cell under conditions permitting the binding of the nucleic acid sequence to ΔN p73 mRNA.

The present invention also provides and includes a method for determining a presence or absence of ΔN p73 molecule in a sample comprising: obtaining the sample; and selectively detecting the presence or absence of a ΔN p73 molecule, wherein the ΔN p73 molecule is selected from the group consisting of a ΔN p73 mRNA and a ΔN p73 polypeptide.

The present invention also provides and includes a method for determining a level of ΔN p73 molecule in a sample comprising: obtaining the sample; and selectively detecting the level of a ΔN p73 molecule, wherein the ΔN p73 molecule is selected from the group consisting of a ΔN p73 mRNA and a ΔN p73 polypeptide.

The present invention also provides and includes a method for determining the TA p73/ΔN p73 ratio in a sample comprising: obtaining the sample; selectively detecting the level of a TA p73 molecule and a ΔN p73 molecule, wherein the TA p73 molecule is selected from the group consisting of a TA p73 mRNA and a TA p73 polypeptide, and the ΔN p73 molecule is selected from the group consisting of a ΔN p73 mRNA and a ΔN p73 polypeptide; and determining a TA p73/ΔN p73 ratio based on the detected levels of TA p73 and ΔN p73.

The present invention also provides and includes a method for predicting tumor resistance to treatments involving p53, p63, and/or p73-induced apoptosis comprising: obtaining a sample tissue or cell; detecting the amount of ΔN p73 molecule or a TA p73/ΔN p73 ratio in said sample; and comparing said amount to a base-line amount in cell types of known resistance to p53, p63, and/or p73-induced apoptosis.

The present invention also provides and includes a method for predicting tumor resistance to treatments involving chemotherapy agents or radiotherapy agents comprising: obtaining a sample tissue or cell; detecting an amount of ΔN p73 molecule or a TA p73/ΔN p73 ratio in the sample; and comparing the amount to a base-line amount in cell types of known resistance to chemotherapy or radiotherapy agents.

The present invention also provides and includes a method for identifying ΔN p73, modulating compounds comprising: obtaining a sample tissue or cell which expresses a ΔN p73 molecule; exposing the sample to a putative modulating compound; and monitoring the level or activity of a ΔN p73 molecule.

The present invention also provides and includes a diagnostic assay for predicting a predisposition to cancer comprising: detecting the amount of ΔN p73 molecule or a TA p73/ΔN p73 ratio in a tissue or cell of interest; and comparing the amount to a base-line amount.

The present invention also provides and includes a method for identifying compounds which modulate the expression of ΔN p73 comprising: obtaining a tissue or cell sample which expresses the SEQ ID NO: 7 operably linked to a reporter gene; exposing the sample to a putative modulating compound; and monitoring the activity or expression of ΔN p73.

### BRIEF DESCRIPTION OF THE DRAWINGS

FIG. 1 sets forth a graphical representation of the splicing patterns, exons, and domains of the various isoforms of TA p73.

FIG. 2 sets forth the domain structure of several p53 family members, including TA p73.

FIG. 3 sets forth a schematic representation of the 5′-end of the human p73 gene depicting the N-termini of TA and ΔN splice variants.

FIG. 4 sets forth an alignment between the human ΔN p73 protein N-terminus (SEQ ID NO: 12), the mouse ΔN p73 protein N-terminus (SEQ ID NO: 13), and their consensus sequence (SEQ ID NO: 14).

FIG. 5 sets forth a Western blot analysis of overexpressed p73.

FIG. 6 sets forth a Western blot analysis of endogenous p73.

FIG. 7 sets forth a sequence for the promoter and 5′ region of ΔN p73 mRNA.

FIG. 8 sets forth a histogram depicting results of a luciferase reporter assay of ΔN p73 expression levels.

FIG. 9 sets forth results of a Real-Time PCR experiment.

FIG. 10 sets forth a histogram depicting the ratio of TA p73 and ΔN p73 in a variety of human tissues and cell lines.

FIGS. 11 through 13 set forth histograms depicting the experimentally measured anti-apoptotic capabilities of ΔN p73.

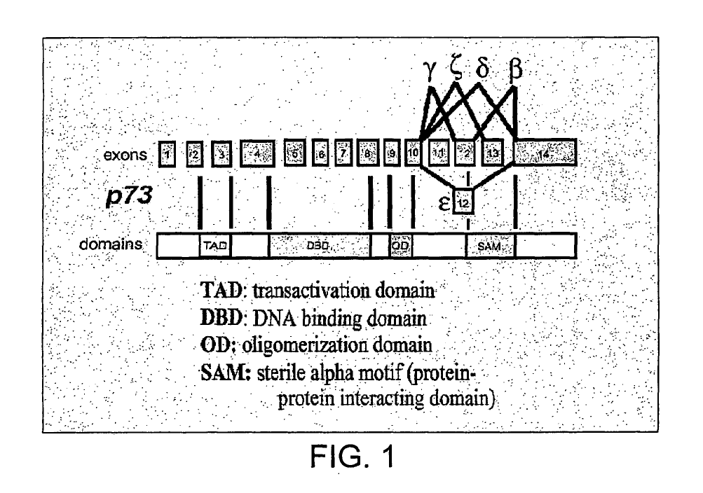

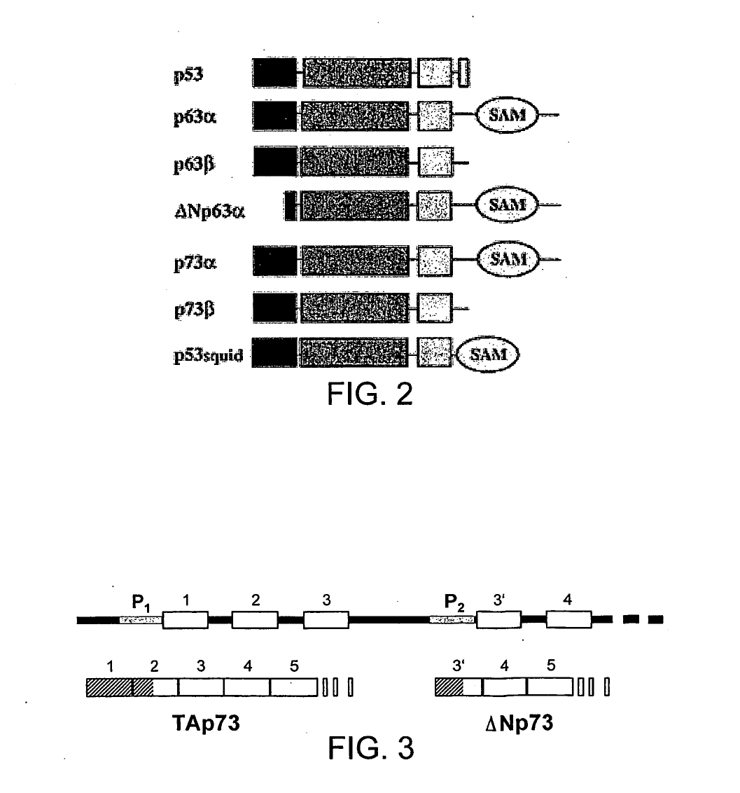

### DESCRIPTION OF THE NUCLEIC AND AMINO ACID SEQUENCES

SEQ ID NO: 1 is a Homo sapiens nucleotide sequence of ΔN p73α.

SEQ ID NO: 2 is a Homo sapiens amino acid sequence of ΔN p73α.

SEQ ID NO: 3 is a Homo sapiens nucleotide sequence of ΔN p73β.

SEQ ID NO: 4 is a Homo sapiens amino acid sequence of ΔN p73β.

SEQ ID NO: 5 is a Homo sapiens nucleotide sequence of ΔN p73γ.

SEQ ID NO: 6 is a Homo sapiens amino acid sequence of ΔN p73γ.

SEQ ID NO: 7 is a Homo sapiens nucleotide sequence of ΔN p73 promoter.

SEQ ID NO: 8 is a Homo sapiens nucleotide sequence of ΔN p73 exon 3′.

SEQ ID NO: 9 is a Homo sapiens amino acid sequence of ΔN p73 exon 3′.

SEQ ID NO: 10 is a Homo sapiens nucleotide sequence of a TA p73 gene.

SEQ ID NO: 11 is a Homo sapiens amino acid sequence of a TA p73 protein.

SEQ ID NO: 12 is a Homo sapiens amino acid sequence of a ΔN p73 N-terminal region.

SEQ ID NO: 13 is a Mus musculus amino acid sequence of a ΔN p73 N-terminal region.

SEQ ID NO: 14 is a consensus sequence of Homo sapiens and Mus musculus ΔN p73 N-terminal regions.

SEQ ID NO: 15 is a Homo sapiens nucleotide sequence of a ΔN p73 5′ region.

SEQ ID NO: 16 is a Homo sapiens amino acid sequence of a ΔN p73 N-terminal region.

SEQ ID NOs: 17 through 29 and 33 through 39 are primer nucleotide sequences.

SEQ ID NOs: 30 through 32 are probe nucleotide sequences.

### DEFINITIONS

The following definitions are provided as an aid to understanding the detailed description of the present invention.

The abbreviation “EP” refers to patent applications and patents published by the European Patent Office, and the term “WO” refers to patent applications published by the World Intellectual Property Organization. “PNAS” refers to Proc. Natl. Acad. Sci. (U.S.A.).

“Amino acid” and “amino acids” refer to all naturally occurring L-amino acids. This definition is meant to include norleucine, norvaline, ornithine, homocysteine, and homoserine.

“Chromosome walking” means a process of extending a genetic map by successive hybridization steps.

The phrases “coding sequence,” “structural sequence,” and “structural nucleic acid sequence” refer to a physical structure comprising an orderly arrangement of nucleic acids. The coding sequence, structural sequence, and structural nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

A nucleic acid molecule is said to be the “complement” of another nucleic acid molecule if they exhibit complete complementarity, i.e., every nucleotide of one of the molecules is complementary to a nucleotide of the other. Two molecules are “minimally complementary” if they can hybridize to one another with sufficient stability to remain annealed to one another under at least conventional “low-stringency” conditions. Similarly, the molecules are “complementary” if they can hybridize to one another with sufficient stability to remain annealed to one another under conventional “high-stringency” conditions. Conventional stringency conditions are described by Sambrook et al., Molecular Cloning: A Laboratory Manual, Second Edition, Cold Spring Harbor Laboratory Press, Cold Spring Harbor, N.Y. (1989); Haymes et al., Nucleic Acid Hybridization, A Practical Approach, IRL Press, Washington, D.C. (1985).

The phrases “DNA sequence,” “nucleic acid sequence,” and “nucleic acid molecule” refer to a physical structure comprising an orderly arrangement of nucleic acids. The DNA sequence or nucleic acid sequence may be contained within a larger nucleic acid molecule, vector, or the like. In addition, the orderly arrangement of nucleic acids in these sequences may be depicted in the form of a sequence listing, figure, table, electronic medium, or the like. “Nucleic acid” refers to deoxyribonucleic acid (DNA) and ribonucleic acid (RNA).

“Exogenous genetic material” is any genetic material, whether naturally occurring or otherwise, from any source that is capable of being inserted into any organism.

The term “expression” refers to the transcription of a gene to produce the corresponding mRNA and translation of this mRNA to produce the corresponding gene product (i.e., a peptide, polypeptide, or protein). The term “expression of antisense RNA” refers to the transcription of a DNA to produce a first RNA molecule capable of hybridizing to a second RNA molecule.

“Homology” refers to the level of similarity between two or more nucleic acid or amino acid sequences in terms of percent of positional identity (i.e., sequence similarity or identity).

As used herein, a “homolog protein” molecule or fragment thereof is a counterpart protein molecule or fragment thereof in a second species (e.g., human ΔN p73 is a homolog of mouse ΔN p73). A homolog can also be generated by molecular evolution or DNA shuffling techniques, so that the molecule retains at least one functional or structure characteristic of the original protein (see, e.g., U.S. Pat. No. 5,811,238).

The phrase “heterologous” refers to the relationship between two or more nucleic acid or protein sequences that are derived from different sources. For example, a promoter is heterologous with respect to a coding sequence if such a combination is not normally found in nature. In addition, a particular sequence may be “heterologous” with respect to a cell or organism into which it is inserted (i.e. does not naturally occur in that particular cell or organism).

“Hybridization” refers to the ability of a strand of nucleic acid to join with a complementary strand via base pairing. Hybridization occurs when complementary nucleic acid sequences in the two nucleic acid strands contact one another under appropriate conditions.

“Isolated” refers to a molecule separated from substantially all other molecules normally associated with it in its native state. More preferably an isolated molecule is the predominant species present in a preparation. A isolated molecule may be greater than 60% free, preferably 75% free, more preferably 90% free, and most preferably 95% free from the other molecules (exclusive of solvent) present in the natural mixture. The term “isolated” is not intended to encompass molecules present in their native state.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence.

“Polyadenylation signal” or “polyA signal” refers to a nucleic acid sequence located 3′ to a coding region that promotes the addition of adenylate nucleotides to the 3′ end of the mRNA transcribed from the coding region.

The term “promoter” or “promoter region” refers to a nucleic acid sequence, usually found upstream (5′) to a coding sequence, that is capable of directing transcription of a nucleic acid sequence into mRNA. The promoter or promoter region typically provide a recognition site for RNA polymerase and the other factors necessary for proper initiation of transcription. As contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed.

The term “protein” “polypeptide” or “peptide molecule” includes any molecule that comprises five or more amino acids. Typically, peptide molecules are shorter than 50 amino acids. It is well known in the art that proteins may undergo modification, including post-translational modifications, such as, but not limited to, disulfide bond formation, glycosylation, phosphorylation, or oligomerization. Thus, as used herein, the term “protein”, “polypeptide” or “peptide molecule” includes any protein that is modified by any biological or non-biological process.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein.

“Recombinant vector” refers to any agent such as a plasmid, cosmid, virus, autonomously replicating sequence, phage, or linear single-stranded, circular single-stranded, linear double-stranded, or circular double-stranded DNA or RNA nucleotide sequence. The recombinant vector may be derived from any source and is capable of genomic integration or autonomous replication.

“Regulatory sequence” refers to a nucleotide sequence located upstream (5′), within, or downstream (3′) to a coding sequence. Transcription and expression of the coding sequence is typically impacted by the presence or absence of the regulatory sequence.

An antibody or peptide is said to “specifically bind” to a protein, polypeptide, or peptide molecule of the invention if such binding is not competitively inhibited by the presence of non-related molecules.

“Substantially homologous” refers to two sequences which are at least 90% identical in sequence, as measured by the BestFit program described herein (Version 10; Genetics Computer Group, Inc., University of Wisconsin Biotechnology Center, Madison, Wis.), using default parameters.

“Transcription” refers to the process of producing an RNA copy from a DNA template.

“Transfection” refers to the introduction of exogenous DNA into a recipient host.

“Transformation” refers a process by which the genetic material carried by a recipient host is altered by stable incorporation of exogenous DNA. The term “host” refers to cells or organisms.

“Transgenic” refers to organisms into which exogenous nucleic acid sequences are integrated.

“Vector” refers to a plasmid, cosmid, bacteriophage, or virus that carries exogenous DNA into a host organism.

### DETAILED DESCRIPTION OF THE PREFERRED EMBODIMENTS

One skilled in the art may refer to general reference texts for detailed descriptions of known techniques discussed herein or equivalent techniques. These texts include Ausubel et al., Current Protocols in Molecular Biology, John Wiley and Sons, Inc. (1995); Sambrook et al., Molecular Cloning, A Laboratory Manual (2d ed.), Cold Spring Harbor Press, Cold Spring Harbor, N.Y. (1989); Birren et al., Genome Analysis: A Laboratory Manual, volumes 1 through 4, Cold Spring Harbor Press, Cold Spring Harbor, N.Y. (1997-1999). These texts can, of course, also be referred to in making or using an aspect of the invention.

A. Human ΔN p73

In the present invention, a human ΔN p73 gene has been identified. The transcription start site of the ΔN forms was determined, and a 2 kb fragment of genomic sequence upstream of it was cloned. This fragment was found to contain a second p73 gene promoter responsible for the transcription of ΔN isoforms of p73, and is sufficient to drive expression in the cell lines that express ΔN p73. A ΔN p73 promoter was isolated and sequenced (SEQ ID NO: 7).

A ΔN p73 gene does not include exons 1, 2 or 3 of the TA p73 gene, but does include an additional exon (exon 3′) (nucleic acid SEQ ID NO: 8, amino acid SEQ ID NO: 9) at its N-terminus which is located within intron 3 of the TA p73 gene. The reverse complement of a TA p73 gene is set forth in SEQ ID NO: 10. The reverse complements of exons 1 through 3 are shown in SEQ ID NO: 10 (the reverse complement of exon 1 spans bases 86801 to 86865, of exon 2 spans bases 86051 to 86171, and of exon 3 spans 61440 to 61682).

FIG. 3 depicts the interrelation of the TA p73 and ΔN p73 genes. ΔN p73, like TA p73, has multiple isoforms with differing C-termini due to alternative splicing: ΔN p73α (nucleic acid SEQ ID NO: 1, amino acid SEQ ID NO: 2), ΔN p73β (nucleic acid SEQ ID NO: 3, amino acid SEQ ID NO: 4), and ΔN p73γ (nucleic acid SEQ ID NO: 5, amino acid SEQ ID NO: 6). These ΔN p73 proteins lack the N-terminal TA domain found in TA p73, but their C-termini are homologous to the corresponding splice variants of the TA p73 proteins.

ΔN p73 proteins act as dominant negatives on tumor suppressors p53, p63, and TA p73, at least in part by blocking or inhibiting their ability to activate the p21 promoter, thereby blocking their ability to induce apoptosis. ΔN p73 also acts to block the p53 and TA p73-induced expression of PUMA, a recently discovered p53/TA p73-upregulated modulator of apoptosis. The ability to down-regulate ΔN p73 and thereby promote the apoptotic activity of, e.g., p53 and TA p73, is advantageous in cancer therapy, in controlling hyperplasia such as benign prostatic hypertrophy (BPH) and eliminating self reactive clones in autoimmunity by favoring death effector molecules. Up-regulating ΔN p73, and thereby repressing the apoptotic activity of, e.g., p53 and TA p73 would be beneficial in the treatment and diagnosis of immunodeficiency diseases, including AIDS, senescence, neurodegenerative disease, ischemic cell death, wound-healing, and the like. As used herein, ΔN p73 activity refers to the activity of a ΔN p73 polypeptide to block the ability of p53 to activate the p21 promoter.

The differential expression of transcriptionally active (TA) and inactive (ΔN) p73 variants may in part determine the function of p73 within a particular cell type or in a particular phase of cell cycle or differentiation stage. The balance between the two forms is believed to be finely regulated at the transcriptional level via alternative promoter usage. As such, alteration of the relative amounts of the two isoforms is believed to be extremely important for its function (e.g., its involvement in development and in carcinogenesis). Since the two forms have distinct (if not opposite) functions, it is important to identify them in humans, to clarify their normal expression pattern and functions and to clarify their differential regulation.

Human ΔN p73 isoforms are expressed in a number of different normal adult and fetal tissues and TA p73 isoforms are expressed 10 to a 100 fold more than ΔN p73 isoforms. In addition, most of the tumor cell lines tested show an altered TA p73/ΔN p73 ratio. Human ΔN-p73 is able to block the ability of either TA p73 or p53 to transactivate the p21 promoter and their ability to induce apoptosis.

The present invention provides a number of agents, for example, nucleic acid molecules encoding ΔN p73, ΔN p73 promoters and provides uses of such agents. The agents of the invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic and thus involve the capacity of the agent to mediate a chemical reaction or response. The agents will preferably be isolated. The agents of the invention may also be recombinant.

It is understood that any of the agents of the invention can be isolated and/or be biologically active and/or recombinant. It is also understood that the agents of the invention may be labeled with reagents that facilitate detection of the agent, e.g., fluorescent labels, chemical labels, modified bases, and the like. The agents may be used as diagnostic or therapeutic compositions useful in the detection, prevention, and treatment of cancer, autoimmune diseases, lymphoproliferative disorders, atherosclerosis, AIDS, immunodeficiency diseases, ischemic injuries, neurodegenerative diseases, osteoporosis, myelodysplastic syndromes, toxin-induced diseases, and viral infections.

B. Nucleic Acid Molecules

Agents of the invention include nucleic acid molecules. In another preferred aspect of the present invention the nucleic acid molecule comprises a nucleic acid sequence which encodes a human ΔN p73 protein or fragment thereof. Examples of ΔN p73 proteins are those proteins having an amino acid sequence selected from the group consisting of SEQ ID NO: 2, 4, and 6.

In another aspect of the present invention the nucleic acid molecule is a ΔN p73 promoter. An example of a ΔN p73 promoter is the nucleic acid sequence set forth in SEQ ID NO: 7. In a preferred aspect of the present invention, the ΔN p73 promoter comprises a fragment of SEQ ID) NO: 7 that itself comprises at least one, preferably two ATG initiation codons and includes preferably at least between 100 and 500 consecutive nucleotides, more preferable at between least 200 and 1000 consecutive nucleotides, and most preferably between 500 and 5,000 consecutive nucleotides of SEQ ID NO: 7. In a particularly preferred embodiment, the ΔN p73 promoter fragment comprises at least 150 bases upstream of the TATA-box. More preferably, the ΔN p73 promoter fragment is at least 349, 503, or 1143 bp in length.

In another preferred aspect of the present invention the nucleic acid molecule comprises a nucleic acid sequence that is selected from: (1) any of SEQ ID NOs: 1, 3, 5, 8, complements thereof, or fragments of these sequences; (2) the group consisting of SEQ ID NOs: 1, 3, 5, 8, complements thereof, and fragments of these sequences; (3) the group consisting of SEQ ID NOs: 1, 3, 5, complements thereof, and fragments of these sequences; (4) and the group consisting of SEQ ID NOs: 1, 3, and 5, complements thereof, and fragments of these sequences.

In a further aspect of the present invention the nucleic acid molecule comprises a nucleic acid sequence encoding an amino acid sequence selected from: (1) any of SEQ ID NOs: 2, 4, 6, and 9; (2) the group consisting of SEQ ID NO: 2, 4, 6, 9, and fragments of these sequences; and (3) the group consisting of SEQ ID NO: 2, 4, 6, and fragments of these sequences.

It is understood that in a further aspect of the nucleic acid sequences of the present invention can encode a protein which differs from any of the proteins in that amino acid have been deleted, substituted or added without altering the function. For example, it is understood that codons capable of coding for such conservative amino acid substitutions are known in the art.

In one embodiment the nucleic acid molecule is a DNA molecule. In another embodiment the nucleic acid molecule is an RNA molecule, more preferably an mRNA molecule. In a further embodiment the nucleic acid molecule is a double stranded molecule. In another further embodiment the nucleic acid molecule is a single stranded molecule.

In an embodiment, the nucleic acid molecule does not include a nucleic acid sequence of at least one of the nucleic acid sequences selected from the group consisting of exon 1, exon 2, and exon 3 of a nucleic acid sequence encoding TA p73. The reverse complement of a TA p73 gene is set forth in SEQ ID NO: 10. The reverse complements of exons 1 through 3 of a nucleic acid sequence encoding TA p73 are shown in SEQ ID NO: 10 (the reverse complement of exon 1 spans bases 86801 to 86865, of exon 2 spans bases 86051 to 86171, and of exon 3 spans 61440 to 61682).

In a preferred embodiment, such a nucleic acid molecule comprises SEQ ID NO: 8 or complement thereof.

The present invention provides nucleic acid molecules that hybridize to the above-described nucleic acid molecules. Nucleic acid hybridization is a technique well known to those of skill in the art of DNA manipulation. The hybridization properties of a given pair of nucleic acids is an indication of their similarity or identity.

The nucleic acid molecules preferably hybridize, under low, moderate, or high stringency conditions, with a nucleic acid sequence selected from: (1) any of SEQ ID NOs: 1, 3, 5, 8, and complements thereof; (2) the group consisting of SEQ ID NOs: 1, 3, 5, 8, and complements thereof; (3) the group consisting of SEQ ID NOs: 1, 3, 5, and complements thereof; (4) and the group consisting of SEQ ID NOs: 1, 3, 5, and complements thereof. Fragments of these sequences are also contemplated.

In another aspect, the nucleic acid molecules preferably hybridize, under low, moderate, or high stringency conditions, with a nucleic acid sequence selected from the group consisting of SEQ ID NO: 7 and its complement.

The hybridization conditions typically involve nucleic acid hybridization in about 0.1× to about 10×SSC (diluted from a 20×SSC stock solution containing 3 M sodium chloride and 0.3 M sodium citrate, pH 7.0 in distilled water), about 2.5× to about 5× Denhardt's solution (diluted from a 50× stock solution containing 1% (w/v) bovine serum albumin, 1% (w/v) ficoll, and 1% (w/v) polyvinylpyrrolidone in distilled water), about 10 mg/mL to about 100 mg/mL fish sperm DNA, and about 0.02% (w/v) to about 0.1% (w/v) SDS, with an incubation at about 20° C. to about 70° C. for several hours to overnight. The stringency conditions are preferably provided by 6×SSC, 5× Denhardt's solution, 100 mg/mL fish sperm DNA, and 0.1% (w/v) SDS, with an incubation at 55° C. for several hours.

The hybridization is generally followed by several wash steps. The wash compositions generally comprise 0.1× to about 10×SSC, and 0.01% (w/v) to about 0.5% (w/v) SDS with a 15 minute incubation at about 20° C. to about 70° C. Preferably, the nucleic acid segments remain hybridized after washing at least one time in 0.1 ×SSC at 65° C. For example, the salt concentration in the wash step can be selected from a low stringency of about 2.0×SSC at 50° C. to a high stringency of about 0.2×SSC at 65° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Low stringency conditions may be used to select nucleic acid sequences with lower sequence identities to a target nucleic acid sequence. One may wish to employ conditions such as about 6.0×SSC to about 10×SSC, at temperatures ranging from about 20° C. to about 55° C., and preferably a nucleic acid molecule will hybridize to one or more of the above-described nucleic acid molecules under low stringency conditions of about 6.0×SSC and about 45° C. In a preferred embodiment, a nucleic acid molecule will hybridize to one or more of the above-described nucleic acid molecules under moderately stringent conditions, for example at about 2.0×SSC and about 65° C. In a particularly preferred embodiment, a nucleic acid molecule of the present invention will hybridize to one or more of the above-described nucleic acid molecules under high stringency conditions such as 0.2×SSC and about 65° C.

In an alternative embodiment, the nucleic acid molecule comprises a nucleic acid sequence that is greater than 85% identical, and more preferably greater than 86, 87, 88, 89, 90, 91, 92, 93, 94, 95, 96, 97, 98, or 99% identical to a nucleic acid sequence selected from the group consisting of SEQ ID NO: 1, 3, 5, 7, 8, complements thereof, and fragments of any of these sequences.

The percent identity is preferably determined using the “Best Fit” or “Gap” program of the Sequence Analysis Software Package™ (Version 10; Genetics Computer Group, Inc., University of Wisconsin Biotechnology Center, Madison, Wis.). “Gap” utilizes the algorithm of Needleman and Wunsch to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman. The percent identity calculations may also be performed using the Megalign program of the LASERGENE bioinformatics computing suite (default parameters, DNASTAR Inc., Madison, Wis.). The percent identity is most preferably determined using the “Best Fit” program using default parameters.

The present invention also provides nucleic acid molecule fragments that hybridize to the above-described nucleic acid molecules and complements thereof, fragments of nucleic acid molecules that exhibit greater than 80%, 85%, 90%, 95% or 99% sequence identity with the above-described nucleic acid molecules and complements thereof, or fragments of any of these molecules.

Fragment nucleic acid molecules may consist of significant portion(s) of, or indeed most of, the nucleic acid molecules of the invention. In an embodiment, the fragments are between 3000 and 1000 consecutive nucleotides, 1800 and 150 consecutive nucleotides, 1500 and 500 consecutive nucleotides, 1300 and 250 consecutive nucleotides, 1000 and 200 consecutive nucleotides, 800 and 150 consecutive nucleotides, 500 and 100 consecutive nucleotides, 300 and 75 consecutive nucleotides, 100 and 50 consecutive nucleotides, 50 and 25 consecutive nucleotides, or 20 and 10 consecutive nucleotides long of a nucleic molecule of the present invention.

In another embodiment, the fragment comprises at least 20, 30, 40, 50, 60, 70, 80, 90, 100, 150, 200, 250, 500, or 750 consecutive nucleotides of a nucleic acid sequence of the present invention.

In another embodiment, the fragment comprises at least 12, 15, 18, 20, 25, 50, 75, 100, 125, 150, 200, 250, 300, 350, 400, 450 but not more 500, 550, 600, 650, 700, 750, 800, 1000, 1200, 1400, or 1500 consecutive nucleotides of a nucleic acid sequence selected from the group consisting of SEQ ID NO: 1, 3, 5 and complements thereof.

In a particularly preferred embodiment a fragment nucleic acid molecule is capable of selectively hybridizing to SEQ ID NO: 8.

In a particularly preferred embodiment a fragment nucleic acid molecule comprises at least 10, 12, 14, 16, 18, 20, 25, 30, 40, 50, or 100 but not more than 105, 125, 150, 200, 250, or 272 consecutive nucleotides of SEQ ID NO: 8.

In a particularly preferred embodiment a fragment nucleic acid molecule is capable of selectively hybridizing to Exon 3′ of ΔN p73 (SEQ ID NO: 8).

Any of a variety of methods may be used to obtain one or more of the above-described nucleic acid molecules. Automated nucleic acid synthesizers may be employed for this purpose. In lieu of such synthesis, the disclosed nucleic acid molecules may be used to define a pair of primers that can be used with the polymerase chain reaction to amplify and obtain any desired nucleic acid molecule or fragment.

Short nucleic acid sequences having the ability to specifically hybridize to complementary nucleic acid sequences may be produced and utilized in the present invention, e.g., as probes to identify the presence of a complementary nucleic acid sequence in a given sample. Alternatively, the short nucleic acid sequences may be used as oligonucleotide primers to amplify or mutate a complementary nucleic acid sequence using PCR technology. These primers may also facilitate the amplification of related complementary nucleic acid sequences (e.g., related sequences from other species).

Use of these probes or primers may greatly facilitate the identification of transgenic cells or organisms which contain the presently disclosed promoters and structural nucleic acid sequences. Such probes or primers may also, for example, be used to screen cDNA or genomic libraries for additional nucleic acid sequences related to or sharing homology with the presently disclosed promoters and structural nucleic acid sequences. The probes may also be PCR probes, which are nucleic acid molecules capable of initiating a polymerase activity while in a double-stranded structure with another nucleic acid.

A primer or probe is generally complementary to a portion of a nucleic acid sequence that is to be identified, amplified, or mutated and of sufficient length to form a stable and sequence-specific duplex molecule with its complement. The primer or probe preferably is about 10 to about 200 nucleotides long, more preferably is about 10 to about 100 nucleotides long, even more preferably is about 10 to about 50 nucleotides long, and most preferably is about 14 to about 30 nucleotides long.

The primer or probe may, for example without limitation, be prepared by direct chemical synthesis, by PCR (U.S. Pat. Nos. 4,683,195 and 4,683,202), or by excising the nucleic acid specific fragment from a larger nucleic acid molecule. Various methods for determining the structure of PCR probes and PCR techniques exist in the art. Computer-generated searches using programs such as Primer3 (www-genome.wi.mit.edu/cgi-bin/primer/primer3.cgi), STSPipeline (www-genome.wi.mit.edu/cgi-bin/www-STS_Pipeline), or GeneUp (Pesole et al., BioTechniques 25:112-123, 1998), for example, can be used to identify potential PCR primers.

C. Protein and Peptide Molecules

Agents of the invention include proteins, polypeptides, peptide molecules, and fragments thereof encoded by nucleic acid agents of the invention. Preferred classes of protein, polypeptide and peptide molecules include: (1) ΔN p73 protein, polypeptide and peptide molecules; (2) ΔN p73 proteins and peptide molecules derived from mammals; and (3) ΔN p73 proteins and peptide molecules derived from humans.

Other preferred proteins and polypeptides are those proteins and polypeptides having an amino acid sequence that is selected from: (1) any of SEQ ID NOs: 2, 4, 6, and 9; (2) the group consisting of SEQ ID NO: 2, 4, 6, 9, and fragments of these sequences; and (3) the group consisting of SEQ ID NO: 2, 4, 6, and fragments of these sequences.

In another preferred aspect of the present invention the protein, polypeptide or peptide molecule is encoded by a nucleic acid agent of the invention, including, but not limited to a nucleic acid sequence that is selected from: (1) any of SEQ ID NOs: 1, 3, 5, 8, complements thereof, or fragments of these sequences; (2) the group consisting of SEQ ID NOs: 1, 3, 5, and 8, complements thereof, and fragments of these sequences; (3) the group consisting of SEQ ID NOs: 1, 3, and 5, complements thereof, and fragments of these sequences; (4) and the group consisting of SEQ ID NOs: 1, 3, and 5, complements thereof, and fragments of these sequences.

Any of the nucleic acid agents of the invention may be linked with additional nucleic acid sequences to encode fusion proteins. The additional nucleic acid sequence preferably encodes at least one amino acid, peptide, or protein. Many possible fusion combinations exist. For instance, the fusion protein may provide a “tagged” epitope to facilitate detection of the fusion protein, such as GST, GFP, FLAG, or polyHIS. Such fusions preferably encode between 1 and 50 amino acids, more preferably between 5 and 30 additional amino acids, and even more preferably between 5 and 20 amino acids.

Alternatively, the fusion may provide regulatory, enzymatic, cell signaling, or intercellular transport functions. For example, a sequence encoding a signal peptide may be added to direct a fusion protein to a particular organelle within a eukaryotic cell. Such fusion partners preferably encode between 1 and 1000 additional amino acids, more preferably between 5 and 500 additional amino acids, and even more preferably between 10 and 250 amino acids.

A protein, polypeptide, or fragment thereof encoding nucleic acid molecule of the invention may also be linked to a propeptide coding region. A propeptide is an amino acid sequence found at the amino terminus of a proprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme.

The above-described protein or peptide molecules may be produced via chemical synthesis, or more preferably, by expression in a suitable bacterial or eukaryotic host. Suitable methods for expression are described by Sambrook et al., supra, or similar texts. Fusion protein or peptide molecules of the invention are preferably produced via recombinant means. These proteins and peptide molecules may be derivatized to contain carbohydrate or other moieties (such as keyhole limpet hemocyanin, etc.).

Also contemplated are protein, polypeptide and peptide agents, including fragments and fusions thereof, in which conservative, non-essential or non-relevant amino acid residues have been added, replaced or deleted. A further particularly preferred class of protein is a ΔN p73 protein in which conservative, non-essential or non-relevant amino acid residues have been added, replaced or deleted. Computerized means for designing modifications in protein structure are known in the art. See, e.g., Dahiyàt and Mayo, Science 278:82-87 (1997).

Agents of the invention include polypeptides comprising at least about a contiguous 10 amino acid region preferably comprising at least about a contiguous 20 amino acid region, even more preferably comprising at least a contiguous 25, 35, 50, 75 or 100 amino acid region of an amino acid sequence selected from the group consisting of SEQ ID NOs: 2, 4, 6, and 9. In another preferred embodiment, the proteins of the present invention include between about 10 and about 25 contiguous amino acid region, more preferably between about 20 and about 50 contiguous amino acid region, and even more preferably between about 40 and about 80, or about 60 and about 100 contiguous amino acid region of an amino acid sequence selected from the group consisting of SEQ ID NOs: 2, 4, 6, and 9.

Due to the degeneracy of the genetic code, different nucleotide codons may be used to code for a particular amino acid. A host cell often displays a preferred pattern of codon usage. Nucleic acid sequences are preferably constructed to utilize the codon usage pattern of the particular host cell. This generally enhances the expression of the nucleic acid sequence in a transformed host cell. Any of the above described nucleic acid and amino acid sequences may be modified to reflect the preferred codon usage of a host cell or organism in which they are contained. Additional variations in the nucleic acid sequences may encode proteins having equivalent or superior characteristics when compared to the proteins from which they are engineered.

It is understood that certain amino acids may be substituted for other amino acids in a protein or peptide structure (and the nucleic acid sequence that codes for it) without appreciable change or loss of its biological utility or activity. For example, amino acid substitutions may be made without appreciable loss of interactive binding capacity in the antigen-binding regions of antibodies, or binding sites on substrate molecules. The modifications may result in either conservative or non-conservative changes in the amino acid sequence. The amino acid changes may be achieved by changing the codons of the nucleic acid sequence, according to the codons given in Table 1.

TABLE 1

Codon degeneracy of amino acids

| | |
|---|---|
| One | Three |

| | | | |
|---|---|---|---|
| Amino acid | letter | letter | Codons |

| | | | | | | | | |
|---|---|---|---|---|---|---|---|---|
| Alanine | A | Ala | GCA | GCC | GCG | GCT | | |
| Cysteine | C | Cys | TGC | TGT | | | | |
| Aspartic acid | D | Asp | GAC | GAT | | | | |
| Glutamic acid | E | Glu | GAA | GAG | | | | |
| Phenylalanine | F | Phe | TTC | TTT | | | | |
| Glycine | G | Gly | GGA | GGC | GGG | GGT | | |
| Histidine | H | His | CAC | CAT | | | | |
| Isoleucine | I | Ile | ATA | ATC | ATT | | | |
| Lysine | K | Lys | AAA | AAG | | | | |
| Leucine | L | Leu | TTA | TTG | CTA | CTC | CTG | CTT |
| Methionine | M | Met | ATG | | | | | |
| Asparagine | N | Asn | AAC | AAT | | | | |
| Proline | P | Pro | CCA | CCC | CCG | CCT | | |
| Glutamine | Q | Gln | CAA | CAG | | | | |
| Arginine | R | Arg | AGA | AGG | CGA | CGC | CGG | CGT |
| Serine | S | Ser | AGC | AGT | TCA | TCC | TCG | TCT |
| Threonine | T | Thr | ACA | ACC | ACG | ACT | | |
| Valine | V | Val | GTA | GTC | GTG | GTT | | |
| Tryptophan | W | Trp | TGG | | | | | |
| Tyrosine | Y | Tyr | TAC | TAT | | | | |

It is well known in the art that one or more amino acids in a native sequence can be substituted with other amino acid(s), the charge and polarity of which are similar to that of the native amino acid, i.e., a conservative amino acid substitution, resulting in a silent change. Conservative substitutes for an amino acid within the native polypeptide sequence can be selected from other members of the class to which the amino acid belongs. Amino acids can be divided into the following four groups: (1) acidic (negatively charged) amino acids, such as aspartic acid and glutamic acid; (2) basic (positively charged) amino acids, such as arginine, histidine, and lysine; (3) neutral polar amino acids, such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine.

In a further aspect of the present invention, nucleic acid molecules of the present invention can comprise sequences which differ from those encoding a protein or fragment thereof selected from the group consisting of SEQ ID NOs: 2, 4, 6, and 9 due to the fact that the different nucleic acid sequence encodes a protein having one or more conservative amino acid changes.

In a preferred aspect, biologically functional equivalents of the proteins or fragments thereof of the present invention can have 10 or fewer conservative amino acid changes, more preferably 7 or fewer conservative amino acid changes, and most preferably 5 or fewer conservative amino acid changes. In a preferred embodiment, the protein has between 5 and 500 conservative changes, more preferably between 10 and 300 conservative changes, even more preferably between 25 and 150 conservative changes, and most preferably between 5 and 25 conservative changes or between 1 and 5 conservative changes.

Non-conservative changes include additions, deletions, and substitutions which result in an altered amino acid sequence. In a preferred embodiment, the protein has between 5 and 500 non-conservative amino acid changes, more preferably between 10 and 300 non-conservative amino acid changes, even more preferably between 25 and 150 non-conservative amino acid changes, and most preferably between 5 and 25 non-conservative amino acid changes or between 1 and 5 non-conservative changes.

In making such changes, the role of the hydropathic index of amino acids in conferring interactive biological function on a protein may be considered. See Kyte and Doolittle, J. Mol. Biol. 157:105-132 (1982). It is accepted that the relative hydropathic character of amino acids contributes to the secondary structure of the resultant protein, which in turn defines the interaction of the protein with other molecules, e.g., enzymes, substrates, receptors, DNA, antibodies, antigens, etc. It is also understood in the art that the substitution of like amino acids may be made effectively on the basis of hydrophilicity, as the greatest local average hydrophilicity of a protein is known to correlate with a biological property of the protein. See U.S. Pat. No. 4,554,101.

Each amino acid has been assigned a hydropathic index and a hydrophilic value, as shown in Table 2.

TABLE 2
Amino Acid Hydropathic Indices and Hydrophilic Values

| Amino acid | Hydropathic Index | Hydrophilic Value |
|---|---|---|
| Alanine | +1.8 | −0.5 |
| Cysteine | +2.5 | −1.0 |
| Aspartic acid | −3.5 | +3.0 ± 1 |
| Glutamic acid | −3.5 | +3.0 ± 1 |
| Phenylalanine | +2.8 | −2.5 |
| Glycine | −0.4 | 0 |
| Histidine | −3.2 | −0.5 |
| Isoleucine | +4.5 | −1.8 |
| Lysine | −3.9 | +3.0 |
| Leucine | +3.8 | −1.8 |
| Methionine | +1.9 | −1.3 |
| Asparagine | −3.5 | +0.2 |
| Proline | −1.6 | −0.5 ± 1 |
| Glutamine | −3.5 | +0.2 |
| Arginine | −4.5 | +3.0 |
| Serine | −0.8 | +0.3 |
| Threonine | −0.7 | −0.4 |
| Valine | +4.2 | −1.5 |
| Tryptophan | −0.9 | −3.4 |
| Tyrosine | −1.3 | −2.3 |

It is known in the art that certain amino acids may be substituted by other amino acids having a similar hydropathic or hydrophilic index, score or value, and still result in a protein with similar biological activity, i.e., still obtain a biologically functional protein. In making such changes, the substitution of amino acids whose hydropathic indices or hydrophilic values are within ±2 is preferred, those within ±1 are more preferred, and those within ±5 are most preferred.

As outlined above, amino acid substitutions are therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine, and isoleucine.

These amino acid changes may be effected by mutating the nucleic acid sequence coding for the protein or peptide. Mutations to a nucleic acid sequence may be introduced in either a specific or random manner, both of which are well known to those of skill in the art of molecular biology. Mutations may include deletions, insertions, truncations, substitutions, fusions, shuffling of motif sequences, and the like. A myriad of site-directed mutagenesis techniques exist, typically using oligonucleotides to introduce mutations at specific locations in a structural nucleic acid sequence. Examples include single strand rescue, unique site elimination, nick protection, and PCR. Random or non-specific mutations may be generated by chemical agents (for a general review, see Singer and Kusmierek, Ann. Rev. Biochem. 52:655-693, 1982) such as nitrosoguanidine and 2-aminopurine; or by biological methods such as passage through mutator strains (Greener et al., Mol. Biotechnol. 7:189-195, 1997).

D. Recombinant Vectors and Constructs

Exogenous genetic material may be transferred into a host cell by use of a vector or construct designed for such a purpose. Any of the nucleic acid sequences described above may be provided in a recombinant vector. The vector may be a linear or a closed circular plasmid. The vector system may be a single vector or plasmid or two or more vectors or plasmids which together contain the total DNA to be introduced into the genome of the host. Means for preparing recombinant vectors are well known in the art.

Vectors suitable for replication in mammalian cells may include viral replicons, or sequences which insure integration of the appropriate sequences encoding HCV epitopes into the host genome. For example, another vector used to express foreign DNA is vaccinia virus. Such heterologous DNA is generally inserted into a gene which is non-essential to the virus, for example, the thymidine kinase gene (tk), which also provides a selectable marker. Expression of the HCV polypeptide then occurs in cells or animals which are infected with the live recombinant vaccinia virus.

In general, plasmid vectors containing replicon and control sequences that are derived from species compatible with the host cell are used in connection with bacterial hosts. The vector ordinarily carries a replication site, as well as marking sequences that are capable of providing phenotypic selection in transformed cells. For example, E. coli is typically transformed using pBR322, which contains genes for ampicillin and tetracycline resistance and thus provides easy means for identifying transformed cells. The pBR322 plasmid, or other microbial plasmid or phage, also generally contains, or is modified to contain, promoters that can be used by the microbial organism for expression of the selectable marker genes.

A construct or vector may include a promoter, e.g., a recombinant vector typically comprises, in a 5′ to 3′ orientation: a promoter to direct the transcription of a nucleic acid sequence of interest and a nucleic acid sequence of interest. Suitable promoters include, but are not limited to, those described herein. The recombinant vector may further comprise a 3′ transcriptional terminator, a 3′ polyadenylation signal, other untranslated nucleic acid sequences, transit and targeting nucleic acid sequences, selectable markers, enhancers, and operators, as desired.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. For autonomous replication, the vector may further comprise an origin of replication enabling the vector to replicate autonomously in the host cell in question. Alternatively, the vector may be one which, when introduced into the cell, is integrated into the genome and replicated together with the chromosome(s) into which it has been integrated. This integration may be the result of homologous or non-homologous recombination.

Integration of a vector or nucleic acid into the genome by homologous recombination, regardless of the host being considered, relies on the nucleic acid sequence of the vector. Typically, the vector contains nucleic acid sequences for directing integration by homologous recombination into the genome of the host. These nucleic acid sequences enable the vector to be integrated into the host cell genome at a precise location or locations in one or more chromosomes. To increase the likelihood of integration at a precise location, there should be preferably two nucleic acid sequences that individually contain a sufficient number of nucleic acids, preferably 400 bp to 1500 bp, more preferably 800 bp to 1000 bp, which are highly homologous with the corresponding host cell target sequence. This enhances the probability of homologous recombination. These nucleic acid sequences may be any sequence that is homologous with a host cell target sequence and, furthermore, may or may not encode proteins.

Promoters

In addition to the ΔN p73 promoters described previously, other promoter sequences can be utilized in a vector or other nucleic acid molecule. In a preferred aspect, the promoter is operably linked to a nucleic acid molecule of the present invention. The promoters may be selected on the basis of the cell type into which the vector will be inserted. The promoters may also be selected on the basis of their regulatory features, e.g., enhancement of transcriptional activity, inducibility, tissue specificity, and developmental stage-specificity. Additional promoters that may be utilized are described, for example, in Bernoist and Chambon, Nature 290:304-310 (1981); Yamamoto et al., Cell 22:787-797 (1980); Wagner et al., PNAS 78:1441-1445 (1981); Brinster et al., Nature 296:39-42 (1982).

Suitable promoters for mammalian cells are also known in the art and include viral promoters, such as those from Simian Virus 40 (SV40), Rous sarcoma virus (RSV), adenovirus (ADV), cytomegalovirus (CMV), and bovine papilloma virus (BPV), as well as mammalian cell-derived promoters. Other preferred promoters include the hematopoietic stem cell-specific, e.g., CD34, glucose-6-phosphotase, interleukin-1 alpha, CD11c integrin gene, GM-CSF, interleukin-5R alpha, interleukin-2, c-fos, h-ras and DMD gene promoters. Other promoters include the herpes thymidine kinase promoter, and the regulatory sequences of the metallothionein gene.

Inducible promoters suitable for use with bacteria hosts include the β-lactamase and lactose promoter systems, the arabinose promoter system, alkaline phosphatase, a tryptophan (trp) promoter system and hybrid promoters such as the tac promoter. However, other known bacterial inducible promoters are suitable. Promoters for use in bacterial systems also generally contain a Shine-Dalgarno sequence operably linked to the DNA encoding the polypeptide of interest.

Additional Nucleic Acid Sequences of Interest

The recombinant vector may also contain one or more additional nucleic acid sequences of interest. These additional nucleic acid sequences may generally be any sequences suitable for use in a recombinant vector. Such nucleic acid sequences include, without limitation, any of the nucleic acid sequences, and modified forms thereof, described above. The additional nucleic acid sequences may also be operably linked to any of the above described promoters. The one or more additional nucleic acid sequences may each be operably linked to separate promoters. Alternatively, the additional nucleic acid sequences may be operably linked to a single promoter (i.e. a single operon).

The additional nucleic acid sequences include, without limitation, those encoding gene products which are toxic to a cell such as the diptheria A gene product.

Alternatively, the additional nucleic acid sequence may be designed to down-regulate a specific nucleic acid sequence. This is typically accomplished by operably linking the additional nucleic acid sequence, in an antisense orientation, with a promoter. One of ordinary skill in the art is familiar with such antisense technology. Any nucleic acid sequence may be negatively regulated in this manner. Preferable target nucleic acid sequences include SEQ ID NO: 8.

Selectable and Screenable Markers

A vector or construct may also include a selectable marker. Selectable markers may also be used to select for organisms or cells that contain the exogenous genetic material. Examples of such include, but are not limited to: a neo gene, which codes for kanamycin resistance and can be selected for using kanamycin, GUS, green fluorescent protein (GFP), neomycin phosphotransferase II (nptII), luciferase (LUX), or an antibiotic resistance coding sequence.

A vector or construct may also include a screenable marker. Screenable markers may be used to monitor expression. Exemplary screenable markers include: a β-glucuronidase or uidA gene (GUS) which encodes an enzyme for which various chromogenic substrates are known; a β-lactamase gene, a gene which encodes an enzyme for which various chromogenic substrates are known (e.g., PADAC, a chromogenic cephalosporin); a luciferase gene; a tyrosinase gene, which encodes an enzyme capable of oxidizing tyrosine to DOPA and dopaquinone which in turn condenses to melanin; and α-galactosidase, which will turn a chromogenic α-galactose substrate.

Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins which are detectable, (e.g., by ELISA), or small active enzymes which are detectable in extracellular solution (e.g., α-amylase, β-lactamase, phosphinothricin transferase). Other possible selectable and/or screenable marker genes will be apparent to those of skill in the art.

E. Transgenic Organisms, Transformed and Transfected Host Cells

One or more of the nucleic acid molecules or recombinant vectors of the invention may be used in transformation or transfection. For example, exogenous genetic material may be transferred into a cell or organism. In a preferred embodiment, the exogenous genetic material includes a nucleic acid molecule of the present invention, preferably a nucleic acid molecule encoding a ΔN p73 protein. In another preferred embodiment, the nucleic acid molecule has a sequence selected from the group consisting of SEQ ID NOs: 1, 3, 5, 8, complements thereof and fragments of these sequences. Other preferred exogenous genetic material are nucleic acid molecules that encode a protein or fragment thereof having an amino acid sequence selected from the group consisting of SEQ ID NOs: 2, 4, 6, 9 and fragments thereof.

The invention is also directed to transgenic or transfected organisms and transformed or transfected host cells which comprise, in a 5′ to 3′ orientation, a promoter operably linked to a heterologous nucleic acid sequence of interest. Additional nucleic acid sequences may be introduced into the organism or host cell, such as 3′ transcriptional terminators, 3′ polyadenylation signals, other untranslated nucleic acid sequences, signal or targeting sequences, selectable markers, enhancers, and operators. Preferred nucleic acid sequences of the present invention, including recombinant vectors, structural nucleic acid sequences, promoters, and other regulatory elements, are described above in parts A through D of the Detailed Description. Another embodiment of the invention is directed to a method of producing such transgenic organisms which generally comprises the steps of selecting a suitable organism, transforming the organism with a recombinant vector, and obtaining the transformed organism.

Transfer of a nucleic acid that encodes a protein can result in expression or overexpression of that protein in a transformed cell or transgenic organism. One or more of the proteins or fragments thereof encoded by nucleic acid molecules of the invention may be overexpressed in a transformed cell or transgenic organism. Such expression or overexpression may be the result of transient or stable transfer of the exogenous genetic material.

The expressed protein may be detected using methods known in the art that are specific for the particular protein or fragment. These detection methods may include the use of specific antibodies, formation of an enzyme product, or disappearance of an enzyme substrate. For example using the antibodies to the protein. The techniques of enzyme assay and immunoassay are well known to those skilled in the art.

The resulting protein may be recovered by methods known in the arts. For example, the protein may be recovered from the nutrient medium by procedures including, but not limited to, centrifugation, filtration, extraction, spray-drying, evaporation, or precipitation. The recovered protein may then be further purified by a variety of chromatographic procedures, e.g., ion exchange chromatography, gel filtration chromatography, affinity chromatography, or the like. Reverse-phase high performance liquid chromatography (RP-HPLC), optionally employing hydrophobic RP-HPLC media, e.g., silica gel, further purify the protein. Combinations of methods and means can also be employed to provide a substantially purified recombinant polypeptide or protein.

Technology for introduction of nucleic acids into cells is well known to those of skill in the art. Common methods include chemical methods, microinjection, electroporation (U.S. Pat. No. 5,384,253), particle acceleration, viral vectors, and receptor-mediated mechanisms. Fungal cells may be transformed by a process involving protoplast formation, transformation of the protoplasts and regeneration of the cell wall. The various techniques for transforming mammalian cells are also well known.

There are many methods for introducing transforming DNA segments into cells, but not all are suitable for delivering DNA to eukaryotic cells. Suitable methods are believed to include virtually any method by which DNA can be introduced into a cell, such as by direct delivery of DNA, by desiccation/inhibition-mediated DNA uptake, by electroporation, by agitation with silicon carbide fibers, by acceleration of DNA coated particles, by chemical transfection, by lipofection or liposome-mediated transfection, by calcium chloride-mediated DNA uptake, etc. In certain embodiments, acceleration methods are preferred and include, for example, microprojectile bombardment and the like.

A transformed or transfected host cell may generally be any cell which is compatible with the present invention. A transformed or transfected host plant or cell can be or derived from a cell or organism such as a mammalian cell, mammal, fish cell, fish, bird cell, bird, fungal cell, fungus, or bacterial cell. Preferred host and transformants include: fungal cells such as Aspergillus, yeasts, mammals, particularly murine, bovine and porcine, insects, bacteria, and algae. Methods to transform and transfect such cells or organisms are known in the art. See, e.g., EP 238023; Becker and Guarente, in: Abelson and Simon (eds.), Guide to Yeast Genetics and Molecular Biology, Methods Enzymol. 194: 182-187, Academic Press, Inc., New York; Bennett and LaSure (eds.), More Gene Manipulations in Fungi, Academic Press, CA, 1991; Hinnen et al., PNAS 75:1920, 1978; Ito et al., J. Bacteriology 153:163, 1983; Malardier et al., Gene 78:147-156, 1989; Yelton et al., PNAS 81:1470-1474, 1984. Mammalian cell lines available as hosts for expression are known in the art and include many immortalized cell lines available from the American Type Culture Collection (ATCC, Manassas, Va.), such as HeLa cells, Chinese hamster ovary (CHO) cells, baby hamster kidney (BHK) cells and a number of other cell lines. Non-limiting examples of suitable mammalian host cell lines include those shown below in Table 3.

TABLE 3

Mammalian Host Cell Lines

| | | |
|---|---|---|
| Host Cell | Origin | Source |

| | | |
|---|---|---|
| HepG-2 | Human Liver Hepatoblastoma | ATCC HB 8065 |
| CV-1 | African Green Monkey Kidney | ATCC CCL 70 |
| LLC-MK₂ | Rhesus Monkey Kidney | ATCC CCL 7 |
| 3T3 | Mouse Embryo Fibroblasts | ATCC CCL 92 |
| AV12-664 | Syrian Hamster | ATCC CRL 9595 |
| HeLa | Human Cervix Epitheloid | ATCC CCL 2 |
| RPMI8226 | Human Myeloma | ATCC CCL 155 |
| H4IIEC3 | Rat Hepatoma | ATCC CCL 1600 |
| C127I | Mouse Fibroblast | ATCC CCL 1616 |
| 293 | Human Embryonal Kidney | ATCC CRL 1573 |
| HS-Sultan | Human Plasma Cell | ATCC CCL 1484 |
| | Plasmocytoma | |
| BHK-21 | Baby Hamster Kidney | ATCC CCL 10 |
| CHO-K1 | Chinese Hamster Ovary | ATCC CCL 61 |

A fungal host cell may, for example, be a yeast cell, a fungi, or a filamentous fungal cell. In one embodiment, the fungal host cell is a yeast cell, and in a preferred embodiment, the yeast host cell is a cell of the species of Candida, Kluyveromyces, Saccharomyces, Schizosaccharomyces, Pichia and Yarrowia. In another embodiment, the fungal host cell is a filamentous fungal cell, and in a preferred embodiment, the filamentous fungal host cell is a cell of the species of Acremonium, Aspergillus, Fusarium, Humicola, Myceliophthora, Mucor, Neurospora, Penicillium, Thielavia, Tolypocladium and Trichoderma.

Suitable host bacteria include archaebacteria and eubacteria, especially eubacteria and most preferably Enterobacteriaceae. Examples of useful bacteria include Escherichia, Enterobacter, Azotobacter, Erwinia, Bacillus, Pseudomonas, Klebsiella, Proteus, Salmonella, Serratia, Shigella, Rhizobia, Vitreoscilla and Paracoccus. Suitable E. coli hosts include E. coli W3110 (ATCC 27325), E. coli 294 (ATCC 31446), E. coli B and E. coli X1776 (ATCC 31537) (American Type Culture Collection, Manassas, Va.). Mutant cells of any of the above-mentioned bacteria may also be employed. These hosts may be used with bacterial expression vectors such as E. coli cloning and expression vector Bluescript™ (Stratagene, La Jolla, Calif.); pIN vectors (Van Heeke and Schuster 1989), and pGEX vectors (Promega, Madison, Wis.), which may be used to express foreign polypeptides as fusion proteins with glutathione S-transferase (GST).

Preferred insect host cells are derived from Lepidopteran insects such as Spodoptera frugiperda or Trichoplusia ni. The preferred Spodoptera frugiperda cell line is the cell line Sf9 (ATCC CRL 1711). Other insect cell systems, such as the silkworm B. mori may also be used. These host cells are preferably used in combination with Baculovirus expression vectors (BEVs), which are recombinant insect viruses in which the coding sequence for a chosen foreign gene has been inserted behind a baculovirus promoter in place of the viral gene, e.g., polyhedrin (U.S. Pat. No. 4,745,051).

One aspect of the present invention relates to transgenic non-human animals having germline and/or somatic cells in which the biological activity of one or more genes are altered by a chromosomally incorporated transgene. In a preferred embodiment, the transgene encodes a ΔN p73 protein or polypeptide which acts as a dominant negative protein to antagonize at least a portion of the biological function of a TA p73. Yet another preferred transgenic animal includes a transgene encoding an antisense transcript which, when transcribed from the transgene, hybridizes with a gene or a mRNA transcript thereof, and inhibits expression of the gene, preferably the expression of ΔN p73 or TA p73.

In one embodiment, the present invention provides a desired non-human animal or an animal (including human) cell which contains a predefined, specific and desired alteration rendering the non-human animal or animal cell predisposed to cancer or apoptosis. Specifically, the invention pertains to a genetically altered non-human animal (most preferably, a mouse), or a cell (either non-human animal or human) in culture, that expresses an introduced ΔN p73 or an antisense sequence directed to ΔN p73. Animals that express an introduced ΔN p73 gene may exhibit a higher susceptibility to tumor induction or other proliferative or differentiative disorders, or disorders marked by aberrant signal transduction, e.g., from a cytokine or growth factor. By way of example, a genetically altered mouse of this type is able to serve as a model for hereditary cancers and as a test animal for carcinogen studies. Non-human animals or animal cells that express an antisense sequence directed to ΔN p73 are able to serve as an apoptosis model. The invention additionally pertains to the use of such non-human animals or animal cells.

Furthermore, it is contemplated that cells of the transgenic animals of the present invention can include other transgenes, e.g., which alter the biological activity of a second tumor suppressor gene or an oncogene. For instance, the second transgene can functionally disrupt the biological activity of a second tumor suppressor gene, such as p53, p73, DCC, p21^{cip1}, p27^{kip1}, Rb, Mad or E2F. Alternatively, the second transgene can cause overexpression or loss of regulation of an oncogene, such as ras, myc, a cdc25 phosphatase, Bc1-2, Bc1-6, a transforming growth factor, neu, int-3, polyoma virus middle T antigen, SV40 large T antigen, a papillomaviral E6 protein, a papillomaviral E7 protein, CDK4, or cyclin D1.

A preferred transgenic non-human animal of the present invention has germline and/or somatic cells in which one or more alleles of a ΔN p73 whose expression or activity is disrupted by a chromosomally incorporated transgene, wherein the transgene includes a marker sequence providing a detectable signal for identifying the presence of the transgene in cells of the transgenic animal.

Still another aspect of the present invention relates to methods for generating non-human animals and stem cells having a functionally disrupted endogenous ΔN p73 whose expression or activity is also disrupted. In a preferred embodiment, the method comprises the steps of:

(i) constructing a transgene construct including (a) a recombination region having at least a portion of the a ΔN p73 gene, which recombination region directs recombination of the transgene with the gene, and (b) a marker sequence which provides a detectable signal for identifying the presence of the transgene in a cell;

(ii) transferring the transgene into stem cells of a non-human animal;

(iii) selecting stem cells having a correctly targeted homologous recombination between the transgene and the gene;

(iv) transferring cells identified in step (iii) into a non-human blastocyst and implanting the resulting chimeric blastocyst into a non-human female; and

(v) collecting offspring harboring an endogenous gene allele having the correctly targeted recombination.

F. Inhibition of Gene Expression

In one aspect the activity or expression of a ΔN p73 molecule is reduced. In a preferred aspect, the activity or expression of a ΔN p73 molecule is reduced by greater than 50%, 60%, 70%, 80% or 90% by the introduction into a recipient cell or host of a ΔN p73 molecule of the invention. In a preferred aspect the activity or expression of a ΔN p73 molecule is reduced without reducing the activity of a TA p73 molecule.

Ribozymes

In a preferred aspect, the activity or expression of ΔN p73 molecule is reduced by designing a ribozyme specifically directed to a nucleic acid sequence found within Exon 3′ (SEQ ID NO: 8). Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting a phenotypic effect.

One commonly used ribozyme motif is the hammerhead, for which the substrate sequence requirements are minimal. Design of the hammerhead ribozyme, and the therapeutic uses of ribozymes, are disclosed in Usman et al., Current Opin. Strict. Biol. 6:527-533 (1996). Ribozymes can also be prepared and used as described in Long et al., FASEB J. 7:25 (1993); Symons, Ann. Rev. Biochem. 61:641 (1992); Perrotta et al., Biochem. 31:16-17 (1992); Ojwang et al., PNAS 89:10802-10806 (1992); and U.S. Pat. No. 5,254,678.

Ribozyme cleavage of HIV-I RNA, methods of cleaving RNA using ribozymes, methods for increasing the specificity of ribozymes, and the preparation and use of ribozyme fragments in a hammerhead structure are described in U.S. Pat. Nos. 5,144,019; 5,116,742; and 5,225,337 and Koizumi et al., Nucleic Acid Res. 17:7059-7071 (1989). Preparation and use of ribozyme fragments in a hairpin structure are described by Chowrira and Burke, Nucleic Acids Res. 20:2835 (1992). Ribozymes can also be made by rolling transcription as described in Daubendiek and Kool, Nat. Biotechnol. 15(3):273-277 (1997).

The hybridizing region of the ribozyme may be modified or may be prepared as a branched structure as described in Horn and Urdea, Nucleic Acids Res. 17:6959-67 (1989). The basic structure of the ribozymes may also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. 245:1-16 (1997).

Ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al., Science 224:574-578 (1984); Zaug and Cech, Science 231:470-475 (1986); Zaug et al., Nature, 324:429-433 (1986); W0 88/04300; Been and Cech, Cell 47:207-216 (1986)). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

Using the nucleic acid sequences of the invention and methods known in the art, ribozymes are designed to specifically bind and cut the corresponding mRNA species. Ribozymes thus provide a means to inhibit the expression of any of the proteins encoded by the disclosed nucleic acids or their full-length genes. The full-length gene need not be known in order to design and use specific inhibitory ribozymes. In the case of a nucleic acid or cDNA of unknown function, ribozymes corresponding to that nucleotide sequence can be tested in vitro for efficacy in cleaving the target transcript. Those ribozymes that effect cleavage in vitro are further tested in vivo. The ribozyme can also be used to generate an animal model for a disease, as described in Birikh et al., Eur. J. Biochem. 245:1-16 (1997). An effective ribozyme is used to determine the function of the gene of interest by blocking its transcription and detecting a change in the cell. Where the gene is found to be a mediator in a disease, an effective ribozyme is designed and delivered in a gene therapy for blocking transcription and expression of the gene.

Therapeutic and functional genomic applications of ribozymes proceed beginning with knowledge of a portion of the coding sequence of the gene to be inhibited. Thus, for many genes, a partial nucleic acid sequence provides adequate sequence for constructing an effective ribozyme. A target cleavage site is selected in the target sequence, and a ribozyme is constructed based on the 5′ and 3′ nucleotide sequences that flank the cleavage site. Retroviral vectors are engineered to express monomeric and multimeric hammerhead ribozymes targeting the mRNA of the target coding sequence. These monomeric and multimeric ribozymes are tested in vitro for an ability to cleave the target mRNA. A cell line is stably transduced with the retroviral vectors expressing the ribozymes, and the transduction is confirmed by Northern blot analysis and reverse-transcription polymerase chain reaction (RT-PCR). The cells are screened for inactivation of the target mRNA by such indicators as reduction of expression of disease markers or reduction of the gene product of the target mRNA.

Antisense Approaches

Antisense approaches are a way of preventing or reducing gene function by targeting the genetic material. The objective of the antisense approach is to use a sequence complementary to the target gene to block its expression and create a mutant cell line or organism in which the level of a single chosen protein is selectively reduced or abolished. Antisense techniques have several advantages over other ‘reverse genetic’ approaches. The site of inactivation and its developmental effect can be manipulated by the choice of promoter for antisense genes or by the timing of external application or microinjection. Antisense can manipulate its specificity by selecting either unique regions of the target gene or regions where it shares homology to other related genes.

Under one embodiment, the process involves the introduction and expression of an antisense gene sequence. Such a sequence is one in which part or all of the normal gene sequences are placed under a promoter in inverted orientation so that the ‘wrong’ or complementary strand is transcribed into a noncoding antisense RNA that hybridizes with the target mRNA and interferes with its expression. An antisense vector can be constructed by standard procedures and introduced into cells by transformation, transfection, electroporation, microinjection, infection, etc. The type of transformation and choice of vector will determine whether expression is transient or stable. The promoter used for the antisense gene may influence the level, timing, tissue, specificity, or inducibility of the antisense inhibition.

One aspect of the invention relates to the use of nucleic acids, e.g., SEQ ID NOs: 1, 3, 5, 7, and 8, or a sequence complementary thereto, in antisense therapy. As used herein, antisense therapy refers to administration or in situ generation of oligonucleotide molecules or their derivatives which specifically hybridize (e.g., bind) under physiological conditions with the cellular mRNA and/or genomic DNA, thereby inhibiting transcription and/or translation of that gene. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA. Alternatively, the antisense construct is an oligonucleotide probe which is generated ex vivo and which, when introduced into the cell, causes inhibition of expression by hybridizing with the mRNA and/or genomic sequences of a subject nucleic acid. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al., BioTechniques 6:958-976 (1988); and Stein et al., Cancer Res 48:2659-2668 (1988). With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. See Wagner, Nature 372:333 (1994). Therefore, oligonucleotides complementary to either the 5′ or 3′ untranslated, non-coding regions of a gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also be used in accordance with the invention. Whether designed to hybridize to the 5′, 3′, or coding region of subject mRNA, antisense nucleic acids should be at least six nucleotides in length, and are preferably less that about 100 and more preferably less than about 50, 25, 17 or 10 nucleotides in length.

Regardless of the choice of target sequence, it is preferred that in vitro studies are first performed to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., PNAS 86:6553-6556 (1989); Lemaitre et al., PNAS 84:648-652 (1987); WO 88/09810) or the blood-brain barrier (see, e.g., WO 89/10134), hybridization-triggered cleavage agents (See, e.g., Krol et al., BioTechniques 6:958-976 (1988)), or intercalating agents (see, e.g., Zon, Pharm. Res. 5:539-549 (1988)). To this end, the oligonucleotide may be conjugated to another molecule, e.g., a peptide, hybridization triggered cross-linking agent, transport agent, hybridization-triggered cleavage agent, etc.

Antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Antisense oligonucleotides may also comprise at least one modified sugar moiety selected from the group including but not limited to arabinose, 2-fluoroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al., PNAS 93:14670 (1996) and in Eglom et al., Nature 365:566 (1993). One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual beta-units, the strands run parallel to each other (Gautier et al., Nucl. Acids Res. 15:6625-6641 (1987)). The oligonucleotide is a 2′-O-methylribonucleotide (Inoue et al., Nucl. Acids Res. 15:6131-12148 (1987)), or a chimeric RNA-DNA analogue (Inoue et al., FEBS Lett. 215:327-330 (1987)).

Antisense molecules can be delivered to cells which express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation on endogenous mRNAs. Therefore, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous transcripts and thereby prevent translation of the target mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art, and can be plasmid, viral, or others known in the art for replication and expression in mammalian cells.

Expression of the sequence encoding the antisense RNA can be by any promoter known in the art to act in mammalian, preferably human cells. Such promoters can be inducible or constitutive. Such promoters include but are not limited to: the SV40 early promoter region, the promoter contained in the 3′ long terminal repeat of Rous sarcoma virus, the herpes thymidine kinase promoter, the regulatory sequences of the metallothionein gene, etc. Any type of plasmid, cosmid, YAC or viral vector can be used to prepare the recombinant DNA construct which can be introduced directly into the tissue site; e.g., the choroid plexus or hypothalamus. Alternatively, viral vectors can be used which selectively infect the desired tissue (e.g., for brain, herpesvirus vectors may be used), in which case administration may be accomplished by another route (e.g., systemically).

Antisense RNA, DNA, and ribozyme molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Endogenous gene expression can be reduced by inactivating or “knocking out” the gene or its promoter using targeted homologous recombination. (E.g., see Smithies et al., Nature 317:230-234 (1985); Thomas & Capecchi, Cell 51:503-512 (1987); Thompson et al., Cell 5:313-321(1989)). For example, a mutant, non-functional gene (or a completely unrelated DNA sequence) flanked by DNA homologous to the endogenous gene (either the coding regions or regulatory regions of the gene) can be used, with or without a selectable marker and/or a negative selectable marker, to transfect cells that express that gene in vivo. Insertion of the DNA construct, via targeted homologous recombination, results in inactivation of the gene.

G. Antibodies

One aspect of the invention concerns antibodies, single-chain antigen binding molecules, or other proteins that specifically bind to one or more of the protein, polypeptide, or peptide molecules of the invention and their homologs, fusions or fragments. In a particularly preferred embodiment, the antibody specifically binds to a protein having the amino acid sequence set forth in SEQ ID NOs: 2, 4, 6, or 9, or an amino acid sequence encoded by a nucleic acid sequence selected from the group consisting of SEQ ID NOs: 1, 3, 5, or 8. Such antibodies may be used to quantitatively or qualitatively detect the protein or peptide molecules of the invention.

Nucleic acid molecules that encode all or part of the protein or polypeptide of the invention can be expressed, via recombinant means, to yield protein or peptides that can in turn be used to elicit antibodies that are capable of binding the expressed protein or peptide. Such antibodies may be used in immunoassays for that protein. Such protein-encoding molecules, or their fragments may be a “fusion” molecule (i.e., a part of a larger nucleic acid molecule) such that, upon expression, a fusion protein is produced. It is understood that any of the nucleic acid molecules of the invention may be expressed, via recombinant means, to yield proteins or peptides encoded by these nucleic acid molecules.

The antibodies that specifically bind proteins, polypeptides, and protein fragments of the invention may be polyclonal or monoclonal and may comprise intact immunoglobulins, or antigen binding portions of immunoglobulins fragments (such as (F(ab′), F(ab′)₂), or single-chain immunoglobulins producible, for example, via recombinant means. It is understood that practitioners are familiar with the standard resource materials which describe specific conditions and procedures for the construction, manipulation and isolation of antibodies (see, e.g., Harlow and Lane, in: Antibodies: A Laboratory Manual, Cold Spring Harbor Press, Cold Spring Harbor, N.Y., 1988).

As discussed below, such antibody molecules or their fragments may be used for diagnostic purposes. Where the antibodies are intended for diagnostic purposes, it may be desirable to derivatize them, for example with a ligand group (such as biotin) or a detectable marker group (such as a fluorescent group, a radioisotope or an enzyme).

The ability to produce antibodies that bind the protein, polypeptide, or peptide molecules of the invention permits the identification of mimetic compounds derived from those molecules. These mimetic compounds may contain a fragment of the protein, polypeptide, or peptide or merely a structurally similar region and nonetheless exhibits an ability to specifically bind to antibodies directed against that compound. Antibodies that specifically bind to human nucleic acid-encoded polypeptides should provide a detection signal at least about 5-, 10-, or 20-fold higher than a detection signal provided with other proteins when used in Western blots or other immunochemical assays. Preferably, antibodies that specifically bind ΔN p73 polypeptides do not detect other proteins in immunochemical assays and can immunoprecipitate nucleic acid-encoded proteins from solution.

To test for the presence, for example without limitation, of serum antibodies to the ΔN p73 polypeptide in a human population, human antibodies are purified by methods well known in the art. Preferably, the antibodies are affinity purified by passing antiserum over a column to which a protein, polypeptide, or fusion protein is bound. The bound antibodies can then be eluted from the column, for example using a buffer with a high salt concentration. In addition to the antibodies discussed above, genetically engineered antibody derivatives are made, such as single chain antibodies.

In one aspect, this invention includes monoclonal antibodies that show a subject polypeptide is highly expressed in neural tissue or tumor tissue, especially neural cancer tissue or neural cancer-derived cell lines. Therefore, in one embodiment, this invention provides a diagnostic tool for the analysis of expression of a subject polypeptide in general, and in particular, as a diagnostic for neural cancers.

Antibodies can be used, e.g., to monitor protein levels in an individual for determining, e.g., whether a subject has a disease or condition. The level of polypeptides may be measured from cells in bodily fluid, such as in blood samples.

H. Pharmaceutical Compositions

Pharmaceutical compositions can comprise proteins, polypeptides, peptides, antibodies, or polynucleotides of the claimed invention. The pharmaceutical compositions will comprise a therapeutically effective amount of either proteins, polypeptides, peptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example, a ΔN p73 molecule or fragments thereof, antibodies of a ΔN p73 molecule, agonists, antagonists or inhibitors of ΔN p73, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the pharmaceuticals compositions of the invention can be (1) administered directly to the subject; (2) delivered ex vivo, to cells derived from the subject; or (3) delivered in vitro for expression of recombinant proteins.

Direct delivery of the compositions will generally be accomplished by injection, either subcutaneously, intraperitoneally, intravenously or intramuscularly, or delivered to the interstitial space of a tissue. The compositions can also be administered into a tumor or lesion. Other modes of administration include oral and pulmonary administration, suppositories, and transdermal applications, needles, and gene guns or hyposprays. Dosage treatment may be a single dose schedule or a multiple dose schedule.

Methods for the ex vivo delivery and reimplantation of transformed cells into a subject are known in the art and described in e.g., WO 93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once a subject gene has been found to correlate with a proliferative disorder, such as neoplasia, dysplasia, and hyperplasia, the disorder may be amenable to treatment by administration of a therapeutic agent based on the nucleic acid or corresponding polypeptide.

Preparation of antisense polypeptides is discussed above. Neoplasias that are treated with the antisense composition include, but are not limited to, cervical cancers, melanomas, colorectal adenocarcinomas, Wilms' tumor, retinoblastoma, sarcomas, myosarcomas, lung carcinomas, leukemias, such as chronic myelogenous leukemia, promyelocytic leukemia, monocytic leukemia, and mycloid leukemia, and lymphomas, such as histiocytic lymphoma. Proliferative disorders that are treated with the therapeutic composition include disorders such as anhydric hereditary ectodermal dysplasia, congenital alveolar dysplasia, epithelial dysplasia of the cervix, fibrous dysplasia of bone, and mammary dysplasia. Hyperplasias, for example, endometrial, adrenal, breast, prostate, or thyroid hyperplasias or pseudoepitheliomatous hyperplasia of the skin, are treated with antisense therapeutic compositions. Even in disorders in which mutations in the corresponding gene are not implicated, downregulation or inhibition of nucleic acid-related gene expression can have therapeutic application. For example, decreasing nucleic acid-related gene expression can help to suppress tumors in which enhanced expression of the gene is implicated. Further, decreasing ΔN p73 expression can help to suppress tumors by allowing p53, p63, and TA p73 induced apoptosis in the tumor cells.

Both the dose of the antisense composition and the means of administration are determined based on the specific qualities of the therapeutic composition, the condition, age, and weight of the patient, the progression of the disease, and other relevant factors. Administration of the therapeutic antisense agents of the invention includes local or systemic administration, including injection, oral administration, particle gun or catheterized administration, and topical administration. Preferably, the therapeutic antisense composition contains an expression construct comprising a promoter and a polynucleotide segment of at least about 12, 22, 25, 30, or 35 contiguous nucleotides of the antisense strand of a nucleic acid. Within the expression construct, the polynucleotide segment is located downstream from the promoter, and transcription of the polynucleotide segment initiates at the promoter.

Various methods are used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Receptor-mediated targeted delivery of therapeutic compositions containing an antisense polynucleotide, subgenomic polynucleotides, or antibodies to specific tissues is also used. Receptor-mediated DNA delivery techniques are described in, for example, Findeis et al., Trends in Biotechnol. (1993) 11:202-205; Chiou et al., (1994) Gene Therapeutics: Methods And Applications Of Direct Gene Transfer (J. A. Wolff, ed.); Wu & Wu, J. Biol. Chem. (1988) 263:621-24; Wu et al., J. Biol. Chem. (1994) 269:542-46; Zenke et al., PNAS (1990) 87:3655-59; Wu et al., J. Biol. Chem. (1991) 266:338-42. Preferably, receptor-mediated targeted delivery of therapeutic compositions containing antibodies of the invention is used to deliver the antibodies to specific tissue.

Therapeutic compositions containing antisense subgenomic polynucleotides are administered in a range of about 100 ng to about 200 mg of DNA for local administration in a gene therapy protocol. Concentration ranges of about 500 ng to about 50 mg, about 1 mg to about 2 mg, about 5 mg to about 500 mg, and about 20 mg to about 100 mg of DNA can also be used during a gene therapy protocol. Factors such as method of action and efficacy of transformation and expression are considerations which will affect the dosage required for ultimate efficacy of the antisense subgenomic nucleic acids. Where greater expression is desired over a larger area of tissue, larger amounts of antisense subgenomic nucleic acids or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect.

For genes encoding polypeptides or proteins with anti-inflammatory activity, suitable use, doses, and administration are described in U.S. Pat. No. 5,654,173. Therapeutic agents also include antibodies to proteins and polypeptides encoded by the subject nucleic acids, as described in U.S. Pat. No. 5,654,173.

Gene Delivery

The therapeutic nucleic acids of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0415731; EP 0345242; WO 90/07936; WO 94/03622; WO 93/25698; WO 93/25234; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. Nos. 5,219,740 and 4,777,127; and GB Patent No. 2,200,651. Preferred recombinant retroviruses include those described in WO 91/02805.

Packaging cell lines suitable for use with the above-described retroviral vector constructs may be readily prepared (WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. (1988) 166:154-165; and Flotte et al., PNAS 90:10613 -10617 (1993).

Representative examples of adenoviral vectors include those described by Berkner, Biotechniques 6:616-627 (1988); Rosenfeld et al., Science 252:431-434 (1991); WO 93/19191; Kolls et al., PNAS 91:215-219 (1994); Kass-Eisler et al., PNAS 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al., Nat. Genet. 1:372-378 (1992); and Levrero et al., Gene 101:195-202 (1991). Exemplary adenoviral gene therapy vectors employable in this invention also include those described in WO 94/12649, WO 93/03769, WO 93/19191, WO 94/28938, WO 95/11984 and WO 95/00655. Administration of DNA linked to killed adenovirus as described in Curiel, Hum. Gene Ther. 3:147-154 (1992) may be employed.

Other gene delivery vehicles and methods may be employed, including polycationic condensed DNA linked or unlinked to killed adenovirus alone (Curiel, Hum. Gene Ther. 3:147-154 (1992)); ligand linked DNA (Wu, J. Biol. Chem. 264:16985-16987 (1989)); eukaryotic cell delivery vehicles cells (U.S. Pat. No. 6,287,792); deposition of photopolymerized hydrogel materials; hand-held gene transfer particle gun (U.S. Pat. No. 5,149,655); ionizing radiation (U.S. Pat. No. 5,206,152; WO 92/11033); and nucleic charge neutralization or fusion with cell membranes. Additional approaches are described in Philip, Mol. Cell Biol. 14:2411-2418 (1994), and in Woffendin et al., PNAS 91:11581-11585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, WO 95/13796, WO 94/23697, WO 91/14445, and EP 0524968.

I. Diagnostic and Prognostic Assays

Agents of the present invention can be utilized in methods to determine, for example, without limitation, the presence or absence of a ΔN p73 molecule in a sample, the level of ΔN p73 molecule in a sample, a TA p731 ΔN p73 ratio in a sample. Moreover, agents of the present invention can be utilized in methods for predicting tumor resistance to treatments involving p53, p63, or TN p73-induced apoptosis, methods for predicting tumor resistance to treatments involving chemotherapy agents or radiotherapy agents, and methods for predicting a predisposition to cancer.

As used herein, the “Expression Response” manifested by a cell or tissue of an organism is said to be “altered” if it differs from the Expression Response of cells or tissues not exhibiting the phenotype. To determine whether a Expression Response is altered, the Expression Response manifested by the cell or tissue of the organism exhibiting the phenotype is compared with that of a similar cell or tissue sample of an organism not exhibiting the phenotype. As will be appreciated, it is not necessary to redetermine the Expression Response of the cell or tissue sample of organisms not exhibiting the phenotype each time such a comparison is made; rather, the Expression Response of a particular organism may be compared with previously obtained values of normal organisms.

Also as used herein, a “tissue sample” is any sample that comprises more than one cell. In a preferred aspect, a tissue sample comprises cells that share a common characteristic (e.g. derived from neurons, epidermis, muscle etc.).

A number of methods can be used to compare the expression response between two or more samples of cells or tissue. These methods include hybridization assays, such as Northerns, RNAse protection assays, and in situ hybridization. In a preferred method, the expression response is compared by PCR-type assays.

An advantage of in situ hybridization over certain other techniques for the detection of nucleic acids is that it allows an investigator to determine the precise spatial population. In situ hybridization may be used to measure the steady-state level of RNA accumulation. A number of protocols have been devised for in situ hybridization, each with tissue preparation, hybridization and washing conditions.

In situ hybridization also allows for the localization of proteins or mRNA within a tissue or cell. It is understood that one or more of the molecules of the invention, preferably one or more of the nucleic acid molecules or fragments thereof of the invention or one or more of the antibodies of the invention may be utilized to detect the level or pattern of a protein or mRNA thereof by in situ hybridization.

In one aspect of the present invention, an evaluation can be conducted to determine whether a ΔN p73 molecule is present. One or more of the ΔN p73 molecules, preferably a ΔN p73 mRNA and a ΔN p73 polypeptide, of the present invention are utilized to detect the presence, type, or quantity of the ΔN p73 species. Generally, such a method comprises: (a) obtaining cell or tissue sample of interest; and (b) selectively detecting the presence or absence, or ascertaining the level of a ΔN p73 molecule.

As used herein, the term “presence” refers to when a molecule can be detected using a particular detection methodology. Also as used herein, the term “absence” refers to when a molecule cannot by detected using a particular detection methodology.

The present invention also includes and provides a method for determining a level or pattern of a protein in an animal cell or animal tissue comprising (A) assaying the concentration of the protein in a first sample obtained from the animal cell or animal tissue; (B) assaying the concentration of the protein in a second sample obtained from a reference animal cell or a reference animal tissue with a known level or pattern of the protein; and (C) comparing the assayed concentration of the protein in the first sample to the assayed concentration of the protein in the second sample.

Any method for analyzing proteins can be used to detect or measure levels of ΔN p73 polypeptide. As an illustration, size differences can be detected Western blots of protein extracts from the two tissues. Other changes, such as expression levels and subcellular localization, can also be detected immunologically, using antibodies to the corresponding protein. The expression pattern of any cell or tissue types can be compared. Such comparison can also occur in a temporal manner. Another comparison can be made between difference developmental states of a tissue or cell sample.

More particularly, in one embodiment, ΔN p73 mRNA in a cell or tissue sample can be detected by incubating ΔN p73 mRNA molecules with cell or tissue sample extracts of an organism under conditions sufficient to permit nucleic acid hybridization. The detection of double-stranded probe-mRNA hybrid molecules is indicative of the presence of the mRNA; the amount of such hybrid formed is proportional to the amount of mRNA. Thus, such probes may be used to ascertain the level and extent of the mRNA production in an organism's cells or tissues. Such nucleic acid hybridization may be conducted under quantitative conditions (thereby providing a numerical value of the amount of the mRNA present). Alternatively, the assay may be conducted as a qualitative assay that indicates either that the mRNA is present, or that its level exceeds a user set, predefined value.

Alternatively, ΔN p73 mRNA may be selectively detected using standard PCR or RT-PCR techniques such as those described herein. The ΔN p73 mRNA may also be selectively detected by an oligonucleotide probe which specifically hybridizes to exon 3′.

In another embodiment, ΔN p73 polypeptide molecules may be selectively detected using an immunological binding assay, e.g., an in situ binding assay. In this regard, an antibody which selectively binds to ΔN p73 may be used. More particularly, the antibody may selectively bind to a ΔN p73 polypeptide comprising an amino acid sequence of SEQ ID NO: 9. Optionally, the antibody may be labeled as described below to aid in detection.

More particularly, ΔN p73 polypeptide molecules can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (for example, in this case a ΔN p73 polypeptide molecule or an antigenic subsequence thereof). The antibody (e.g., anti-ΔN p73) may be produced by any of a number of means well known to those of skill in the art and as described above.

Immunoassays also often use a labeling agent to specifically bind to, and label the complex formed by the antibody and antigen. The labeling agent may itself be one of the moieties comprising the antibody/antigen complex. Thus, the labeling agent may be a labeled ΔN p73 polypeptide or a labeled anti-ΔN p73 antibody. Alternatively, the labeling agent may be a third moiety, such a secondary antibody, that specifically binds to the antibody/ΔN p73 complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol., 111:1401-1406 (1973); Akerstrom et al., J. Immunol., 135:2589-2542 (1985)). The labeling agent can be modified with a detectable moiety, such as biotin, to which another molecule can specifically bind, such as streptavidin. A variety of detectable moieties are well known to those skilled in the art. A preferred label is a fluorescent label.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Generally, immunoassays for detecting a ΔN p73 polypeptide in a sample may be either competitive or noncompetitive. Noncompetitive immunoassays are assays in which the amount of antigen is directly measured. In one preferred “sandwich” assay, for example, the anti-ΔN p73 antibodies can be bound directly to a solid substrate on which they are immobilized. These immobilized antibodies then capture the ΔN p73 polypeptide present in the test sample. The ΔN p73 polypeptide is thus immobilized, and is then bound by a labeling agent, such as a second ΔN p73 antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

In competitive assays, the amount of ΔN p73 polypeptide present in the sample is measured indirectly by measuring the amount of a known, added (exogenous) ΔN p73 protein displaced (competed away) from an anti-ΔN p73 antibody by the unknown ΔN p73 polypeptide present in a sample. In one competitive assay, a known amount of ΔN p73 protein is added to a sample and the sample is then contacted with an antibody that specifically binds to the ΔN p73. The amount of exogenous ΔN p73 protein bound to the antibody is inversely proportional to the concentration of ΔN p73 polypeptide present in the sample. In a particularly preferred embodiment, the antibody is immobilized on a solid substrate. The amount of ΔN p73 polypeptide bound to the antibody may be determined either by measuring the amount of ΔN p73 polypeptide present in a ΔN p73/antibody complex, or alternatively by measuring the amount of remaining uncomplexed protein. The amount of ΔN p73 polypeptide may be detected by providing a labeled ΔN p73 molecule.

A hapten inhibition assay is another preferred competitive assay. In this assay the known ΔN P73 protein is immobilized on a solid substrate. A known amount of anti-ΔN P73 antibody is added to the sample, and the sample is then contacted with the immobilized ΔN P73. The amount of anti-ΔN P73 antibody bound to the known immobilized ΔN P73 protein is inversely proportional to the amount of ΔN P73 polypeptide present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Western blot (immunoblot) analysis may also used to detect and quantify the presence of ΔN P73 polypeptide in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the ΔN P73 polypeptide. The anti-ΔN P73 polypeptide antibodies specifically bind to the ΔN P73 polypeptide on the solid support. These antibodies may be directly labeled or alternatively may be subsequently detected using labeled antibodies (e.g., labeled sheep anti-mouse antibodies) that specifically bind to the anti-ΔN P73 antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev., 5:34-41 (1986)).

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize a ΔN P73 polypeptide, or secondary antibodies that recognize anti-ΔN P73.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

In clinical applications, human tissue samples can be screened for ΔN p73 molecules, or can be screened to determine the TA p73/ΔN p73 ratio. The results of such screenings can then be used in a clinical setting to predict the samples predisposition to cancer. Additionally, the results of such screening can be used to predict a tissues resistance to chemotherapy as well as its resistance to p53, p63, or TA p73-induced apoptosis. Such samples include needle biopsy cores, surgical resection samples, lymph node tissue, or serum. For example, these methods include obtaining a biopsy, which is optionally fractionated by cryostat sectioning to enrich tumor cells to about 80% of the total cell population. In certain embodiments, nucleic acids extracted from these samples may be amplified using techniques well known in the art.

As described herein, it has been unexpectedly discovered according to the present invention that presence of ΔN p73 in a tissue or cell can inhibit p53, p63, and TA p73-induced apoptosis. Such inhibition can be correlated to a tissue or cell's predisposition to cancer, as well as its resistance to chemotherapy or radiotherapy agents. Further, it was discovered that the TA p73/ΔN p73 ratio in a tissue or cell is relevant to the predisposition of the tissue or cell to cancer.

Thus, in one aspect of the present invention, a diagnostic assay for predicting a predisposition to cancer is provided comprising: (a) detecting the amount of a ΔN p73 molecule or the TA p73/ΔN p73 ratio in a tissue or cell of interest; and (b) comparing said amount to a base-line amount of tissue or cell types with a known disposition to cancer.

In another aspect of the present invention, a method for determining the TA p73/ΔN p73 ratio in a sample is provided. Such a method generally comprises: (a) obtaining a tissue or cell sample or interest; (b) selectively detecting the level of a TA p73 molecule and a ΔN p73 molecule as described above; (c) determining the TA p73/ΔN p73 ratio based on the detected levels of TA p73 and ΔN p73.

In yet another aspect of the invention, a method for predicting tumor resistance to treatments involving p53, p63, or TA p73-induced apoptosis is provided comprising: (a) obtaining a sample tissue or cell; (b) detecting the amount of a ΔN p73 molecule or a TA p73/ΔN p73 ratio in the sample; and (c) comparing said amount to a base-line amount in cell types of known resistance to p53, p63, or TA p73-induced apoptosis.

Likewise, a method for predicting tumor resistance to treatments involving chemotherapy agents or radiotherapy agents is also provided comprising: (a) obtaining a sample tissue or cell; (b) detecting the amount of a ΔN p73 molecule or a TA p73/ΔN p73 ratio in said sample; and (c) comparing said amount to a base-line amount in cell types of known resistance to chemotherapy agents.

The diagnostic and prognostic methods described herein can, for example without limitation, utilize one or more of the detection methods described herein, including but not limited to northern blot analysis, standard PCR, reverse transcription-polymerase chain reaction (RT-PCR), in situ hybridization, immunoprecipitation, Western blot hybridization, or immunohistochemistry.

In one aspect, the method comprises in situ hybridization with a nucleic acid molecule of the present invention as a probe. This method comprises contacting the labeled hybridization probe with a sample of a given type of tissue potentially containing cancerous or pre-cancerous cells as well as normal cells, and determining whether the probe labels some cells of the given tissue type to a degree significantly different (e.g., by at least a factor of two, or at least a factor of five, or at least a factor of twenty, or at least a factor of fifty) than the degree to which it labels other cells of the same tissue type.

Alternatively, the above diagnostic assays may be carried out using antibodies which selectively detect a polypeptide of the present invention. Accordingly, in one embodiment, the assay includes contacting the proteins of the test cell with an antibody specific for a ΔN p73 polypeptide and determining the approximate amount of immunocomplex formation. Such a complex can be detected by an assay for example without limitation an immunohistochemical assay, dot-blot assay, and an ELISA assay.

Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

Where tissue samples are employed, immunohistochemical staining may be used to determine the number and type of cells having a ΔN p73 polypeptide. For such staining, a multiblock of tissue can be taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissue samples can be fixed by treatment with a reagent such as formalin, glutaraldehyde, methanol, or the like. The samples are then incubated with an antibody, preferably a monoclonal antibody, with binding specificity for the marker polypeptides. This antibody may be conjugated to a label for subsequent detection of binding. Samples are incubated for a time sufficient for formation of the immuno-complexes. Binding of the antibody is then detected by virtue of a label conjugated to this antibody. Where the antibody is unlabeled, a second labeled antibody may be employed, e.g., which is specific for the isotype of the anti-marker polypeptide antibody. Examples of labels which may be employed include radionuclides, fluorescers, chemiluminescers, enzymes and the like.

Where enzymes are employed, the substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In one embodiment, the assay is performed as a dot blot assay. The dot blot assay finds particular application where tissue samples are employed as it allows determination of the average amount of a ΔN p73 polypeptide associated with a single cell by correlating the amount of marker polypeptide in a cell-free extract produced from a predetermined number of cells. The diagnostic assays described above can be adapted to be used as prognostic assays, as well.

The methods of the invention can also be used to follow the clinical course of a tumor. For example, the assay of the invention can be applied to a tissue sample from a patient; following treatment of the patient for the cancer, another tissue sample is taken and the test repeated. Successful treatment will result in either removal of all cells which demonstrate differential expression characteristic of the cancerous or precancerous cells, or a substantial increase in expression of the gene in those cells, perhaps approaching or even surpassing normal levels.

Yet another aspect of the invention provides a method for evaluating the carcinogenic potential of an agent by (i) contacting a transgenic animal of the present invention with a test agent, and (ii) comparing the number of transformed cells in a sample from the treated animal with the number of transformed cells in a sample from an untreated transgenic animal or transgenic animal treated with a control agent. The difference in the number of transformed cells in the treated animal, relative to the number of transformed cells in the absence of treatment with a control agent, indicates the carcinogenic potential of the test compound.

Another aspect of the invention provides a method of evaluating an anti-proliferative activity of a test compound. In preferred embodiments, the method includes contacting a transgenic animal of the present invention, or a sample of cells from such animal, with a test agent, and determining the number of transformed cells in a specimen from the transgenic animal or in the sample of cells. A statistically significant decrease in the number of transformed cells, relative to the number of transformed cells in the absence of the test agent, indicates the test compound is a potential anti-proliferative agent.

J. Modulator Screening Assays

Another aspect of the invention is directed to the identification of agents capable of modulating one or more ΔN p73 molecules. Such agents are herein referred to as “modulators” or “modulating compounds”. In this regard, the invention provides assays for determining compounds that modulate the function and/or expression of one or more ΔN p73, TA p73, p63, or p53 molecules.

“Inhibitors,” “activators,” and “modulators” of ΔN p73 molecules are used interchangeably to refer to inhibitory, activating, or modulating molecules which can be identified using in vitro and in vivo assays for ΔN p73 activity and/or expression, e.g., ligands, agonists, antagonists, and their homologs and mimetics.

Modulator screening may be performed by adding a putative modulator test compound to a tissue or cell sample, and monitoring the effect of the test compound on the function and/or expression of ΔN p73, TA p73, p63, or p53. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds. In a particular embodiment, apoptosis can be induced in the treated and untreated cells to determine the effect of the modulator on p53, p63, and/or TA p73-induced apoptosis. Methods for inducing apoptosis are well known in the art and include, without limitation, exposure to chemotherapy or radiotherapy agents and withdrawal of obligate survival factors (e.g., NGF) if applicable. Differences between treated and untreated cells indicates effects attributable to the test compound.

More particularly, in one embodiment, a method for identifying ΔN p73 modulating compounds is provided comprising: (a) obtaining a sample tissue or cell which expresses a ΔN p73 molecule; (b) exposing the sample to a putative modulating compound; and (c) monitoring the level and/or activity of a p53, p63, TA p73, and/or ΔN p73.

In a preferred embodiment, the sample tissue or cell which expresses a ΔN p73 molecule is a sympathetic neuron of the SCG, and the activity and/or expression of ΔN p73 is monitored by withdrawing NGF from the sample to induce apoptosis. Once NGF is withdrawn, apoptosis can be monitored to determine if the putative modulating compound is affecting the ability of ΔN p73 to at least partially inhibit or block p53, p63, and/or TA p73-induced apoptosis. See Pozniak et al., Science, 289: 304-6 (2000).

In another embodiment, a method for identifying compounds which modulate the expression of a ΔN p73 molecule is provided comprising: (a) obtaining a tissue or cell sample which expresses the SEQ ID NO: 7 operably linked to a reporter gene; (b) exposing the sample to a putative modulating compound; and (c) monitoring the activity or expression of said ΔN p73 molecule. The reporter gene can be any reporter gene known in the art including, but not limited to, green fluorescent protein and luciferase.

Desirable effects of a test compound include an effect on any phenotype that was conferred by the cancer-associated marker nucleic acid sequence. Examples include a test compound that limits the overabundance of mRNA, limits production of the encoded protein, or limits the functional effect of the protein. The effect of the test compound would be apparent when comparing results between treated and untreated cells.

The invention thus also encompasses methods of screening for agents which inhibit promotion or expression of a ΔN p73 molecule in vitro, comprising exposing a cell or tissue in which the ΔN p73 molecule is detectable in cultured cells to an agent in order to determine whether the agent is capable of inhibiting production of the ΔN p73 molecule; and determining the level of ΔN p73 molecule in the exposed cells or tissue, wherein a decrease in the level of the ΔN p73 molecule after exposure of the cell line to the agent is indicative of inhibition of the ΔN p73 molecule.

Alternatively, the screening method may include in vitro screening of a cell or tissue in which a ΔN p73 molecule is detectable in cultured cells to an agent suspected of inhibiting production of the ΔN p73 molecule; and determining the level of the ΔN p73 molecule in the cells or tissue, wherein a decrease in the level of ΔN p73 molecule after exposure of the cells or tissue to the agent is indicative of inhibition of ΔN p73 molecule production.

The invention also encompasses in vivo methods of screening for agents which inhibit expression of the ΔN p73 molecules, comprising exposing a mammal having tumor cells in which a ΔN p73 molecule is detectable to an agent suspected of inhibiting production of ΔN p73 molecule; and determining the level of ΔN p73 molecule in tumor cells of the exposed mammal. A decrease in the level of ΔN p73 molecule after exposure of the mammal to the agent is indicative of inhibition of marker nucleic acid expression.

Accordingly, the invention provides a method comprising incubating a cell expressing the ΔN p73 molecule with a test compound and measuring the ΔN p73 molecule level. The invention further provides a method for quantitatively determining the level of expression of the ΔN p73 molecule in a cell population, and a method for determining whether an agent is capable of increasing or decreasing the level of expression of the ΔN p73 molecule in a cell population.

A method for determining whether an agent is capable of increasing or decreasing the level of expression of the ΔN p73 molecule in a cell population comprises the steps of (a) preparing cell extracts from control and agent-treated cell populations, (b) isolating the ΔN p73 molecule from the cell extracts, (c) quantifying (e.g., in parallel) the amount of an immunocomplex formed between the ΔN p73 molecule and an antibody specific to said ΔN p73 molecule.

The ΔN p73 molecules of this invention may also be quantified by assaying for its bioactivity. Agents that induce increased ΔN p73 molecule expression may be identified by their ability to increase the amount of immunocomplex formed in the treated cell as compared with the amount of the immunocomplex formed in the control cell. In a similar manner, agents that decrease expression of the ΔN p73 molecule may be identified by their ability to decrease the amount of the immunocomplex formed in the treated cell extract as compared to the control cell.

mRNA levels can be determined by Northern blot hybridization. mRNA levels can also be determined by methods involving PCR. Other sensitive methods for measuring mRNA, which can be used in high throughput assays, e.g., a method using a DELFIA endpoint detection and quantification method, are described, e.g., in Webb and Hurskainen Journal of Biomolecular Screening 1:119 (1996). ΔN p73 molecule levels can be determined by immunoprecipitations or immunohistochemistry using an antibody that specifically recognizes the protein product encoded by the nucleic acid molecules.

In another aspect of the invention, modulators of ΔN p73 can be identified by monitoring the function of p53, p63, TA p73, and secondary genes regulated thereby. As such, p53, p63, and TA p73-induced apoptosis can be monitored and correlated to the activity and/or expression of ΔN p73. Alternatively, the expression and activity of genes regulated by p53, p63, and TA p73, e.g., p21 or PUMA, may be monitored.

Agents that are identified as active in the drug screening assay are candidates to be tested for their capacity to block or promote apoptosis.

K. In Vivo Methods and Therapeutic Applications

The pharmaceutical compositions of the present invention, including antisense formulations, may be therapeutically used in clinical settings to affect cellular apoptosis. As described above, the N-terminal deletion of ΔN p73. removes the transactivation domain of TA p73, but not the DNA binding domain. Thus, ΔN p73 can bind to p53, p63, and TA p73 responsive gene promoters, but not elicit transcription. This binding confers an inhibitory effect on the transcription of p53, p63, and TA p73 responsive genes by acting in a dominant negative way to prevent binding and transactivation by p53, p63, and TA p73. Further, it has also been found that ΔN p73 at least partially inhibits or blocks expression of PUMA, a recently discovered mediator of apoptosis.

As such, according to the present invention, it has been shown that ΔN p73 at least partially inhibits or blocks apoptosis induced in cells by p53, p63, and TA p73. ΔN p73 is a also a key natural feedback inhibitor of p53 and TA p73, which moderates and controls the effect of increased p53 and TA p73. Additionally, it has been shown that the promoter of ΔN p73 contains a p53 response element, and that p53 and p73 induce the expression of ΔN p73. Thus, ΔN p73 is coordinately regulated and in balance with p53 and TA p73 in normal cells, and in normal cellular response.

As used herein, “at least partially inhibiting” refers to the reduction of a particular event, for example without limitation, the function and/or expression of p 53, p63, TA p73, and/or ΔN p73. In a preferred embodiment, to determine whether a particular event is “at least partially inhibited”, the sample of interest subject to a particular method or agent is compared with similar sample of interest not subjected to the particular method or agent. In one embodiment, an inhibition of a particular event is statistically significant. In a particularly preferred embodiment, a particular event is inhibited in a sample of interest by 25%, 50%, 60%, 70%, 75%, 80%, 85%, 90 %, 95% or 100%, as compared to a similar sample of interest not subjected to the particular event. More particularly, as used herein, “blocking” refers to inhibition of a particular event in a sample of interest by greater than 90%, as compared to a similar sample of interest not subject to the particular event.

Accordingly, one aspect of the present invention is directed to the use of the ΔN p73 to at least partially inhibit or block apoptosis mediated by p53, p63, and/or TA p73. ΔN p73 thus has application in protecting cells/tissues undergoing apoptosis following chemotherapy or radiotherapy, such as GI tract or haematopoetic cells, or in extending the life and thus protective effects of haematopoetic cells following bone marrow transplant. Additionally, ΔN p73 has application in at least partially inhibiting or blocking apoptosis in acute diseases such as myocardial infarct, ischaemia or sepsis. In sum, ΔN p73 may find application in any disease where treatment through at least partially inhibiting or blocking apoptosis is a therapeutic paradigm.

Another aspect of the present invention is directed to the use of antisense ΔN p73 as a therapeutic molecule to at least partially inhibit or block (knockdown/knockout) expression of natural ΔN p73. The consequence of at least partially inhibiting or blocking expression of natural ΔN p73 would be to induce apoptosis in cells, or sensitize cells to apoptosis mediated by p53, p63, and/or TA p73. A particular application would be for the treatment of cancer, particularly those where ΔN p73 is elevated. A further application would be for treatment of cancer in combination with chemotherapy or radiotherapy where p53, p63, and/or TA p73 is increased, or where ΔN p73 is elevated. Yet another application is for the treatment of inflammatory disease where particular haematopoeitic inflammatory cells are in excess, or where there is a therapeutic paradigm for treatment of inflammatory disease through increasing apoptosis.

More particularly, in one embodiment, a method for at least partially inhibiting apoptosis in a cell is provided comprising: (a) providing an expression vector comprising a nucleic acid sequence encoding a polypeptide selected from the group consisting of SEQ ID NOs: 2, 4, and 6, operably linked to an expression control sequence; (b) introducing the expression vector into the cell; and (c) maintaining the cell under conditions permitting expression of the encoded polypeptide in the cell.

In another embodiment, a method for at least partially inhibiting the expression of at least one of a p53 molecule, a p63 molecule, and a TA p73 molecule in a cell is provided comprising: (a) providing an expression vector comprising a nucleic acid sequence encoding a polypeptide selected from the group consisting of SEQ ID NOs: 2, 4, and 6, operably linked to an expression control sequence; (b) introducing the expression vector into the cell; and (c) maintaining the cell under conditions permitting expression of the encoded amino acid in the cell.

In yet another embodiment, a method for at least partially inhibiting the production of a ΔN p73 polypeptide in a cell is provided comprising: (a) providing an isolated nucleic acid molecule comprising at least 10 consecutive nucleotides of the complement of SEQ ID NO: 8; (b) introducing the nucleic acid molecule into the cell; and (c) maintaining the cell under conditions permitting the binding of the nucleic acid sequence to ΔN p73 mRNA.

### Deposits

The following clones were deposited:

Clone ΔN p73α (Accession No. 01091401) was deposited with ECACC, CAMR, Salisbury, Wiltshire SP40JG UK on 13th, Sep. 2001. Clone ΔN p73α contains an 1746 bp insert in pcDNA3.1/V5-HisTOPO. The insert was obtained from JVM-2 cells and contains within it the coding sequence of human ΔN p73α.

Clone ΔN p73β (Accession No. 01091402) was deposited with ECACC, CAMR, Salisbury, Wiltshire SP40JG UK on 13th, Sep. 2001. Clone ΔN p73β contains an 1665 bp insert in pcDNA3.1/V5-HisTOPO. The insert was obtained from JVM-2 cells and contains within it the coding sequence of human ΔN p73β.

Clone ΔNp 73γ (Accession No. 01091403) was deposited with ECACC, CAMR, Salisbury, Wiltshire SP40JG UK on 13th, Sep. 2001. Clone ΔN p73γ contains an 1273 bp insert in pcDNA3.1/V5-HisTOPO. The insert was obtained from JVM-2 cells and contains within it the coding sequence of human ΔN p73γ.

Clone ΔN p73δ (Accession No. 01091404) was deposited with ECACC, CAMR, Salisbury, Wiltshire SP40JG UK on 13th, Sep. 2001. Clone ΔN p73δ contains an 2240 bp insert in pGL3 basic. The insert was obtained from JVM-2 cells and contains within it the promoter for human ΔN p73δ.

Application of the teachings of the present invention to a specific problem or environment is within the capabilities of one having ordinary skill in the art in light of the teachings contained herein. Examples of the products and processes of the present invention appear in the following examples, which are provided by way of illustration, and are not intended to be limiting of the present invention.

### ILLUSTRATIVE EXAMPLES

### Example 1

### RNA Extraction and Reverse Transcription

Total RNA is extracted from sample cells (i.e., JVM-2, HACaT, MCF-7, etc.), using, e.g., the RNeasy kit (Qiagen, Basel, Switzerland). Generally, 5-10×10⁶ sample cells are lysed in 350 μl RLT buffer (10 μl 2-mercaptoethanol added to 1 ml RLT buffer before use) and homogenized through a QIAshredder column. Then, 350 μl of 70% ethanol is added to the homogenized medium and applied to a RNeasy mini spin column. The column is washed twice with 700 and 500 μl RPE Buffer and eluted with 50 μl of PCR-H₂O on an eppendorf centrifuge 5417 C. The RNA concentration and purity may be determined using a photo spectrometer at 260 nM/280 nM (GeneQuant, Amersham Pharmacia Biotech, Dübendorf, Switzerland).

During the extraction procedure, contaminating DNA can be removed by DNase pre-treatment, wherein 600 ng of RNA is treated with 0.25 μl 1M NaAc, pH 5; 1 μl 10 U/μl DNase I, RNase-free; 0.5 μl 50 U/μl RNase inhibitor; and 2 μl 25 mM MgCl2 in 6.25 μl PCR-H₂O for 15 minutes at 37° C., followed by 5 minutes at 90° C. and immediate placement on ice for at least 5 minutes. The DNase pre-treated RNA can then be stored at −80° C. for future use if desired.

Once extracted, 200 ng of total RNA is reverse transcribed in a 20 μl reaction volume, using 1.6 μl 2 μg/μl p(dN₆) random primers; 0.8 μl 24 U/μl avian myeloblastosis virus reverse transcriptase (AVM-RT); 1 μl 50 U/μl RNase inhibitor; 2 μl 10 mM dNTP mix; and 4 μl 5×AMV-RT buffer (all reagents from Roche, Basel Switzerland). The reaction volume is incubated for 10 minutes at room temperature, then one hour at 42° C., followed by 5 minutes at 95° C. Afterwards the mix is centrifuged and put on ice for at least 5 minutes. To check for contaminating genomic DNA, RNA samples may be processed identically as for cDNA synthesis, except that the reverse transcriptase is replaced by the same volume of water. The cDNA may be stored at −20° C. for future use if desired.

### Example 2

### Standard Condition Polymerase Chain Reaction (PCR)

Standard condition PCR may be carried out in a total volume 30 μl containing 9.24 μl PCR-H₂O; 3 μl glycerol heated to 95° C.; 3 μl 10×PCR buffer with MgCl₂ (Roche); 3 μl 1 mM dNTP's mix (Roche); 0.8 μl primer sense; 0.8 μl primer antisense; 016 μl 5 U/μl Taq DNA Polymerase (Roche); and 10 μl Template. By way of example, the following primers and probes may be used (all 10 μM, Microsynth, Balgach, Switzerland): oligonucleotides specific for cloning 7S—forward primer: 5′-GCTACTCGGGAGGCTGAGAC-3′ (SEQ ID NO: 17), reverse primer: 5′-AGGCGCGATCCCACTACTGA 3′ (SEQ ID NO: 18); oligonucleotides specific for cloning TA p73—forward primer: 5′-ACGCAGCGAAACCGGGGCCCG-3′ (SEQ ID NO: 19), reverse primer: 5′-GCCGCGCGGCTGCTCATCTGG-3′ (SEQ ID NO: 20); and oligonucleotides specific for cloning ΔN p73—forward primer: 5′-CCCGGACTTGGATGAATACT-3′ (SEQ ID NO: 21), reverse primer: 5′-GCCGCGCGGCTGCTCATCTGG-3′ (SEQ ID NO: 22).

Amplification can be performed using a PCR cycler (GeneAmp PCR 9600, Perkin Elmer, Rothrist, Switzerland) with the following cycle conditions. For TA p73, 1 cycle for 5 minutes at 95° C.; 35 cycles of 30 seconds at 95° C. (denaturation), 30 seconds at 55° C. (annealing), and 1 minute at 72° C. (elongation); followed by 1 cycle for 5 minutes at 72° C. (final elongation). For ΔN p73, 1 cycle for 5 minutes at 95° C.; 38 cycles of 30 seconds 95° C. (denaturation), 30 seconds at 55° C. (annealing), and 1 minute at 72° C. (elongation); followed by 1 cycle for 5 minutes at 72° C. (final elongation).

12 μl of PCR product is loaded with 3 μl of 5× Loading Buffer on a 1.5% agarose gel (Invitrogen, Basel, Switzerland) with 100 μl 10 μg/μl Ethidium Bromide/100 ml (Merck, Dietikon, Switzerland)), and run at 80V for 60 to 90 minutes for analysis. PCR products can be stored at 4° C. over night or at −20° C. for longer if desired.

### Example 3

### Cloning & Characterization of PCR Products

Cloning of PCR Products

4 μl of a standard PCR product, 1 μl of the salt solution, and 1 μl of the TOPO TA Cloning® Kit pcDNA3.1/V5-His-TOPO vector (Invitrogen) are incubated for 15 minutes at room temperature then put on ice. 2 μl of this reaction is gently mixed with the One Shot® TOP10 chemically competent E. coli (Invitrogen) and first incubated on ice for 15 minutes, then heat-shocked for 30 seconds at 42° C. and immediately transferred on ice. 250 μl of room temperature SOC medium is then added to the bacteria, and the mixture is incubated for one hour at 37° C. on a vertical shaker at 225 rpm. Varying volumes of the incubated mixture are spread on Agar-plates (25 μg/μl Ampicilin) and incubated at 37° C. over night.

Insert Screening in Clones

Colonies from the Agar-plates are then picked and a plasmid mini preparation is performed. To screen the colonies, 5 μl of LB-medium with grown E. coli are diluted in 995 μl of Q-H₂O, and a standard PCR is performed as described above with the reverse cloning primer and the T7 primer, to check for insertion and direction of the PCR product.

Plasmid Mini Preparation for Sequencing

1.5 ml of LB overnight culture (L-Broth with Ampicilin (100 μg/ml)) is centrifuged at 14,000 rpm for 20 seconds, and resuspended in 250 ul of Buffer P1 ( 20 μl RNase A (100 mg/ml) added to 20 ml P1 Buffer before first use)(Qiagen). 250 μl of Buffer P2 is added to the resuspended mix followed by 350 ul of buffer N3. The total mix is then centrifuged for 10 minutes at 14,000 rpm. The supernatant is applied to a QIAprep column (Qiagen) and centrifuged using a Minifuge T (Heraeus, Zürich, Switzerland) with rotor 3360 for one minute at full speed. The column is then washed twice with 0.75 ml of PE Buffer, and the DNA is eluted in 40 μl of PCR-H₂O.

Plasmid Maxi Preparation for Standards

The day before the experiment, 50 μl from the plasmid miniprep described above or from Stock (−70° C.) is added to 200 ml L-Broth with Ampicilin (100 μg/ml) and incubated on a shaker over night (230 rpm, 37° C.). The day of the experiment, the bacteria are transferred to centrifugation beakers and centrifuged for 15 minutes at 5,000 rpm. The supernatant is discarded and the bacteria pellet is resuspended in 15 ml Cell Resuspension Solution. 15 ml of Lysis Solution and 15 ml of Neutralization Solution are added and mixed by carefully pipeting up and down. The lysed bacteria are then centrifuged for 20 minutes at 5,000 rpm.

The lysed supernatant is filtered through a sterile coffee filter and half the volume of isopropanol is added. The solution is mixed well, transferred to corex tubes, and centrifuged for 20 minutes at 7,000 rpm using, e.g., a Centrikon T124 centrifuge (Kontron Instruments, Basel, Switzerland) with rotors: A6.9 and AS4.7. The supernatant is discarded and the pellet dried. The pellet is then resuspended in 2 ml of Tris-HCL pH 6.4, and 10 ml of Resin Solution (dissolved at 37° C.) is added and mixed. The solution is transferred into a Maxicolumn connected with vacuum (Promega Wizard Plus Maxipreps, DNA purification system, Promega, Wallisellen, Switzerland). After the solution passed through the column completely, 25 ml of Column-Wash-solution followed by 5 ml Ethanol (80%) is passed through the column. The washed column is then dried for 5 minutes under vacuum and placed in a new 50 ml Falcon tube. 1.5 ml of PCR-H₂O (70° C.) is added on the column, incubated for one minute, and then centrifuged for 5 minutes at 2500 rpm using, e.g., a Minifuge T (Heraeus) with rotor 3360. The eluate is then sterile filtrated to remove Resin particles.

5 μl of the filtered eluate is diluted in 95 μl of Q-H₂O, and the plasmid concentration and purity is determined using a photo spectrometer at 260 nM/280 nM (GeneQuant, Amersham Pharmacia Biotech, Dübendorf, Switzerland). The plasmids are then aliquoted and stored at −20° C.

Digestion of Plasmids

5 μg of plasmids obtained as described above are mixed together with 3 μl 10× restriction enzyme buffer (Roche), 2 μl 10 U/μl Restriction Enzymes NsiI (Roche) (1 U is the amount of enzyme required to cleave 1 μg γ DNA at 37° C. in one hour), and 20 μl PCR-H₂O. The mixture is incubated for 90 minutes at 37° C.

Electrophoresis

The linearized plasmids are then analysed by electrophoresis on a 1.5% agarose gel (Invitrogen) (1.5 g agarose, 100 ml 1×TBE, 10 μl 10 μg/μl Ethidiumbromide (Merck). 1.2 μl of linearized plasmid is mixed with 2 μl 5× loading buffer, 7 μl of Q-H₂O, and loaded on the gel. Electrophoresis is then performed with 80 V for about 90 minutes.

Precipitation

28.8 μl linearized plasmid, 70 μl PCR-H₂O, 10 μl 3M NaAc, pH 5.2 (Merck), and 275 μl EtOH (100%) is mixed and incubated over night at −80° C. (or 10 minutes at room temperature). The solution is then centrifuged for 30 minutes, 14000 rpm at 4° C. The supernatant is discarded, the pellet washed in 1 ml EtOH (70%, −20° C.), dried for 5 minutes, and resuspended in 11 μl PCR-H₂O.

RNA Synthesis

11 μl of linearized template is incubated for 15 minutes at 37° C. and immediately put on ice to prevent circulation. 6.8 μl 5× RiboMAX T7 buffer (RiboMAX Large Scale RNA Production System (Promega, Wallisellen, Switzerland)), 6.8 μl 25 mM rNTP mix (1.7 μl of ATP, CTP, GTP and UTP (100 mM) each), 6 μl PCR-H₂O, and 3.4 μl T7 enzyme mix are then added and incubated 4 hours at 37° C.

mRNA Isolation with Magnetic Particles

400 μl Lysis-buffer (mRNA Isolation Kit (Roche) is mixed with 30 μl of synthetic RNA, incubated for 5 minutes at 65° C., and put on ice immediately. 15 μl of 20 μM biotin-labelled primer (Microsynth) (5′-TTTCCACACCCTAACTGACA-3′, SEQ ID NO: 23), is then added.

The magnetic particles are removed from storage medium and washed once in 500 μl lysis buffer. The RNA and primer mix is then added to the magnetic particles and incubated for 10 minutes at 37° C. The magnetic particles are washed three times with 500 μl washing buffer, and the specific RNA is eluted in 25 μl PCR-H₂O by incubation for 2 minutes at 65° C. The magnetic particles are then removed with a magnet.

4 μl of the purified RNA is diluted in 76 μl of DEPEC-H₂O and measured photospectrometricaly at 260 nM/280 nM (GeneQuant, Amersham Pharmacia Biotech, Dübendorf, Switzerland). The purified RNA is then aliquoted and stored at −80° C.

### Example 4

### Real Time Polymerase Chain Reaction (RT-PCR)

Each RT-PCR may be carried out in a total volume of 25 μl containing cDNA reverse-transcribed as described above from 25 ng and 0.375 ng total RNA for p73 and 7S respectively. Dual labeled (FAM/TAMRA) gene specific probes and TaqMan Universal PCR Master Mix (Applied BioSystems, Rotkreuz, Switzerland) may be used for the RT-PCR. By way of example, the following primers and probes may be used.

Primers (all 10 μM) (Microsynth) specific for 7S RNA: Forward primer: 5′-ACCACCAGGTTGCCTAAGGA-3′ (SEQ ID NO: 24); Reverse primer: 5′-CACGGGAGTTTTGACCTGCT-3′ (SEQ ID NO: 25). Primers specific for TA p73: Forward primer: 5′-GCACCACGTTTGAGCACCTC-3′ (SEQ ID NO: 26); Reverse primer: 5′-TCCGCCCACCACCTCATTA-3′ (SEQ ID NO: 27). Primers specific for ΔN p73: Forward primer: 5′-GGAGATGGGAAAAGCGAAAT-3′ (SEQ ID NO: 28); Reverse primer: 5′-GTGGACCGAGCGGGAGAG-3′ (SEQ ID NO: 29).

Oligonucleotide probes (FAM/TAMRA labelled) (Applied Biosystems): specific for 7S RNA—probe (300 μM): 5′-TGAACCGGCCCAGGTCGGAAAC-3′ (SEQ ID NO: 30); specific for TA p73—probe (150 μM): 5′-TCCGACCTTCCCCAGTCAAGCCG-3′ (SEQ ID NO: 31); and specific for ΔNp 73—Probe (150 μM): 5′-CAAACGGCCCGCATGTTCCC-3′ (SEQ ID NO: 32).

Generally, in performing the quantitative real-time RT-PCR, a Primer/Probe mix including 7.75 μl PCR-H₂O, 0.75 μl forward primer, 0.75 μl reverse primer, and 0.75 μl probe is prepared along with a Sample/PCR buffer mix for detecting TA p73 and ΔN p73 transcripts including 12.5 μl TaqMan universal master mix and 2.5 μl template (cDNA). A Sample/PCR buffer mix for detecting 7S RNA transcripts including 3 μl of template (cDNA) diluted in 297 μl of PCR-H₂O is also prepared. 2.5 μl of the dilution is then mixed together with 12.5 μl of TaqMan universal PCR master mix to form the 7S Sample/PCR buffer mix.

10 μl of the primer/probe mix and 15 μl of the sample/PCR buffer mix are then pipetted into a 96-well reaction plate and covered either with optical caps or with adhesive cover. The 96-well reaction plate is then centrifuged and placed in the ABI PRISM® 7700 Sequence Detection System. Amplification can consist of 1 cycle at 50° C. for 2 min, followed by 1 cycle at 95° C. for 10 min, 44 cycles at 95° C. for 15 sec, and final elongation at 60° C. for 1 min. The data analysis can be performed using ABI Prism software. Further, all measurements may be performed twice, and the arithmetic mean used for further calculations.

### Example 5

### Cell Culture Growth

Generally, in performing experiments described herein, cell cultures and samples can be grown in Dulbecco's Modified essential Medium (DMEM) or RPMI-1640 supplemented with 10% (v/v) Fetal Bovine Serum, 1.2 g bicarbonate per liter, 1% (v/v) non-essential amino acids and 15 mM N-2-hydroxyethylpiperazine-N′-2-ethanesulfonic acid, at 37° C. in a humidified atmosphere of 5% (v/v) CO² in air. All cell culture chemicals and reagents may be obtained from Sigma (Buchs, Switzerland). Suitable mammalian host cultured cell types include, but are not limited to CHO, BHK, HeLa, PLC, Jurkat, HT-1080, Hep G2, ECV304, COS-7, NIH/3T3, HaCat, LCL, HUVEC, NSO and HL60.

### Example 6

### Western Blot Analysis

Protein extraction can be performed as is known in the art (see, e.g., Zwahlen et al., Int J Cancer, 88: 66-70 (2000)), and 30 μg of total cellular protein can be size fractionated on an 8% SDS-polyacrylamide gel and blotted onto nitrocellulose (Protran; Schleicher and Schuell, Dassel, Germany). Equal loading and transfer efficiency can be assessed by ponceau staining. A polyclonal p73 antibody (AB7824, Chemikon, Temecula, U.S.A.) can be used for the detection as is known in the art (see Peters et al., Cancer Res., 59: 4233-6 (1999)). As standards for the Western blots, ΔN p73α/β and TA p73α/β (the latter 2 subcloned from pcDNA3-HA plasmids (see De Laurenzi et al., J Biol Chem., 75:15226-31 (2000)) can be synthesized in vitro by TNT T7 Quick Coupled Transcription/Translation System (Promega; Wallisellen, Switzerland) according to the manufacturer's protocol. To assess for protein quality, 30 μg of protein can be fractionated and blotted as above, and detected with a rabbit polyclonal antibody against actin (A2066; Sigma, Buchs, Switzerland).

### Example 7

### Reporter Assays

The luciferase reporter assay can be performed as follows. 10⁵ Saos-2 cells are plated and transfected with Lipofectamine 2000 (Life Technologies, Basel, Switzerland) according to the manufacturer's protocol. Indicated amounts of a reporter plasmid containing firefly luciferase under control of the p21^{WAF1/Cip1} promoter, together with p53, TA p73 and ΔN p73 expression plasmids can be used for transfection. A total of 2 μg of plasmids are transfected using pcDNA3 (Invitrogen) to adjust for equal amounts. Generally, 10 ng of a renilla luciferase expression plasmid (pRL-CMV, Promega, Wallisellen, Switzerland) is co-transfected to normalize for transfection efficiency. All transfections may be done in triplicate, and the Dual-Luciferase reporter assay system (Promega) may then be carried out after 24 hrs from transfection according to the manufacturer's protocol.

### Example 8

### Cloning of Human ΔN p73 Isoforms

In order to clone the human homologues of the mice ΔN p73 isoforms, a BLAST search in the Genbank database can be performed using a sequence from mouse exon 3′ (y19235). This search allows for the identification of a genomic clone (AL 136528) containing the entire human p73 gene. Based on the human p73 genomic sequence, forward primers within human exon 3′ (SEQ ID NO: 8) can be designed to amplify the entire coding sequence of various ΔN p73 isoforms including the α, β, and γ C-terminal splice variants (SEQ ID NOs: 1, 3, and 5, respectively).

By way of example, human ΔN p73 isoforms can be cloned by the methods described above, or by that set forth below. PCR is carried out with 100 ng of the transcribed cDNA in a 50 μl reaction with, e.g., Expand High Fidelity PCR System enzyme mix (Roche, Basel, Switzerland) according to manufacturer's protocol. By way of example, amplification can consist of 1 cycle at 94° C. for 2 min, followed by 35 cycles of 94° C. for 15 sec, 59° C. for 30 sec, and 72° C. for 1.5 min with a cycle elongation of 5 sec for cycles 11-35, and a final elongation at 72° C. for 7 min. The primers designed from the identified genomic clone can be as follows: 5′-ATGCTGTACGTCGGTGACCC-3′(SEQ ID NO: 33) and 5′-TCAGTGGATCTCGGCCTCC′3′ (SEQ ID NO: 34), which allow for the amplification of different C-terminal splice variants.

The PCR product can then be cloned into, e.g., the pcDNA3.1/V5-His Vector (TA Cloning Kit, Invitrogen, Groningen, The Netherlands) according to the manufacturer's protocol and ΔN p73α (SEQ ID NO: 1), ΔN p73β (SEQ ID NO: 3), and the ΔN p73γ (SEQ ID NO: 5) variants can be sequenced completely in both directions.

FIG. 3 illustrates a schematic representation of the 5′ end of the human p73 gene giving rise to TA and ΔN splice variants. Distances are not proportional to genomic distances. White boxes represent exons (numbers are indicated above in arabic), dark grey shading represents 5′ untranslated regions. The light grey shading indicates the two promoter regions: P1 (coding for TA p73) and P2 (coding for ΔN p73, SEQ ID NO: 7).

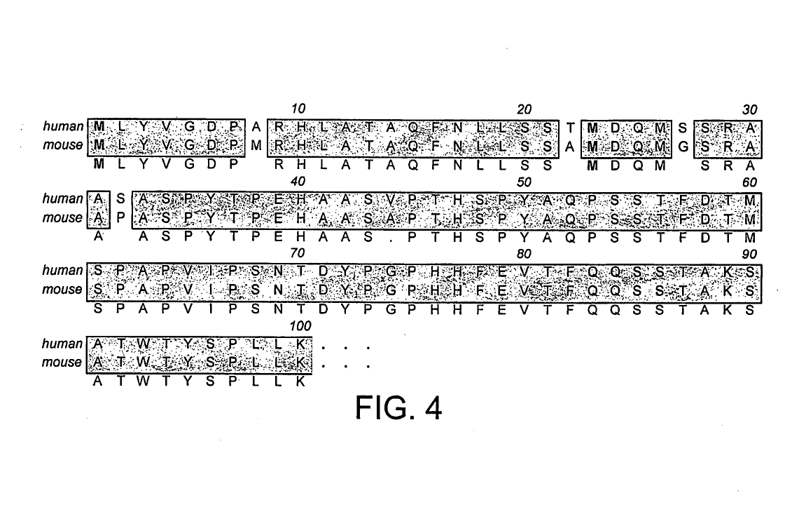

As shown in FIG. 4, the human ΔN p73 isoforms are homologous to the mouse ΔN p73 isoforms. More particularly, FIG. 4 illustrates the alignment of mouse (SEQ ID NO: 13) and human ΔN amino-termini (SEQ ID NO: 12), wherein four amino acids differ between the two sequences, only one of them is encoded by exon 3′ (SEQ ID NO: 8). Initial methionines are in bold. The consensus sequence is shown below (SEQ ID NO: 14).

### Example 9

### Characterization of Exon 3′

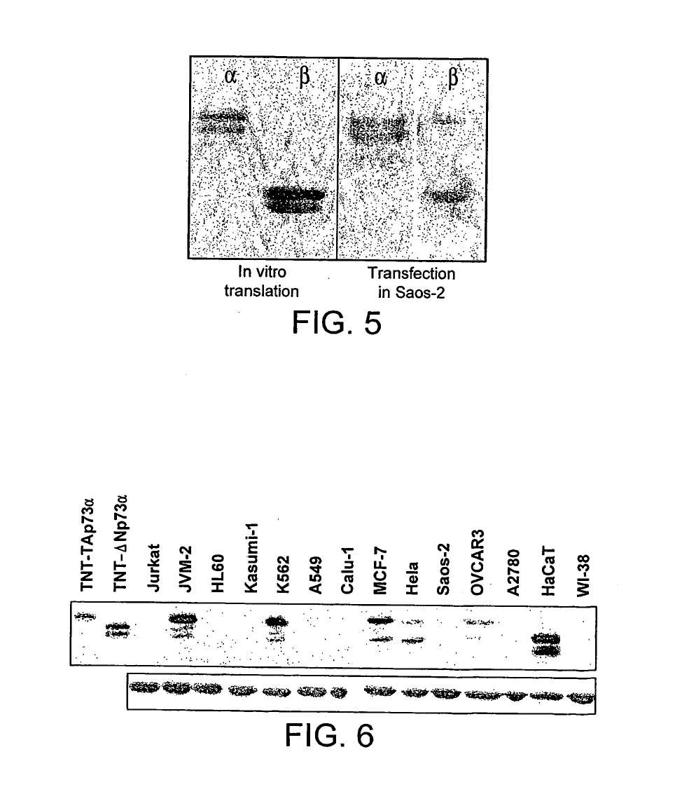

The sequence of exon 3′ (SEQ ID NO: 8) contains two different in frame ATGs and translation can start with either one. The existence of two different translation start sites can be confirmed by in vitro translation of a ΔN p73 construct (FIG. 5) and by Western blot analysis of over-expressed (FIG. 5) and endogenous p73 (FIG. 6).

As mentioned above, FIG. 5 illustrates a Western blot of in vitro translated (left) and over-expressed (right) ΔN p73α (α) and ΔN p73β (β) proteins, showing that two different forms derived from two different ATGs exist. In addition, the use of both ATGs can be confirmed by in vitro translation of ΔN p73 in which either one of the two ATGs are mutated and showing that only one protein band is present.

### Example 10

### Cloning and Characterization of ΔN p73 Promoter

To confirm that the transcription of ΔN p73 isoforms is driven by a different promoter (SEQ ID NO: 7) located upstream of exon 3′ (SEQ ID NO: 8), the beginning of the mRNA can be determined by 5′ RACE, and then a genomic fragment upstream of the transcription start site can be cloned by PCR using primers designed on the genomic sequence previously described (AL 136528). Amplification of the 5′ upstream region of ΔN p73 is performed using the following primers: 5′-GCTGGGCCTTGGGAACGTT-3′ forward primer (SEQ ID NO: 35); and 5′-GGCAGCGTGGACCGAGCGG-3′ reverse primer (SEQ ID NO: 36) designed on the genomic sequence of clone ALI36528, with High Fidelity Taq (Life Technologies). Amplification consists of 1 cycle at 94° C. for 3 min, followed by 40 cycles of 94° C. for 45 sec, 60° C. for 45 sec, 72° C. for 2 min, and final elongation at 72° C. for 7 min on a GeneAmp PCR 9600 (Perkin Elmer, Rothrist, Switzerland). The fragment is first cloned into PCR 2.1 Invitrogen, then digested XhoI HindIII, and cloned into pGL3-Basic.

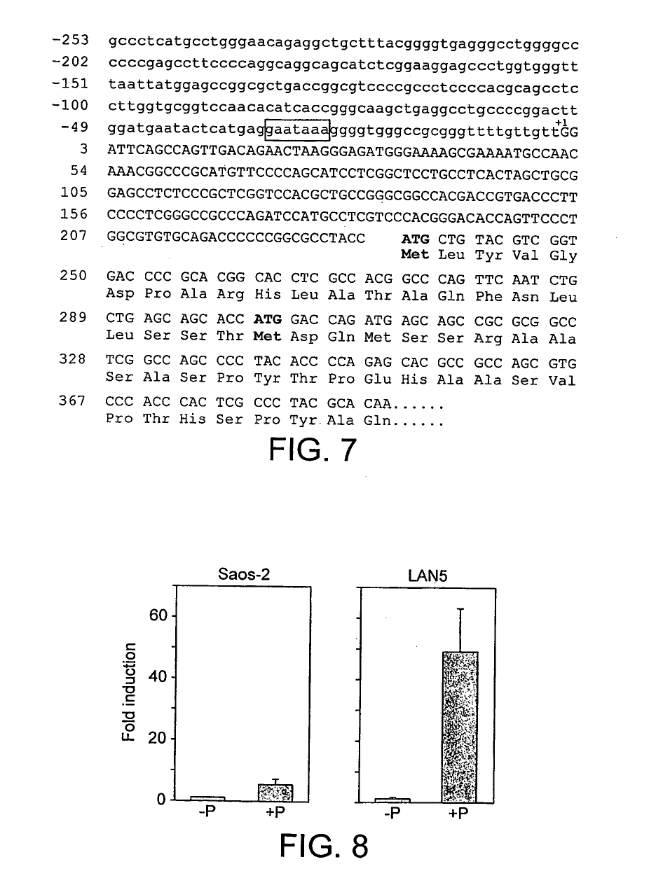

As shown in FIG. 7, the mRNA contains 253 nucleotides of 5′ untranslated RNA which is not interrupted by introns. A putative TATA box is in position −25. More particularly, FIG. 8 shows a partial sequence of part of the ΔN promoter region with the transcribed sequence capitalized, the first transcribed nucleotide numbered as +1. The two initiation codons are in bold.

To demonstrate the ability of the ΔN p73 promoter to drive transcription, an approximately 2 kb fragment (from nucleotide 43580 to nucleotide 45728 of sequence, AL 136528) of the 5′ flanking sequence is cloned into the pGL3-Basic vector upstream of the luciferase gene (ΔN p73Luc). Transcription of the luciferase gene is then be monitored in a reporter assay, as is known in the art. This fragment drives the expression of the luciferase gene when transfected into cell lines expressing high levels of ΔN but not in those that are negative or low expressing.

FIG. 8 illustrates experiments performed in two representative cell lines: low ΔN p73-expressing Saos-2 cells and high ΔN p73-expressing Lan-5. A luciferase reporter assay of Saos-2 (left) and Lan-5 (right) cells transfected with pGL3-Basic alone (−P) and with the same vector containing 2 kb of genomic sequence upstream of exon 3′ (+P) is shown. The histogram represents the results of five distinct experiments.

### Example 11

### Characterization of C-Terminal p73 mRNA Splice Variants

RT-PCR of the entire open reading frame of p73 using forward primers specific for either TA or ΔN, followed by a nested PCR spanning exons from 8 to 14 common to both p73 variants, shows that mRNA for all p73 C-terminal splice variants exist both as TA and ΔN variants. To determine whether ΔN p73 is expressed as all three C-terminal splice variants, 100 ng of cDNA obtained as described above from various different cell lines (e.g., JVM-2, HaCaT, and MCF-7) can be amplified by PCR with forward primers specific for TA p73 and ΔN p73, and a reverse primer common to both variants. Exemplary primers include: for TA p73 5′-AAGATGGCCCAGTCCACCGCCACCTCCCCT-3′ forward primer (exon 2, SEQ ID NO: 37); for ΔN p73 5′-ATGCTGTACGTCGGTGACCC-3′ forward primer (exon 3′, SEQ ID NO: 38); and common reverse primer 5′-TCAGTGGATCTCGGCCTCC-3′ (exon 14, SEQ ID NO: 39). An Expand High Fidelity PCR System enzyme can be used as described above, but with an annealing temperature of 61° C.

Following amplification, 0.1 μl of the first PCR product is used as a template for a nested PCR using a forward primer in exon 8 and a reverse primer in exon 14 (26, 37), 15 cycles are used for TA p73 and 18 cycles for ΔN p73. The amplicons are then blotted and detected with a probe spanning from exon 8 to 10. The detection by blotting and hybridization is performed as is known in the art (see Tschan et al., Biochem Biophys Res Commun., 277:62-5 (2000): Zwahlen et al., Int J Cancer, 88, 66-70 (2000)).

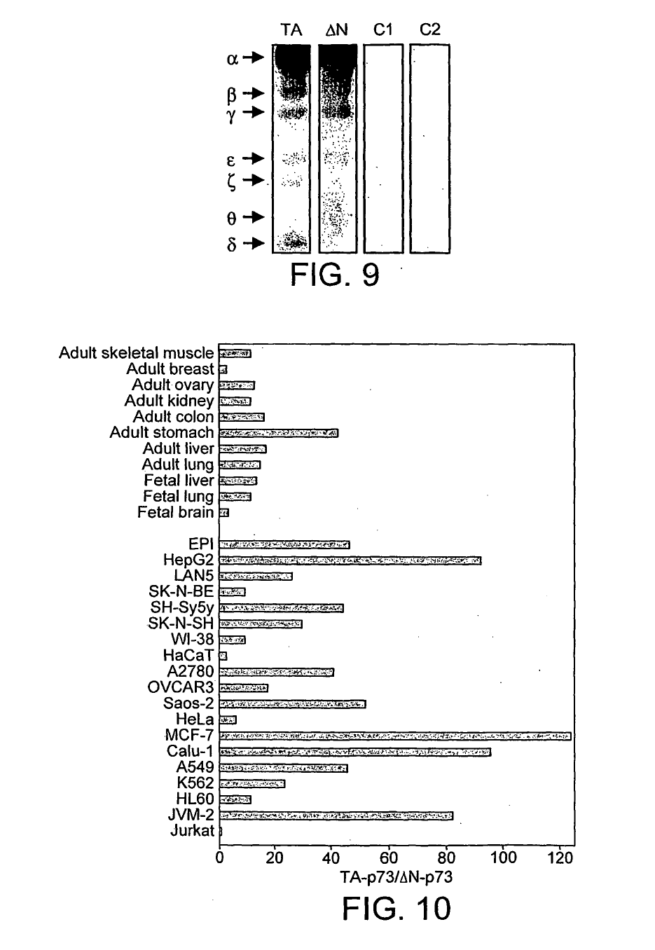

A representative experiment for JVM-2 is shown (FIG. 9). FIG. 9 illustrates the results of a RT-PCR demonstrating the existence of all different C-terminal isoforms with both TA and ΔN amino-termini although at different levels. Controls omitting the first RT reaction (C1) or omitting RNA in the amplification mix (C2) are shown.

Further, Western blot analysis with an antibody directed against the C-terminus of p73α shows that ΔN and TA p73 proteins is detected only in a small subset of tested cell lines, and that with the exception of HaCaT cells, the TA isoforms are the most represented. FIG. 6 illustrates Western blots of protein extracts from different cell lines. 30 μg of protein extracts from each indicated cell line is separated electophoretically, blotted and revealed with an anti-p73 antibody as described above. A representative experiment of three performed is shown wherein TA p73α TNT and ΔN p73α TNT indicate in vitro translation of TA and ΔN p73α respectively. The bottom lane represents actin control.

### Example 12

### Expression Pattern of Human ΔN p73 Isoforms

Since it has been previously reported (see Kaghad et al., Cell, 90: 809-19 (1997); De Laurenzi et al., J Exp Med, 188: 1763-68 (1998)) that in most tissues and cell lines p73 is expressed at very low levels, a very sensitive quantitative real-time RT-PCR method to evaluate the expression levels of TA and ΔN isoforms in different tissues and cell lines was developed. More particularly, quantitative real-time RT-PCR is used for absolute quantitation of p73 N-terminal variants using 7S RNA as an internal standard as described above.

For the determination of absolute transcript number analysis, cDNA is amplified from a cell'sample (e.g., JVM-2 cells) according to the standard PCR method described above. The amplicons are then cloned into, e.g., a pcDNA3.1/V5-His vector as described above and the constructs are verified by sequencing. After digestion with Nsi I (Roche), a T7-dependent RNA synthesis is performed with the RiboMAX™ Large Scale RNA Production System (Promega, Wallisellen, Switzerland) according to the manufacturer's protocol. The synthesized RNA is extracted with a 5′-biotinylated oligo and a mRNA Isolation Kit (Roche), and quantified photospectrometrically. Molecular concentrations are calculated and random-primed cDNA synthesis is performed with the purified RNA adjusted to 200 ng with yeast RNA. A series of dilutions is prepared and measured by real-time quantitative RT-PCR as described above.

Exemplar results are summarized in Table 4 show that normal human tissues and cell lines TA isoforms are the most represented. While normal tissues have a ratio always below 20, cancer cell lines are almost always above. FIG. 10 illustrates the same results shown in Table 4.

TABLE 4

| | TA/7s | ΔN/7s | TA/ΔN |
|---|---|---|---|
| normal tissues | | | |
| Adult skeletal muscle | 9.48E−07 | 8.67E−08 | 10.9 |
| Adult breast | 1.06E−05 | 4.25E−06 | 2.5 |
| Adult ovary | 8.18E−06 | 6.29E−07 | 13.0 |
| Adult kidney | 1.22E−06 | 1.05E−07 | 11.6 |
| Adult colon | 2.21E−06 | 1.39E−07 | 15.9 |
| Adult stomach | 1.77E−06 | 4.13E−08 | 42.9 |
| Adult liver | 4.35E−06 | 2.61E−07 | 16.6 |
| Adult lung | 8.76E−06 | 6.01E−07 | 14.6 |
| Fetal liver | 2.28E−06 | 1.71E−07 | 13.3 |
| Fetal lung | 5.00E−05 | 4.42E−06 | 11.3 |
| Fetal brain | 2.70E−06 | 9.15E−07 | 2.9 |
| cell lines | | | |
| EPI | 1.63E−06 | 3.47E−08 | 47.1 |
| HepG2 | 5.65E−05 | 6.10E−07 | 92.5 |
| LAN5 | 5.93E−05 | 2.26E−05 | 26.3 |
| SK-N-BE | 1.44E−07 | 1.68E−07 | 8.6 |
| SH-Sy5y | 1.47E−05 | 3.31E−07 | 44.5 |
| SK-N-SH | 4.09E−05 | 1.37E−06 | 30.0 |
| SK-N-AS | 2.04E−08 | ND | — |
| WI-38 | 1.93E−07 | 2.03E−08 | 9.5 |
| HaCat | 2.49E−06 | 1.05E−06 | 2.4 |
| A2780 | 3.35E−05 | 8.10E−07 | 41.3 |
| OVCAR3 | 3.78E−05 | 2.15E−06 | 17.6 |
| Saos-2 | 9.03E−07 | 1.72E−08 | 52.4 |
| Hela | 3.47E−07 | 6.06E−08 | 5.7 |
| MCF-7 | 1.77E−05 | 1.43E−07 | 123.8 |
| Calu-1 | 5.02E−06 | 5.24E−08 | 95.8 |
| A549 | 6.77E−07 | 2.87E−08 | 23.6 |
| K562 | 2.94E−05 | 6.33E−07 | 46.4 |
| H160 | 4.84E−07 | 4.34E−08 | 11.2 |
| JVM-2 | 3.37E−04 | 4.07E−06 | 82.8 |
| Jurkat | 1.33E−09 | 6.30E−09 | 0.2 |
| Kasumi-1 | ND | ND | — |

More particularly, Table 4 shows mRNA expression of TA p73 and ΔN p73 determined by real-time quantitative RT-PCR. Values are expressed as a ratio between the number of transcripts measured for p73 TA or ΔN and 7s ribosomal RNA in 25 ng of total RNA. The same RNA sample is used for all three amplification reactions (TA, ΔN and 7s). Individual cell lines are grown as indicated above, and 25 μg of mRNA is used for real time RT-PCR analysis, as described above. ND indicates cases in which no amplification of the gene are obtained under the experimental conditions used.

Fetal tissues express 10 fold more p73 (both TA and ΔN) than the corresponding adult tissues, underlining its important role in development. Interestingly, breast and ovary show the highest expression levels in adult normal tissues. In all cases TA isoforms are more expressed than ΔN in the same sample and the TA/ΔN ratio is always higher than 1 (FIG. 10). Moreover, while in normal adult and fetal tissues the ratio is always below 20 (with the only exception of adult stomach), all cancer cell lines show a much higher TA/ΔN ratio, suggesting a possible role for this gene in cancer. The most striking difference is found in MCF-7 a breast cancer cell line in which TA is expressed more than 120 fold more than ΔN. HaCaT, a transformed non tumoral keratinocyte cell line, shows a TA/ΔN ratio within the normal limit.

Expression levels of the ΔN and TA isoforms detected by PCR do not always correspond to those measured by Western (this is particularly evident for Hela and HaCaT cells).

### Example 13

### Characterization of ΔN p73 Isoform Function

Mouse p73 and p63 ΔN isoforms have been shown to act as dominant negatives, thus regulating the activity of the full length family members. See, e.g., Yang et al., Nat Rev Mol Cell Biol., 1:199-207 (2000); Yang et al., Nature, 404: 99-103 (2000); Pozniak et al., Science, 289: 304-6 (2000 ). In order to show that the human ΔN isoforms are also functioning as dominant negatives on TA p73 and on its homologue p53, the different ΔN isoforms can be cloned into a mammalian expression vector (e.g., pcDNA-3.1) under the control of the CMV promoter, and co-transfection experiments can be performed.

To estimate DNA fragmentation and thereby analyze apoptosis, Saos-2 cells are plated to approx. 50% confluency and transfected, using Lipofectamin 10 2000 reagent (Life Technologies) according to the manufacturer's protocol, with either TA p73α or p53 in combination with pCDNA3-HA or ΔN p73α, together with a GFP-spectrin expression vector at a 1 to 5 ratio. Cells are collected at 800×g for 10 minutes and fixed with 1:1 PBS and methanol-acetone (4:1 v/v) ‘solution at −20° C.

Hypodiploid events and cell cycle of GFP positive cells are then evaluated by flow cytometry using a propidium iodide (PI) staining (40 mg/ml) in the presence of 13 kU/ml ribonuclease A (20 minutes incubation at 37° C.) on a FACS-Calibur flow cytometer (Becton Dickinson, California, USA). Cells are excited at 488 nm using a 15 mW Argon laser, and the fluorescence is monitored at 578 nm at a rate of 150-300 events/second. Ten thousand events may be evaluated using the CellQuest Program. Electronic gating FSC-a/vs/FSC-h may be used, when appropriate, to eliminate cell aggregates.

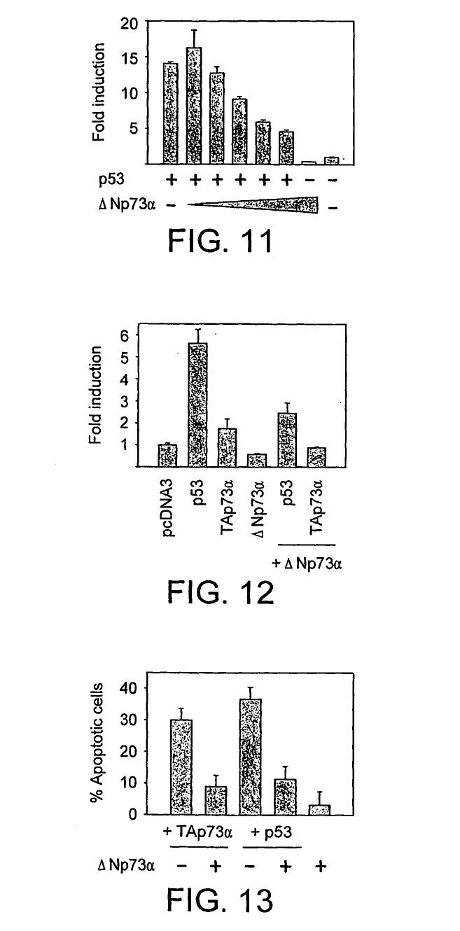

As shown in FIGS. 11-13, ΔN p73 is capable of blocking the ability of either p73 or p53 to transactivate the p21 promoter in a dose dependent manner. FIG. 11 shows a luciferase assay with Saos-2 cells transfected with 1.8 μg of p21-luc, 20 ng of p53, and increasing concentrations (from 30 to 180 ng) of ΔN p73α. The histogram reports the results of three distinct experiments performed. FIG. 12 shows a luciferase assay with Saos-2 cells transfected with 1 μg of p21-luc, and 40 ng of p53, or TA p73α alone or in combination with 360 ng of ΔN p73α. The histogram reports the results of five distinct experiments performed.

The ability of ΔN p73 to interfere with a highly important cellular function of p53, namely the ability to induce apoptosis, can also be investigated. In fact, ΔN p73 is able to significantly reduce apoptosis induced by over-expression of TA p73 or p53 when co-transfected into Saos-2 cells. FIG. 13 shows an evaluation of hypodiploid apoptotic events after PI staining of Saos-2 cells transfected with 200 ng of p53, or TA p73α alone or in combination with 200 ng of ΔN p73α. The histogram reports the results of five distinct experiments performed demonstrating that ΔN p73 is able to act as a natural dominant negative regulator of TA p73 and p53.

### Example 14

### Antibodies

Polyclonal and monoclonal antibodies against ΔN p73 or fragments thereof (e.g., exon 3′) are generated by standard techniques known in the art. Rabbits or Balb/C mice are immunized with glutathion S-transferase (GST)-ΔN p73 fusion protein for polyclonal and monoclonal antibody preparation respectively. The ΔN p73 antibody is designed so as to minimize the likelihood of generating antibodies that cross-react with p53 or TA p73. The antibodies are screened based on their ability to detect the original immunogen by Western blotting and immunoprecipitation analysis.

### Example 15

### Transfections

A lymphoblastoid cell line, C3ABR established from a normal individual is transfected with ΔN p73 cloned into pMEP4, wild-type p53, mutant p53, and pMEP4 alone. Transfections are carried out as is known in the art. Selection is carried out with hygromycin B (Roche) at 0.2 mg/ml and stably transfected cells are usually obtained at 3-4 weeks after tansfection. Anti-Fas and cisplatin-induced cell death in transfected cells is analyzed at desired time intervals by morphological examination.

Saos-2, p53-null osteosarcoma cells, are transiently transfected with pcDNA-ΔN p73 and other expression constructs by the calcium phosphate method. The DNA precipitates are left on the cells for 6 hours. Whenever needed, an empty vector may be used to maintain a constant amount of DNA in each transfection mix. Cells are subsequently shocked for 1 minute with medium containing 10% glycerol. Twenty-four hours after the removal of the precipitates, the cells are harvested for CAT assays.

For apoptotic assays, cell may be collected 60 hours post-transfection. Floating and adherent cells are combined, fixed in methanol, and stained for p53 using a mixture of anti-p53 antibodies (DO-1 and Pab1801) followed by an FITC-conjugated secondary antibody. Samples are analyzed in a cell sorter (FACS Calibur) using the CellQuest software (Becton Dickinson, Basel, Switzerland). The apoptotic fraction of the transfected cells is determined by quantitating the number of cells possessing a sub-GI DNA content as is known in the art. The effect of TA p73 and ΔN p73 on cell death may be determined by tagging the transfected cells with GRP. Cells are co-transfected with p73 expression plasmids together with a GFP expression plasmid. Cells are collected and fixed as described above and then subjected to flow cytometric analysis.

### Example 16

### Detection of ΔN p73 in Cancer Cell Lines

The presence and amount of TA p73 and ΔN p73 in various cell lines can be determined as described above. For instance, the following breast carcinoma lines may be used in the study: BT 20, DU 4475, MCF-7, MDA-MB-23 1, MDA-MB-453, SK-Br-3, T47D, UACC-893, ZR-75-10, ZR-75-30, MAI1, KPL-1, MDA-MD-435, and MDA-MD-468. However, the method is generally applicable to any desired cell line. All cell cultures are maintained in RPMI-1640 medium supplemented with 10% fetal calf serum (FCS) with the following exceptions: KPL-1 is maintained in DMEM with 5% FCS, and Mal1 in 1:1 Ham's F12:RPMI-1640 with 10% FCS. The HBL100, SV-40 transformed breast epithelial line derived from a nursing mother is used as a control. Total RNA is isolated and reverse transcribed as described above. RT-PCR is then performed as described above for TA p73 and ΔN p73, and the presence of TA p73 and ΔN p73 are thus detected.

### Example 17

### Diagnostic Correspondence Assays

The detected amounts of TA p73 and ΔN p73 described above are correlated for various types of normal and cancerous cells, with respect to tumor response to therapy, as well as different stages of tumor development. A particular cell sample's predisposition to cancer could therefore be predicted by measuring the level of ΔN p73 and TA 73 present in the cell and comparing the detected amounts to the known base-line amounts in various normal and cancer cell lines.

Additionally, detection of the level of TA p73 and ΔN p73 in various cell lines is correlated with observed sensitivity/resistance to chemotherapy or radiotherapy. In this regard, increased ΔN p73 or increased TA p73/ΔN p73 correlates with severity and resistance to chemotherapy as increased ΔN p73 (with or without TA p73) inhibits apoptosis, especially following chemotherapy or radiotherapy (which act through p53 and/or TA p73 mediated cell death). As such, a particular cell sample's sensitivity/resistance to chemotherapy could therefore be predicted by measuring the level of ΔN p73 and TA p73 present in the cell and comparing the detected amounts to the known base-line amounts in various sensitive and resistance cell lines.

### Example 18

### Vector Construction

Expression vectors can be constructed for efficient expression of ΔN p73 or fragments thereof (e.g., the ΔN p73 promoter operably linked to a heterologous nucleic acid, exon 3′, etc.) in mammalian cell lines. These expression vectors will generally include the ΔN p73 nucleic acid sequence operably linked to a promoter/enhancer sequence (e.g., the CMV promoter, p21 promoter, p53 promoter, TA p73 promoter, or the ΔN p73 promoter). The vectors can also be designed to confer antibiotic or toxin resistance through expression of resistance genes under control of a second promoter. Illustrative vectors include pcDNA3.1 and pMEP4.

### Example 19

### Antisense Therapy

Stable and effective (>95%) antisense RNA mediated inhibition of gene expression has been demonstrated for endogenous cell proteins (Hambor, et al., PNAS Vol. 85, pgs. 4010-4014, 1988). Plasmids expressing antisense RNA are generated by inserting the entire ΔN p73 cDNA or fragments (e.g., exon 3′) thereof into an expression plasmid (e.g., the pcDNA3.1/V5-His-TOPO vector described above or any suitable vector known in the art, such that the coding strand is in a 3′ to 5′ orientation relative to the location of the transcriptional promoter sequence. In this manner, the RNA which is produced by transcription of the inserted DNA will be complementary to the RNA produced from a ΔN p73 expression plasmid. The antisense plasmid is transformed into, and amplified in a host cell or sample cell of interest, as described above. Since the antisense RNA is highly amplified in the host cells, each cell contains many more copies of the antisense RNA, which thereby causes a hybridization arrest of translation of ΔN p73 protein. The host cell or sample cell can then be monitored for ΔN p73 modulation.

Generally, the antisense RNA can be use to determine if knocking out ΔN p73 kills cells per se, kills cancer cells vs. normal cells, makes cancer cells more sensitive to chemo/radiotherapy, or blocks growth factor mediated survival (especially NGF but also extending this to other systems). Further, the antisense RNA can be used to determine if knocking out ΔN p73 blocks cell-differentiation-mediated resistance to chemotherapy. Such determination can then be used to develop therapeutic antisense compositions for use in the treatment of cancer and other diseases.

### Example 20

### Pharmaceutical Composition & Delivery Thereof

ΔN p73 proteins, fragments, antisense RNA, gene therapy vectors, or ΔN p73 modulating compounds can be administered directly to mammalian subject for modulation of ΔN p73, TA p73, p53, or p63 in vivo. The compounds of interest are administered in any suitable manner, optionally with pharmaceutically acceptable carriers. Administration is by any of the routes normally used for introducing such molecules into ultimate contact with the tissue to be treated. Suitable methods of administering such compounds are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e. g., Remington's Pharmaceutical Sciences, 17^{th} ed. 1985)).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, by orally, topically, intravenously, intraperitoneally, intravesically or intrathecally. Optionally, the compositions are administered orally or nasally.

More particularly, the compounds of interest, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

The formulations of compounds can be presented in unit-dose or multi-dose sealed containers, such as ampules and vials. Solutions and suspensions can be prepared from sterile powders, granules, and tablets of the kind previously described.

The dose administered to a patient, in the context of the present invention should be sufficient to effect a beneficial response in the subject over time. The dose will be determined by the efficacy of the particular taste modulators employed and the condition of the subject, as well as the body weight or surface area of the area to be treated. The size of the dose also will be determined by the existence, nature, and extent of any adverse side-effects that accompany the administration of a particular compound or vector in a particular subject. In determining the effective amount of the compound of interest to be administered, the circulating plasma levels of the compound, potential toxicities, and the potential immune responses can be considered.

### Example 21

### Study in Xenopus Model

The role of ΔN p73 in blocking p53-mediated apoptosis in the reductive cell differentiation stage of Xenopus development can be investigated to elucidate the role of ΔN p73 in apoptosis. ΔN p73 can be microinjected in Xenopus embryos, and apoptosis of the embryos can be monitored using ELISA methodologies known in the art. In this way, the role of ΔN p73 in early cell stage p53-mediated apoptosis can be monitored.

The above description, sequences, drawings and examples are only illustrative of preferred embodiments which achieve the objects, features and advantages of the present invention. It is not intended that the present invention be limited to the illustrative embodiments. Any modification of the present invention which comes within the spirit and scope of the following claims should be considered part of the present invention.

All references, publications, and patents cited herein are specifically incorporated by reference in a manner consistent with this disclosure. Reagents and compositions (e.g., nucleic acid molecule, amino acid molecules, vectors, host cells, antibodies, etc.) related to p53, p63, and TA p73 can be made using methodologies known to those of skill in the art or may be obtained from commercial suppliers.

1. An isolated nucleic acid molecule comprising a nucleic acid sequence selected from the group consisting of SEQ ID NOs: 1, 3, and 5 and a nucleic acid sequence complementary to a nucleic acid sequence selected from the group consisting of SEQ ID NOs: 1, 3, and 5.

2. The isolated nucleic acid molecule according to claim 1, wherein said nucleic acid molecule is an RNA molecule.

3. The isolated nucleic acid molecule according to claim 2, wherein said nucleic acid molecule comprises a nucleic acid sequence complementary to a nucleic acid sequence selected from the group consisting of SEQ ID NOs: 1, 3, and 5.

4. The isolated nucleic acid molecule according to claim 1, wherein said nucleic acid molecule is a double stranded nucleic acid molecule.

5. The isolated nucleic acid molecule according to claim 1, wherein said nucleic acid molecule is a single stranded nucleic acid molecule.

6. The isolated nucleic acid molecule according to claim 5, wherein said nucleic acid molecule comprises a nucleic acid sequence complementary to a nucleic acid sequence selected from the group consisting of SEQ ID NOs: 1, 3, and 5.

7. An isolated nucleic acid molecule comprising a first nucleic acid sequence selected from the group consisting of SEQ ID NO: 8 and a nucleic acid sequence complementary to SEQ ID NO: 8, wherein said first nucleic acid molecule does not include at least one of a second nucleic acid sequence selected from the group consisting of exon 1, exon 2, and exon 3 of the nucleic acid sequence encoding TA p73.

8. The isolated nucleic acid molecule according to claim 7, wherein said nucleic acid molecule is an RNA molecule.

9. The isolated nucleic acid molecule according to claim 8, wherein said nucleic acid molecule comprises a nucleic acid sequence complementary to nucleic acid sequence SEQ ID NO: 8.

10. The isolated nucleic acid molecule according to claim 7, wherein said nucleic acid molecule is a double stranded nucleic acid molecule.

11. The isolated nucleic acid molecule according to claim 7, wherein said nucleic acid molecule is a single stranded nucleic acid molecule.

12. The isolated nucleic acid molecule according to claim 11, wherein said nucleic acid molecule comprises a nucleic acid sequence complementary to SEQ ID NO: 8.

13. An isolated nucleic acid molecule comprising a nucleic acid sequence with an identity of at least 90% to a nucleic acid sequence selected from the group consisting of SEQ ID NOs: 1, 3, 5 and complements thereof.

14. An isolated nucleic acid molecule comprising a first nucleic acid sequence with an identity of at least 90% to a second nucleic acid sequence selected from the group consisting of SEQ ID NO: 8 and complements thereof, wherein said first nucleic acid molecule does not include at least one of a third nucleic acid sequence selected from the group consisting of exon 1, exon 2, and exon 3 of the nucleic acid sequence encoding TA p73.

15. An isolated nucleic acid molecule encoding an amino acid sequence selected from the group consisting of SEQ ID NOs: 2, 4, and 6.

16. An isolated nucleic acid molecule encoding an amino acid sequence of SEQ ID NO: 9, wherein said nucleic acid molecule does include at least one of exon 1, exon 2, and exon 3 of the nucleic acid sequence encoding TA p73.

17. An isolated nucleic acid molecule comprising at least 10 but not more than 1500 consecutive nucleotides of the complement of a nucleic acid sequence selected from the group consisting of SEQ ID NOs: 1, 3, and 5.

18. The isolated nucleic acid molecule according to claim 17, wherein said nucleic acid molecule comprises at least 12 but not more than 1500 consecutive nucleotides of the complement of a nucleic acid sequence selected from the group consisting of SEQ ID NOs: 1, 3, and 5.

19. The isolated nucleic acid molecule according to claim 18, wherein said nucleic acid molecule comprises at least 15 but not more than 1500 consecutive nucleotides of the complement of a nucleic acid sequence selected from the group consisting of SEQ ID NOs: 1, 3, and 5.

20. The isolated nucleic acid molecule according to claim 19, wherein said nucleic acid molecule comprises at least 18 but not more than 1500 consecutive nucleotides of the complement of a nucleic acid sequence selected from the group consisting of SEQ ID NOs: 1, 3, and 5.

21. The isolated nucleic acid molecule according to claim 20, wherein said nucleic acid molecule comprises at least 20 but not more than 1500 consecutive nucleotides of the complement of a nucleic acid sequence selected from the group consisting of SEQ ID NOs: 1, 3, and 5.

22. The isolated nucleic acid molecule according to claim 21, wherein said nucleic acid molecule is capable of selectively hybridizing to a ΔN p73 nucleic acid molecule.

23. An isolated nucleic acid molecule comprising at least 10 but not more than 272 consecutive nucleotides of SEQ ID NO: 8.

24. The isolated nucleic acid molecule according to claim 23, wherein said nucleic acid molecule is capable of selectively hybridizing to a ΔN p73 nucleic acid molecule.

25. A nucleic acid probe comprising a first nucleic acid sequence of SEQ ID NO: 32, but not at least one of a second nucleic acid sequence selected from the group consisting of exon 1, exon 2, or exon 3 of the nucleic acid sequence encoding TA p73.

26. A vector having a nucleic acid molecule comprising a nucleic acid sequence selected from the group consisting of SEQ ID NOs: 1, 3, and 5 and a nucleic acid sequence complementary to a nucleic acid sequence selected from the group consisting of SEQ ID NOs: 1, 3, and 5.

27. A vector having a nucleic acid molecule comprising a nucleic acid sequence of at least 10 consecutive nucleotides of the complement of SEQ ID NO: 8.

28. The vector according to claim 27, wherein said vector is capable of replication in a mammalian cell.

29. The vector according to claim 28, wherein said vector is capable of replication in a human cell.

30. The vector according to claim 28, wherein said vector is capable of expressing said nucleic acid sequence comprising at least 10 consecutive nucleotides of the complement of SEQ ID NO: 8.

31. The vector according to claim 27, wherein said nucleic acid sequence comprising at least 10 consecutive nucleotides of the complement of SEQ ID NO: 8 is operably linked to a promoter selected from the group consisting of a p21 promoter, a p53 promoter, a p73 promoter and a ΔN p73 promoter.

32. The vector according to claim 31, wherein said nucleic acid sequence comprising at least 10 consecutive nucleotides of the complement of SEQ ID NO: 8 is operably linked to said ΔN p73 promoter.

33. An isolated nucleic acid molecule comprising a promoter which comprises SEQ ID NO: 7 operably linked to a heterologous nucleic acid sequence.

34. The isolated nucleic acid molecule of claim 33, wherein said promoter has a transcription start site located at position 2086 of SEQ ID NO: 7.

35. The isolated nucleic acid molecule according to claim 33, where said heterologous nucleic acid sequence is capable of being expressed at a high level in fetal tissue.

36. The isolated nucleic acid molecule according to claim 33, where said heterologous nucleic acid sequence is capable of being expressed at about a ten fold higher level in fetal tissue than in adult tissue.

37. The isolated nucleic acid molecule according to claim 33, where said heterologous nucleic acid sequence is selected from the group consisting of a p53 coding sequence, a p73 coding sequence, a toxin, and a reporter gene.

38. The isolated nucleic acid molecule according to claim 33, where said heterologous nucleic acid sequence is capable of being transcribed as an antisense RNA.

39. The isolated nucleic acid molecule according to claim 37, wherein said antisense RNA is capable of binding to a nucleic acid molecule having SEQ ID NO: 8 under physiological conditions.

40. A host cell comprising a nucleic acid molecule of claim 1.

41. The host cell of claim 40, wherein said host cell is a non-human mammalian cell.

42. The host cell of claim 40, wherein said host cell is a bacterial cell.

43. The host cell of claim 40, wherein said host cell is an isolated human cell.

44. An isolated polypeptide comprising an amino acid sequence of SEQ ID NO: 9.

45. An antibody that selectively binds to a polypeptide comprising SEQ ID NO: 9.

46. A pharmaceutical composition comprising a pharmaceutically acceptable carrier and an isolated polypeptide of claim 44.

47. A pharmaceutical composition comprising a pharmaceutically acceptable carrier and an isolated nucleic acid molecule of claim 17.

48. A method for at least partially inhibiting apoptosis in a cell comprising:
providing an expression vector comprising a nucleic acid sequence encoding a polypeptide selected from the group consisting of SEQ ID NOs: 2, 4, and 6, operably linked to an expression control sequence;
introducing the expression vector into the cell; and
maintaining the cell under conditions permitting expression of the encoded amino acid in the cell.

49. A method for at least partially inhibiting the expression of at least one of a p53 molecule, a p63 molecule, and a TA p73 molecule in a cell comprising:
providing an expression vector comprising a nucleic acid sequence encoding a polypeptide selected from the group consisting of SEQ ID NOs: 2, 4, and 6, operably linked to an expression control sequence;
introducing the expression vector into the cell; and
maintaining the cell under conditions permitting expression of the encoded amino acid in the cell.

50. A method for at least partially inhibiting the production of a ΔN p73 polypeptide in a cell comprising:
providing an isolated nucleic acid molecule comprising at least 10 consecutive nucleotides of the complement of SEQ ID NO: 8;
introducing the nucleic acid molecule into the cell; and
maintaining the cell under conditions permitting the binding of the nucleic acid sequence to ΔN p73 mRNA.

51. A method for determining the presence or absence of ΔN p73 molecule in a sample comprising:
obtaining said sample; and
selectively detecting the presence or absence of a ΔN p73 molecule, wherein the ΔN p73 molecule is selected from the group consisting of a ΔN p73 mRNA and a ΔN p73 polypeptide.

52. The method according to claim 51, wherein said ΔN p73 molecule is a ΔN p73 mRNA.

53. The method according to claim 52, wherein said ΔN p73 mRNA is selectively detected by PCR.

54. The method according to claim 52, wherein ΔN p73 mRNA is selectively detected by an oligonucleotide probe which specifically hybridizes to exon 3′.

55. The method according to claim 51, wherein said ΔN p73 molecule is a ΔN p73 polypeptide.

56. The method according to claim 55, wherein said ΔN p73 polypeptide is selectively detected by an antibody.

57. The method according to claim 56, wherein said antibody is a monoclonal antibody.

58. The method according to claim 56, wherein said antibody selectively binds to a polypeptide comprising SEQ ID NO: 9.

59. The method according to claim 56, wherein said ΔN p73 polypeptide is specifically detected using an in situ assay.

60. The method according to claim 59, wherein said antibody is fluorescently labeled.

61. The method according to claim 56, wherein said ΔN p73 polypeptide is specifically detected using a Western assay.

62. The method according to claim 56, wherein said ΔN p73 polypeptide is specifically detected using a sandwich assay.

63. A method for determining the level of ΔN p73 in a sample comprising:
obtaining said sample; and
selectively detecting the level of a ΔN p73 molecule, wherein the ΔN p73 molecule is selected from the group consisting of a ΔN p73 mRNA and a ΔN p73 polypeptide.

64. The method according to claim 63, wherein said ΔN p73 molecule is a ΔN p73 mRNA.

65. The method according to claim 64, wherein said ΔN p73 mRNA is selectively detected by PCR.

66. The method according to claim 64, wherein ΔN p73 mRNA is selectively detected by an oligonucleotide probe which specifically hybridizes to exon 3′.

67. The method according to claim 63, wherein said ΔN p73 molecule is a ΔN p73 polypeptide.

68. The method according to claim 67, wherein said ΔN p73 polypeptide is selectively detected by an antibody.

69. The method according to claim 68, wherein said antibody is a monoclonal antibody.

70. The method according to claim 68, wherein said antibody selectively binds to a polypeptide comprising SEQ ID NO: 9.

71. The method according to claim 68, wherein said ΔN p73 polypeptide is specifically detected using an in situ assay.

72. The method according to claim 71, wherein said antibody is fluorescently labeled.

73. The method according to claim 67, wherein said ΔN p73 polypeptide is specifically detected using a Western assay.

74. The method according to claim 67, wherein said ΔN p73 polypeptide is specifically detected using a sandwich assay.

75. A method for determining the TA p73/ΔN p73 ratio in a sample comprising:
obtaining said sample;
selectively detecting the level of a TA p73 molecule and a ΔN p73 molecule, wherein the TA p73 molecule is selected from the group consisting of a TA p73 mRNA and a TA p73 polypeptide, and the ΔN p73 molecule is selected from the group consisting of a ΔN p73 mRNA and a ΔN p73 polypeptide; and
determining the TA p73/ΔN p73 ratio based on the detected levels of TA p73 and ΔN p73.

76. The method according to claim 75, wherein said ΔN p73 molecule is a ΔN p73 mRNA.

77. The method according to claim 76, wherein said ΔN p73 mRNA is selectively detected by PCR.

78. The method according to claim 76, wherein ΔN p73 mRNA is selectively detected by an oligonucleotide probe which specifically hybridizes to exon 3′ (SEQ ID NO: 8).

79. The method according to claim 75, wherein said ΔN p73 molecule is a ΔN p73 polypeptide.

80. The method according to claim 79, wherein said ΔN p73 polypeptide is selectively detected by an antibody.

81. The method according to claim 80, wherein said antibody is a monoclonal antibody.

82. The method according to claim 80, wherein said antibody selectively binds to a polypeptide comprising SEQ ID NO: 9.

83. The method according to claim 80, wherein said ΔN p73 polypeptide is specifically detected using an in situ assay.

84. The method according to claim 83, wherein said antibody is fluorescently labeled.

85. The method according to claim 79, wherein said ΔN p73 polypeptide is specifically detected using a Western assay.

86. The method according to claim 79, wherein said ΔN p73 polypeptide is specifically detected using a sandwich assay.

87. A method for predicting tumor resistance to treatments involving p53, p63, and/or TA p73-induced apoptosis comprising:
obtaining a sample tissue or cell;
detecting the amount of ΔN p73 molecule or a TA p73/ΔN p73 ratio in said sample; and
comparing said amount to a base-line amount in cell types of known resistance to p53, p63, and/or TA p73-induced apoptosis.

88. A method for predicting tumor resistance to treatments involving chemotherapy agents or radiotherapy agents comprising:
obtaining a sample tissue or cell;
detecting the amount of ΔN p73 molecule or a TA p73/ΔN p73 ratio in said sample; and
comparing said amount to a base-line amount in cell types of known resistance to chemotherapy agents.

89. A diagnostic assay for predicting a predisposition to cancer comprising:
detecting the amount of ΔN p73 molecule or the TA p73/ΔN p73 ratio in a tissue or cell of interest; and
comparing said amount to a base-line amount.

90. A method for identifying ΔN p73 molecule modulating compound comprising:
obtaining a sample tissue or cell which expresses ΔN p73 molecule;
exposing the sample to a putative modulating compound; and
monitoring the level or activity of ΔN p73 molecule.

91. A method for identifying compounds which modulate the expression of ΔN p73 molecule comprising:
obtaining a tissue or cell sample which expresses the SEQ ID NO: 7 operably linked to a reporter gene;
exposing the sample to a putative modulating compound; and
monitoring the activity or expression of said ΔN p73 molecule.

92. The method of claim 91, wherein said reporter gene is selected from the group consisting of green fluorescent protein and luciferase.

93. A host cell comprising a nucleic acid molecule of claim 7.

94. The host cell of claim 93, wherein said host cell is a non-human mammalian cell.

95. The host cell of claim 93, wherein said host cell is a bacterial cell.

96. The host cell of claim 93, wherein said host cell is an isolated human cell.


SEQUENCE LISTING (39 sequences)

SEQ ID NO: 1 - DNA, 1998 bp, Homo sapiens
  CDS (235..1998): Coding sequence for delta-N p73-alpha protein
ggattcagccagttgacagaactaagggagatgggaaaagcgaaaatgccaacaaacggcccgcatgttccccagcatcctcggctcctgcctcactagctgcggagcctctcccgctcggtccacgctgccgggcggccacgaccgtgacccttcccctcgggccgcccagatccatgcctcgtcccacgggacaccagttccctggcgtgtgcagaccccccggcgcctaccatgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcactgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactga

SEQ ID NO: 2 - protein, 587 aa, Homo sapiens
  peptide sequence of delta-N p73-alpha protein
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 3 - DNA, 1904 bp, Homo sapiens
  CDS (235..1587): Coding sequence for delta-N p73-beta protein
ggattcagccagttgacagaactaagggagatgggaaaagcgaaaatgccaacaaacggcccgcatgttccccagcatcctcggctcctgcctcactagctgcggagcctctcccgctcggtccacgctgccgggcggccacgaccgtgacccttcccctcgggccgcccagatccatgcctcgtcccacgggacaccagttccctggcgtgtgcagaccccccggcgcctaccatgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcactgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactga

SEQ ID NO: 4 - protein, 450 aa, Homo sapiens
  peptide sequence of delta-N p73-beta protein
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVRTWGP

SEQ ID NO: 5 - DNA, 1515 bp, Homo sapiens
  CDS (235..1515): Coding sequence for delta-N p73-gamma protein
ggattcagccagttgacagaactaagggagatgggaaaagcgaaaatgccaacaaacggcccgcatgttccccagcatcctcggctcctgcctcactagctgcggagcctctcccgctcggtccacgctgccgggcggccacgaccgtgacccttcccctcgggccgcccagatccatgcctcgtcccacgggacaccagttccctggcgtgtgcagaccccccggcgcctaccatgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcactgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattga

SEQ ID NO: 6 - protein, 426 aa, Homo sapiens
  peptide sequence of delta-N p73-gamma protein
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPPRDAQQPWPRSASQRRDEQQPQRPVHGLGVPLHSATPLPRRPQPRQFFNRIGVSKLHRVFHLPRVTEHLPPAEPDH

SEQ ID NO: 7 - DNA, 2245 bp, Homo sapiens
  Sequence of p73(delta)Np clone deposited as ECACC 01091404
tggaggaggcgctaagggccacgccaagggggtgtggccccagatcccctgtccctgtcctctgcaaggctgggccttgggaacgtttgcagaaagctgggtgccgctctggggcagaggccagtggttttgggtgcttttgagttggaaacgtgtagctcagccgcactgggatccccgcagcctggcccagatgctaagggtggagagatgcggggtctcaggcacggtgccctgggcatgggtggggctcgtgctgaaggcagcctggctgtcttccttcctcacgtccttccacttggcgctctccttttggctatttataaaaccatcaggccggccctgtgcatgggactcgcctgagtctccttttcaatgcatcattccctttggcaggagaggacaccgcctacagaggctgaggatgtgccctgtgggggtcgggagcggaacccaggccccgcctcggccctgctctgagggtctgtccatccctggggagcccgcccccaacccaagaggggtcccaggctcagaagcagaaggcaccctcatccccagggcatccccgatcccagcaggagtctcctagtgctcgccctgggctctcctgcaaggaggctgctgctttccccagaacatccagtctgggccccagccgaccccctgcagggggcttcccagagacgcccttcctgaacctgatctaccagacaaaactgtctttttctcagtcgtctcctcctgagtgctgctgcccttcctgttgggggctgagatcctctgccacaggaagagacgggcgtccaggactcacctgctgcctcccggccctagggccctgagctgggctctccaggccccagccccttggggcacaacacctggaatcgtcctttcgtcctcagcccggcctgctggtggggcagggcgggtccccagggctcctcaggcagctgcagtccaaacctcccctgccctcacccagctctgcccgctctcccgggggtgggggtggggagcgatgaggcccctgccggctctcggtggggacgacagggaggaaggaagctggggagatggagacaagagaaagcaggcaggtggtttgggatttggcaggaaaaggttggaaggaaaggggaaagggtctccgcatggatttctcagctccccatggatttctcagccctcgtgagagccacggcgccctggggactggaagtgtgggtccgcaggccccagtccccaggtttgtctgagcatagatgccctgcctgcttccagggggactcgggcccctctgccagggtcaactttgtacccaagacggctgaaatacaatggaaattcagacggcccaacagggagtggcagtcacctcaaaggccccactagacgggtgcggggcaccactgcagagcccctccctggctgtgccaaggccgtccacgcctgcagggggccccactgccgggctgttctttggcaacagtggcttgtccctgtttcctgggggcttggccagtgccagggtgggctccaaacgcacggctctgggctcttggactcacccctgctttgggcaggcagtggaaggcaggccccacaagagctgctcactcccgtcacctgtctccctcgggggtctagggtcgaacctcctgtgagcccctcctctccatgcagcccttggactggtcctggcggaccaccgagttccccgcgcagggggcaggtgcgccccacctgggtgccaagggaggcgacaccatctctcccccttggggtggcccagccttgcctaccatgatctccagggccggggctcagccctcatgcctgggaacagaggctgctttacggggtgagggcctggggccccccgagccttccccaggcaggcagcatctcggaaggagccctggtgggtttaattatggagccggcgctgaccggcgtccccgccctccccacgcagcctccttggtgcggtccaacacatcaccgggcaagctgaggcctgccccggacttggatgaatactcatgaggaataaaggggtgggccgcgggttttgttgttggattcagccagttgacagaactaagggagatgggaaaagcgaaaatgccaacaaacggcccgcatgttccccagcatcctcggctcctgcctcactagctgcggagcctctcccgctcggtccacgctgccgggcggccacgaccgtgacccttcccct

SEQ ID NO: 8 - DNA, 272 bp, Homo sapiens
  CDS (235..272): Coding sequence for exon 3-prime of delta-N p73 protein
ggattcagccagttgacagaactaagggagatgggaaaagcgaaaatgccaacaaacggcccgcatgttccccagcatcctcggctcctgcctcactagctgcggagcctctcccgctcggtccacgctgccgggcggccacgaccgtgacccttcccctcgggccgcccagatccatgcctcgtcccacgggacaccagttccctggcgtgtgcagaccccccggcgcctaccatgctgtacgtcggtgaccccgcacggcacctcgccac

SEQ ID NO: 9 - protein, 12 aa, Homo sapiens
  translation of exon 3-prime of delta-N p73 protein
MLYVGDPARHLA

SEQ ID NO: 10 - DNA, 138941 bp, Homo sapiens
  gene (35325..116666): reverse complement of gene for “TP73” aka tumor suppressor p73 as reported in GenBank Accession AL136 528
gatccttgtctgcctgtacacccgcagccgcctccccagaggtagctaatcgtgcctcccaggaagctgcagaggaaacccctccaatctgcaccccaggagcctcagacaggaaccccttgagttatgcggcacgctcagcacgagttcctcgtccagaaaccctgacgacgcagaggtgccccacaggagctgggcccgagaggctcagccttgcctccgagtggaagcatgtccacactgccgctcggacaggtttaagtgacagcaggaatgtcatgggcccctggcctgtgtgaggagtggaatcctttccttctttccagtatttattgagcgcccaactttgtacaaacaccgtgccaggtgctggaggtcgtgaggtcacaaggggctgggcctctgtactgggggtccctccccagggtagaggttggcagctacccggggtgtccagcctcacttccccacacccgtggggcctcaccatcaggcttggcgctagttcctgctcacccggacttagcttcacaatatttctcagctcagctttccaggctccaccaggtcagtgcaggctggcgcacaaatggacacacctagaccgcctctggtctaacgctttcaccaggactacggctggatcccaaactgttttatcaagggataccaggggtgggtgcctcagtctgacccagccggaaggaggtggactcaagcaggaaagaggacgcagttgagttgcagttggcatttctggtgggaacctgacttcaaaagcctccatacatgaccttgacctttgcttaattttggattctaaatcaaggtcagggccaggcacggtagctcacacctgtattcccaacacttcgggaggccaaggcaggcagattgctggagcccaggagttcaagaccagcctggacagcatagtgagaccgtctctacaaaaagcttttaaaattggccaggtatggtggcacatgcctgtggtcccagctactcaggaggctgaggtgggatgacgacagcttgagcctgggaggccaaggctgcagtgagctgttatcaccccactgcactccagactggatgacagagaaagaccctgtctcagaaaaaaaaaaaaaaaaaaaaaaggtcagctctttattgatttcctttttggcatggggccctcacaagcagggcacactctggccttgctcacgtttcatatgacaccccagtggagccctgcagttgagactgccaaatcccagcaccaccgcaaagcaggtctgctgtccagggggactgttcatcctttcctatttgtctactaagtgaggatcatgataactacttcgcagtctggttaaaggagctacatgactttgataccaagaaggtacttagaatggtgcctggtccagcaagcacgctatgactcagagatgaggggggcaacaccatgcctccctatccaggtgccctcctcctttccagccagtgatccaaccccttcacaatgcaacccaagacccgcagccctgcccatggggctccgcccaccactgccattgacgaacatttccgggctactctgttgcactctcactccgtcctctacccacatttcttgagtgtcaactctgtgtggatgctggtgtacacggcagaaccagcccgagccccagcgtacacagaccccggttctacctcggtgggcggtgggcagaggcctgggtgctcggccgccagctgctgggacagaccctcaggaggaagggctgggcataccctggactgggagctctccagggccacgcgggtgtgctgctctgcctgctgaatcatctccttcttgctctgcaaatcctcctgcagctgcttccacctctgcaggtggttctggtccagttccacctgctcctgcagctggctgaccttggtgtccaactctgagatgacacagtcccgcttggtgagctttctctccagttccgacacttgctggggacagccggggagaaaaaagaggtggggtcatttcgcccaacctgagtatgtgtggcacccatccactttttcctaaaagatggccggcggccaggtgcagtggctcacgcctgtaatcccagcactttgggaagctgcggtgggcggatcacgaggttaagagattgagaccagcctgaccaacatggtgaaaccctgtctctactaaaaatacaaaaattagctgggcatgatggcaagtgctggtagtcccagctactcgggagactgaggcaggagaatcacttgaacccaggaggcggaggttgcagtgagctgagatcacaccactgcactccagcctggtgacggagcaggactctgtctcaaaaaaaaaaaaaaaaaaaaaaggactgggaacgcttgctgggtccctccagtccagtctggacccagcaattgcgaggagccttctgtgtggacgtggggtgcagagatctgtcgagtggcatgctaccgaaggggtcctggctcagctgtgaagacgctccagccccgtcttgccctttgctgtgttgggacttacagtaagaacagtggcctggctgatcatttaacgacacccaggctgtcacctgtcggaggagctggttgtccttttcaatgagccccatcatctcttggtgtttcttatcttctcggaggttggagaactggaaggagacagaagggttggcttgggggactcccaggtttagataacagactgaattccggcatctaacttttttccttccccaaagctccctaaaataatagcagcgggatttctattgaaggcagggagaagagaagaagaaaaaggactaaaaatattttggagagtttgaaggtggctgagtggtatctgacatcccagatccaagaaagccaaatagccggcagtaggaaaagcagaaaataagcccaattcacacttgagttcctaaaagtctcagacattgttggtctgggttcttctaaacatggaggctaaaatgaggatgattggtggaatgttatttcagaaatgaagagccctaggccacgtgcagtggctcacacctgtaatcccagcactttagaaggctgaggtgggtggatcacttgaggtcgggaatttgagagaagcgtgggtaacatagatcctatctctacaaaaaataagaacattggttgggtgtggtggtgtgcacctgtggtcccagctcctcaggaggctgaggcgagaggatcacccgactctggagaggctgaggctgcagtgagctgtgatcacaccactgaactccagcctgggtgacagagtgagaacccatctcagaaaaaaaaaaaaaaggagaaaagaaaagaaaagaaactctccattaaaatgccaaagcaaagcacactgctctgccagggagcacagagtggtcacctgtgtgctcagagtccagtcaactcgaacattggagcaaagctctcagatgaatgacagacaccaaaagcaaacttgtaggaaatagaagtggtgcaagaagttagaaaactaacagtatgatgaacgctgtcatcaagggaaaatactgcaattgtgaagccaaacaagagtgcgcttcaaaaaaagaaaaatggaacactcagagaacatcagagtaaaatattataacaaaatgaaaagagctgttgataaagttaaggaaatcttctagaaagtatcggtaagagacaaagagacaaaaaggttgagataacagataaaacaagagcgggacaggaggtccaatagtgtgagagaaggccataaaaaaacaataaaaccaaagaaaattccagaactaaaggacatgagtttccagatggccactgagtgcccaatacaacagatgaaaacacaccctcaataaagcacaactctgggaggtttcagatcacctgggacaaagataggatcttacaactttccagagaggaacaaccaacccagaaggaaccatgaacgactttggacttatcatcggccaacctggaagacaacagaggaaggcctctgtgatgctgaaggaaaatggttttcaaacaggaatgctatatccaacccagatgttatgagggtatagagtaaggacatttgccaacatatcctgtccccaaaactttacccctgttcatcgtttttttttttttttttgagatggagttttgctcttgtcgcccagactggagtgcaatggtgcaatcttggctcactgcaacctccttctcctgggttcaagcgattctcctgccttggcctcctgagtagctgggattacaggcatccaccatcacaccggttaatttttgtatttttagtagagctggggtttcaccatattggtcaggcgggtcttgaactcctgacctgaggtgatccacctgcctcagcctcccaaagtgctgggattacaggtgtgagccatcgtgctcagcctcatttatcctttttgataaaactactggaggatgtgctccaccaaaaataagtgaagagggcaagaaagcaaaaggtgtgggatgcagcagttttaaagttgtggtttatgaatcttagtactgttctgtaaaacttattttatttatgtatgtgtgtgagacaaataactgtcctaatgatgtcagtgcaacattgattgtgatagaatgatacgaggaggagggtggatggaaaggctgcccttgtgtgtgttggggggcggtggcaagggaggaaaaaaaaggaagcattaaataatgttaagatggagaacaatctagaatggccactatgagcatgctatttagagatttggagatagattcataaagaacgtgctggaagaatttaaaatgatgccttaggagagggcgaatgagagggaggaggtcagaggctcctggttttcatgacaggcctcgtggaactatttgattctttaagcgatgtgcatgaataactttgataagagtaaaaacctaaaacgcttaacaaaaacgacaagagtgggctcagtggagtagcagtctctctaagcacggatagggcgttcttacccccaggactcaggggccaggagatgcttgaggagtggccaagagcacagcggctcaggagacagggacacttgcctccagcccagggacagggcagagctgtggctgggcacatgggctccagcctcccacacaccttggggaatcctagcggggccgctatcagccgcacaaccctgggccagctgcctggacaccccaggctcaaagtctccatcagtagatgggacgggcagcgcctgccccgagctgccgggggttaatgaagcctggggcccatgctcatggagcggaggccggtactgagcgcacggccaccttgtcggtcatggcttgtagctgctgcctccgctctcggagctccttctgcagcgtctcattttccgtattgatctttcgcagctgggtttcttggaaatccacttgttgttgtaaagacgcaatcactcctgggcacagaggcagaggcggtgagcaggagcaagcaggggcagtgagggacagaaggagagggccgggccactgcggcagctgggagggctctgccagcctgggtgaggctgccgacacctgcccctgctccagctcactgaccagagcaggcagcgtctgggcgtcccagctcctgggccgggaaccactgagtggccaaagcccagtgccccccgccgaggccctacctgttgcaagggagtatctggtcctggtggcctccaggttgaactggaagtccaggatgacctgctcgtactcctccagctgggccagcagctcctcctcaaacgactcggccagggaggaggcccttctcctggggatctcctcttcctccggcagctccctttcctctgagtcctcatccctgtccccttcctcctcttcctcctcctcgcttccctcctcatgcacggatcccatctggctcacacctccccaggcacccgtgtcctccaccccatcgggctccccttccaggccctcgtcctcctcgccacctcccaggtcgggctccttgcccctctccggtgccgcagactcctcctgcatctgtaagatgggtagggcaggggctgggcagggctgcggggcacccccagggcagagggctctaatgctcatggcgggggcaccggggagggcgcctccttccaggctgctgcagggtccagggagtgggagagcatctgcctggcaaggtcaccaacagtggcccaggcactgtggcctctgtcacgggtcctccctctgtcagtagactgcacctgacctgtgccttcaagggggctaacgacccacccacctcacctgctcccctggagtggggcaccccaggtcagggctgcagcagggacacttggggcctgggcctcttagttaagcagcctaggtggctcctgggcccgggtcccccactgcgtgcctgctctccaggcggcccgcacctgccaccagtgctgcagctcctcctcatgtctggagaaggccttcaggaggctcagcctgcccagcttcagcgaagcgtctgagagctgctcctgcaagacgcccgtgggaccctcagctttccagcccctcctgcagcaccccagggacagaccactgcctgtcctagaagggacagcggcctctcatgtctggggaggggacctgcatcctcctgccaaggggctgggggatgtgggtgctgtggcagtggagggcacaggaacctgcggatgggcagttctggggggcagtggggctctaggcacccccagagcagcccccagcaagtggccttgcagtaaaggacaaggccagccagccttacacacgcccactgtgcatgtcattgcctgggtggcccaccctcagacacccacccagcagcctggggaggacagagagtggaagccgccggctggccacctttccctgcccccgccctgtgtgtctaggcagccttcccttgaggcatttagggcagttggcatggtcaggggccttccccctggttcctgcttcatctctggcccccagcataaactcaggctgtctcctcaggtgtgaaagaaatcttaggaagatcaggtgaccgcccctgggcacaacacagacctgcctcgtccagtcctctgcccacacctagaggtggggtagcatccccacctgcatccccacctctaccccacccccaccccaaccccatgcccaacctcgcagggggcttcacagccagagagtggggagaagggattcctggccagggcagggtaatacaagctaccactgcagatgatgcacagagccaggcccggggaatccgcctgcagggtggcctccatgccccaggatttcacaggtgagctcagccctcattttatggataaggaccctgagccccgacagcgttgtgccttgccccattcagaaagcaagtcagaagccaggtgcaggtgcctagacatcggctcacggctctcatttgtcattttgcgcctccattcatctctcccccccctgttgcatcagctcatggctctcatttgtcatttcgtgcctctgttcgcctctcccctgcctgttgatcagaaccctgagaaccggtccacgttccagtgaggacctgataggatccagggggcctgggggtttgccaggggcctgtgtgtcactagcagctttggacgctttgaaaacaaccgtcttagagctctgcgctgtgccccgctggggctggctcaccctcctgcctggggacatggatgtctcttggcctttgcctttcagaaggcatttcagctgcatcttcagggcctccctctcctcctggagcagcaggatctcctcgtctttcttctgaacctgtatctccagctcagacaggcagtgatcctgggcagaaaaccaagggcagcccccgtgggccatgcagccacacactcttcccctgacctgcctccagcccacccaggaggagcagatctgctgtcgtggtaccccaaaaccccagggtcccagggatttcaagataggtactgcggtaaagagctagcactacacatctggagatcaagatgtgccagctctttactgcagccatgcctggcacctccagcgcctctgcacccatcctaagaacagatcctaaatcctcgtccgaggagttgcaaggcagcttccctttcggacctggcccgtggtgtgtccctgcaccccactataccacaggacaccagtcttctttgcagccccgacacttaaccggcaacaccctccaagcttccctaggggcctgtcggggagctcctcaaacttttgttctcagaatctcaactccctcaattcttcaaaattaaagacaaaaataaagatgttttcaggcaaacagaaattgaaagaatctgtgaccagcagatctgaacttagagaaatactaaagggagttcttcaggtggaaggaaaatgatcccaacagaatattctggaaggaaagataactagaaagtcccctaaatgtttggaaagtaagtgataggcttctgtgtagccaatgggtcaaaaaagaaatcacaaggaaagctagaaacattttaaactgataatgaacatccggacacatcaaagctcgtgggacgcagttaaagttgtgattaatggcaaatttatagccttaaaatgcatataccaggagggaaatggaaaatcatgacccaagtatctatctcaagagttcagaaaaagaactaaaattaatcccgcagaaagtagaagaaaaggaataaagataggtgtgaaaatttatgatataggaagggagagagggagagaggagcaagggggagagagaggagagaaggataaagcaaacgtggccacatttaacatttggggaatggaaaagtacatgggaatctttgtagtatggattcttaccactttatgcctgaaatacaaagttaaagggaaaaaaaagtgcagaaatcacatttcatcacccaagccatggtcccgagacacttgcacgtctcaaaatagtgcaaggactgctggaagggagatccgatttcctgttagagctgccgagttcacacaatgccagctgcaaacttgttgctgcaggtggctctcttgccaccaaatgagaagagtttatctatgacagcccagacgaaggcagagaggagataaagaaaagcctgatgagccccttggagctcctggaggcagccaagcctgcagcccttcccttggatggttcatacgcatgacctggtaaatcccctccctgctgcctgcatgaataacaacagcatatctgcctgactcttggctaatgatatgctcagcctatttctcagaaatgaaattacttggttaaagggtaggaacactcttgaagcagtgaaatctataataatgtacaggtagagttgcaggagtaaacaaaatgaatatcatggacccccaggagctgggactggcaagcccccttgtgtctccattcagtcatatgaccccccaacacctctgtgccctgcaccaaatcaccaacattttgtgactcatgtccataattttcatcaaaactttaccagtttcaaaagaatgcagagaaagaaggaagcagacactagagaaaaatcaacaaaatctaaagctgattctaagagaaggttattaaaattgataaaactccaatgagaccaatggggaaaaagaaaggaggcacaaggcagccacgtcgggaatgagacagaggccgtccccaccctgcccaagctgcatcaaagctagtaattatccttgtaccacttacagccttaggctgatacataaaaaatcttatgtgaaatagatgcattcctagaaaaacacagaacaccaaaactgaaactagaggaaaaagaaaatctggatatatttttaaataattgaatttgtgatttttttttttttgagacaaagtcttgcactgtcgcctgggctggcgtgcagtggctcgatctcagctcactgaaacctccgcctcctggattcaagtgattctcctgcctcagcctcccaagtagctgggattactggcacctgccaccacgtccagctattttttttttttttgtatttttagtagagatgaggtttcactatgttggccaggctggtcttgaactcctgacctcatgatccacccacctcagcctcccaaagtgctgggattgcagaggtgagccactgtgcctggcctaatttgtgatttttaaaactgctcctgtaggggcccagtacagtggttcacacctgctatcccagcactttgggaggctgaggcaagaggatcacttgagctcaagagttcgaggcaaacctgagcaacatggagaaacctcgtctccacaaaaaaacgaaaaaaattaatgggcatggtggcgtgtgcctgtggtcccagctattcgagaggctgaggtgggaagattgcttgaaccctgggaggtcaaggctgcagggagctgggatcactccactgcgctccaacctgggtgacaaaatgagaccttgtctcataataaattaaataaacaaattaaaaaataaaagtgcccctttagatggaggctgccgtgagctctgatcgtgccattgcactccagcctgggcaacagaatgagaccctttctctaaaaaataaaggctgagcgcagtggctcacgcctgtagtcccagcactttgggaggccgaggtgggtagatcacgaagtcaagagatcaagaccatcctgtctgacatggtgaaacccgtctctactaaaaatacaaaaattagctgggtgtggtggtgcgtgcctgtaatcccagctacttgggaggctgaggcaggagaattgcttgaacccgggaggcagaggtggcagcgagctgaatggcgccactgcactccagcctggcaacagagagagactctgtctcaaaaataaataaataaattaattaataaaataagagtgctcctgtgaggaatccccagacacagaagccttcaatggcaaattcttcagaattcaaggaagaaatcacatcagtcttatacaaactcttccagagaatagaaaaagaaaaaaggtgctgggtgcagtggctcacacccctaatcacagcactttgggaggccaaggcgagtggatcacttgagcccaggagttcgagacctgcctgggcaacatggggaaaccccatttctttttttttttttttttgagatggagtctcactctgtcacccaggctggagtgcagtggcgcgatctcagcacactgcaagctccgcctccctggttcacaccattctcctgcctcagcctcccaagtagctgggactacaggcacccaccaccacgcctggctaatttgttgtatttttagtagagacggggtttcgcgtgttagccaggatggtctcgatctcctgacctcgtggatctgtctacctctgcctcccaaagtgctgagattacaggcgtgagccacctcgcctggccggggaaaccccatttctacaaaaatacaaaaagtagccaggtgtggtggtacgcacctgtagtcccagctgcttgggaggctgaagtgggaggattgcttgagctcagggagctcaaggcttcagtgagctgggatcacaccaccacactccagcctgggtgacagagtgagaccctgtctcaaaaaatactactactactaataataataataaaataaaaaaagaaaataaataaaaagaaaaaaggaactgttttccagccttttctttctttcattttaatgaggccagagtaactttgatactaaaatttgatgacggaattataagacagtgaaaatataggttagtctttcttataaatatagatttaaaaaagaataacactacaatgaatcaacttgtgcataaatcaagctggttattcctgttatgatttttattatttagaatacattctctgaagcagatactgtgggtgcaaagatgtgtggacggttggaagtcatgcgtgccggtgagtctgagttccatgccggcgtaactgccacccactggggtgaggatggacaggggtgaggagctttgtcaaagcagggggtctggaggtcattttaggccatggggacctgcttgatgcagctaaccagatactagaaccagagcctgtcagggagcagggctgtcctcccagccggacgcagagagaggggtggggggccgggatttgcaccccctccgccagtaccttctcgtggatgactgagaggtaccagggcatcctctttctcaggcagctctgagatgcgactgagctgctggtgaccggactcaaagtctgggattttctcctcttccgcaagtaatcgaactcgcagatgctcttactgaagggaaggaggtaccctgtggcgacagcggaggccaagtgagaaatgagacccttccctgccgagctcctctggcgtcttcgatgacgacatccacccttccgagacttccctcggaaacccctatctgcccagacagactccaatctgtctcctaactcttgtcaccctcattttgaggacacagtgtggtgctgctgtgaacacgatggggcgcccacacccagcacagcgcgtgctgagggagtgcaggaaggtgcggaagctctggccgagacacccaggacaaacgagccggagctgggagtcgggacgagggggtcatcctgatgctgtctctgcacacctggttgcttgtcccctcctggagcctcagttttcccatctctctttttttttttttctttttgagatggagtcttgctctatctcccaagctggagtgcagtggcacgatttcagctcactgtaacctctgcctaccaaattcaagcaattctctgcctcagcctcccaagtagctgggattacaggtgcccaccacatgcccggctaatttttttgtgtttttagtagagacggggtttcaccatcttggccaggctggtcttgaactcctgaacctcgtgatccacccgccttggcctcccaaagtgctgtgattacaggcgtgagccactgcgcccggcccagtttctccatctttaaactgagaggagggaggagattctccaccttcctgaggttgaggggagctggtcactacagactctaggtttcctccagttaaggcaagaaaggagacatggtgctgggaatttcctcttggtaaatggaaacaggcacacctgtctgcgggagcagtgtcccggggggtctctacccacagtgagggatgcacccatcagccactgggtaaaggccaaggggacaaaactttcaactacgatttttactaaagacaaggggccacggtcctggggggactgcagcctatgctgtcagggaaggcagggctagttctgcacaagggaggcccggaggcctttctgtgtgagtcctcaggaggcagacagtcccgggagggaattccgcagggcacaagcagcgaccctgcaagaagcaggcctcgagctcaggggcccagcaggtgcaggcagaggccctaggggctccctcaccgtccacgttcgtgtctgagccccgcctggccaagaacaggtcctgggtgcctctccaggtctccgagctgttgtcaatctctccggacaaatcggcctcagtgggtgaacctgtagagtgttaaagatgccatctggtgactgctgcggtcttgtctccccaagatctccagggcagactcttatttatccttaaacaccaggtagggacacaggtggctttttttaatggatgcatgggactgagcaagggctgaaccaaagggtgggcttcatcctcctgcctgccctggtgtctccacctcagcagacccaagtgtggctgaatttctctagctgatgccccctttcccccggaggggcagaccctactttccttcggtaaaaatgtgcctcctaaaagtcgaacggaagctcccctgcctccagaccagcccatccaaggcagaccctgtttcccctaaaccatcacaaattgccccaatggcaggtgcctatgagcagtatgtagacgcagaccccacgcccaggacaccgtgtggcccctcagcctccccagagacctgcccacaaaaatgcctggtcctggagccctggccaccgtggagaccctcttgctgaggcagtgggggcctgaagcctagaaaggtgagggccctgcttctacctgatgctggctgagattggtggacagcacgggaccccctccccaacccccccaccccacccttgctactggttataggaatttagctcaagctcgccatccctaagaatggtgtcttgtgtggggacatggaaccagtccaggtccaggagcagatgctgggatgaagccttggctctgggcagcatcccccagatgcccatggagctcggcagctgcctgctccaagcctcctgttctcatcagagcagtgaagggtttggaccggatagctgctagggctccccaggggcactgtccctaatgagctatctggctttatggggttctttcctgaagcttccagaatcttcaaccatttccactcccctagcccctgaccctactgaatttttagtgggtgaaacaatagaaaagcaagaaggctggggagacaaatcataatcatagtggccaggagaagtgggcccccctccacccccagcttcattgctgccctggctccctgagcacagagcggatttcaaaagcagcagctggggccactgtggaggccaactctggcggctgctcgcaggctgaagggtgcaggaaggcctcccccggcccctcgactctgacaggcagtttcacaggcggtcctggtggggggctcttaccacagtgggacatggatgtggccctggtgctgaactgggggcagtcaggatgcgtggggaagacccttcgaagttccattttggacatgaagccggtgaggctggcggcatccttgggttcactctggcagagcggggaaagtaagacagacaaggaaggcccctgactgtaggccagcccatccaagggcagaccctgtttccctaaaccatcacaaatcgccccaatggcaggtgcctatgagtcgtatgtagatgcagaccccacacccaggacaccgtgtggcccctcaggcctccccagagacctgcccacaacaaccccctggttctggagcactggctgcccaccgagagcttcttaggcatcctgaagcctagacagacgggggccctgcttctgcctgactgtcggctgagattcacagacaggccccccgaccccacccacacatgctgctggtcacaggcactaagccactctcccactggggttgaaagggcctgcgtgtgtgcaggctgcagcagtgtgtcccctctgtcccctctccagcacactggatgctcagcaagagccacgcagacccactttggaaaccatagtccagccgggcatggtggctcatgcctgtaatcccagcactttgggaggccaaggcgggcagatcacatgaggttgggagtttgagaccagcctgaccaacatggagaaaccccatctctattaaaaatacaaacaaacaaacaaataaaattagccgggcaaggtggcacattgtaatcccagctactctggaggctgaggctggagaatcacttgaacccaggaggtggaggttgcggtgagccaagatcgcgccattgcactccagcccgggtgacaagagtgaaactccgtctcaaaaaaaaaaaaaaaaaaaagaaaaagaaaaaaagaaaccatagtccactaggtccaggcaatgcattcacccaggaagctgccaggagccggggtgaccatctcgggctcccccagtacacggtggcacagcgtacctgactggctgaggtcccctttcacattttctccccttcactctccttttcaagatgcaccttgggagtcttggggcaaactgtacatcttctccccttggccaaccagagacaccgggagccaaggtctgcaagaggggaggaccaacccaaagcagctaacccagaagaagccggtggcatgtggggactaaggcaccaggcccttcagaaagggagaggaccgcagggctctttccacccctagcgctcaattcgttgcctgcctggaatccccctttggtaacccatcaccagaaggcctgtggaatgctttgtgggctgacagctctatgggcctttggggaggagaagaaacaggtcacctcaaggctcgtggcagccctcctcccgggaccccataccgtcttcctgtagtagcggctgatggtctggaccgacttgctgagcgtgcgtggcttttggtgcggtttccattctgggcaccgggcgtcccggctggccatgctctggaggagcaccagggccctggccatcgagctggacatcgaactgtgcctctgagatgggctggcctccttaggtaacatgaggcgtgggatgcaaagaccaagtgagctttgttgcctgaatgtggacctgaatgaggacaggcccggcttttcccgtggatctctcttcagcctggcttggggtgtggaggggaagatggcctcgaacatgaacctgctggctggtgcagtgggaggagatcagcttccagagctccaggtcctgtgggctcctgggagcagtggtggcatcacagatgtctccatagaaaccgttattgagcaaaaggggcccgcggctggatgcgctggaggccagtgctgtgacaccaggtttttgagaagagcaaagctcaatattgaaggttgccttctgggctgggtgtggtggctcacacctgtaatcccagcactttgggatgcccaggtgggaggcatgcttgagcccaggagttaaacattttaattagccaggcatggtggcgtgcacctgtagtcccagctcctcaggaggctgagatgagaggatcacctgagcctggggaggtcgaggttgcagtgagctgtgttcacgtcactgcactccagcctggactacagagtgagaccctatctccaaaagaaaaaagaaaaaaagaaaaaaaaaaagaaagaaaagaaaaaaagaaaagtcagctcccaaggacacaggagtccagctcaaatgtctccatgtgctggcttgaagacagtaattgtattagaaaaggttaggctgggtgcggtggctcatgcctgtaatcccagcactttgggaggccgaggtgggcggatcacgaggtcaggagatcgagaccatcctggctaacacagtgaaatcccgtctctactaaaagtacaaaaaatgagccgggcgtggtggcgggcgcctgtagtcccagctacttgggaggctgaggcaggagaatggcgtgaacccaggaggcggagcttgcagtgagcagagatcgcaccagagcactccagcctgggcaacagagtgagactccatctcaaaaaaaaaaggtttagggggtgggctctgggattagaaacatttggccaagaaatacctagaagagtagataagagtagcctgactccctgggaatgattttccaggcttcaaatgcctacctcaagacatcttttaaggagagaaagacaaaacccctaccttcagaagccttgattagggtctgaatgggaagaaaggcttctctgcttgctgactgaaggaaatttgaagggggcctgccccatccacacctgtgggtatttctcttcaggtggagatgagagactgagaaaagaaacaagacgcagagacaaagtatagagaaagaacagtcagcccaggggaccggcacactcagcatgcaaggacctgcatcggcgctggtctctgagttccctcagtatttatggatcactgtttttactaccttggtgaggggagtgtggcagggcaacagggtgatggtggggagaaggtcagaagggaaacgtgagcaaaggaatctgtatcatgaataagtttaaggaaaagtactgtgcctggatgtgcacgtaggctagatttatgtttcactttacacaaacatctcagtgtagcaaagaggaacagagcagtattgctgccagcatatctcgcctccagccacaggctggttttctcctatctaagagtagaacgaatggtcggctttacaccaagacattctgttccgaaggatgagcaggagatagaaatcttcctcttatctcaactgctgcaaagaggcctccctctttaactactcctcctcagcacagacccttcatgggtatcaggctgggggatgtaaggtctttcctttcccacaaggccacatctcaggctgtctcagtggggggaaaccttggacaggctttcttgggcagaggtccctgcagctttcctcagtgtattgtgtccctggttaatagagaatggaggatggcggtgacttttaccaggcatgctgcctgcaaacggatggttaacaaggcataccctgcacagccctaaatccattaaaccttgattcaatacagcacatgcttctgtgacacagggtttcggctaaagttacagattaacagcatctcaaagcagaacaatttttcttcatacagatcgaaatggagtttcttatgtcttccttttctacatagacacagttacaatctgatctctctttcttttccccacagaaattcatgaccccatcctttccaatatgactttataaaaaatatttatcatccaaaaatctcttcagttttttgcatctaaagtacaaaacaagcctagtatatatttgaatttttgacctacagtcatataccaaataatgaaattttggtcaatcatagaccaaatgacagaccaatacaatggtagtctcttaagaatctaatactgtattttggccgggtgtggtggctcatgcctataatcccagcactttgggaggctgaggtgggtggatcccaaggtcaggagattgagaccatcctggctaatatggtgaaaccccatctctactaaaaatacaaaaattagctgggcgtggtggtgggcacctgtagtcccagctactcgggaggctggggcaggagaatggcgtgatcccgagaggcggagcttgcagtgagaccagattgcgccgctgcactccagcctgggcgacagagcgagactccatctcaaaaaaaaaaaaaaaaaaaaaaatctaatagtgtattttcactgtagcttttctgtgcttagacatgtgtagatacacacatacgttgtgttccaactgcctgcagtactcaggaacatgctgcacgggtttgcagcctcagcaacaggtatagcatatagcctaggcatagactgggctgtaaaccacgtaggtttgtgtgagcacacggtatgacgtctgcaggatggtgaaattgcctaatgatgcacttctcagaacatatccctgttattaagtgacacatactgtattttaataagaaataaagcacttttaccagcctcaaatactgagaaagaattcttgccttgccaatttctcttttgttgattattttgtagtcttccatatgaatctctaattgttttggggacttggaccacaggaaatgttgtcacattggcctgaggtccagcatgtttgaaaatcacatgtattatgctttgtttttacattggtctctgaaacgctgttcaacagactcctcgtctattaaaacactcaagtaaatttctggttcagttaacagaaactcatggtgacaatttgatcgatagcgaagccaatattaaatagaaagcttatcttgggaccgacattcaatctatacaaacaaatcaaagcttctcgtcttgtcacttcatatgaaaatggctagatttcccccagatctagaaggtatgtttggagttggcttaaatgcagaccagtggctgtgggcggagggttacccaggtgccgaggcaagagactgaaggcacaaactgtttcagcataataaagaaaatagttagaataaggatagtcatgatacaaattagatatagagatgatgaacaattatcaatcattattaatcattagcttttaatattactctttgttgcattactaacataatctagtaataaccagcgggtgcagggtcaggtgctgaagggacattgtgagaagtgaatagaaggcaagaggtgagccttctgtcacacccgcataagggccgctttagggttccttggtcaagcggtaacgccagcgtctgggaaggcacccgttacttagcagaccatgaaagggaatctcctttccttggaggagtcagggaacactctgctccaccagcttcttatggaaggctggatatcatccaggcctgcccacagtcatccggaggcctaacccctccctgtggtgcttcaatgctcatgctccttgtccactttcatgcccctcccgtactcctggttcctctttgaagttcgtagtagatagcggtagaaggaatagtgaaagtcttaaagtctttgatctttcttataagtgcagagaagaaaacgctgacgtatgctgccttctctctctgccttgcctacctaaaatgtatcctgtaatcacatgacttgcttcaccttgtcaatcacttagaagattcaccctccttaccctgcccccttgtcttgtatgcaataaatatcagcgagcccagccgtttggggccactaccagtctccacgtcttgatggtagttgtccctggggccagctgctttctatctctttgtcttgtgcctttatttattacaatctctcatctctgcacacggggagaacacccgctaagccccatagggctggaccctacaagtggcaccagaaaatgtatctgggaagcgaggcagccctggaagtctggccatcgcggggggcaaggtgtgtgtgtgttagctacaggtatctcctgctgcgtcctcatttcccagaacaaggagtgggtgggccagtgtgaagtgagggtggagtgtgaaaggcacacgtgcgtctatttgctacctaggggtcgtgtgagctcggcatccctcccctgggcccacacctcccacaggtgttgtgttcttgtactcacggaaaagcaacctgggccttcagggcctttattgataaaactcgcgcctgtagtcccagctacttggggggctgaggcaggagaattgcttgaatctgggaggcggaggttgcagtgagccgagatcacgccactgcactccaacctgggtgacagagggagactcggtctcaaaaaaaaaccaaaaaccaaaaaccaaaaacaaaacaacaaaaactgagtggaaagtcaaacactaacaagcctaagatttttggttactgtaaaaaaaaaattgtaaattgcaaagtcaattttattttcttaataagaaaataaaaccctcttgccaatcagggaaatcctgtggggagagcggctccacttggagataacgggcccagagaaaccgcctaaggtggggcagtcagcacctgggtgcaggtgcatctgggacccgtcagatctctacgccccgcgcaggctccgcccccacgcccaagaccccgccccgcgcccggagcctccctcgacttggccctgggtctgcaccgaaggccccacccctgccctcgcccagccgcgaccccgcgcttccggatccgggttgttgcgggatctcacaggctttaaaccgcggcgccgcggcgcccgggtgtggatccctagatgggagccggggatgggccgggtgcctggtgggtggcagtcggggctgacggcggcggcactttgccgcctcaggccctggacaccttcaccccgccgcctgcccaggcgggccggccctgcccgtccaccggccgccgagagtccccggccttgggtccccggggccgctgactggcctcggtcacctcccggggaaggctcccgcgcctccatctgcccccgcaggaagggaccctcttctcgcccgcgaggcttctccgggtgggatcgtcctggcccccagccctaagggatccgccccctccgagcatccgccgcccctcggagaccactccagctcggacggacccactccagcccccgctgcacgcggaagcgctcatcctccccgcctgccccgttccctcccccttctcctgtgggacaaccagggaccgcagctccccgctccccaggtgtgggggctccgacacgaacgcctctgctcgcagggcggtgagcgcagatcccacgggtccctcggtcgggggtcgaggctgcttccgtttccatcccggacccgacaatgggcgggaaaaagaaggctttacacgactacgcggcggagttcaccgacctggtggtgaagcacctgattgagcacagtgactctggggacacgtctgtggtggagaccctttactgcagggcctgcgagctgcccgtgcgcgtgcggagggaccgcatcctggaacacctgtcctcgggcaggcagcacggcctgcggacgcccattctcatgtaaatgtcagtgccaacgctggtgtttcaggagtcatcccagcgggctgcgggctattttaggattctctgccctgcaaacgtttccaaagtacgtggacaggccgcctgatgacactacgtttacgggatctgctagtggcttgcctacttagggagtaaaccctgtgaagtctcgcagttttgttaaagtgtgcgtggccacctgaatgctgccttatcacaagccagatacatactggtctgtagggtaactccccactgttgatcctctgagatgattgtggactgggtgctgtgagtcctgccactttgtttaagtgaatgtgtcttttgtccagctcagccgcctcggatctcgctgccaccagccttactgcacacccgtgccacccgcccttgccccgtcagcctcagcctcagctcattctctcaggcagtcccagcattggcacggtacctcctcccgctgtgggccacacgtctctgctccctgtcaaccctcctgccatcagcaccaccaccagcgacttgtctgcccgggaggatgcaacaccatctgcctccaccggccacctttcagtgtttcctgctttccaagtaaagataccagcagtgccctcagagcagaccagccagagtttttctgaagcctcccacagggtgctccccggaggaggcccgagatgctctcgtgactttggagccggggtggctggccaccttggcctgggcatctttggggtgggcttcgggagcccggcactgctgcagagtgtggtggatgagaacagctgctgcttgctgtacgtggtggaggaccagctgtgtgatgtggagcaagccttcagagctgagcatttgggccaacaccagagtggttcgggaacaggatgcagacattgtcctcaacgaccagcggtacggtttcagttgtcagcagaggttcactgaacacctgctgtgtgccgggcaccgtcctgggggctggagaaatggctctgaatgctgcaaagtccccatgcttgtgctgctgaattgggggcagaggcagatgcgtagatctcaacacatcagtagcaataagctctgcggagagaactcacgctgagtcgggagagggaggctggtgggagtgctgttttaggtgagatggctggggaagacttgaggagggggcattggagcagggacctaggtgggtgagtgagatggccgtgggggcatccggggacgagcacactctacaaagaggacactgagtcttctccaggccttggtgtagccaagtggccctgctggcaggaacggagtgaatggggtgagggctaggcaaggaggtcccaggcccaaggagcctgggggccacccggaggctttgaaagccgccaggggttggtttggtttgagctcacactcagggggcggaggagaaggacgcagggacaccagtgaggggtattggggggccagtggggcatgatgggcccggaccagggcagcagtatggctggatttgggatattttgaatgtacagcctttgcaatggggtcctggcttcctcccttcactcgagcagctgggtgtgacatgtccaccctgttcatgagcggtgtctccagcagctagagcagcatctatacttgctgtccacagggtgtggacagccacagcaggtaaacgggctcgcgggcagccacccagccttggggtcaggcgcagccactgtgttcaggcctgggctgggcactgcctggtgtttcgcagagctggccggtgctgctcacaggtcaggcctggctcacctttggtgtgtccaggtttggacatgctcttactagtgtagcaaggggagagtttgaccccaggttgttaacgcatgtggagggctctgaacgtattagcggatggtgttgtgtctcagcaaagcccattgccactgcagaagaacgttcgtgaatgttaatgctttgtgtgtttttggaaagttggcaaattttatttatttatttatttactttatttatcttttgagacggagtctcactcctctgacggagtcttaccctgacggagtctcactctgtcgccaggctggagtgcagtggcgcgatcttggctcactgcaacctccaccgctgggttcacgccattctcctgcctcagcctcccgagtagctgggactacaggcacctgccaccgcacccggctaatttgtgtttttagtagagacagggtttcactgtgttagccaggatggtctcgatctcctgaccttgtgatctgcctgcctcggcctcccaaagtgctgggactacagatgtgagccactgcgcccggcctgcaaattttagttttcagttatctgtggcatctgctttaggcaggttttcacacttttcactcgggtatcacattcacagaatgcacacgttcatacatgatacacacagcaaagtgcccgagtcggtgtgttttttacgaactgcacgcacctgtgtggccagcctggagatcacagtcctcggaagtccccgggctcctgtccagcccacacctgaggctgacctctgtaggctgaggaagggagcatcttttttttttttttgaaagagtcttgctttgtcacccaggctggagtgcagtggcgcgatcatggctcactgcagcctcaacctccttggctcaagtgatcctcctacctcagcctcctgagtagctgggactacaggtgtgtgacaccaagcttggctaatttttttttagtattttttctagagattggtttttgccaagtttcccaggctggtcttgaactcctgggctcaagtgatcctcctgcctcagccaaatctcaaagtgctgggattacaggcgtgagccactgcatctgtccctgaggatcgtaatagaatttgactcacagaagcctattctgtgacttggctgggatctgtattttgttcctcaaaattagcaagttaaattctgaagtaagtcataccggaaatgttttcacaatttactttgttgaggcctttttcttctgttattctcgtgcctgagatggtttatagagtggtgttagcagcatggattctggaatgttctaggtctcaaagcacgctcttaacattccactttactcctgcagcgtctctggagtagttgtttgctgcccgccggaggcagcttctgaaatcgtgagggatgctctccgagcagctatgggctgggctggctgaggtgatgtctgcacctgtgcccaggtgtctcagggagaatgggcttcggggaataggcttgcctgcttcatctcctgactcctttgctaggcactttgatccggttttccagttcttgcacaagcaagtttgtgtcagccacgccctggggaggatccactggatcaccgccgtcactgcctgttcccggcggcctctccggcttccctaaagagagcaggtgggcatctgcgtttttgggggaatgcacagcgttgtggcatctgtgtccatgtcacgtggttccctgtgacccagaaccctccaatgcttctcgccagtagcttccctcgagaccagaagccagagctcaccctcccttgtgtccaggtgggactttctacaacgctgctgtgcacgacgtggatgccgtatgcatgttgctgggggaagccactccggacactgtgttttctctgggacatgtcttctgcccaggtaagagtgggcctcactgttgcaaggtgctcctatggccaccgaggctggttgaagatgtagcacaggctggggcccctgttcaggattcctcccccactcccagctttggggagcagataaagtaacatggagcacatgtaaaacagatgactaaatggtcgcgtcactgaatccctcaggcatcatgcaggaagtgtttcacatttgatgaactttgagagtcactgttgtagtaaccatttctccagcccctgcgatgtgacaggcccctgtcgtttctcaggttgcaaaggtgaagaaacacagtcccggctggaagtcacagccagggtgggatgcacacagattagcagatcatcagcgagcaccaggtgaaggcggggctggggatgctggttcctaggtcctggcctggcagcagaagctcagggtgggtcctgagggctgctgaagaggacagattatctgttgctttatgacacaccacacacttgaaacaacagccactgggtagttaggattttgcagcctggactgggcttggctggagtccctcttctggtctgcccggtggtcactcacgtgactacagccagctggtcagcggcctgcctggaacagccccaccagggtcccagggccgggccccgctgtcacctgggttctcttccgtgtcgtccccacgcctcctcaggtccccacagcagcgtggccacacatttgatggcctcaggggaccaaagtagaagcactgagacatcctgaggcttgggccgggactgacgtggtacccctcacaccacactttgtgtactggggcaagtccaggcccagccagtgccagtgctgacttctctcagtgcctgcttcactctgtctcgtcagcacccccttttggcactgcctcccccgtctgcccaaagttcacccacacaccctgctccgctggccaccaccccgatcccagccatgtcctcatcctggctgtggccatctgcctgcaatctcttccctgtaagacctcagttggaccaggtgcccacctgctctgagctctccagagcacaggtggaggccatggtctccgcagaccccgcgggcctgtcaccggtcgagggtcttgactgcacattgtccaggttcttggccttttgaacaaagaattggacaaaacacccagcaaagcaaagaaaggatgaagcaacaaaagtacgaaagcagggatttattgaaaatgaaacacactccacagtgtgggagcggcccgagcagcagctctagggccggatacagaatcttcttgggcccaaataccccctagaagtttcccattggccacttcatgctcacctcatgtaaacgaagtggttgcaaaaagcaaccagtcagaggctggagtgaagttacaaagttatacttctgtgcagatgaaaactcagcctgtaatcagtctgattggtgtgaacagcgaccattcagaggctggagtgagtgaagttacaaagttgcaaaggaagacttgacccacaatcagtctgatttgttgcagacagccagtttcccatctgccaggcagaaaacgtgagtggggtttgcaaaggcctccggtccttttgttacttaggagtggaaagttagggttttcccttcaacatagttcaaggaagttgtgaaacagccttaggttccctgcctccagaccctaatctcctgcctcaggctcacgggtgccgccccccgcaaccccactgtcctcttgtccacttctcgctcttcctcagaaggccagacaagtacctgtctggggcccgctccccgggtgggacatggggactggctctgctaggcccatggctgtactcccagggtccagaaggtgcctgggcccgggaccgccaacgctacagaagcttagcaaacacggaattggactgcttgctgggcagcgtccccgtgcctctcagggacccattttggaacaatttaattttctattaaggaccagtttctgtgtcactacttggctatgccctccgcccactgcttcagttaaagccgtgagttcaggccgggtgtggtggtattcccagcactttgggaggcccaggctgcaggatcacttgaggtcaggagttcgaaaccagcctggccaacatggtgaaacccgtctctactaaaaatacaaaaaaaatcagccgggtgtgatagtgaacgcctgtaatcccagctacttgagaggctgaggcaggagaattgcttgaacctgggaggcagagtttgcagtgagccaagatcgtgccactgcactccagcctgggtgacagcaagattctctcaaaaacaaaaccaaacaaaacagtttctcatttgcctaaatcaacttttgtatcatctaaagttttcttctgcttagatatggccgccttaaaagatgcagatgctgttgtgatcagcatgaagtttccctgtgaggccgtggttagcgtggacatcagccagcactgcacagacagctgcgaccaggacgtcagccagcactgcacagacagctgcgaccagagactggaggtgacccttgcccttttccacccttcgccagtccactgtgaaaagctgggttgattgtgcgggttagatagaggtcatcagccgggttgattgtgtgggttagacagaggtcatcagccaggttgattgtgtgggttagatagagatcatcagcctggtagattgtgtgggttagatagaggtcatcagccaggttgattgtgcgggttagatagagatcatcagccgggttgattctacaggttagatagaggtcgtcagccgggttgattgtgcgggttagatagagatcatcagccgggttgattgtgcgggttagatagaggtcatcagctgggttgattgtgtgggttagatagagatcatcagctgggttgattgtgtgggttagatagaggtcgtcagctgggttgattgtgcgggttagatagaggtcatcagccgggttgattctacaggttagatagaggtcatcagctgggttgattgtgcgggttagagatcatcagccgggttgattgtgcgggttagatagaggtcatcagccgggttgattgtgcgggttagatagagatcatcagctgggttgattctgcaggttagatagaggtcatcagctgggttgactatgtgggttagatagaggtcgtcagccgggttgattgtgtgggttagacagaggtcatcagctgggttgattctgcgggttagatagaggtcatcagctgggttgattgtgtgggttagatagagatcatcagctgggttgattgtgtgggttagatagaggtcatcagctgggttgattgtgtgggttagatagaggtcatcagctgggttgattgtgtgggttagatagaggtcatcagctgggttgattgtgtgggttagatagaggtcatcagctgggttgattgtatgggttagatagaggtcatcagctgggttgattgtacgggttagatagaggtcgtcagccgggttgattccgcaggttagatagaggtcgttagctgggtgtgtgggtcccaaggcgtgctgcagatggagaatcagaattccttgggcatcaccgggcggggcatgtccctgtcccttcgctcccagacccaggctagccgctaccaggactcctatcgagagctcttcagacactttgtcagaacccttaaaggtgggtgacgttttcaggaggaacctgggatgtcctgatgctcaatgggtagatgccttccaggctgcagtcagtgcagatgatatgccaatttcaggtggaaaattgcattccagataccgggttcagatttgaaattgtccatctcagtgatcatttacgttgcgtgtacttgagggagattctgagagatgctcctttggggttctgtgcgtgtaccatgttgcctgacacgtcctgagaaatcttcttaaactctgagtttataaaataactacttctgaactcctgagatctagtggataccatgtatcctggaagataggacactttctaccctctgtcagtcctgggggtgactgggaaccagagaggttgagcagaattcctggacctgggtggggcttgtccaagaggggcaggtgggcctctctaggatcagtgtcccacccagaggccagtggccccttcacatgccccaacaagaggaagactgtctggctccgcagtgttgggttttgtcctgaatctgtgcaaatgtgcagggaaataacctcctgaaatcaccaaagagcagttcctcagggccttccgggtgaccgtggccgtggagcagtcgtggtgaaaccggtcagctgtggacctgccctgcgaatcggcagaggcctccgtggtgaagacagaagctccgtgaaccaactcgaggctggagccaagatcctgcctgatatccagttgcctgtttcacttgttactgttactgggagagggagacaaaacctcatggcaatcttgtcctcactggagactcagaaggggaagcattgggaaaccatgtatgtttcatttctggtttaaccaggcttttcaggatgggacttcagaccaaggacacaagttgggcttgcttaggtgttttgtgttgtgcgcaggcctcagagatgctcttccggtcagagttcccttggcagggccttggaggcctctggttggctctctcaggaagagggcaggtcagggattgggcggtgtaaattgactcagaaacctgcggtttcagcttttctgtggcaaacagggccgagggctggtgaagctgttaaggctaaatgtcgcatgcatgaggcgggctgcaggcctccttaggccttttttggggtaattacaccagtatttaaaaacatttttttttgtttgttttgagatggagtcttgccctgtcacccaggctggagtgcaatggtgcgatctcggctcactgcaacctccgtctcccaggttcaagcgattcttctgcctcagccttctgagtagctgggattacacgcgtgtgccaccacgctcggctaatttttgtatttttagtagagatgggatttcaccatgttggccaggctggtctcgaactactgacctcgtgatccacctgccatggcctcccaaagtgctgggattacaggcgtgagccaccatgctcggcctaaaaaacaattttcttgaagaatccgattttgtgcatagtaatgacacagcaccattcctgagaaataggaaatatatgtgtgtggtatgaaaagcccatattctgttcttgtcacctgaatctaggcttcctctttggatgtgaacgcgggaatgaacagtggctgttttccacccaaaggggcgtggagaccttttggaaacgggctttctcctttccattttgcagccgggggcgggtggaaaccttccttcgaagggtagtgccttggggagcagcagacctgctctgagtccagcttagctttccaaaattctgtgtggaacttactggagatgtttttatatatttgaaaataatggcctgtatttctcacgctatacttaaaaaaaataactaaccttttaaacaaagatattcaaactcacccttgtgctgaaagcactcacatttctgtgttcattcctgaaagggcgttttagagtccccgtttctacgttctatgtggcaccctcttgccgagggaacatccagaatccctccacttcctgtctagtttgaggcctcaacaccttcatgatccagagtcctgcggctatgagggggtgggcccaccaccgcccagccctccttggtggacgtggggacaggtaggagcctagggaaaggggtgggcgggctctggtgtgacggcgtgagcgctagccaggaagatggggccagggcttggcatggcacctcacctgtgggggaactcggcggagcttcctggcggcatctccccctcttgccttggtcctttctatctttgttttaggccaatttctaaaaagactgaggcctccttcaaggttgaacagatttctagagccttgtttttgctgtgacaagtccctagtccctgttcacaactctgaaaccaagaaacctgagaaccaagagaactgaaatgaagcccgttggtctcagcctggcttgagccaacatgaggctctctgctgccttttgttttcttaggttttgctgagaaacgggagtgcgttctgtgtcggtgtttgtgacgccctgagaccctgcttggcctagctaaagtccagaaggcctgggccccacagggcttggatgagggctcatgggcctgtacttcaggaggcctgagaggcccggtcagtcccatgaggctactcggcttggcccgcaggtcctccgaggcacagggcagagggacaccccagggacccatcagactcacgacacagagaaactcaagtggggtgccagggccggccacagaggccacgggcttcctgcctgtgaggagccgccctgtttgtctgagccctttggagatttaggttgagtcatgagaaccggtcattggaacatacactttattatgttacaaaaacaaaaatccccactgaaacacagctaaaaaaataacacattttcccaagattacattaccaaaaacagttgttatgtcattggagggcgtccattaatactgctcggagaagcacgatcttacacgaaaaacacggatgggatttcgttttcaccttaaagcattaaagtgctttaactggtaacagaggagtggatgtttgtgcgttggagacactgactgttctgagtgacaagtggctctgtgccaggatactgcgggtcgggctcctgggtacagcaagggcttcccagcagcccaggcctggtgggaacgctgggggaatgggggagagtcgctgtttctgaccagagcgacttggacagatgtgagctgacctgcctggcgagctccttctgccttgcctctctgagcagactcaggaccactgcctgtcccccttggccccaggagtgccagcagcctccctccaacccctgcactgctgggaaaaccatccgccttcaagatttatttgttgatttgtttagttttataaaaacatatcacttaaaggattcttgttcctgggaatttgcattttctcctgaccacggcgaagtctcaactctgtctactttcacagctgcttctccagtgcaaggaggctgcatgggctccctagacaggtgcagctggggccatctggctgctccgtgttctggccggcttccttggacatctggctggggttccagtgacagctgcccctgggcctctcccagcccccccagcccccccagcccttcccaggagcacactacaggggctcttgggacagcctcactggactcagggtggacaggtaacgtacctaagaaacaaatcaacaagccctctggaactgaagcacatctgcgctgggctgaggaaccccgggtgtcaggatgggcccccgcggctcctgaagtggaacatgggggaagagtgggggcttcaggaggtggcctcgggtagcaggtgcccagcccccaagagatgggtgtggtgcctggggcatgggtcttagcctttctggtctcagcttcctcatctacaaaatggcaataagatggagagctgaggtgacatgcagctggggtggctgcgccaccctgggaaagccccgagtctgctgctgctgccgctactgactgatgccctgggcgtgggcacctttctagggagatggaaccctagaagctaagcggccagagcttcctgcccttcagctggaaggagccggaaggagctggaaggaagggccaggccctcgccctggctgcagccagatcccctgagagccgacagccccgttcctgctgcccccacaagcgtgcacccaggcgactgtgctgtcagcggcccccggggaggtggacggtgggagagggatgcctgcgtcttcgtagaccaggtacctgcggcaacaccagtcgctgcttttaaataaggggaggctggtgacggtgatgccacttagcaagatggagtcctgatctggggatgggggctgtgtctaaccttgctgtgccctgaggggcttaactgctttttctgggagatgttagtaggggaagctcctagcccttaggaccacacataccctaggcagggggtgtggagctggccgtgccagaaggggccaggcagcctgcactctgggtggtgtctcggcttgcgtgggtgagggctcccattggcccacctgcctgcaatgaagggcctcgtcacacccgtgaggccccccaggggctccggttccctgccctgaagctgtgctccgggacggtcaaggttcggccaccaaggctcaggctgctcctcgtggatggaggtggccccagaaagggcctgtgcagactctgtctccccggggcctactaactccctggggtgctccacgaaggcctagacccccctacacctacctcagtctgttccctgactgccaccgcctagaaaggaaagcaatgccctgtggaccttgcagtgctagagaaggtggccgtggcaagggaggagctgctcccatggcagagcattcctgcagatttgcctggatttgcccttcctggggttccctctaggcttcctccccacccggccttcctgggaacaagtcccgcccacctggcttctcactcagcttcctcagcctggctcatgggcgctaggttgaactccaaagagaatgctgagaaagaaagttaaaaaaaaaaaaaaaaaagctgacccaccatgtagaaaagggtatcattttatcaaatattgacaatttccagtcctctaaggacaggctgtccctttctgctgaggggtccccgaggagaggcgactgtggccaggctgaggggcactggctgaggcggtaccacatgcttccccgcgcctggggggggttgcgccctccccagttttctccaatttcagcaacactgtggttgttgttaggggcaggaaggaggcggtcctgcagaatacagcgtgctgggggcagaggccagtcccaagtccagcagagagagtccccagcccccggcgcgggcaagacgcagttagggacattccctgaaaatacactggtgtttcagacacagtcacttggctgctcactactatccagaaccaaaagatgtcgcaaaatactttttggcagtccatcagcaaagaggatcctttcctttggaaggacctcgcctggttgggagatgacagtctctaggatgaagcctggaagtcggagccgacgctgcggtccccacccacgcctgccatggagcacgccggggcagtggcagggctgagagtgggccagcccccgccccacaagcccagcactagggcagctccagaaggttctgcaggtgactcaggctgtccacaggcggcttcggtggctgggcctttcctggggcctgtgaggtgcctggggatgggccggaccttgcctttccccgggcacagcccgaaggtcctgcctcctgaggcagttttggacacacaggaaggagaggggaggcagcttgggtctctgggcggtggcgcaggctgcagccaggcgaggccctcagtggatctcggcctccgtgaactcctccttgatgggctgcttgcgggccttgcagtcgggcaggtcgaagccgaagtccgcccactcgtcagggccgccgcctgggccgccgcggttggggatggtgatggtgtggcgcacgcggaagtgcacggcctccatgacccgctggcgctgcagttcccctgagccgccgatggagatggtggccgcgttgctagagcggagcagctgctgcgcggtgctgtagtcgtggccctgcttcaggtcctgcaggccccgccagatggtcatgcggtactgctcggggatcttcagggcccccaggtcctgcgagaggccggcgcattaggggcagggggcagtggagcagggagagcagggggggtcgccctgggcctggaggtctggccacaccacaccctggcccctacacccccaaccagagtaggcaggagacgggggagggggagagcccaccagagtcactcctggcctgtgcggggcctgggccctgacctgggagtgttagacatggagccccctgagcatctacctggccctgggggaggaactgcctcccagcctcagggcagggccctccttggtctccatgtccctgccccctggagtggacccccagctcatccctgagctcccaacaccctctgggcaccacaccaagcacacacatcactcaggggaggcaagggttaacgtgacctggagctggctcagcgtgggtcactggggacaggttgcggggagggcggttagtggacacagacgcggccactctctgcacgagggacagttaggctgagcagctggccctagctcgcactgaccctcatctctgtctgcagccccaggctggggatggtctgggacgggccttggacctgggccgcggcactggctggggggcaggttcccaccagagggcttgctcttccttgactgacggtgccctgtggttcacaccgtcctaggctaccagcaggctttgtactccactagtctctaacccaaagacaagccgtggctgtttaatcatggtgcctgcagtcagctgccatgggtgccaagagctgtgtggcctcgggccaggtgcttgccttccctaggtcccctttcctgtctgtgtaaatggcgctgacctctctgtaaatggcactgaactggactgactccctcggggccttacattcttctgttctttgtgccttggaaacagcccatcccctaaaatcaaaggcacactggctgctgggagtccaaaaaggctcaaatgaccagttcagcatccggaggcctgggcctgtgtcccgcgccaccagccacgtgcctggctgcatctgcctaagctctcaagagtcaaatcatcgcatctgcttgcccgtgggaaagggtttccaagtttcccagccacgagagcaaggaagagaaggctctttgccctccggacaggggcccctccagctactgcctctgcagagcggggtccacccgggcgttacctcaatggtcaggttctgcaggtggtaaatgctctgtaacccttgggaggtgaaatactcgatgcagtttggacaccccaatcctgttaaaaaactgcagagagaatttgagaaaggaagcagcattagcttccgagcacagggctgcgcgagcccccatctctgagagtggcctggctggggcggagactgccgctgtctccgaagggtggctccagcccacaggtggccacggcatccctcagggaggggacgtgctcagctggggacaggtcagtggaggaccccggctttttgtgcttttcctctaagtggaccatttccccagcatctgcagatgctcctggcctgactgcccccgcccgcctgtctgatccagggttgcgagtggtgagcacacagggtggggcagggcagctgacatggccagaaggacagaggtgaacagacagggtgacagcacatgctcaggccctcggcagcccacgcacctgacgaggctggggtcggcgtggtaggggggtggcggagtgcagtgggaccccgagaccatggactgggcgctgtggctgctgctcatctcgccgttggctggcactgcgtggccatggttgttgagcatcccggggcctgggtgagaggcaggtggggccatcagggcagccaggctgggcatccaggtgcagcctccacccaggctccagacacctctggacaccagggagccatcccaaggcctggcgatgtgacgctgggcatcacccctgccagctagaggcttgagcaagggctgaacacctggggccctgtctcctcgccccgcctagtagggacgggggtgaccgtgcgtacggggcactgggttgtaagggcaatcgcagctggcactggagtgctggccacatcccagggcctcaggaaacacctgggggtgcttgctcacggcactgggaacatgcttggtcctgcccacatgaggaccttccaggggaagggggcccaaactctatccaatggagcccccggcttgtgctggggcccctgatgaggtcttgcaccatttattcctcacaaggagtgagcctggtacttctgtggccatttgctgatggggaaacaggctgtgtaatgttaagagccttccccaagctccctcgcctgccaagaggcagtgcatgcatgtgggttccacgctcttaaccacgcccccctccacctcccacagccaggagtgggcagagagggctatccgtggctgcgcctcctgcccagagggtggaaccgctggaaggagccagccacagagacacctgccctgggtcccgagctgggtggtgtgtcctgagctaggcgcccccagtgggcctccccgccctgcccgtgggcccgcactgcccaagggactcacccacgggccccaggttgggtgtagctgccgaactgtgcgggggaggctggcccaccagctggttgacggagggcagcttgttcatgcccccgtgcaccttgttcatgggcgagaggaccggcccgtaggacgggggctgtaggtgactcctgctcagaagcaacggctcgcatgggtgggaagcaaggcagcctcgtgcccaccgaccacccatgcccgtgatgcccccatgctgcgaatgggtggagcctctgatgtcagagagtggcttcatcctcccccagcctccctgtccccaaccatggccatgcacggccactgggcctgcgcctgagaaggacactggccatggcggggccgggaggcagggggatgacctgtgccactggaatcggactctggtcttggtgggtcatggtcactctggccacccctcctggcctttccactcagcagacatttacaaggcccctcagtggcagaggcatagcaagggccatgacccagggtcccagggcagccccaagtgctagaaggcttcctggaggaggagatgtggggtgggctctgctctggaccctgctgcctgtccctggcagatgatgccttctggaggggtttctggtcctcctggccttctcctggggggttaagccatgtcctccttccccacactgatggtgggctagggctgactcacggcctctgtaggagctgctgctgctgccgataggagtccaccagtggctgcggcaccaactccatcagctccaggctctctttcagcttcatcaggatctcaaagttctcccggcctcgcacctgagcacagaggagcagatctctgcccacgtgccccagacccccacgggaggggggtggccgcagcctcaggaggacctgggcttggcaaagcatctgtcctttctgggggttccccagcaccccagaggctcaggatcccctgatcttctttcctcttcccaagaagcaggtctaaccagagcccctgactcagtgggcaggtatcctgtccccaagaaccatgcctgctccttgtggcccccaagcctccctcttcctcccctaccaacagatactgggtttttagaaaccaagtctgtctctctctgtctctctgtctctctctctctctttctttcgacagcgtcttgctctgtcgcctaggctagagtgcagtggcatcatcatagctcgctgcagccttggactcctgggctcaggcgatcctcctgccctcaggtgatcctcctgcctcagtcttctgagtagctgggactgcagctgcacgccaccaccacgcccagctaatttttgtagagatgggttctcaccatgttgcccactttggtctcaaactcctgggctcaagtgatccacccgcctcagcctcccaaaatgccgggattacaggcatgagccactgaccccaactggggctcattctggagctcaaaaaacagaaagttcacatcatggaagatgactcccagccaagcctgtgccgatttaattcacctgttggaggccacatgtgagaggcacactcatgggggctggccgtggccacgggtcctggccaacgttagggtcctggattcttgctgaacttctaccctggtggcactccgcaccaccctacctgtgctatgtacacatggcttcattgtaaatggcatgacgctgggtttttgggactgaggggactctcaggaatgccctgcagttgggtcctgctggtctggctcttgcaagcaggttctgccctctggaccctctcacacagggctgggctcctcccgccccctccaccatggtctccagagtccctccccatggcccagcctgggctcagcagacgacagaggtgaggcaggtctcccggctgccgtgcaggagcacacactcacctgaaggtagtacgtgtcctcgtctccatgccgccgcttcttcacaccggcaccaagggcggggacggcaggggggctctgcttgaaggctggaaggccaggcagggcaggcggggtgagcaggcaggaccagagggtgaggcctccttccgggaggagggagacctgccccacccttggggccaatcgattctccaggtgggtccggtggtgagcaacccacagggacgggcaaacctgcggtgccacccaggcctctgaggtgcccttggcccaggacaagcgacttatatgggggctgctctcaggctccgtcagggaggagtctgggcgtggggggccgttcagggactggccctgtccctgggacgcgggctcacattgtgtcctgccaatgtgtccccttctcctcccacacgcgtccagttcccctggccggccgctcaccacgcttgctggcggccccgttcttggcggagctctcgttcagggcctgctgctcccggtagtggtcctcatcagcttttcggtcgcggccaggacaggcgcagatgcggccctcaaaggaccggcggcccagcacctgcccactgtcgggtgggcacagccagaattgtgagctcaaccctgcccaccctcaacctgcccaccttccccacccacctcggaccctgcaggaccggccttgagagccctcggtcaccacagagcacccagtgccggcagcgagtgttcccgctagagggagtcagaggctcaaagctgtgaggctgccctgagacctcgccggggactcccacctccctccggaggcccagcttgtgcccaggcaggccctgcaggccagggggcggttccttcctccagcagcacaccccaaagtctcttccccaagctagccttggcctgcaggtctccatgacagctccccttctcccccggccgtgcctgcccagccctgtcccacctccaccccgtgtgcccgggactcactcccgcatctccagggtgatgatgatgaggatgggccgccggttcatgccccctacacagctgctgttacacatgaagttgtacaggatggtggtgaattccgtccccacctgcacatgggggagcagggagaggggctagcatcagcacgcagccccaatccactaaggtggcagcccccagctccatgtctggtcctggctccacgtaggcagggggatgccctgctctatagttgttgccatttgtatatctgtccactcattcatccatccaccaacccaccccatccatccatccatccgtccatccatccatccatcgaacccatttatctattcatccatccatccatccatccatccaccaaccccatccatccacccacccatctatccatccatccatccactcccccatttattatctacccatccatccacctacccatccattcaatatccatccatgcatccatccaccaaccccatccatccacgaaccccatccatccacccaccccatccaaccatccatccacccacccatccattcaatatcaatcaatccatccatccatccaccccatccatccacccacccgtctatccatgcatccatccaccccatccatccatccatccactcccccacttattatctacccatccatccacctacccatccattcaatatccatccatccattcatccaccaaccccatccatccacccacccatccattcaatatcaatccatccatccacccatccacccacccacacgtccacccccaacacccaccccatccatccatccatccatccactcccccatttattatctacccatctatccacccacccatccattcaatatcaacccatccattcatccatccacccatccacccactccatccatccatccatccacccacccacccacacatccaccccccacacccacctcatccatccatcctatccatccatccatccacccacccaccccattcatccatccatccactccccccatttattatctatccatccatccaaccatccatccacccacccatccattcaatatcaataaatccatccatccatccacccacccacccattcaatatcaataaatccatccatccacccacccacccacccactccatccatccacccacacatccacccccacacccaccccatccatccacccatcccatccatccatccacccatccaccccatccatccatccactccccccattatctgtccatccatccaaccatccattcacccacccatccattcaatatcagtcaatccatccatccatccacccacccacccactccatccatccactcacccacccacatatccaccccccacacccaccccatccatccatccatcatcctatccatccatccatccacccccccaccccatccatccatccactcccccatttattatctatccatccatctaaacatccatccacccactcatccattcaacaatatcagtctatccatccatccatccacccaccccatctacccatccctctacccacccatccattcaacatccctctacccaaccatccattcacccacccacccatccatccattcacccacccaccccatccatccgcccaccctattcatccatccgtccacccacccatccattcacccattcaccactccatccattcatccactcacccacccaccccatttatccatccatccatccacccacctacctacccatccatccattcatccaccccacccacccccatccatccacccatccacccactcaccccatccagccacccattcatgaagatcactgaaccccaagcactgggctggttctgggttctgggagggctcagacctggtctctgctctcagagaagcacagagtcctgtggtctgtttgccccaaccttctcccctgacacaggctggaggtcaagcccagatgctgggtgtgagtgtgcctgcacagattttgtggccctacgtgtgtgttgcatgctgcgggctcacctgggcagaaagaggaaccagagctgtccccagcgaacccacccccagcacactcctgtttcccagggattttgcagtacagctgagacatgaaccaaaactcttttttttttttttgagacggagtctctctctgtcccccaggctggagtgcaatggcgcgatcttggctcactgcaacttctgcctcccgggttcaagcgattctcttgcctcagcctcccaagtagctggaattacaggcatgcgccacgaggctcggctaacttttgtgtttttagtacagacagggtttcgccctgtgggccaggctggtcttgaactcctaacctcaagtgatccacccgccttggcctcccaaagtgcttggattacaggcgtgagccactgcgcccagccatgaaccaaaaccgttgatcaatgagtgtccccagaccacaatgtgggcagttttcatgtcaccctggggacaaatacaaaagctattaggggtttgggaaaatcttggcctttctcagaactgctgtcacacggcagccaccagggggcgatagagctctgccttcctggcaggcctaggcaggtgagggtgggacatgggtttcagggagccctcccgggccctggttctgctgacataaggggctgctcttagaggatggtgggatggggacgtcgaagggggcctgggtacagggtagaggcttgtggaggacctggagactgggtacaggagtctggggtgtcctgggggtctggtacagggatggggacctggtgggggaggctggggctgggtacagcatggagcctggtctcagaaccaggtacagggaaggcagaggcctgccattgcctgccttcccctgggggcctggtacccactcctcacctcctgttctgcccctgccccaccatcccgcagtaacttcaaaccctctgtttccccctaccccagcctttcacaccagttgggccccacatcagctccagaattgtgtcccagaaacttatgctgcctttgggccaaaagtcaaagcccccagcctctccgtgtcccctcccctccagcctatatgccccccagtcccctgcaccccccacccacgtggtgcccagcggggcctcccggctctccaccctgctgaccagcctcagcctcctgcaggccacctctcctgggcccctggcctggcctaggagtctttccaccatccctcaaccccctttgtagaatttgccaccccttcaggcgctgttgctggtttggttctgccccagccaatccagggtctaaatgggcggggacctccccatctcccaaatccctgtgtctgggagccccaggccggaaccagtgttgggcaccaacagagggcaaggctgaagacccactccccactatggagctgggcacactgaggccccgagagtgcccgaggcggatgtggggacagagcagcagtgtgcacagtggggcgggtgtgggtgaggaaggagccgccgacacagacttatcacagagcagggagccctggacagatgccgacaggacaggcaggcagatcagccaactgaggagggacaggggctgcctcaggggacccctcccccgacccgtacagctgactgcagggccctgggcacagcctggctcctggcctacctgtggtggctcatagggcaccacgacgctctgcctgccggtgacagggtcatccacatactgcgagagattattgccttccacgcggatgaggtggctggctggagcagactgtcctgccgggaggggtcgacacttcagagaggtgcatagaggtgcccacccctgtgctaggtgcagagaggtgcccagccctgtgccgggttcaaagggtgcccagccccatgtcaggtgcagagaggtacccagccccgtgccaggttcaaagaagtgcccagccccatgtcaggtgcagagaggtgctggccccgtgccaggcacagagaggtgcccagctctgtgccacctgcaaaggtgcccacccctgttaggtgcagaggtgcccagcccagtgccaggtgcagagaggtgcccagctgcatgccaggtgcagagaggtgcccaccccggtgtcaggtgcagagatgcccagccccatgccagatgcagaggtgcccagccctgtgccaggcacagagaggtgtccagccccatgccaggtgcagagaggtgcccactcctgtgctaggtgcagaggtgcccaccctgtgccaggtgcaaagaggtgcccagccccatgccaggtgcagagaggtgcccggccccatgccaggtgcccacctctgtgtcacatgcagagaggtgcccagtcccatgccaggtgaagaaaggtgcccagccccatgccaggtgaagagaggtgcccagccctgtgccaggtgcagagaggtgcccacccctgtgccaggtgcagagaggcgcccagctgtgccaggtgcatcgcagcagaactgagaccaccaggcccagtcctgtgggcttccaggtgaggctcaggccagctggcagcccttgtgctctgggggtgcctgcccagtggctctgccccctgattctcctgctaatccttggggtagcccgtaggccctgccaggcacactggagagaggggctatgactcataggatctctggggccccagaaggccgctgcccctcagactctgcccagaccacttgcccctgcccaggagcatgggcccccccaggtcccactcagaactgggaaaatgggggctggtggaggcactgaaagccctccgcaggggaatggctaagcccatgctgtcagccccactctctcccagttaggcagcccccggctgtgctgtccgggatgctgggcaaagtgccaccgtgggcagaggagctgggggccctcaccttcgttgaagtccctcccgagctcgtggttggggcagcgtttcacgacgtcggtcacgtgctccgctttcttgtaaacaggcatggcccggatggcggtgcctgggggtggcggggtggacaccttgatctggatggggcatgtcttggcgatctggcagtagagtttcttcaagagcggggagtactggagtgggggagagggagcgggtgagaccagtggtcccaactgcacccctgtccaggccaccccccattgggagccacgtcctccccatgggtgccccttccacaacccccagatgaagactgccccaagcagcccctctgctgaatccctccaggtgtctgtcctttgaggcgtgaatccaggcacagctgccctggcacccactccacagccatcgggtcatttgggctccatgacagcaggggtgtgagtgccggtggcagtcaggcagagaggctttaggtttatggatggggacaagaaaggccaggagagcaagaaggggctgctggagagtgagggcgggaacgggggctgggagccaggcctgtggaagccatgtccctgactgcggcattttgaagaggtgggtttgcagcattgaatctgtttttaagaccagagctaggcttctctggcatttgcacagagcaagtggttaatgtgtgatgggctggttgcccactatgcattcaagcttccatagagagctctctgggttggtgtgaaggcctctcatggcctcagcagccctggtgactctgacaccctctcctgctggggccaccaagtctgagcagccgagctgtgtggcttggtcttggccactctgggggcccctctgggctcacagccccagtatccccagcttgggactcgaggagctaataggtctgtctggagctggcgtgggccctgctgcaggaggactggatgacacccttcctgagccagcccctgcagaggcctccagcttctttgcctcacagggcccggctgcccatctgaggacccaggccggctctcatgagttctaaaagtgaccccgctgaggccgctgcaggggcgtccactcacttccaagctggctgatttcccaagcgtctggctcctggtcacctgctgggagtcctcatgctctctccccaaagggccatcaggaatgatccccagtctcaggctcagagaatgacttctgctgccaccccatgccctctgtgcccctcaccagcccccgatgactcagaatcacaagtcactgcagccgcaagtgccctggagaatgtcccatcgcactccaccgtggaacagttggggaaactgaggtcagagacagggagctactggcagaaccagggaagctgcaggtggagtcttccctttgccaaggaagttagggggccctgaccccctgacccccactggccaccagtactgcctctctggccatcaagggacatgaggggctctcaggagccacataggtccttggcaggtgcaggcacacgaaaagccaggaaggaaaaaggaaagagacaggggagatggagctgacggaggctgcgagccatggtgggtggggcctgggtccccggcaccctgagtgcccttgttcctgtgcagtgggagcggtcccctctctggtgagctacttgggctccccgaatgagacccagccaggaacgctggtctgtccctgccatgggtgtgagggggctggagggacaagtggaagggggtccttcagtggcctcctcaggatgggaattggataccaaagagctggcccctcctaggtcctccgtccacctggagcccctggagccagagtggctccactgcccctgcctgcgacccccaccttgggcacagggccttaggcaggaagccgcccatccccagtcctgtttaaaacacaggaaggaaccatcatgcacagccctgcaggggaggctgggcgctgtttagaaaagcaggaaacgggctgagggcggtggctcacgcctgtaatcccagcactttgggaggccaaggcaggcagctgaggtcaggagttcgaccagcctggccaatatggtgaaaccctgtctctaccaaaaatacaaatattagccaggcaaggtggcgggcgcctgtagtcccagctactcaggaagctgaggcaggaggcaggagaatcgcttgaactcgggaggtggaggttgcagtgagccgagatcgcaccattgcactccagcctgggtgacaaagtgagaatccgtcacacacacacacacacacacacacacacacacacacacacacacaaagagaaaaaaagaaaagcagaaacagccttgcagagctagcagcccacccctgtgcgggcgcctgccctggactgtcggagctgcaggcttgggcacagcgggaagtacagcagtctccttgttgtggggtcagtctggcttccctggaggtgaccactgatgtacagtgtccccaccattacaaatgccccctttgctccccacagcatcacttggaaggataagcaggtagccctgcccccagggcacatgggaacagctcgcagcacagaccctctgggactcggaccctcaggagactcagacagggagggctcttggcccaaacttgcagtggcctgtgtccaggcactggtctcttggacaaaggccctgcctgaagctacacaccacgcctggtctccatctgtgctggttggggacagaaggaggaggggccaggtggtggcaggctcttgtctgccagtgccccgagcaggggagaggaacacagggccggcagccgtgtcgccaggcttctccgaggccagggcagcctgcagggcgagctgcccattgctgagcatttatggagcacctcttgtgtgcagagcacacaggccccctggaattcaggcacctggctctgtccccctcgtctctcctcagtaacgtgtagcagatggaggtacacaggtggtactcaacaaatgccatccatcctagccgctccctagggaaaccacagagcagggagggcgcggcagaccccccttgaaacagaccccggccggggtggctcaggtcagagtctcaggggcggcaaaaggagaagggcctctgaggtccggccctgtgggtggcagttttggctttgtgctcgtcagctgggatgcctctagccactgcacaagcctctccacctgcatctctgactcactgcgtgtcccctgggaacacagtggcctgcaggtgttagtggctactgcgccctcgttattctcagcaccaggtgaggaggtgttggttgcggggggctggagccacacaggccccgcttccctacagttccctttccggggctgagtcccccgaggcttcttccaccctagtgccacagcccatggcctgagatgggggtcattagatccaccaaggagccccagacactcagggggaggggaggccgcctgggctcctcagccagttggacacagatggagtaaggctctgggtgcaaccaagcctgcgggcagaggccccggcagccgtcgctccctgacagccccatggagtctcaacagcgtgaggggtttcacagctcctgcctggcccctgcagcccctcccctaggcccctgccccagcacccccggccatagtcctctgcggagcctgagctcccgcccgcctgggcctcagggtcagcagtgctcccatttcgcagacctaggcagcggcctctcagcagcgccctgctgagcccgccccactccctctgggtctccgggcagggtgaagtggcgatccttgctctggaagctgcgtggtggggcgtcccaggccagggcgctacgaagtgggtgcagggcccagtgcgtgctcccgtctctgcactgtctgagggactcccgtctgcaagaggtcctgggggcccaggcgcggggagcactgtggatgccccgccccgccccgccccggggaggagcttgggcgcgccgggagctgtccctggtactcggtgcaggcggcggccggaccacccaccccgggaagtgggggctcggactggagtgtggagcggcctagaacggacaggtcttcgacctgggccagtggcggagggaggcccaggcctcagggtggagagcaaggaggcccctgtcctcaggcgatgtgagcacttctcctcccctctggccagtaaggccaccaaggtcctgggcctggctctcctccccacaggtgtccccagtgcagtgctcacatggcctttggggcatacatgcctggcagacctcaccctggcactaactggaccccaggtgcccagccgggccggagctgggtggcctcggggaggatgtctgtgatgtcccctgccctggcagcatctgggggtgggtgacacaggtggccacaggagggaagggagccctgagagatccaggtccttctagagggtggctgagcccaggacggctccagcgctcccccagctccttcctcctgtctccccagcaggcctctaggcttcaggcctgggcttggccctgtggaaaacgggcaggaggaggaggactccggtggagcctattctataggaaaggtcacgtggagacacttcccctccgccgcccccctcctgccttctccttccctagaaccgctagatggatgtgtttcaacagattccgagctgataaaatcgtatttatcttcggctgttgacacggagccgcctggacttgcctgcccagggtgcgtgcaccgtcctgcttgcaggaaagggtttgcccaaacgccactgtgcccatccctccccacgctgccggctcctctccgaccctcctctccttcccctcctcccccgccccagtacaacatgggcccggcctggctgggagccctgggcctggcagggctggtgcgggggtgggtgaggggagggtgcagcggggggctgctaatttccttgtgcccgctcctcgctccccggcagctgggtgcccggcacgggcaggtttgggcaggaggacagcggagaggtgataataacttgtctgtcattttcctgggacgtgtagcatcttgcaaaccgcaaataccgttaaccagcgtgggtgaatctgttgcaacagattctggaagatggggcctgcagtgggggcttttattttttaaacgattttttttattgctcacttttatccagttcccttgcttgtgtgtgcgtctgtgtgtttaggggtgcatctctctcctctctggggttacgggcctctttgagcccaccctctgccaaggccctggacccttcctctccctagaaaagctcctggggctgagccacagaaccctttccattaaattcgtgggttcacagatggaccccctaaaacacatgcctgagccagggtcaacagcctgtggattagggtcctcctgggtccctttctccttctgagagaggaagggtatcctcaggtggcagccacagggcccccttggtgacagcccccttcgtgggaggctgtggccttcactggtgcacagtcactgtcgccctcagagagctgctgggagcacaggctgggctctgacagatgtcagggaggcctggcaggatgtcacagggctggtccctccctgccccagggaagccactagcctggggagggggaaaggacacatgtggcaatcgcccctgtttcagaacaccttggtgttaagctggggctcccatggtcgaggcagtgacggctcactcaccaggacatccacagcccatgggccaggcagatgagaggcccaagcgagcttcagaatgcctgtgaggccacagccagtgccaggcccagcgctgcccaagaaaagaggctgctgtgaaagagctcctggctgggggggctgcaggagacagatgtgtctgctggcacccccatgctggccagccggggctcccaggccaggagctctatggaggatgaaccagaggcagaaacatcctctgcagatgccaagcgatcctcagccctgacggaggcctgctagagccccagaggctgtggacaagaaactgctcactggggagggcaggccaggggcaagtgggaaccctgaggagctgggcagtctgccccaccacgaccttgagccatgtcctaaacaggtgagcaggagccacacgggggactctcaacggcctggaagcgcggctccctgggagacactgagtgcacccagaccacaggagctggagcctgctcgatcctcgtcccatgcacggctgcctctagggatctccatcctgggctatgatcggggtaggggagggaagcaggtatgtgctgagcccaggatagacttggtggctttgtctccagtgcatagtgaaggcccaggggtccagtaggagacgagggccacaggcagaggagataagaggggcctagtgttgggggaggctgcgaggcagcgtttgcccaaggtgtctgcctctgttggtgtggagacctggggagacagtgagtgctgtccacctgccagggaatggggcacctgggtccaccctcatggccacatccactgcccactggtgcctgccctgcgcggggccctcacctgaccttggccctgggaggtcaggcactgtcactccgtttagtgacccaaacctgcctgctaagctggggtggaccagcacccagaccatagctgtctgacccccaagctccatctcccagccctttgtcactgctgggcttgacttgggtcctggggcctggggtcggggtctaggcagagacccgccttaagcacccctgggttccaggaagacggcactgcagaggcgtcacccctgggttccaggaagactgggcactgcagaggcgtcaccccgggttccagaaagacggtactacagaggtgtcaggtgggacatgggcttcttaccccgtggctggacttaagccccttaggggtaaggggaacaacagccaggggcctcacctgccctggtcctgctggggatgggtctccatggacaatagagactgagactcacacagactgccttgtgtctggctgggattgggtggcccagcctggacagctccagcctccactttgctgccgagatgctgtgtgaggagctcagggcccagtccccacccggtcgccgctgtcctgattcactgagaccgcgtgcgggtgccctgtggctgctactatcactgtcactctgcccaagcccgggaaggaatgagctgcccaggcagggcgaggaactccatctgctcttattccaggacccaggtaccccatctggggccttggggacagagggggtggcagggcagtgccctgggtccgcagagacaaaagctggggcttgcagagggccccggaatgcccaccagcagctgcccctccgctctgaagacacagagtgttcaaagcaccacactcagcctctgccctgggccgggaccaagcgcagggtcctctgcccgcaacttgctttttgacagcgccagctggagggcccgttgccagtagcctggcctctcctccggcctctggcaaggacctagctagcgggactgtgggccaggccctggagctgtcccacgaggggcccagcaggctggcaacaggcctgctctgctcactgcagagagggccccttaactcctggggaggaagcccccaccatccttgcccccaccatcctcacccccaccctgactggtggctttgaggagaagccaggattaacccagccttttgaggcccggctttgtcagctgtggggattggaagggtgccccagggtcagtgggtgccctctgctcacaggacagtcccggagtctctggccacagcttcctgggacagccttgggggctggaattggcagcagctggaggtggccggagacagcggagggaggggagtaagctgtggacatggtggggtggtccctcttcagcttttaaggtaatagttggctagaatctaggtttttgctaaactttgtaaatcttcattgaaatgcctgggggaaggagaattttggcctccgaatctctgacccctgcagagagtggtgtctccccaacgccaggtgcaaggagctgggtgcctcgctccagtggcatggctgggggaggggtgggtttgcaacccttggggtggctctgggctttaggtaatccccctgtgcggtttccccatgggctctgacttctgcctcactgaagcctggagtgctgttaccttctccaaaagggtctttctgccctaaaatgccctgttccgctcacggcctctgcagccccatcgtaacccctggctgtggacaccagcctcggccgccacgtaatctctgagcctcacgctgtccgcctggagtgcagagaagatgcttttctttccagccgggacaccctggaattgtctgtccccctcttcctgaggctcctcggaaaagggacagaaggtctggaggtcgctcagggccttctctacccagaggcaggggctgggcctccagggtccgggtctccagggtccgtggcttcaacttttggaatctcagaaaatcccagaagtgaccactggcctggggtggcagaaacccaccagaccgggaagcaaaactaactcactgcccagctgagccccccaccttgccccgagtcccacacggcccaaggtccaggtcacctacgaccgcatcttggcgtcagcactgggtccaggagaagcctgggacggccctcccctgtgggcctctctttccccctgtcaacgggagttagtggcagtgaggccaggcctgggaggggtgaaggcagagggggcagggaggcaccaggagaggccggtccccaagcccagctgcctggcttctcgcccatgaggctcgccagcctcggagcactccagtgataaagcgttcttgagggattttcctttgtcaacaaaaccaagcagtgaaagcagacagggacagctctccacccccagtcccagtttgattcactggcagacagcagtggaaggctcatgggggccaaggtacagcctgggctccccccacaccccattcggtccaaggctttctcccctagggacaggcaggtccctctgaggtgtcagcacctccttatcactggttcccctgggggccctgagggtcacagggacctgccccacagagctgggagcagagggtggggcagggggcaggggctgctcgtggggacctgtccagggacctgtaggggccggggtggggcaccacacgggctgcaagggcaaggttaggggtctggctgctggggccaggtccggctctgccctctgctgacaggtgaccctggtgtgccctctgggagcctcagtttcctcatctgtaaaatggggagtgtgggaggccctgcacataggggcaatgctgctgccaacgctactaatcagacatgctgggtccacagggctgtccccaaactggaaagaagtcctagaaaatagatgattcttcctaaaacgtaaattggatcatgcctctcccccaacagtctcatgcctatggaagggaatcctgtcccccaaggggttctccctcctgtctcctccaagaaccaggcagtgtggggtggggcagcaggacccagggtctcagagctggggcctccccagcctcacttggggcctccaaggctaagttctgtgacctccagggcccaagagtgagttcctctggctgctccagaaagtgaagagagaagagccaaccccggctcgggccacgagccccgaggcgctgaccacagccagagcaggcctgctggaagctccgtccctgctgcagatccctgcgccacatgatatcagcaattgctgcaatttcgccctaagtactcttgtttcccctaataatccacgtttcatctccacaataaacattgttagaactcttccctcagctttacgcacatataattgcttctggtgccagtaaaaggtattaattccactcatgcattccataaacttttaggataatgcaattgataccatgatcgtgttgggacacctccgctattattctcctgcttgcaagcctccgaggcgctgggaaggttgctccagacccattagtgttgactcagcctcattctccccaggaatcccgtgcagagcctttgtgcttccctggccgtccttccatatgtccctcctggacggggggctttcctacccgtggagccagctctcccctaagacaaacatgtgtgcacatgctcgcgcacacacacacacacacacacatacacacgctcgcgcacacacacacgctgagtctgagagcagctggagggcactcagggggctgtgtccgggccatctttctgtctggaggaatgctggggcagctggccatcgcacctgtgtgtgtcctcaccatcctgccatggcagcctgcctcgtgggctgtgggtggtgtcactcagcatgccctgaggtgcccacagatgctgtgctgggccagggcttccaaccccacactgccttcttgcctgacctcagagccccgtccccaaacttcccaaacaccaaggacagccaccttccctgatgaggctgagctgggggcagaacggctgagccggggtgtcacagcatccgatgcccatcgcgtcgggctgttctgacagctagaggaactcctgttcattctctgccccgagggcctcctggaaccagctgtccacaccactgccaacctgcagggctcccaggacaggagtggccagcagggctcagaggccaggtcatgcatgccctgcagagagctctgcccacactctgagcccacactgtcccacagagggtgttgaactgggagaagggcactggtgtcaccatcagcactgcactcagagctcacgtgtgtggagcgctccccaggtaccctgccctgacctaagaagtcccaagagccagctcctttgtccccaccatggctggcaggccatacctctctttgcaggtgaggatgccagggcagaggggcgaagcagaaggtgctcacgggcagctctgggattgggaccccagcagacgccagctccacccctctcaccatgcagggtccctctcaccaaatgccggccttgccctgtcactggtcactggttacactctcgtggctaaacacaaatccttcctctggctgctcggttacccacgactcgcctcctcctcctcctgggtttccatggggtgggagctactagaaccctgggcagtgatggggggaggcctgtgcatgtacacgcatgcctgtgtgtttacatgtgtctgcgtgtgcataaggtacttgtgtatccacgtgtgtgcatgcatgtgggagggtgctgtgtctgtgtgtgtgcacatgcgtgtgtatgcacacatatgcacagacctgtgaaccgcagcctccagacctctcagtgcccaaggagatctgaggtctgatctgtggtcacttgggggctggtgagggtgtttttcctgatcggggagaaaggagggcggcagccaatgtggacttgtacttgagaaaaatatgaattatctatcagttaccagccggctggcgtgccgggagccccaggagtcacggtgcagaggccggacgcaagcacaggcagcagggctgggcagcacccgcatggtgggaccccagagctgggagcaagcgggctgcccggcggccccgttctgcactgtgagccgaccacttgcctgcccctccctgggcaaggaaagaagccctggtggagggggttctggccccagagagctgaggtgccaaaaatctcattatcaatgtttgtcccaccccctggatccacagggcttcggaggaggagccctgggtgcaggcgacagcattttcctccccagttcgcgcccccccaccctccccccgccccagcatccacagagcccagcagggagggagcgaggaggtgtcactgctcctctgctgggcaccaggcacagctttcccctcccgctggtgcagctgaccagcccgggatgcctggtctgagcagaccagcccatcccacagggcaaggccaccccgtcctgcaggacaaggcagttgagacaaaaccatgaagtcccttgaacctgatgccacacacctggcagggggtgggggtggcagataccctagatgggggaagccagccagagagggggttgctcctgtccttccatgaggatcagctcagaccacatgcagtgcattccaagcatggtgaagaccccatcagcatcctcagaccatgggaggaagggaggcagagctcccacaagctgcctgccaggactccagggagacactctccagaaaccaccagcatcataaaccccagggcaggaggtcagtgtccttagaggggagagagtggacttaggcaacaagcctgagtgtgggagagaagcagtggtgaggctcctgctgctggaggtgtgcaggtgaggatgccttggggctttctgacctggcttcagacctccaagtggggtggaaggataggcctggtcccccttgccccacaggcaggtgcagctgccacggaaccactcatttgggaggcccaggccagcacaccaccaccacaattaagacaacgcatgggcctcctgctgcgagctaagcaccgtgctggccacagccctggcacggtcttcccatccttatctccatcgtgcagtaaggtgcgtggaggagtaatttccaaatggagaaaacggggtgttagcactggggtcggcattgcctgccagatggaggtgggaggccccggaggaggtgccaggtcctctgcccctcagcccaacagcccacagagccccctcccatcaagcccagcgagtccgggctgagccttctcttagcccggagccagcagctgggggagcccgtggctggagtgagaatcagaaacaaccgcaggtgggggcgggcggggcctggaacccagaccccagaccagcccggcaggactgtccggctacctgcagagcctgacagtctaccttaggctccgccctgcccctcggccctccaggagcgtgggaaaaccgcagctatggccagcagggcagtgggacccgattcttaaagcggaaaaacctgaggggccccttgctcagcactcccgacagggggtgggaatgcggccgccctgcatggacttcagggggccagcactgagctgtgcccggggaggcggggcctcgtgggggaggggacgctgcccagtgggccggaccaacatgtgtcccatagagcccctcctgcagccatggcctgggtgcccaggcagggctcacttgccccctcctcggtgcaggaggggagacccggagggctcttggggctgcttttcccgcccagcccctgcgtgccctccactcctggctggggtcatgcagccttcaccttgcccctcctgacagaccccctcccctgaagctgtctgggcagggcagctcagtggttaggaatacagatgcagaggtggctgcccccacttactatgagcagaactctgggcgaaccacttccctccccgagcctcagcctccttggctgtaaaaggggagcagtgatggagtcagccctgtgcgttgttggtgggtgcaagagttagctcaactcaaagccctgagcatggtgcctggctcgaggagactggccttgtgcacaggtgatgttatcagaattagcaagatccccaccgtgggccagtctgtctccaggggcaccgcagtgaggacctgagcccttggcacggtggttccaccccatctccttggtgcatgccaattcctggaaatgacactccagagacagtgccagcaaagcccaaatccacccacccgcagatgcgtctcgctgcctggaggaggcaggttctgcagaccctagggctgtgggcctgtctcggacagacaactcttggcagcagagcccgtgcctctgtccaggacaggatttcaaaccaaagatcccacctgagcatggggaagccgctggccagtcttggcagagatctcaccagcagggccaagtccgtcccctctgtggccctttggaggctgactggctctggccactcacgtctagaggggtgggcacacccagactgcccggccctgagacccgcctcctgcccgatgctcctcctggcccgggccaggtgggaaccccgagcagacagtggggcgagctcagacctcttgaggtgcggggaacgcagccacctcctctccagcccgcagcccgcagcccgcagccctcagggactaggggaactcaccgtccaggtggctgacttggccgtgctggactgctggaaagtgacctcaaagtggtggggtccggggtagtcggtgttggaggggatgacaggcgccggcgacatggtgtcgaaggtggagctgggttgtgcgtagggcgagtgggtgggcacgctggcggcgtgctctggggtgtaggggctggccgaggccgcgcggctgctcatctggtccatggtgctgctcagcagattgaactgggcctggcgcgggaaaggggaggagggagggggaaacacacacagtcagtcgtcctgtcccgtctaggaggtgtcctgttacaggagccttaaagggccagcggctccagtcatcccaaatatttccccgggtctctcccccatcccaacaggccactacttgcattgggataaactgacatattcacctcgccctcccctccatcagcctcccggtgccagggtttctggggatccacccagccgttgatgaggcggaatagactctctgcacacctggaggggaggcgtcgggtgggggaggctcccaggccctgatgcccacacgggcttcactgtaccctccgggcggcatcggggtctggacctcacagctcaaagtggggagatccagccccagggagccaggcagtgggggaggtgggctgggcacaagtgggccctgagtgcagggtgcgcacacctgggcttgggtgcgtgggtgcccctgaggtgctggagaccagatccgggctctcactaggtctggccctctaggtggtgggataaggtggaactgctgttcaatggctgagcccaattttcccagttgggcggcctcatctctcggtgaacggcctcaccccgactggaaaggccgctccacgctcaggggccggcaaagcctcacccctgctgccccctggcccggggttctctcgttaggcccctacccgcagctcccccgccaggaccacacaccaaggaacaggaacccctgtgggtgtgtcccctgcgccaccgtctgtggcgccccgggaccccagcatggatcagcctctgccaggagcttaagggaggccccagctgagggagggggtgctggtctggcctcgatgtctcagatccagggcagggcagacctcacttggtgggctgctgaggaagaccctccttccaaatccagaggcgggtgctggaggaagactgcacatgtataaacagcgctttccctgtgacccccgaggctcaacgcaaacaaatgcctttacccccctctccccgggcctgcggcgggcaccgtgattgtccctattacccctctccccgggcctgcggcgggcaccgtgattgtccctattacccccctccccgggcctgcggcgggcaccgtgattgtccctattacccccctccccgggcctgcggcgggcaccgtgattgtccctattacccctctccccaggcctgcggtgggcaccatgattgtccccattttacagatgaggaaactgagctatggggctggggaaacgattgaccccagtgatctagcggccccagcagacggcacaaaggtcaccagcagtcagcaaggtgcaggctgagacccccagaccgtgaactgggattgcagggcaggaaggggcagagccaggcggaggcgggccgggggcggggggcggtcattctgccctgtgtccctctttagagaagctgtggtgtgctgtgagcacactcttccctcccaccagcctgccccctccaatccaaaaaacaaaacgtgcactgcccgcacaggagggtgcaggggcctcgccgggactcccaggctcagccccttcctgcacctcagccttctccatcagaggtggttgggtttagtgacccagaggccctcggctctgcaggtcctaggacacacatccactgaggtccctgacgagggccgcatcccccggcccctccagcatccaatggagaaggacgccaggcaagtccagagtttgcgtcctctctgagtcctctactgagtcctccactgagtgcagtccaacgtggaggcagctcagcccgggatgccggaggggcaggaggcaggggactgctgctcaccagagccatcaccactgttcccagggccaccaaaggccaggcaggccctctccctgtgtgctccagcccaggacagccaacagccccagcaggcctgagccgtcaggggcgacccgtgccctggctccactgcaagggtcttggcacgaggcctcggctgaagctgccatcatctggcaccattaagctggctgtgagctgtcaacctggggagcccacccaccagtggaacggggaccccctgcagcacgggccctgcccacctctccggcaccactgcagcctccttcctggtctgtcgcgctccccacaacctccctaacttcctgcaccacaggtccttcccaggagcccagggttcactgaatggcaaatggataaagaaacagtgtgtctgtacagtggcctcttcctcggccataagacagaaggaagctcccacacgctgccacgtggataagccatggtgccagcttaaaaacatcacggggggacgcccgtaatcccagcattctggggggcagaggcgggaggatctcttgagcccaggaatctgagacggcagagagccaagatcacgccactgccctccagcctgggagacagggcaagactttgtctcaaaaaacgaaacaaaatataacatcccagggaagggtgaccctcgcctctcctggctcctgggctattgtgagggggtctggggaagcagccactgcccccctcagagaaagatgtagaaaggctactcctgctcctcctggtgtctggttgggtgtcaccctgggcaccggcgagctgccagtaggcttctggcacagaggatggacccctgcctcagctacacacatgggaggttgggtgatggtgggggtgacaaggggcagtgccaggctctggtcgcacagcccaggccccgccccagagtccccggctcccagactcctcaccgaggcccaagaggcctctgaggtgtctgagacctgcttacgaccacgggcccaggcccttcctggaagcctgcacttgtggtctgcacacgacccttcaccagccctgtccgggctcagaggtcactgttccgaggggtcagccctggccacacgggcaggagcagcgcccaggcctcaggtgttctctttcgtcacccggtttgagcatctttctagccattctccggaagggcaggctcgcatgtcccccatggcttctgtctcccctactcgactgtgaacccacgaggaccctggatacggtggccactggccacacagggtgggggtgtgtgaaatgtggatggacctgagcccagtgtgtgagcgcaccccgattccaagggctcaggaccacggaaagaacggaaaacacctcgctcaccatttcctactgattacaggctgaaacgatcacagttacgggtgaatacaggaaataattaaatgacttccgccagctcctttccacctttttgaatgcggctactagaaaaagcagaattacacacacggctcggagccgacaccctcagtccgtgctcctattacagcgtggctgcttgaaaggctcgctgaacaagcgagctggcagaacaaacgaacaagcgaatgcaggacggctcccccagtccagctgcccaggagtgagtgtgggacatgagccggcccctcccatcaccgggtaccgggtgccagcctgcccaccctcccctctccgtggtgccctctctcttccaggtgaccagcggtggccttcctggaaaggtcttccctgttttgggcccggggatggcctcagggatgcacgacttccccattcctgactcatttgagtcccccgggcaagaacatcccggggctcaagggctgcccagctgaggcttctcaggtcaggatgacaggcaagggcaggctccagggtccccctgcccgactcccatccctgctccatccctgagaacaggacagccagtggggcaaaaagcccatgcctcccttgtttgggagaagcaggatgcagacccacgacaacgtgtttcatattttaacttgtccacattcttgtatgatctgaggaaggtgccaaggaattagtcattgaggcaagaggagaagagctgataggatctctgcgcctagcacagcccagcgggagtgagccaggcccttgcggcaggcggccccacgcgggaatcctaattagtggacaagtgtccggacagcaggcctgaagccctctcggtgccgtcctctgtggcccagggccccaggcaggctcccttagcagggccttcctcaggcagggccccgatgtcggcctctccctctggtcccctctcatcctcaggctcagtgacacggggtgggggcctctgaagccccctcctccccagggttactgggcctgtcctctgcccaggggacgctgtcctctcctcctgcttccaggggacctctctccctgcacgctggccctgcctgtggtcaccgcccctgtcacacccccagtgggtggcagaggctttgcagctgcagttatgaatgcagctgggaacgcagcatctgggaagtgtagggggctttctgggtctctgacctcccccttaggcctccccaggcatcctccacaagcccctccacctggctcgggggactctggtcttccctttcgcttcctgcctggcggcaaggagctttccctggggcctggctcctgctcggggtcaagctgctgaaacagggaaccctgaacctaacacaggcctgcacaggagcccggggtctcccacaaccagagggcccccggaagccacggggaggaacccgggcacaaccaggacttttttggtttgttttgctttcaacaagagctcccaagatagtgggctaattaggacacgcccggcccgcggcggccacaagtgcctacatgtgtgtgcagggctcctgggcctgacccaaggagtgcgcgggagccagagagccagcgggtggccttcccgccagcctccaggccgcggacgcaccaggccctgctggggccccagggccttgttgcggggcaggagtgctctgccaggggaaatactgcagcccccacgcacccacccaggaggcctcatctctcctccctgcggatgggttggggacctccctgggtgcacctgtgctggagattagctctgggctgggggctgggggctgggggctggggtgtgggcagcaggcagtggtaaatggggctgctctctcgcctgggccagtcacaggtatcaacacagttcccagacctcactggtttgatccacccatgcccctggggtggcagcaggccagtgaccttgctccaaaatgaggggtcagagactgagaggccacccagtagctgggccccctccaggagggctgtggagggtcccgcctgccccctggcctgcctgtctgagagcagagaggagcagtgtgggggaggccagagctcagcccagccctgaagccccatcccactggctggacaccccaatccaagcaccagcatctaccgtcggccaagctggggacggtgatggctgaggtctgggggttcccacaaggtgggcaggaagtggtggccagggctgcccatcccagaacagcaggttcttctgggtctactgatgcccctacgccaccatcctggaaaccaggcctctctgcccatccaggagaggggcaggagagacctcccagggacctggggggccaagcagggctctgaggtcaggagtcctggggccaagtctcagctccacctgtgtaacctggcccatcgctccacctccctaggtcttcgttttctcatctgcagagtggggatgaaatcaacatgtactggccaggcgtggtggctcatgcctgtcatcccagcaatttgggaggacgaggcaggcggatcacctgaggtccggagttcaagactggcctagccaatatggtgaaaccccgtctctactaaaactacaaaaaaaaattagctgggcgtggtggtgggcacctgtaatcccagctacttgggaggatgagcaaggagaattgcttgaacccgggaggcagaggttgcagtgagctgagatcacgccacagcactccagcctgggtgacagagcaagactctgtctcgaaaaaaaccaaaaaagaaatcaacgtggacttcgtaggatggttgtggggatcaagagggtccagaagtatggtaacctgagacttcatcggaagtgactctcgcccgggagccctcctggaaccaatgtccaggaagctcccatgcacgaggcatgacctccacgccttggcacaaaaacccagccaggaggaacgacggctgggcaggctccgccgaggaacagggacgggaatccagattcactacgcagaggacagtcgctaagccctgggcacagggacggatggggaagagcaggtgctctcgggggcaggtgctcacaggggtcggggtctctcctgttccccgaggtgtccagatagccagtaccgacaggacgctcagaaactcttggttggatgaaacagaccctgaaatccacgcgggccatccaactgggggaagtggccacggtgtgccactgcccgccatgtcggccacagccccaagcccagacagtgcccagggtccaggctgccttcctcagaatgtggagggcggggacagccccttgaaagactctttgtcctcaccggccaggagctggaggggagctgggccccatcatcaccctcccgccgcatgggttctgttctatttcagattaagttggattttgtggttttgttaaaagattcccattgcacctgttgactgtttaaaatgtgtaaaccgtctcaagtgactttttttttttttgagacgaagtctcactgtgtcgcccaggctggagtgcagtggtgggatctcggctcactgcaacttccacctcctgggttcaagcgattatcttgcctcagcctccccagtagctgggattacagacgctaatttttgtatttttagtagagacgggggtttcaccatgttggccaggctggtctcgaactcctgacctcaggtgatctgcccacctcagcctcccaaagtgctgggattacaggcgtggggcatttgagtgattttttttacaagtagatgggaaataaatctttttcaagtttgtgtccctgttcccccctagtccactcctctcgcttcccggtatccacagtgccttttatcagagcctaggatatttaagacacaatgcaaagcaaggcatgggaggtcacgaccgtgccatcggatgacaggggacagatgtggtactgtccagaacggaacgtattttttagtggaaatgacaacagagaaacctgccagcaaaattcacaatgactctggccacatctccaaaggccacggttttggctgactcctccagcggaggagatgtgaggcggggaaggcacatgcagcctggtccctgcattgggcctggcaggggtggtggggagggcggggcacaaatgctcattcggaaacctttgtctccacaagtgcagaggctcgaaagacggagctcctgccccgtagacagcaataacagcttcccgagggaccccatccttgggagctgaggccaggacagctcccaaggatggggccgcttcctggggccctgggcttggcggagatcaggagactggggcccgagcccacggtctggccatgcagtcccccttctcttggaaacagagattcctctgatcctaatcacttaggatccgagtcagagagggaggcacgtgaaaaagcgtagtagggatgtcagctgaaatcctgacgccccgctggcccctccccagctccacctccgcctccctcgctcctgtcacctctccagctcctgcccccgcccgagcatccctgctccccgcccggcatttatcccgcactctcattcattctgttcaactaggacaggaaccgggagctccagacggggcagagacatgctcttcttcatccaccactggcagccatggcatggtgcatggtgtgcccgtgacagacgtttccagaaggcagcatgtttgagggctggagtctggcatcggggcctgcctggagtctcctgagggctcagctggcctccctctggcctcctggccttctccctccagcctggacaggggctctgagctgtgaccccagctcttgtagaacaaggaggtccgtgtcctcgttccctggagaatattgagaccagactgttgtggttttttgagagtatgccgctcctcaaactccctgatctctcgtgggcatgtctgaacttgggccccaacctcacccaggggtgagtgactgacaagcagcctgggcttcggaggaagagagcccatcccaccagggcaagacctccgtcctgccggtacttcccacagcatctcccggagcccgtgattaaacgaatggaaatgccaacaagattcgacatgcggctggggggaagcctccctctcccgacaatgtgcacacagcaccctctgctctcatccacagcaccctctgctctcacccacagcaccctgtgctctcacccacagcaccctctgctctcacccggggctgaggcgaggccttgcccaatcccgtgccagggccatgtggaactttccacacctcacaccggagaaacgcgcccccaggagcagtccccagacgtgtgtctgcaggactgcgaggcccagggccaccgctgctatcctgcctgcacctgcccgcagcggctgagcccgtctgcagcacccactctgtgcttagcgccacatgggggccttgcagatgcatgtccctccacacgcccaggtgacttcagatgactccagggtagcctggtgccccccgggcctccacggacccacatttgagccgcccactgtggcctggtgcaagggcctcgccccaccctctcctcaaccctcactgccttccacctgctaaccgccccggagtccactcacggtgccgccgcctcctcccccagacccttctccacgtccctcagcttctgccccctgcctcccggatcccgctcaaagcccacctcccctgagaggcctcctccagagggctggcccaccagcccctcccttccaggttgtggcgaggggcagcctggaccctgagtgtctgcgggaggccccagtggggtatccaggctgtatccccatcaggccctgtgctgctgcctatgagaggcaggtggccaacaggatgcacagactttatttaaccacctggaaacgggctggtccaggccctcccacccagaatgccgagtgcccgggaagacgagggggaaggcacagctcacacacgcttctctcccctctccgaaccctgctgccagcaccccagagcccaacccaggtgtcagccctgacatcctgtgtccttgcacacatgcccctgcccatctgtctgcccatccatccacctgtcctgcagatgtgaatcaggcgccacctgtaacagcctcctgctgtgctggagacactgacagatgcttagcagggcttcctggacaccttccagcacacattagtggctgagccgagaatgggtggctgagccgagaacttgaattatgacacgtgcagtgcagggtacagggagggggaggcagtcacggggcgtgccacctccgatggccctcggtgataagcggacattggagcaggagcccaaaggtggagaaggcggtccaggacggctgtggggggccatcgaggcagagggcagaggccccagagccagagagacagcagcatgcttgctcaggaatcacccagccccgctctgcctgtcccctccccagttagtgggctcaggagtgacggatggtcccgcccagtgcactggccttggtcagctcctgcaaaggcaggtctcctcttcacacccatttcctaggcttgacatcctagcaccctcccaggtgcaggaagatggcagccggcacgagacacgaccccggccctccatgctgacgacagtaaacaaaccagccagcaagcgtgcaggggcttgggatcaaaagtggtctcagaaacacagagccaggcacaggggccagggctggcgtccaggcaaggacgggctcgggaaaaggtgaaggcgggcctgcaggaagggaaagatctgccccaggtattgggtgggcctgggggtgtttccaggctggggaccagcagggcagagagcctgcagccagaggaagggccgagggcctgtgcagggagagacggagcccagggggatagcgggagaggtggtcggagctgagcagagacgcccggggctgcaaagcgctccctctggctccggtgttgagaacacagggcgggcgtcagcaaaggcagcaattgtgagatgtatctgagcagtgggtgcgaggtgggccgcgggtgcgacgtgggccgcgggtgcgaggtgggccccgcaggggccccaggcacatttgcaggcaggattcacaggacctcaggcatggagagtgcctccagggctctggcctgagcaaaggcgagagtggatttcctgctggctgagacacggcaccatcccctctcaggggaagataagtttagtttgggtttagcaagttgaggtcctattaggctggtgactggggggaggtccaatgggggacagaacaaaggagacagaaatagcagctggccgagggtggcaggggggccggaggcgggcgtgcactaggcacagcaggcgggtgagtggggtggctcctctgatccctggatctccagttaggacaactggtccctcccctggccccacatggcttgagccccaccgctgctcggggtctttcatgacacaaggcctggagaggatgctgagggctcccttggccatgctcagcaatcccatgagccattcccagttgggccctgccctctccaaaccgtgggaactcagcagcctctgggttgccaagccccagtcccttttctgttctcccagccagcctctcgtgcctcctggcctcccaggctgcccctctgcatctccccctctgcttggtgtctcggatgtcgtagtgcccagtgctcagctccagccgctgtctctccctgcctccgtgcagatcccccagagccctgactgtaagtgcccatgtgcaggactccctagaggtcgtctccacagcctgcctgcccacaccagcaactgagtgtccacatggccgctcctgccagtgtggccccaactccttacccacctcacatccagaccctgccggcctttcctgccttggggagcagcagagcctcctgcaagggtgcaaatggaacctcagcatctttctggaatcctcccctgctcttcctctaaggtcaccttcaaactccaccctaagcctgaccaccacccaccacctcctctgatgcacacaggactgagccaccaggtggtcacacttttcaggccccaggaccactggaaacccctccctcatggggctttgggaactgctcgctcgtcgtcctcctccctgcatgcggctccttctcagctatgcaatggctgccgggcatctccaacacagccacggccgaccgcggcgttttcacccctgctctgctgcagttctaccaatctcagggcagaaaacttggggtctcccccaccccttccctcatatccagcccatcagcaaatcttgcccagaatcccacggtgtggtaccacctgaccggcggggctgccccattccctggatcgtcccagcctgcaggcggctcctggccttggccctgcccccctgtggtctatccccagcactgcagccgggggccctcccagaacgtctgccagctgcgtcccttggttccgtagccccacgtacagagtaggtgcctagtaactgcagcttgctgcactgattggcttccccccgccccagggccctgtcctggaggcccatcctgcccagaggtttcactttctgcaggaacagagtggacactggctccatggggcgcacgggagtgggggtgtccaaagctccagatccgtccgtgctgctcctcagcacaggggtggccacgttcccgcaacgcatggatgcccaccatgcggaggggcagggtgggaacaggccaggcccaagggtgcacagcccaggccctgcggcgagacaggctccagcccctccatagagagggcgcaggagagctaggggtgtctcccatcaccctggctaacctcctgagaccctaaagctgaagaacagcggccccaggaggaagtggggggcaaggggccatgaactggacatttgggaagtcagggagactcctgggtgttggtagacagcacccagctccgacgtctccagcgctcagcctcttctagaggggccccactcatccagccatcggctccttccttgctacaggcagaagtgggtaatgtctctgtgtccacctcggggttgggcattgatcaaccccagtccgtgggagccgcagactcagaacgtggccttgtttggccagagccaccacaggattatccagtcccccatgtggaaggacagtgtgggaagcaagtgaagagcatggctgggattgcgggaggaggggctgaggctgcatggctgggattgcaggaggaggggctgaggctgcagggctgggattgcgggaggaggggctgaagatgcatgggtgggattgcaggaggaggggctgaggctgcatggctgggattgcaggaggaggggctgaggctgcatgggtgggattgtgggaggaagggtctgaggctgcatggctgggattgcaggaggaggggctgaggctgcatggctgggattgcgggaggaggggctgaggctgcatggctgggattgcaggaggaggggctgaggctgcatgggtgggattgcgggaggaggggctgaggctgcatgggtgggattgcgggaggaggggctgaggctgcagggctgggattgcgggaggaggggctgaggctgcatggctgggattgcgggaggaggggctgaggctgcatggctgggattgcgggaggaggggctgaggctgcagggctgggattgcgggaggaggggctgaggctgcatggctgggattgcgggaggaggggctgaggctgcatggctgggattgcgggaggaggggctgaggctgcagggctgggattgcgggaggaggggctgaggctgcatggctgggattgcgggaggaggggcggaggctgcatgggtgggattgcgggaggagggggctgaggctgcatggctgggattgcgggaggaggggctgaggctgcatgggtgggattgtgggaggagggggctgaggctgcagggctgggattgcgggaggaggggctgaggctgcatgggtgggattgcgggaggagggggctgaggctgcagggctgggattgcgggaggaggggctgaggctgcatggctgggattgcgggaggaggggctgagaatgcagggctgggattgcgggaggaggggctgaggctgcatggctgggattgcgggaggaggggctgaggctgcatggctgggattgcgggaggaggggctgaggctgcagggctgggattgcgggaggaggggctgaggctgcatggctgggattgcgggaggagggggctgaggctgcagggctgggattgcgggaggaggggcggaggctgcatgggtgggattgtgggaggaggagctgaggctgcatggctgggattgcgggaggaggggctgaggctgcagggctgggattgcgggaggaggggctgaggctgcatgggtgggattgtgggaggaggagctgaggctgcagggctgggattgcaggaggaggggctaaggctgcatgggtgggattgtgggaggaggggctgaggcggcagctctccaggccaggcccccctgcgtccccatctgcagcaagtaaccagtggctcccattgggccccgcagccgagcagctacggtgaacaccgtcctcatcatagaggagactgaggctcagagagcggcgtcactgaccaaagactcagctggcagatgccagagagacagaccccaagcccacacctggaaccctgtgccaggctgggggcaattggcgggcccagctgggggctcagacagtgtcctgcaacccaggggcctcttcccagggccaggctggcccagcgggctccggagcagctactgtgctttgataggaataaccagggcctcacaaagccaccaactccaatacaactctattcgctcactttgggtttaaaaccctttgggccaaataccactttctctctcaaaatacgttcctgcagtaagatgaggtcatgaatactctctctggcacagaaacagggaaactgagtcaccactccgcccattaggatggcccagcctagcccacctccccccgagagggaagaggtgctggagacatggctggctctgggcactgcctccacctgctcctggcactcagcctaacacacaaaacgtgcccagttaacacttgctgatgggatgaatgaatggacaaatgaacgaacgaaccacagtaatccttggtgtaaaaagaaacagtgatggcacagctggccattgctgacgcccagggcaccgggcagcagcagccgagtggacaccagcagctcctgcaggcagtgagggtctgcaggccagggcagcagggctccacctccccacccctcccgggaatggcgtctgcgcaaccacatactcaaccttggtggagtcccccgagagggtgagtcacatggtgacatctgctccattcaagggaaaagattgtactagagtcccccaggtccccaggcttctcggctggccaggcagcgacactggcctctccacccgaggcttctggatccacctcgggttccaggcggtccagcagccgggagatagcccctcccagctcacacacctcctgggagagtctgggggtacaaggcagccccccaggagccccaggcctgctgtgcgtttctaatagcaacgtgttggtggatacatgtatcaatgcacaagtgggtgacagccagacaagggagagagggacaccccatgcctgatgtagccgttccacgctctgggctacagtcccagcactgggtttcagataaaaagatcacgcaggcgtgtggagaggccatggtgccgccagactcgagtgcatggacccagcatctgggcaggctgtggggctgggtcacccacaccctcacccaccctctgggccacacacacacactgcctctcttctcaaagccttgcctgggaacacacagctacaggcacagacatccatgtatccatcgcaacacacatacggacacacgtgagcacctacccacccaaatcacacaatcacacctgcagtaccaatctgctcacacgtacacacaccacaacacacaggcaaacacgtgtacatacacacacaccagcctgtacacacacccataacatacacacacacatctgcacatatcctcgcctgcgtattgcatgcacatccacgcttagacacacacatgcacgcacaccaatacccacccgccacatgcacattgatgtgcacgctcacacacatcgctgtcacacacaccctggtcacactgcctggggagcccacggctccaaaggcctccgtccccagatctttgggcctctacatcccagtgccagcagagcttcggggcacttggactctgcctatctgcccgctgccccctgcccatggctgagtttcccgtgtggcacaggtacccatggtcccagctgtctgcttccctgggaggaggaacggccccacgtggagcagctgtgccgagccttccaccttctcaattagcagctggtgcctgcctggcccccagggacctggcaagtctggaagtgaggccaggtccccagggctcaggctgcaggggccaaccacaagctgcagacccttccctcccctcttccaagccagcagcccactgcagggggcgggtggcgcaagaggcctcaagcccagtttgccacaggtcctggccctggagctcagacccacaccgagaagagcccgtggggctgacaagaagcaccgtctgccactgctgtcaaatgagcaaattccctgatgagggcgggggcagcttcacacccaagcccaggagcaaagatgacttctagggtgcactgagctcctggcagctggggcagggcagggcagctcggaaactctgagcctggaccaagccgccagccccaaagccaaggagcatcagagacccccgagcacgaggcagaaacaccagcagccccatcggcatggacccggcctcctctccctggctgcgggtccctgcccagcaggtgggcacaggctgttggagcccgaatctgcttgcctccatgcctgggacctgcctctagcccctcaacgcctcccttcctacctctgctcaccccaactggcagcagactccaggccaggaaggcaggcagaaggatgcccctctgaaccctacactgcagcaaaatcccccgcaaagtccccgaagagatcctatgaatggcgcgtcacacctaccgtggcgaggtgccgtgcggggtcaccgacgtacagcatggtaggcgccggggggtctgcacacgccagggaactggtgtcccgtgggacgaggcatggatctgggcggcccgaggggaagggtcacggtcgtggccgcccggcagcgtggaccgagcgggagaggctccgcagctagtgaggcaggagccgaggatgctggggaacatgcgggccgtttgttggcattttcgcttttcccatctcccttagttctgtcaactggctgaatccaacaacaaaacccgcggcccacccctttattcctcatgagtattcatccaagtccggggcaggcctcagcttgcccggtgatgtgttggaccgcaccaaggaggctgcgtggggagggcggggacgccggtcagcgccggctccataattaaacccaccagggctccttccgagatgctgcctgcctggggaaggctcggggggccccaggccctcaccccgtaaagcagcctctgttcccaggcatgagggctgagccccggccctggagatcatggtaggcaaggctgggccaccccaagggggagagatggtgtcgcctcccttggcacccaggtggggcgcacctgccccctgcgcggggaactcggtggtccgccaggaccagtccaagggctgcatggagaggaggggctcacaggaggttcgaccctagacccccgagggagacaggtgacgggagtgagcagctcttgtggggcctgccttccactgcctgcccaaagcaggggtgagtccaagagcccagagccgtgcgtttggagcccaccctggcactggccaagcccccaggaaacagggacaagccactgttgccaaagaacagcccggcagtggggccccctgcaggcgtggacggccttggcacagccagggaggggctctgcagtggtgccccgcacccgtctagtggggcctttgaggtgactgccactccctgttgggccgtctgaatttccattgtatttcagccgtcttgggtacaaagttgaccctggcagaggggcccgagtccccctggaagcaggcagggcatctatgctcagacaaacctggggactggggcctgcggacccacacttccagtccccagggcgccgtggctctcacgagggctgagaaatccatggggagctgagaaatccatgcggagaccctttcccctttccttccaaccttttcctgccaaatcccaaaccacctgcctgctttctcttgtctccatctccccagcttccttcctccctgtcgtccccaccgagagccggcaggggcctcatcgctccccacccccacccccgggagagcgggcagagctgggtgagggcaggggaggtttggactgcagctgcctgaggagccctggggacccgccctgccccaccagcaggccgggctgaggacgaaaggacgattccaggtgttgtgccccaaggggctggggcctggagagcccagctcagggccctagggccgggaggcagcaggtgagtcctggacgcccgtctcttcctgtggcagaggatctcagcccccaacaggaagggcagcagcactcaggaggagacgactgagaaaaagacagttttgtctggtagatcaggttcaggaagggcgtctctgggaagccccctgcagggggtcggctggggcccagactggatgttctggggaaagcagcagcctccttgcaggagagcccagggcgagcactaggagactcctgctgggatcggggatgccctggggatgagggtgccttctgcttctgagcctgggacccctcttgggttgggggcgggctccccagggatggacagaccctcagagcagggccgaggcggggcctgggttccgctcccgacccccacagggcacatcctcagcctctgtaggcggtgtcctctcctgccaaagggaatgatgcattgaaaaggagactcaggcgagtcccatgcacagggccggcctgatggttttataaatagccaaaaggagagcgccaagtggaaggacgtgaggaaggaagacagccaggctgccttcagcacgagccccacccatgcccagggcaccgtgcctgagaccccgcatctctccacccttagcatctgggccaggctgcggggatcccagtgcggctgagctacacgtttccaactcaaaagcacccaaaaccactggcctctgccccagagcggcacccagctttctgcaaacgttcccaaggcccagccttgcagaggacagggacaggggatctggggccacacccccttggcgtggcccttagcgcctcctccaggttccctccctgctgggtcctccattctgctttcattctgcttctgactcaccggggattccgcctgactttccgcaggaccctgggccgcaggacaggggggaacgacgctgccgcaggccacggcactcttcaggaaccagttagtgggaggggttatgtgccagaacagacagggggacttcgtttcctggtgccagtgatgtgtcagttcaaggccaaactcatggcacagggtccctgctgggcagctggcttcatcccaccgcatggaagaggccgcagaagctgcaaggctctgagacctgggtgctgggtggggttcacttctgtgcagccccaaagtgctttgtcacgaaagcccaccaaaggaaaggctgagtgggggctctctgggagggggtaacgggtgggctggagtctcagagctgaccgtggtgagctgagccccctccctgctcccacgcatgaccccacccttcctccagctacagatcctccctggaagggaggggtgtccttctccctctgtccgcctgccctttcctgggggccacacatgcacgggacctgcgcccttccttctcctgggcctggggagaatacctctccctgaagtcaccaaggcctatctggggtgacctctagggtctcaagcccccaagtgtccgaaggttctgaccccgggtgtctgtcctgccactgggttcccggggctcccagggtagatcagcctgccttctgccccaccagctgtagaccccgctctgtggcccacctgcccacaacacctgttgtcagagaaaggcgccaggagaggcaggaaggggcacaggggaggggccagccctggaggaggctctagggacagagaggggaggggagaggacgggagcggagcagagtggaggggaggggagggaggagacacttgcctctctctccttttctcccctcctgtgtcctccctccttgtggagcatcctgagagccagcaagggccgcaggtctcattgccctgcgctggttttgtccgaggggacgctagcgctcagaggggcagggattcgcttccaagcccaggcccagcactgcagagactcctcgggtgggtccgggtgaggataggacccccgccccaccatcacacccacactgcctcctcgcagcctgcgagcctacgagggagctttatcctctgcagctcttcccagacggtgtgggggtggccagggcagaccccacccaggtggcatccagaagcccgtcaagccttcctcattcagggaaccagccagggtcctgagagggcaggggctgggccagttttagctgtggctcctcggcccaccctccccagcccatggggaggcccgggccacaggcttccccctgctctcctcttgcagaacggggccctattttagagtgaggtccctgtgagccatgtagggtcttcagggacagccagagcctaaggcacgggacagctcaggcaggaactgtgccaggcaaaggcagcaggccctggtcccagatcctatcctaggccctcctggctctgctgttctggtgtccgaggagcagctttcttaaacacagggcctcctccctgccctcaccctcacacctagcgtttgtcacatactcccgatgggcccggcaccactggacatcctcacatcgtgggtgacttggagcctctcagtagcgccatttatagacaggaaactgaggcccagagggctatgtaattctttaaaagttgcccagctcagaagggaaggggtaggtcctgagctcagcaaacctggcctcggagcccccagccacacccacctcggcctgctggactttctgggccacctcagtcagatggccacagtgaccacggggtctgtttcctgctatggccacacagagctcacagtctccccaataagtctgtggcctgggactcctctagagcccactgagggttcaactgctcttgcccacaccccactctcctgaactgcctaaactcagcctcctctgcaggggccagtgtggcaggagggttgtccgaggcccacggggttgggcagagccagcacccagcacctggcatcccagagtctatgccacctcccaaagggctctctgctctgggctcctaccccctgtcaccccagcttcaccagcaaggacatggaggctcagagaattgtgggagcttgaaaccacctgctccgcctgtctcccacggctgctaagcaggtggctaaggcatccctcagacaccctgtgtccaacgggaaccaatctgcctgctcgtggttcggggagacccacgtcccccagatgcggaaggcacgccccactgcagaggaccagtgtcctgcgctcaagcactgtgcctgcccgagctgaggcccctttgctggatgagtcccagacccgcccctgagtatgggagcagccagatgccaccaacacccagggacaggtggccagagggagccgccaggggcaggtgggggcaggtgcgtgtgcagccacttccacaagcaggccccccacctgagccacgaactcaggactatcggtagccccggaaacatccggcactagaaccgcctgtctgacccgaggccgggcctgtgtggtgtttcttgtccaaggtcaccagcctcgtggtggggctagaactgggcccctgagctgcaggtggcagttttccagggggcactcagagccccccatgcccgcatccgcactgaaccgggtccaggtccccagccccagcacagcagcttccctgagctcctgggctgaactttaaaacaatgccccccagttcagctccagccccactctgggacccctggagttagcgggccaggggccattctcaaggcagctaagccctgggccctgtgccaggctgcccgagcccacccgttggcgcccttcggagtctctgtctttgtacctggcgctcccatgggagtcaggggtaggggacagcatgctcttggcccagcagccactgttgtgagggggctgggccctggggtcagaatgaggcttggctctccccacgcaggcctgggcctgccttcattccagggaatcccccatctccgtgagtcgagctgtgcacctaggaatttcatacatggctttgggaggaggcgtgtcgctgccagacagatccagccaccttgctgtttgcggaggttcgaggctcccactgtcaccctgtggctggggagtggcacgttccagcaggcgggggctcggaccagaccccgatatggagctcagggaaccctccggggctggggaggacaagcacgctccacccactcaggaatgtagaactgctccttctgcctccctgagtccaggtcctcctcccgacacagggtgggtctgtgccatcttaggggccttctgccctctgcagggggagtggctgagcaacttggggttgggaggtggcttctggatctcaggggacaatcttatgggtcttcctccctccctccctggggtccagcggccagttcctgcccctccccagattggaggacacggatgtccagaaggggacctggagggggtggggtcaggacccttgtccccagagagcagccggcagaactgttccaaagcgagagaggggagaggttgcagtctcactttcttgcactacccggccccatccccagcccaccctttcccagcatctgcctcggtttcctcacccataacacgggactagaacacgcgcagacactgtcgagtgtttccactgggacggtctcctgagcaggggtgggagggcaggggcaggcggtcgaaccctcagatacaggctgaggggctcctcggaggaattgaccccccgggactgtggctgcccaaggaggtgccttctctggccccacatggctctgggccagcccagcatccgcaatggagacggaacaggcaacacggcccctccacctctacactcccccaccccgcttcactctgttcgttctgactggagccccacagcccccactccctccagtgctcagggtggctgggccgcccacccccatgcctcagacagtcagtcgtgcgcaggggcaaggaaggggctccagctcctggaatggggacttgagggaggaggagggccaggccgggtgctctcaggcccccagaggcccagcatcgcctccctccctttcgggctgagcataagtgagggaggcccgaccctgccccagacgcgctgttccgcgtatccccacagttcgggctggttttccgcagcgcaaacaaattccccacctacgaatccctgggcggcggaggcggggcggggcagccagcctgtgggcggggccgagcagctgacgagtccagcggggacggctggcctcgctgggcctcccaggtcgggatctttgctctctcctcacgtgtggtgcccacctgccatccacctcccggcacagtcgaggcctggctatgggcagccagagaggtaggcaggagcgtggcccccaccagctcccacagcctggccttttcccccagaggagcagccaatggggcagggcttgagactcccaggaatgggggagcctgccaagatggggtgccggaggcctcactggccctgcctgccccctatccagtcctcttctcctggaaacagcacctccatcttcctttgggggtccctgtgtggggaggcccaccccactccattccattgtcaccctgttccactgatggtggaggccaggccaatcagaggcacagatgtggctcagagatgggcccatgaccacattcagatcaacgggaggcaggactgctggactatgggaaaagagggatctcctctccacagggatagggcatgctggtggagcctggtactcttcaccatgagaggagatgcggcctaaaaacaaagtcactgcaggggaaagcagaccaagagactgagcgaggcagctgtgaagcacctggacacagccatgcccaaagccctttctatacctggatccaccagtagaagcccttcctacacctggacccacccatgtccaaagcccttcctacatctggatctagctgtgcctgaagcccttcctacacctggatctagccatgcctgaagcccttcctacacctggatccagccatgcctgaatccattcctatctctggattgacagttacatgagctgatacattttctcctttgcaatttgaagtgtcctcagaaacccacaggggaagctgtacaaccaagaggctatctcctggttctgccaggctcccaagacaggacaggccacatttgttgtccccactccagtcctcttgcagagggcagcccccccactcaccatgacagatgtagtcatgccctccaggtggaagacgtccatgctggaatccgttccgcccaccacctcattattcccccggcttgactggggaaggtcgaagtaggtgctgtctggttccctggggcagggagaggaaaatagaagcgtcagtccccagtttgggtgtcaccccaatacctcccaggcccagagctcaaggctagcctgagactcctggtgaggcctacgtctcctgcccttcccattccctctcatcccatctctgtcctccacctgccattccagggctgctgtgagtgccagagaggcagagccagtcatgggctctgttattctgggcctctgctggccatgggccctctccagagctggccagagaaaagagtctgaattttggtcctgcatccccagggaaggcctagggcagcagtccctcttccctgcccctccgacccgccctgtccccaagctgatgagggtggctaaggctagcccaagactagtgtggagccacctctcctgctctggtccagttagtgcagcaagggtggtcgaggctagcccagaatcccagtgagggttgccaagtttagcccaaagtctcagtgagggtggctggggctagcccagagtcctcccggggccacccctcctgctctggcccagttagcccagcgaaggtggctgaggctagcccagagtgcctcccaggggggcccccagctctggccagccaagcgcactcacagagagctccagaggtgctcaaacgtggtgcccccatcaggggaggtggcggtggactgggccatcttccccacgccggcctccgagggcagctcgctctgcaggaaggaaggaatgacacctgagcaccctgggaactcgtgctctgtcctcctgtgacagcctcggtgggggcggcaggcaagtgggcctgcctggggcatccctggagcaggtgcccccgccttgccctccctgtgccctctccactgtcatttcgtccaccctgttccccgcggctggaaaccgaaggccaggtgggcagctctgccttggaggtggcgggcctggatcagacccatccgtgggggtgccctgggcaggcaggagcttctcagagccgtcgccttggaagagggggtgacagcaacacccgctcctggggctgccttgagggccttcaggagagcagactctttgagatgcttgggccgggacaagcgtgtgaggcctgtggtctgttatgatccactgtgtctaggtttggcctgggggatgcttgggccggaacaagtgtgtgaggcctgtggtctgttacgatcccctgtgtctgggtttggcctgggggatgcagccaggtttggggtctccctgatgcccgggacctggaggtggctccggtcccggtcaccccatcttgggaacagcgtgtgtgagggctgcgctggaggctgcagccaagcccccggctagagcctagctgtgcccactgctgcaggacacagacgccccctcgccttctgagctggggctccttcctgcaccccagggccacagggaagcctggcaaggctgggaacttgaagctgccctgtcctgcagccggccactgcctgggtggtcacgccttgagaaggagacacccagggattggagagctctgaccccagcgcccgtccgctggtctttgtctccaccattgctcccctgagaacccctgggcccagctcccagtggccctggacacccatgaggaggtggcagtgtccaggcagagcacagctgggggaggggacagggaaacccgagcccaccctgcccacagcccccagccccggctgaggtgacctctcccctgggccagcctcccaggtcctcggtgaggtgctatgggacaggcttggccaccgccctgcaatgctcaggcaggcaggcctggacctgcccacggccagcgggggtgcaggccatggcctggaacctgtcgtttttgtccttccccaggcaggcgggagctgatagaagcctgtggctgccacgtgccgggccctggagaagtggcacaggccaggctgaggggcggtccgtgagccatgatggcctgggacaagcagcttagagggtgctggcccaagccacctctcccaggtgggcgtgaccgctccatccacttttcaggagccccaaggctcagaggggttaagtgacttgtgtgaggtcacacggctgtaagtgctggtgatgaatgctccaggggctcttagaattagaagttaatttggttgccctgccccaccccacttcaccatcacgtggccaggtgggtgtgcccttgcaggcctccgatcctgggctgggctaaaaccccagtggagctgagggtgtgcatggggaccgcaccattccgttaatttaaaatgggagagtgtgacaggaagcagaaccgcaggtccagctgaggctggcacagggccccgggggagagcagaacccgcccttgcccataccccacggtgggtccctttgggctgagcagtatgagtagggtagcctcttggtgcaggtatgtgggccaaggctgggtgggcacaagtgaggggtcccaggcatgtttcctgaagggcaggagcactggaggggctggactcggaggaaagaaattcccttagatccaagaggacaagcctgggaaggttttgccgccagaggcctctgggtcgcatccccttcattttgtagatgggccacctgagtccagagatggaaatggacttgccatccacagcaggtggatgacaaaatcccagctcccttcacagcagcaccattcctggtagctgaacggtgcacatagcccagacgcccatcagcagagggccgggcacaggagatgtggtgccagcaagcaatggaacattacgcggccatgaaaaggaacgaagctctgacatgctacagcgtgggtgagacctgaaaacctcatgctcaggacacatgacctcatctgtcagcctgtgagctgtcccctcccaaggctaaggtgggacaaagacagagagagacagagacagagagacagagagaaacaaggacaaagagagacagagacatagagacagggatagagacaggaagagagagacagagagagagagacaaggacaaagaaagagaaagacaggaagagagacagagagacagggacaaagagagagacaaagagagacagaaagagacagggaaagagatagagagagacaggaagagagacagagagacggagagagacaaaggcagagagagacagggacaaagagagacagagagatacaaggacaaagagagacagagacagagacagagagacagggacaaagagagacaaaacagagagacaaggacaaagagagacggagagagacaaaggcagagagagacagggacaaagagagacggagagagacaaaggcagagagagacagggacaaagagagacagagagatacaaggacaaagagagagagagacagagacagagagacagggacaaagagagacaaaacagagagacaaggacaaagagagacagagagtgagagagacagagacagggagagacaaggacaaagagagacagagagatggagagagacagagacagagagacagggagagagacagagagagatggagagagacagggagacagagacagacagagacagacagagaaacagagggaaagagagagggacagggagacagacagcgtgggagccgacggcatcccgccgcacccctactggaggtccaggggttccgtgaacaggtgtgcacggggcatccctccgcgcagggctcagggctcacaggaactggtcggcaaaacatgatttgtgatcttatgcctgggacatgggcgttggctgtattttcaagtcggcatcctgtgaactccccctgaagcgacaccctctcttcccactgaaccctgaatgaggaacagcgacccttcttgtcagcgcgcctctgcctgccggagacccagcttccacatcccctgaccggcacccactgtgcactgcaggacaccagcttggcctctcatccctgcctgggcacaggacagaggccagcgagcgtggtcgtgggtccccgatcccacccgtccggccctccaggctcacaggacaggccaccaccaggagcatgtggggtgggaaaggggcttcctccacgccaggagttgggtccccaaacatccccaggcctgtttgcctgagatgtgggcagtgggcctccaggtgcaggcgtgtggaccagccggaagccctgggcagatgcatcttccaggacacccctccccacagtggggctggggcagggcccaggaatctgcccctttaacaagcctccaggggagtcatgaagagcctgggacaggccccctgaggaggacactgtcatgagggccagccttgccactgctcggggccctgctgcagccaccggcctcccccatgggaatcgggttcctcattagccaaaaccaggacccggaaacctgcccagacttaaagcacacggaggaacttgaaccggaagccacgagatgcctgattcccaacttcctgggaaaacgccaacgctggtagcattggtcccagggccccacaggaacactgctcacaggcacaggcgccaggcaggggctcccccaaccggcctcacacacctgagctgatgactggctccgcaggcatctgggagtactgggcatgaggaacaggaagctctgttgggggtgcagggacgctgcgctggggcagggggagtatgtgccctctgcccacagccgcctttcaggcaaggatcccagtggcgagaaaaataagtcaattctaataaaatggcctttcaattaaatccaattttcccaaccatttgccttttaatttgaaaattcgttcacaaggggatcacggttctgggacaaaacagaaattgcttttgaaacgttgccaatagttttaaaaatcatgtgtatcaatggggaaaggagaccaatttcataagacatctttaggaagttttatcaacatcataaaatgcaaatacattattggaagtgtaggtttttctaagaagatttagtctaggagggatgggctgggtgcagtggttcataactgtaatcccaacactttaggaggccaaggagggaggattacttgagcccaagggcttgagaccagcctgggcaacacagggagacctcagctctccaaaaacttaaaaaaatattagctgggcatgagggcgtgatggcatgtgcctgcagtccaaaccacgcagggtgctgaggtgggagaatcacttgagcccaggggctcgaggctgcagtgagccatgattgcaccactgcactccagtctgggcaacagagctagaccacaactctaaaaaaaaaaaaaaaaaaaagggaaggaaggaaatgaaataaatgagttctcagttgtaacaggggcgtccagtgtgtattaccacaggtcttgcagtggaaggctttgtgtggccacagcatttgggggtggtcattggggagctgctggccggcacccccccagccctggcaccatcaagtcagggcacagagttgcaaaggacagttgtgctgggtgttcctggttacgctgtagccaggctgtaggctgctggcacacccaggaagcagggcacctcgaccctgccccaacttccatgaggcctcagacaagtcccctccctttgccaggctggaaaggtgaccagcgttccaagtcctacagtgcccacagcagtgggagcaggacaaagaccagccaccgagggccctccggcaaacccagacctcagcctgcttggatcctcaactccaggggccgctgtaccagctgcaccacagggagagcccccacacacgtgtgactcagaacaggaaaggaactcacaccagcaattaggcccccacagcttccccgggacccacggagcacaggtgggctggggaggggactgtgtggcccagaaaaccagcctgcccaagctccctggagctgaaaacaaggctctctgatgacttctcggtttgactcagggccttctgaggtctcagcctaggggttctcaccaggggcagttccacccccagaggatattcccatgtggggacattttccacggggtcagagggtgctgtggaatctagtgtgcaagacagggatgctgcttagcaccctacgttgctcagggcagctgcccacagcagaacgcacgcccagctgtcggggccgtggcggagagggggtgctgtgccatggctccgagctgaggctggccaggcactttgcagggcctcaggctcacacgccaaccctgccccagttcagagatgagcaagtggaggccaggaggcccagtggctggcccaaagctactgagggctgaggagctgggactccagccaagcccaggtcctctgatggcctggccgcacccgaatgctcccctctgccctgtggggaggagtccgcagccggtgaggggctctcagcctctctcatctcaccctgcccaacccctctggcctcgtcttccatagcttcccacgaaagccacagccccctgtccccctgccctgtcccaagcattccctccctgtccccaagcatcccctccctgtcccctgttcctctgtcctctccctgtccccctgtctccctgtcccccataggttccttccctgtcccctttccctttgtcccctagccccccatcttctatcccctccctgtctgcatccctctgtcccctccccatctcctgtttctctgtcccctccctgtcccttgtccccatagatccctgccctatccccatgggtcccctccctgttccctgtccctttgacctctccttctcccatcctctgtccccgtcctagctcctgtttctgtgtcccctccctgtccccgtcctcctgtcccctccttgtcccccatcctctgtccctccccgtctcctgtccctctgtcctctccctatctcttgtccccatgggtgccctccttgtcccctgtccccagtccccacatgcctggctatgtgctctctgtgttcagcctttgtctaccacccccttcctgccctcccatctcatccctatctctccaggccaggccaggacccagcctcctctaggaaccccgtctccggctgacgccccatcctctcccctcaccacctgacaggaggtgggagaccagacatttccttggccatgggcccacaggtaaggcctcgtcagttccccaaccccttccacctctgcccaggcctggggcagacagggctccacaaccacctttgataagccaggacactccaccccgactgagcccactctggaagaggcactccccatggggacacccactcccacctgcaggggccctggccgcagggctgctgttacctacccacccctcggtggagctctgagaccgagccgtccgcactcagcctgggcaggagacacaggcccctctgccccgtctggcccagccatcccggacactggctcttaagggggatcacaggaggggtgcagggtgtcctcctccattcagggaggagggaaaagcccccaggaactccacccgccccgggccacccctaaagcaaacatctcaatgcacttctgattccggtggttcccaaaccaccaatgattttacctgttatttcagcagatcaacgattccaatctgaagtggcaaagaaacctggggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagacacctgggccattgtgggaggagaccgtctcaatccccttgtgcccttttcccagggactcaggcaggtcccaggacccagaacccagaagagcccccgcctgggcagaggggtcgggcggtcagcgcaccagaatgaagccaggcaggctggccctgtgtgagacagcggctctcccaggcagcagcccagcggaattcctgcagggcgtcatcagagcccagcctcctggggccctctgtgccaaggggcctcactctcccctccagccaccgcaagaagaggggccgctctcgcttggcaggctggggccaaggacaggggtcccccggagtgagggccaggagggcctccctctcaggggcaaggccctctgccagcattgcctgcccctcccgacagaggctaaggaccatgtggcctccctctgtgggtacgatggggccaggtccttcagacagggttgccaggacggcttcgtcagcaggtcaggggcagggacgaaggcctgtccgcagggcctggtgaggaggatgcggctgagagcccccctcaccgccacagatggcacgcctgctgccccactcccctagtttggggaagcccagcatggggatgaaagacaaacacagcctcccacggtggggtagcgtgcaccgtgtgaaatatgcggacagctggaggtgcctgagcaagctgccctctctctcctgggccgtggcccacgggaagcgggaggggaggggaggcagatgcccaggacagtctgggggaatccctgagcctcaccccagaagctttgaggccatgaccaaaccaaaatgcccgaccccccgtgggtgggggtgcccccaaccagcttcctgggtcagttcacactcagccaagtgaaatgtagaaacaccaaagcatggctaattcccggggattccagccaccccacgacggcccagtgaggccacacacctgttaccaaccaggagacatgcacagagagggaagaccagccctccatgggcacacagctggtcagggtggaggtcgaacccagcagcctggctctaccctggcctgacctggagctgagcaagggaggcacgggcccggctgcaatcccctccccagtccgtgttcccaccgcacagccacaccccgcaccctgcctgggaagcccctctctggaaatcccctgcctgtccatctatcttcaccggtacattgaggcccaatccacatccagacatccaagaggcccgccttgtcctctgctgcctgcccaggccttcttgctgtcatatatgtgtttgggtcataccaagacacatgattcctgccccaccgcttcgtgccctgaagcccagcactgccgggcacacactccagttccctctcctgctctgcccacgcagcccacctctgccgcgctctagctgtgtgacctcagatagatgtcttgcccattctgagcctctgtttccttgtctgtaaaatggagataattgtagtgcctcctagaagcgctaggtggaatgaagtaactctacgcatatcaaaagcgaaagcactcctctatctgggcacaccgtggatgccccataaaccgtatcgagttgccattgaccgacggttatttagtatcaggtggtggcagtggaggaagaaccaatgaggcccgggtcccaaaggcaccaggctgagcgacagaactgcacacacgggggcggaaaggtggggtctgctggctgccgggtccctggctccttcgagtgcctctccatttcatggtcagcacgcgtgtctcaccccacttttacagtggtctccgcacccttttgcagagcaccagcctatcctcagtctctcagggaatgaggcgtcctcgaaacgggtagtgcccagttgcagcggcagccggtgccagctccatgcccgggggcatgcaacgcggtaaacagataaccctggcccactggaggcaaatcatgtatgtctgtgaggccctcagaggctgggtatgaggagggtgggtggtggaagagaagtctgcaccccagccccagcttgcttgcgccttggagtgggcggtgctcctgggggtaagcgggtcaggagggtttggaggtggggtcagaagcatgagcggcctgatgggctgagcttcctctaagaaaggggtggaaggggtgggcagggacatggcccttggggggacgaggttggcggcaggagggaagtggggccctgtcagggaactggggcgctgagcttggtgatgtttcctgccaggagcccacctgttagaggaagggcgaccctggcgggggtggtaggagcagatctgtcactaagggaggttcagaccagcacagtgggcacaggaagaagacccagaggaccgactgccaaggatagggggtgaggggtccagggctgggacagagcggagcttatggccagagtgggtgtctgagtcgatacctgtgttcacaggtgatgagtacagcgctgccctcagcagggcaggtgactgatggggccgcagccgccttcgctggagctgccacagcctcgttctgcacagacacatccctgtcctctgccccctccgatcctcctctcacaccccctttctaccccaagggatcacaccgggtgtcactagcctttgtgcggccagcgcccatgttggaagtgcctgtgctgtcctccagaatcttctttctttccccagagaagcacttccactcctccaaccaagagagaatgacccttctcaagctgctgtccccacctggccaccctcctctggacacccttggtggggatgctccactctgagatgtccccaccctacacatacccctcacccctggctctgggcttttctctaaggggtctgggacacgcccgctgcaccagtggacatgtctccctgctgaccactccctaccctgcggtctgagcagacgtgcccttccagacagctgtcaactccctgaacttgtcatgcgtccctctgagtcccgccctgactcagtgagcaatcctccctcctggcgtgttcccttcctgttctggagtcagccgctcagccctgtgacctcccctgcacagtggtggccgctcagccccgtgacctcccccgtgcagcgatcacgggggtccacccacccctcactccttccagaggcagccactgctgttcctcagagtggctgtccgctggctctcagggttgcctttctgatattgctgcccagcattccctgtgccaaatcctccatattttcaagctcattcaatcctcagaggaacccagcgggaagtccccaggtgttcccagaggccccgagagggcgagcagcctgcccagggccgcacatctcagaagcaccacagctggcttaggctgggctccggtgagctccaccttcccgccaggcagcccgtcctctggaggccccggctcctgcagggcagacaacgatccagcctttgcccccgtcacgaagggggctcccgacgatggctgggtcagcccaagagctgcctaggccctgggcagggctgtgatcttgtgggactcacaagcgaatggaggggcactggccatggtggagctcggccaaggggcagcctgggcagccagtaggtcagggagaggcaggaacagcaactggagctggacagggtacaggaccaccaaggtcacaatggatcctaggctccatgctactccaggtctgagggggctgcttcccgtcacgctcccagctcccacaccccacgcctctccagggacaggcccacccctgccatccctgaggcagttccagctggagccctggaaccctgaaacctggacaggtgccagccgacctgtgcttccagcccagccctgagggaggctgtgcagccagaccccactctgaccagcatggcacccacgagggacggctgtcaggaagcctcactcctcagggcccgcgcacccccatttaagagacgctggtcttccccagacctgccagggctcctccctcccagcacccatcgcatggagagacattcacatgctgactctttgcgggccagaccaccaagccctgactgctctctgcgctgcagccccagcacccgcctggcacacagtaggtgctccgtgaagaagcgatgacagaatccatgatttggtttgagcgggcggggcccagcaggtcaggtcaggacaggaacagagtgactgctggagaagcaccatttggggttaaattgctcgacttcctcctcatttctcctcactttttctttttcttttcctatcttttttattttattttattttttttgagatagagtctcgctctgtcacccaggctggagtgcagtggtacgatctcagctcactgcaacctccgccttccaggttcaaatgattcttctgactcagcctccccagtagctgggattacagctgtgtgcgccaccacgcccagctaatttttgtatttttagtagagacagggttcaccatgttggccaggttgctctcgaactcctgacctctgttgatccacccgcctcagcctcccagagtgctgggattacagatgtgagccaccacgcctggccttcttccccacttctaaggggctcctgtacatccccacctggacatggggctcttgagtcttgagttcagactgcgggaccccagggagacgacacccacattccagaagcctgtgcccatgtggtcccccggccccatcatcatccctgccttacctgtgcccatgtggtcccctggccccatcatcatccctgccttagggatgagcagagagatcctcagagtggtgtaaaacgcacccagagcgacacagcaagccaggaagagcccatggtctcgccccccgagccacatgcccatccaggaggccaggctggagactttgcaaagagccaagggaacaggatccagaggccctggcccctcggacattcccccaaggcttcagggaggagctggtcactcagtttccctacccaccctctcacctctagcaacacacttgcacatacatgcacatggacacacctattcacatatgcgcacacatgtgtccgcaggcactgccacgtacacactctgccctcctcacagcaaatccccacaagcccctcccctgtcccccagtttgatgggtccctgaggattccccagcctgggagtccagccctggaagcaggacccaggcagaaggaacaggacatgcccagccccttcctgccgtccacgtcatggtcaggcttgggagtagctgcgtgagtgccggggaggccggggcatggcttgctgttgccgggagaggctccggggccacaaaagagtgagggggctggggagaactgggaggttctggatcagctgtgtgaggctgaggatggcggcccccagccgggaagcccccggacgtgccaggctggggcagtcactctgcgcctccttggcaggaagtggaaagagctcctggtgggtccaaggggtggaggtggcggtaccagagccacttctgcccccgatcctagggtcacacgaggagccctgatgtgccacttggggcagatggctccccagagctgtggcttgtcccatcaggagaactgtgggcaaggcctccccaccctggcctgaacagtactgcatttccaagccgcctcctgggaggccgtagaggggctctctgcaccccagggtgggcccccggagccactgcacccacggtggcaaggagactccccccgactcaggccgagaggagggcgagccaagcccaagcctaccttggctaagcctgggccccggagggtcggtggggccaggtggaagtggccctcggctggctcctggctcccgcaggcgggcggtggcagcagcgtccctgtcacctaggtgatgacattggagttgcgaaactaggcggcagcgtggccggcctggagctgcagggcagctgggggcggagcatggcctggagccctggggctggagacgggcctggagccagcacctgccgcctgccacctggcatgcacgcgccaccctgcatccctgctccccccaaccctcctcaaggcgactgacagggccgagctcagggcagccaggacagggctgtctccacccagctttgtactcgtttctactcctcattgtggacacttccaagcacactcagcactacagggaacggcccaggaacccatccacccgtccccagctctcatcactctgacctctggctttttgtttcctggggcatttcacagcaaattccacctatgagaccgtctcatccgtaatacaccagtacatgcttccagcaggtaagaacctctcttttaaaatattaggtcaaaccatgaaattgctgataattaaccgttttggtcctaccaacaatacacacacacacacacacacacacacacacacgtgcagcaagttcagataattcaacattatatgcagccataatatcaaattctgaatctttaatgctggtcagaggattttgaggagccccgccccaattttggagagggaagtgtgtgtcgctgccactgtgccaccttcctcccccacaattgcagcagcacggagtggacagcacggatgtgtgtctaggtgctgcacggatgtgtgtgacagctactcatggctgttggctccaggaggggagatggcccctcatcgcagccgggtgactctttggaaagtgtatccatgggtcattctcaagcacaccaatttgggttccttggctgcagtgacctcctggcaggcactcctcctcccccacccctagttcataggaaagtgaggggagaggaaagacctgaacccagggaggctggtgcctggctcctctcaacatccttgttggagacaaaaggtgggtgggtgacaggaggagcaatagcagctggaccctggccatagccatggccaagtctgttccctggtctgaagagcaactagcacagagtgacagtggccatgagccctggggggacactgaagggggagttcaaggagaatttttgcacacctgtgcaccgtaacacaagggttgcaaagctattcctgaactcccctgccctattttaagctgggtggtccagatgtgaactacagggcaagcccttgtttatttcctttttttcttttttactttttttctttctttttttttttttttgagacagaatctctctctgtcgcccaggctggagtgcagtggctccatcttggctcactgcaacctttgcctcccaggttcaagcgattctcatgcctcagcctcctgaatagctgggactacaggcacgtgccaccacgcctggctaattttttgtatttttagtagaaacggggtttcaccatgtgggccaggatagtttcaatcttctgacctcatgatccacccgcctcggcctcccaaagtgctgggattgcaagcgtgagccaccgcgcccagctgccctcgtttatgcctatcaggcaagagctggagaggggaccatgcggatgtgcgcactgtctgttttctgtggctgccgtaaccaatgaccacacaatgagcagcacagaatgagataaatttactctcttacagttctgtaggtcagagtccgagatgggtaccagggagctaaagccaaactgcgggcagggctggctgcttctggagaccccgggagaatacatttccccctcttcctgtctttggggggccctgggagaatccctactcccctgtcttctcttggttctggaggccctgggacaatccctacctccccgtcctctcctgcttccgcaggcatattccttggcatgtgacactttcctccatctctgaaatgcatccctccaacctctgcctccatcatcagctccctctctcacccctcttgtggggccccctgatgacactggacccacctggatcctgccagatggcctccccatctcaaaatagctgaatcacggtcccctttgccatgggaaataacatagacacgggttctggggattgggacgtgaagacctttggggagccacttttttttgccaaccataaatgtccctcattgaaaaacatcttttctgagccctctgtttgggggtcagtctgaaagccgccctgggagagacgcccgcatggcactgagtcgcgtctgctcagagcaccatccccaccgccaatgcttactcacttccgagctgaagggcttctgggacggtttgttcctgctcagtgagacacacaggacaggcctctttgaggtcaaagggcaccctagcctgcagccaaggccagggaggtgggcaggaggtggtggggagggagccagcagagggaccattaaaagccccccgcccacgcacccaccttcactgtcagaccccttcctttctccccacagctcttcagtatttgccagcaggtccctctgtgtgcatcagtgagttcctgagggcatgccgcagaagtgaaactgtcaatatgacccataattacaccccaccaacacacagcttactctgcaccaggcaccattttaagattttacattcattagtgtgggatacatagcaagaccctgtctctaaaagcaagaaacaacaaaactaaattaactggatgtggtggtgtgtgtctatggtcacagctactcaggacacaggcaagaggatggcctgagcccagcagttggaggctgtggtcagctgtgactgcaccactgcacaccagcctaggctgcagagtgagattctctctctctctctctctttttttttttttttttttttttttttgagacagagttttgctcttgttgcccaggctggagtacaatggtgcgatctcagctcaccacaaactccgcctcctggcttcaaaagcgattctcctgcctcagcctcccaagtagctggaattactatacccagctaatttttgtatttttagtagagatggggtttcactgtatgttggccaggttggtctcaaactcctgacctggtgatccacctgccttggcctcccaaagtgctgggattacaggtgtgagccaccttgcctggtgattctgtctcttaaaaaaaaaaaaaaaaaaaaaggccgggcacggtggttcatgcctgtaatcccagccactcaggaggctgaggcaggagaatcgcttgaacctgggaggtggaggtttcagtgagccaagatcatgccactgcactccagcctgggtgacagagagagaaattgtctaaaaaaaaaaaaaaaagaaaagagagacaaaccaaaggacctaggggtcagagttatgaaacttcaaaatacgaatgatttccatgcaccccactcatatcaagtccttccggttttaaagttctgttttgcataaattactaaccgtatacttcatcgtcaagagtagactctttctgttattctaatactttcaaagaccagactgaccagctgaacattagacaatgaattcttggagttatactggcctcaatcttacaacggtgtattttgctatttttatcagaattcatcattttcatattaatccaattagcatcctgtttaccaatacctcattttttaatttgcagcttccttctaaaagcttgggggggactttcacaactcgatctgtcctccctcattactgccaaataattagtcaaggagccgtccctgatcaaaaattaagaacccagaagcaagttggagcatgcctcgctccagaagaaaacatgacaggagactcttgtaattatgaggggactggttggtagtcatgatgcagccggcaaagactgtgatagcaggaaggagagacctgcactgaatcgtgagcgtggccgatcacagctcattaactagcttggagtttgcatctcctgtttaactgacaaagaccgcagagtctttcctctgtaatttgggggctgacttcaccctgcattgtgcatggtgcaaattgcgaggaccataaagagaggatggtgagttgccttgcccactgaatcgtgcaccttatctcggtcatcggattcacaggaatcacctgcctctaaagagggagcacatcttccctttgtagcctgaccgcatctggtaagggtggctgggttttctgcagaagagatacccctctctgaggcccagccgaggcctcccacagactctacaacccctgccccccaaccatctcacagtcaggaatgacccacactgaagcaaggctgctccccgtcccctacaaccaccaggccccagtctggcgggtgggcaccacccaggtggacccggaggccaggaggctgtggaagagaaagctgcgggtctggctcatgtattcggactggtggagaaaatggagccaggaagtcattgctaagatcacaggaaatggtgcagtacgggcgagcaggagcttccctgccctttccaaaggacccagtgaaatactgcacctctcctgcaggaggggcagcctgccccctcagcccagccttccccaggtgccacccccaaccctgcctgacacctcagagcagcaggtgagcaattagagcccccacccctcctcacaggcaccctgagccacggggggcctgccagctggtactgggcagggaaacaggtgggtccttcctatccaatgcagcgcctctggtagggggacacaccctgggctgggtggaagccctgccggctcaggccctgcatctgggctggtaggaaggtaggagtcgcgatgcccaggggcccgtgcccagactggcagggcagaggggctgcaccagcctcacttgggcacctcaagtctgccagtgccacccttgggggttctcaggtgtggtcccataggcagctcagatcgagctcacagtggggatacagcagtggcctctccagagagcctgtggcatggagggaggggggccggccacgacccccagggtactgattgggctcaagacataccactaccccaaaacctggcacggtggcatctgagaagccagggaaagcaggtatgctctctcctgctccctgtcaccctgaggcaggccataaaagagttctctggccttcctctgaattaggtcacagaccctcatgtgccctagacccagaagaaaggaagatcagagccttcagacctggccccagcccccggttcaggatgtctgtatcctttctttctttctttttttttttttttgagatggagtctcgcattgttgcacgatcttggctcactgcaagctccaccttcctggttcacgccattctcctgcctcagcctcccgagtagctgggactacaggcgcccgccaccacgcctggctaattttttgtattttttagtagagatggggtttcaccgtgtgagccagtatagtctcgatctcctgacctagtgatccgcccgactcagcctcccaaagtgctgggattacaggcgtgagccaccacgcccagccgcctgtatcctttcatcacgctccgtgctgtccacaccccacaaacccaggaggaaaatatgcagagttccccggttcgatgggtgttccattctcaaggctcctgtgtcacatggaacttctcgtaaataagcctgcacggccctcttgttaacttgtctttcattactggtacggcagccaccaggctcgtgataggcagagggtcctcttgttaacctgtctttgtaggtgccgcagccaccagcctcgtgaagaagagaggccccttctcccctcaggcatgggtggcccggcctgcagctgatcttggtggcatgaattgatctcccaggcttcagcctccccatctgtgaatgggacagtgatcactgtttccagataatgatcagcaaagccctcatctgtatgatgccactgcagtgaggaaagcgccgccctcagattactcatgccagtgcccaaagggcaacacatgggaccagagacaagtttctcttataaaagagaatttcgtttttctctgagagcgctcggagctgctggaagaggcagccccatgctgtcctcacactggggacccctccctgcgctccccagccaggtctgtcctggggcctggtggggagctgattccatctccaaggcccatagaggcccctctaggcccccttgcaatgtcccctccatagccctgacacctgggttggggctcaggacacgccaccccaccccaaaacatggcttgagggggcagaatgtgccaccccaaacatgccttgggtatatccattatttcgagtaactgcagacacaggagtggctctgcccagctgcctgtctgggagagaaatgaaagacatttccattcgtaaaggtctctatgccgggaggagagcggctccaggtaactctgatcgcctgagagacgcatgtccgcgtgacgaaaccaccttcattcaccagtcaactcccctgctatagctggttcagcctgcagcagccccacagtcctgtatcttcctgcagctgaggatgacgcagacgcctccgccatctggccccttcccccagtttcatctccgtgtgggactcccattcgtggacatatcattaaaatgggttttctcctgttgatctgtctgatgtcacttttcctcccctgcagcttggtgacatgaaaggacctccgtggctggccttactccctgtggatgccacagctgcaggatcctggggccccaacagaagccggtgaaggaagagttcctgctccagcaggtgcctggtctccaccttcaggggctgctccaggagagtggcaatttggggcagcaggaggaaacatgggtgtgaactagttctttttttttttgagacagggtctcactctgtcacccaggctagagtgcagtggcaccatcatagttcatcgcagcctcaactcctgggcttcaagggatcctgccacctcagcctcccgagtagctgggagacaggtgggcaccaccacacccagctaattaaaaaaaataaaaaataaaaaataaaataaaaaatatatattgtgtgtatatatataatacatatattatatatatagcgtgtatatatataatacatatattatatatagtgtatgtatatatataatacatattatatatagtgtatgtatatatgtaatacatattatatatatatatatatacacaaaatacatgtgtgtgtatatacagtttgttgctcaggctggagtacagtggtgcgatctcagctcactgtggcatgaatctccctgggttcaagggatcctcctatctcagcttcccgagtagctgagattacaggcatgtgccaccatgcctggctaatttttgtatttttggtagagacggggttttgccatgttgcccaggctggtctcgaactcctgggctctagtgatccgctcatgtcagcctctcaaagtgctggaattacaggcatgacccaccatgcctggccagatctaagagtattttaaactatctaactttctgcatttctctttgaatatttcattgttactttgattaaatgagaattaagtattatttcataatgatctgggattctatttaatcaagtgctttcaaccttttgacattttgacaactttccaaaatcacacactctaaattaagtctttttgaccctgaattaacgtttgggatttcctagattggcccctggaatatctcaaaaggatttattttctctccttataaaaacatagattattttgccaatgttaaagggaaaattgttttagataaaataaaaagatattaaataaattgggcttatttgatcaactaaatttgcatggaagcactgtaaaatatgaagtgatgattaaccttctctgggtgagttatatttgcatggatatatattcatataagagttccagaaattatatgaaattcttagaaatctgatacattctatattagttgtaattctagttattatgtgaaagtatacaccacaggaataaccaaacttcctcatcagttacattgtaatgatgtcatcacatctttagccaaggccattttaagcctttctgtcatacacagttaactgtttggctctgatgctttcccgaaagcttttgcaagcaactataattctaaaatgttgtgtcttcgagaagatttcatggggaagactttgaccagtacaggtttctgatacattaagatcacaccactggatggcaagaatttccagaatgtaaatgaagaaactatttataacttacagcattctggtacattacacttttataagcaaaattgaaatgatgacttttctccctacctgatctctccagaatttagaaactattagcatttttatttttacggccatagagttatttgcttaagttcaagatgaatctgctgccttgtaataggaaacaactggaaatgttggttagctcaccaagtgtttgaccggaatatcatatttgagagtgatgtgcataaaatcagatgtgagtagctttaaggaactaaggttgactttatggagccaatgaaacccgttggaggaaaacaaaattgcccttgtacctggattaaagggcttctgcagcctcacgggttgaggaaagactgtgattttttggcagtaattctcaagaatacgttgggaaccttgagaaagaggaatccacccaaatctataggtattgcaggtgaagtctgatatcaagtccttggcttgacttcttagcctcaagaggcttttgaaagcctaacctgagatttattaaaagttccagcaaagctgacctaaagaaagcctagctggccacttactattcttgctgcacttgtgcaaataatcaggccaattttaatgaggctagacttactttacaaacaagttaatctgactaggctgggcacagtggctcacgtctgtaatcctagcactttgggaggccgaagtgggtggatcctgtgaggtcaggagttcaagaccagcctggccaacatggcaaaaccctgtctctactaaaaaaaagtaataataatacaaaaattagtggtggtgcatgcctgtaactccaggaggctgaggcaggagaatcacttgaacccgggaggcagagtttgcagtgagtcgagatgatgccactacactctagccttgagcgacggagtggaactctgtctcaaaaataataataataataataatcttactgagattatttttagtagaaagaggggtgatgtagaaagaacatatatttcagtagaaagccatattgtgcgccccatattagattcttgtcctgtttaacgtctttggggttttgttatctattgatgtctacaaactggacgggttcctgatgtttctagtttcctccagtatctggctgcaactcccaaactaacactttcaattttctcccaccgttcttattggaatcacgaagaacaaaactgccctttgcccagaaatcctgcaaactgaggctggacgacttgaaatactcatcagcgagattaccacgaagcccaggtgcgagggaccctcacgcctgttgctgcgtgggccgcccagacatcgccagagaccaaaccgcagaagatgctcggagcccaacatctacaaacctgccccgccacggcccccaggctcagaaactggtttttagtcagctccaatgattagctttttttttttttcttttgtttccatagaaatgcctcttcaaagcattatgggacaacacacccacccagtttctgtaccgtggagcttcttggggaagtttcagactaggaaacaatggggctcaggacacatcaccccaaagtatgactgtaggagaccagaatacgccacccctaaatatgactataggagaccagaatacgtcaccccaaaatatgactgtaggagaccagaatacgccaccccaaaatatgactgtaggagaccagaatacgccaccccaaaatatgactgtaggagaccagaatacgccacccctaaatatgactgtaggagactagaatacgtcaccccaaaatatgactgtaggagaccagaatacgtcaccccaaaatatgactgtaggagaccagaatacgtcaccccaaaatatgccatgttgacatgttgattactttgagctaaaagcaactgagaaccagccaacagaaaaaaagctcttcagctccctaaaataaagtaaaaaactctcttttctaaagaaaattgacatctataaaggacattttcattagtaatggtctgtgtgccaggaagagagccgcgcgccaagacgcattttctcaccatgagactgatagctgcaccacgaggcagcctttatgcccagacgtttcctccctcaccctctcctcatttgtctctaccgctccccagaagcccaggccccctttcctttctgtcacagaggacggacatctgcaccgctcccatctggttactgctgagtctcctgtttttatggcacttccgggcgggggcacagccttaaaactgttgtctcttgtgagtctgtcttttgtcaatcgtttattgacaagaaccccgtagggaagagggaggcaactttcccttcccctctccacagcttgctctttggggaacccgatttaatctccaaggcccacggaatcttccggccactgtccatccctaatgtgtcccccatgtcccctccacggtccctgctccatgagacctgccttccgtgcccttgctctggccccagcagggcccttcctgccacctggtctctccaggcctcactggaggccaccactgggacggcgtctcctctctgtccaactccctgagaggtgcctctccagacccttccgcgcagaccgatgtcccccgaccttctgtagcacccacggccaggtccagggctggggcccgggtctcgcctctcccggtggggcttcttgttcaatacctctggtttctgtccctaactgggggctatgcggcccaagcagtccccaggtagaggagctgctggagtgtgtggaacagtctgttttccaggatgcaggggctgtggaggaggcgccaggcgggaggtgctgggggagaggggggctgcccatccccggctgtgcccaaccccgaccttcagtgtccgggttccaagatcctcgtcctgaaaagaggggcgtggcttagaccaggtttcttccccgtttgcgccatggcccctctggcagcctggggaatctcagacgccttgtccgggagaatgttttaaatacatgaagcaaaatacacaggatcacgagttactgaaacatagccactctagtgggttggatgcagcctctgggaacgagcctcagaatgggatgttatttggaatcagggtctttgcagatgccacttaggtaagggtctcaagataagatcattctggattttccaggtgggcccgaaatccaaggacaggtgtccatctaagggacaaaaaaggagagacacacagggacacagaggagaaggccatggcaagatggtggcagagactgggtggtgcatctgcatgcctgggaaccacaggatggccggcagcccccagatcctaggagggagacaggaggcgagccccctctcagagcctccgagggaacccgaccctgcccacacctggcttcagattcctgcctccacaaccgtgagacaacactctctattgtttccagccacttaatttgtggtcatttgtaacagcagcctgagaaaacttacaaagtgaccctaatatttaaaaagcaaaactgggaggtgatcatatgtgcgcttctttgttcatacattaagtcgtaagcctgagagcagatctaatcaccgccataattgcaacggagcgacaagcacaaaccactttttaagatgctgcagctattaccggacaccatgaaaatatcagtggttcccactggtgaggtcacaggtactgctatgactgtagtttgctgcctacattcaaaactagaggaaatgctgaaatttgctttgaggttaatacaaggtgccatttttccccatccaggctcacagcaccctggactctctacccctggcttagatgttctggaagggcccttccggcctgaatgttccacggtcctaggtgtctgggggccgtgtctgagccaacggcagacccaggcaaaggatggtttggcagccccagtcccagcttcgtggtcatagctggagtccccactcccgtactttgcagctgtgtgaccctgtgcaagtagaatagccagttgaagcctcagtttcctcatctgtaaatgggcataattatagtatctacttcaacgggctattgtggggattacagggaaaaaaatgtaaactttttagccaagtaccagctcacagccaccatgggctaaatgtgagttattacaggaaacattaaaaaacaagagttgggctggattatctttttcttctttgggtctgtgatgtgcaacttgtccaggaaaagaaagcactttggatgagcgagtgtttagcctgcaaagaaatactgagcacagagtggactgccagggggcaggcagaaagtctgtaaacatgtgcatatgtctgtctgagcccactttggtataagctgtcttacactcaaatgccaatgagcagagggattttttttttttttttttgagacgtagtctcgctctgtcgcccaggctggagtgtagtgcgtgatctcagctcactgcaagctccgccttccgggttcacgccattctcctgcctcagcctcctgggtagctgggactacaggtgcccgcaccacgcccgactaattttttgtatttttagtagagacggggtttcaccgtgttagccaggatggtcttgatctcctgacctcgtgatccacccgcctcagcctcccaaaatgctgggattacaggcgtgagccactgcgcccggctgagtagagggaatttttagaataaaataccttctgcacgacccacagagaaggattttcgaggcatcctgtgtaacccacttaagaaggcaaacttctaagaaagccattgtgtgttttcattatacacgagtggatgttgccagctacaagtgtgttttgtgtatttgttaaaggaatctgggcagaacatggaagtattaaatactggtaggagtaagtgatcactcagcctacttactggtgataaaacattacatttagaaaactttcaccacattcagatacaaataacacgggaattcaaaagaaaagtaggcgttattgtcatttagtaactcatctcagcttttttcttttttgcttttaaagcaaaatcacgaggaaataacatacagatatcttaaaaggttgccttagccctccccacactggaaaacaaaggcaagccgcaaaatggcagctcgttacatgaaacacacaccaaaagtattttttgcgtgtttttttgagacagagtctcgctctgttgcccgggctggagtgcagtggggtgatctcggctcactgcaacctctgcctcccaagttcaagcgattctcctgcctcagcctcctgagtagctgagactacaggcaccagacaccacccccggctaatttttgtatttttggtggggacaagatttcaccattttgcccaggctggtcttgaactcctgatctcaagtgatccacccacctcggcctcccaaagggctgagattacaggcgtgggccacggcgcctggccttaaaggatgttttgaagactttttctgactttttgcaaacacagggttcaccgtgtacgcgatgtcactttggtgtgctcggaatctcagggttaataccgccgctgggcaagggcagggcctcgtccgggttctggagtgggtgtgcggctcctctctcggccctcagcctcttctcttctctccactttagccgtgcctttcacacgtgctcagtgttatcaatccattcttggacctgggtggggcatgccctataaataaatactaaaccaggcatccttgccccagtgcatacttctgacagatgccatcagcttccacccacgccttgggcagatgcctgcctcactgccatccctcctgccctggagatacctaccagatactaaggagccgggtctctccccagggctcctggctctcccagctctggaacagcacctgggacccgaagccatcatgtgtgaggacactgagtgggtgccaggcactctcccaagcccttcggacggcacctcactgactgttctccaagcctctgaggcaggaactactattacccgactctgcagatgggtaacctgaggctccaagtggttaagtgacttgctggagtaaatggggaagaggagatttgaaccccagcattctgacttccatactaccccattaaccactgtgccagaccagcttctccatgcgactggccttcgggatttggagacagctactgttcgctgtagctttcacgagaaggactgcaaggcagggctcacatcccgagccattctctggtggccgccgcgcatagacaagccccgctcaggtccgccttccgttcaccccaggctacctctgattgacaataggcagctgccacactgcagcctctgacaccaacttttatcttatttcatcttattttttgagacagagtcttgctctgttgcccaggctggagtgcagtggcatgatatcagctcgctgcaacctctccatcccaggttcaagcaattcttgtgcctcagcctcccgagtagctgggagtacaggtgcctgccaccatgcccagctaatttttgtatttcaagtagagactgggtttcaccatgatggccaggctggtctcgaactcctgacctcaggtgatccacccaccttggcctcccaaaggctgtgagggatgacaggcgtgagctaccgtgcccagccaactttttatcttattttaaaaactacatacgtaatgcatacattcatatatggcaacatttcaaacaccgtgtcctgttcttaatgaaatgctttttaatctttccccgaataaatccaaagaggggtggctcagcgctcagtaagtgccagaggccacgcgctttgtttactcctcccccagagctaaggctgaacaaataaacagcaatgacagcaacaaaaaacaaaacaacaaaaaccaaagcattgtgttaatgagcctgagccatttccaaatgtccagacagtgaataaacagatggcagcatctttggaagcaaatgacctctggtcagaagtttggggtgcattgcttttccctgcacccttgctggttgaagatcgtctatctgtcccctgtttatgtagcagacgtgtgcatactacatgggcttatctcctagcgtccaggccaaaatacaagctgagcaaaggtaatcatcgctccccaagtccaagccccgggaaaggcaggggctgcaggaggaggcggcccaggagctaagggggcgtgaggtgtctccctccaggccggcccgggccatttcttcacgggctttctctacctgggaccgctccggagacaggccaggagccctcgacagacaaactcagagccacagggagctgtccctcgggaaaccatcgaaaccgcttagtaaccaactccatgggtcaaggcccccgcccagatccttctccctgtccagaggggggcctggccctggctcctcagggaagctgaggcctcgggattggcacagccctgcaggtcggagggagcgcgcgggcggggaggagcttgctggcgatttccacctgcacccgcgccccgcaccccggcctcttcccggggttaccctcccaacggccctcggagcccggggtggagaggggacagcaggaggagggccctcccggaacccctgcggggctgtctttggcgccaaaggcggccggcgctgagggcaggacccgcgtctcctcgcaggctagactctggccgctccagcctctcgcgagcgccaagtcccaggggccgatccaactccgagggagcccctgtgtcgccttgtcccagctcgtccccgatcgctctcccctcacctcccagcttcggagctgcgagcccaggtgcctgcccagcgctgcgctccagcccccgcccgcggagccgaaccgccccccggccctgggcctcctacctgcagccgtcgcagccccgggcatcggcgtccgtcccggctggcctggcgcgggccccggtttcgctgcgtccccttcgcgggcgcgcgagcctccgggcgggtgcgcgggcggggaggcagggcgggctgcccggcccctaggcgggttatatgggcgcggggaggggaggcgccgcggggagtaggcggccgcgggcgttagcgcctttttagcccggcgcgcgggaaggcagcgcgggccaccgagtcgcggcggggccaaggtctcctcccggcgctccgcctctccagctggccgcgtccaagtcggggtcccgggcgcgctctgccccgcctccttcccgccgccttttggcgcgcgtcgctcctgcagagcccggcgaggccgcccacgcagggccgaagcaggtccggcgccagggcccctcccgcgggccagacccgaccgcgatcttcggcagagctggggacgccggtgccgactctgtgcccgacgcctgcgtttcccccggactccctgctgtcccccagacggcccatctttcctgacacccgggtctctcctggccgccgccactagcgctgggctcctcggcgcgcggggcgcttggaatccaaggggcaggggatgtggccggcggggaacaggggtgaggggcggggaacaggggtgaggggcgccgccccaggcccaggaggaggggaacggctgctttccgccaagcagggcttgccacccacttctcctgtggagccacggtcccgcccggcagaggatcccgcggagagcctctgggggctgctctcccaaccccagccggagtttgggccccacgtccttccaggggtcacccaggacgcgaacactcccgggcggcggccagcaccctgctccctcccccggccgccgcctgtgggctcgggctctggatcccgttcggtgtttggaaggagctcgcgggcgtcggtgcgcttggagttgttctctgcgcgcaccagacctgcgctaggtcaggccgaagctctctaccggccagcgacacccgccgccccgcccgcctgctcctcctaggccatcggggcttccagggccctcgaccacgtacaccaggccctggctaggggacccgacatgctgggcgagttccgagcggctctcagtggcggcgctgcttatggtctgatgcttatggggcccgtgatctggctccgaaccgatgtcctctgcccatggtgacccccacgtcttggaaagtcgcgtcagtgaagcccgtggcttcgcgagcgagccgggctggcgcagactgggttctctggccgcagcgggtccgcgcctgacggggagggtccggctgactcgcacatcttcatggctcacccactgtttgctgggcgcacggggtggggacccaaagacgaatcccatgcagaggtttgggtctagttttggcaacagaaaaggccggcaccgccggagtaggacgcgcgcgcaccaccatccattcggccggggaggttggggcgtccgcggggagcagacccgaggctgggcgcccggaggtacgagcaggaccctggccggctctcagctcccagaacccagggccaggcggggatgtcagggtctgcgcgatctggggggccctagaggcgccggcggggcctggcgcggactactagcggcctgcgggagccaggggtggagggggccgctgtctagggaagaggaccggggtccggagtgggggtgagaacgaggcacgcccctgagcctgcgaaacctgcggaccgagcgcccgcgcaggagctcgctccctctgggcccccaggtcccagacccgcacgattcttcgcccggtgccagcccagatgcgcgcgggtgatgaacgggcggtggccgcgatccacgcgctcgctctgcccagtcagcggcaaattcgttctctccttgatctcagtggcactggagcgtgaggcaagggccaggcggcaggtagaggggtcggactcagcaaaagcaaacaaaaccccaagcaaacgaaataaatacttatacattacatacacgatataatatatgtgcgacagacaccgtattacttattaaagatatatttgtaggtaaacatatatttacatgtaaacatacatttacatataaatataaatatatatatatgcatgtgtacacacacgcagacacacaggtcgctcaattaaatttgaatttcagactagcgagtaatttttccgtattagcatgtcccgtgcaatatttgggaacatacttataactagatttgttgtgaatctgaaattcaaattttactgtgctttctacatttttatcagcagccctagctggaggtgctcaaaggcctcccggcctttgaggtaaggttctcgggtcggaaatccctggtcggaagaaacgtgggcgtttcctttaaagccctgcggcacaagaactacaagccccacaatgccgcgcggaagccggcggcgctgataggctgcagttcacttccgccttctgacttccggcctcggaggccggcgtcggctgttgcagcctgctgcgcgcccaggggtcccgcgggttttcgggcgcagggtggcgcccgcggcaggcggcggccatgaacttctccgaggtattcaagctctccagcttactctgcaagttctccccggacggcaagtacctggtgagcggcggggacctgggccacgggcaggacatcctcggtgccagcgtccccggcgggcagggccgggggcggccgcgtgggtggaggcagcttggggatcctgcaggtgcggtgcccggacaggtgcggggcccgggcaggtacccggggcaggtgcagtgtctgagcaagtgccccagacaggtgcgggacccaggaagataccctagacaggtggagggcccggggaggtgccccaagcaggtgcggggcccaggcaggtaccccaagcaggtgcggggcccaggcaggtaccccaagcagatatgggggccggacaggtgcaggaccccggcaggtgcccgggcaggtgcgggacccaggaagataccgtagacaggtggggggtctggggaggtgcccttggaaaggtgggtatcggcagtgccgactgcacacaggtgagggtataggagatgtcggctctttgttgttactttgtcacaaaaggcaaattgatgattctgttgcctgtaaactggtaagtggtgtggctactttgtcatctctacttctcctctaattctcttctcctctaccacatgccgaataaatgatttaaatactagtattctatccccaggtaaggaagtagttgtcctcatggctgggatttgaggtcttcgttccatgggtggcagaagggtgcttaaatcacagagctataactttaagcttcaaaacatatagtgtatgtacctggatgcctagtagtgtatatcctcagaagagagggataacccggatgcctagtatgtatgtcctcagaagagggggacaattggcattggaaagccactaagaagaaacatgcctcgtaaaaagtgggcttcgttttccttatccaccacctgtactaactgactgatgtatcatgatgctttttcaggttttgccactcctgacattcctgtgctctgtctagcttttgagagtttaactctcaaattacgttttttacctcttcagggaggttttattccaaatcacataaaatagcatcagctacaaaacagaaaacctcattagtttaaatggcatctcagccttggtatattttcaatgtgcccccctcctcaattaaatgagaggtttgttttttgtttttaccatttatgaaaatttcagtgaattataaggagaaagattacatctgcttgcttaacttgggtgtttttttccagcttgtacactctggtcaaaataccctgaaacacttggtaatgatctgttactggaatcatcttcttgaaagccctgaggactggggagagcaggtccatgtttctagttttcatatcttgcagtagagtttatgtggttcctcaaggtgttggtaaaaatattccatttcataaggaattcactgtaactgatcacagttttcaaagctacagtgtgagaaagtaagtgctgtggtagctccttgttccaatctgccgtcacccgcctgtctctcttttctcccacggaaaactaacgtggctttcttttttgatatcggttcataaaacacaaactactttgaccttgataagtaaaactaacgaagcagttaatatttaaacatcagttcttgatcttgaaactgtagtaaaatatacattcagcaggtgagtactcaggcgttatttttacttgaaattgtttgcttggatagggacataatgaacaaatatggacaagaagaaactatctttaagaaaatagtactaacacttaaacatttcccgtttcgtgttaggaataggttaatggagataaatgagcatttgcgaagcatctttatgtgtgcagaagtgaaagcctgtcttttgatcttctgttgttttggctctgagttcggcactgcataagaagattgatgcattccttgccattttcgttgtcatctgtgaagcctcaattcttaccctttataaatttgaggaaattgtccaaaagtgtttataaaactagcagaggattttggttattagttcacagaagagctttctcccaatgctacttcccatttaagtttagtttttaacatacagataagaagggacttaaaaatgtcttaaagctttaagtagcgagttaaaacatacttttatactttacaatagagtatagcaatattaaaacctggcttgtatatttcaacattacccttgtatgaactgaactgatctttaggggctggggatgtcactggtgatctagacatgaaacctctggcaggagagaaggccacagtcactgaaggccagggcagtagcattccgaaggcccccctcttttggagtggagaatgtcaggtgcttgctttctcatattttttaggcttcctgtgtccagtaccggttagtggtccgggatgtgaacacccttcagatccttcagctgtacacgtgcctagaccagatccagcacatcgagtggtcggcagactcgctcttcatcctgtgcgccatgtacaagcgagggctggtgcaggtgtgtgctgccgtgtcgcttagaatctggacaccttctgagggcccctgggaaggaggggttttgttctgtgcctttctagtttgaaaaaaaagtccgtgcagctcaaagccagttttcttttgaaattataacgttctctaaagtttaagaacatttaaactaaggaaactccaacgtagtcttaatttctaagcaatatagttgagaaacggaggtgttgacagcaatctagccacgcggatgtggtgtgtttcaggaagtcctaagaagtcgtgttttcttctgtgtccaaagcttaactaaatgaggtcttaggaatgacaatgtgaataaacagttttaaaacaacaacgaaatgaaatcaccaagaagcgtggcttacttttacactggaatccagcaccagcccttcatcatgaaaggttctaaaagacaccctgtgtgatcgggtctctgctggttttaagggagttgtctctcggcgaggcctcgctatagactgctgatgagtttgaagacaccctgtgcggtcgggtctccgctgcgtttaagcgagctgtcctcgctgtctctcggtgctgatgagtttgcacttgtgtgtttctttcttctctcaatccaggtctggtctttagagcagcccgaatggcactgcaaaatagacgagggctcagccgggctggtggcctcgtgctggagcccggacgggcgccacattctcaacaccacggaattccatgtaagtgtcagccccaagacaccttactggccatgtgatcctgtcctctgctctcgaaacagctggggagtgcgagagggggtgtgtttggagtatgctagtctccccttatcctcaggggatgcattctaagaccaccaggggattcagattgtaccaaaccctgtatttcctgtgttttttcctatacagcatacctgtgatacatttaatttataaattagatacagtaagagattagcaacaatcatagtatagaacaaggataacaatatactgtttataattgcatggatagaagatttggtcttactgaagatctgagcaacaacctcagcataggattttttctttccttgttgcgtcaagaactttcagctcgtcatttaaaggaagcactttgtggcttctctttggcgtatccagattgcgagccccaccgcccgtgctttgggctgctactgagtcagataagggtgtcgtgaacacaggcactgtctgctgccctgggtctgatgactgaggtggctcccgagtggctggtagattggcggggttggagggagccggggagcgtatatagtggtgtgtatattttaggtcagagcattataagccactaagtattattgtctgatcttttgtccagttttgaagaaaaaacattttaaaaatcttaatttagattttttaggatttcggcactccctttaattcaacgatagaaaatgcgctgtgatatgagccggctcgcacctttccttcaggagcctggcctgggggcgtggctcggggcggtcggcagccgcccattcacctgctgtaggtaccgcacaacagcctccgtgctgagctcagcgcggaggggaatgttgacatccgagaataaatcagagggatctgggataggagactgcattttaagttaaaattaggcatatgtcaagtgtttagataccaggtctttggtgtccgcagtgtctttttaagatacagaaaaatggacgaattcctttaataatgtgcttttctgatggagttggatcccagtgtttgggcccaagtccactgagtgctcatagctgtctgggctgctggctgcacctcggtaacccgcccatcccgctgcgccgcgcacaccacggggcaacccgcccatcccgctgcgccgcgcacaccacggggcaacccgcccgtcccgctgcacggcgcacaccacggggcaacccgcccgtcccgctgcgccgcgcacaccacggggcaacccgcccgtcccgctgcgccacgcacaccacggggcaacccgcccatcccgctgcgccgctcacaccacactgcagccctcccgtcccgctgcgccgcgcacaccacgtagatgatggcacctgggaggatttctcccagcgtgtggctgcagatgctctgtagggtttcattcggcttttttcaagctggcatcggctgggataaagttttatccagccatgagggttgctgaatcaactgcttcagatggctggatggctttacatatttttcagtttcgattttaaaaaatactccaaatctaatgtctgctattatgggctgtcagagaataagacaggaaacaaaggcccaccaatgtctccgcactatgtattgacattggaaggcactttcggcctgagtgtttcagagaggataatttgagcttttcacgcagccgtgtctaacagtcttcaggcctgtaatgctttgggcccaaatctgcccttgagaagggctcatgtcatggggtcagccttgttgtattatcttattaaatggcccagaaggaaaactaccatctatagagtttagaaaccttaaaagatggataaaagcctggttttgttgagcatcccatgtcacacacaccaatattgaaaacacctttgctacagaacaaactgatttttcatagagtacatttagatgacattgttcaacctaagactaaggtcatgaccactttgctggtggccttagaggaaacgttaagaccaggtggtggatgaggaccggtaacctcaaggcatggggcatcttgaccaagatgtcaaaatgtgtctccagctttgcatgcgaactttaatggttgcaagtgaggaaacaaaaccggaattccagacgtgctggccaggaagtgagctgcgggcaccgtccatcagtctcattcacaagagcaaggaccagacccgttcgaggattagcggggccactaggtccccttctgaaggcgcgaccccatgtccggggctcagcggggccactggatccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatgtccggggctcagcggggccgctgggtccccttccgaaggcgcgaccccatttcgggggttagtggggccgctgagtccacttgcgaaggcgccaccccacgtgagggtgctgtggagcatgggagacgtccctgcctcttgcattttaacgtgaaaaggcaagcttgcgcccattagacgaggttcagtgagtcatcaaaagcggctcacccctaggcctgcagcggaacactgccgcgctttcagagcccttctcttcctcgtagcaatgccctccccacctgagggcacctttgccccacgtcttgcgtgaatcacatcctcactgttcttgagtctcactgtacagatgtgtctaaacaccacattgcttgctgtgcagtgtaaagttgatttataggggcttgtactttgcagtctctgctgttgctgttccccgtcactgcttttagactcctccgtgttgacaggcatcgctgggccttcccttcctcgtgaggaacgccattcagtcctcgcctgctggacgtgtgcgtcaccacggacggtgtggctgtgaacgtgttgtccccgtctcctgggcacatacccggagagcctctctaggagagccttgcttggccgtagcagacggaggggtttcatttgccaaggcttttgtggcaggattacaatgattccatccaggcgactaaatgatactgcagtttggatttagtttgcgtttccctaattctcggtgaggttgagtgctttcctatcttgtttcattacacacagacgctcacacatgctccatttaaatgaagcaggttcacgtagtggggcacactgtggggtctgaatccatttctaccttttcccagcagcatttttttttttttttttttgagatggagtttcgctcttgtcccccaggctgtagtgcaatggtgtgatccaagctcaccgcaacctacgcctcccaggttcaagcgattttcctgcctcagcctcccgagtagctgggattacaggcatgcgccaccacgcctggctaattttgtatttttagtagagatggggtttctcatgttggtcaggctggtctcaaactctcgacctcagttgatctgcctgcctcggcctcccaaagtgctgggattacaggtgtgagccaccgtgcccagctcccagcagcatttatttagaaggctatcttcaccagctatagcaggagctcagttccattatacctgcaggatcacttctggtttccattccagtccactgttggtccacctatgtgccagcaccacactgtctacattattgaggttctatggtgtgtttgaatatctgatgggactgattcctcttcattgctcttgtagtctgtcagattttttaaaaacaatttttttgtagagttggggtctccctttgtttcccaggctggtctcgagctcctaaactcacgcaatccttccttcccagcctcctgtatagctggcattacaggtgcacaacagtgcgcccggctctgccagatttttaaatgtatttttgagactttgttgttagctgcatacaaacttttaactgtttgtaaacagatttcctagtgaagcggttcccatgattccgcggtggcacaggccttttgttgtaaagtattcctctttatgtctcacggttctttttgtttcagagtctgatttatcccgtacgaatgcggcagtgccacctttctcttggttaggattttcttgatatgtctttttctatccttttaccttctacatttctgcatccttaaacttgagtgtgtatgttgtaaatagtctatagttagctttaaaaatatatccagactgcagactttttcttatatctgaataaaggcaattaaaacaatttataagcaagagaattcacccactttaagagcacagttgagtgagtcactttgcacgtcattgccgccacaatcaggaccaagactgttcgcccgtggcatggggctcctcgtgccccttgtgctcagtccgtctcccacctgtgtccggcagccggtgcctgctgcggggcgagcagcactgcctactcggaatcccatctgcacagagccatggtgcgtggagctgtgcgtctgcttccttttccctggcatcttgctggttttgagtgtgctgggttgtggggtggatcagtggcccgttcctccatcttgagagtgctgggtcatggtgtggagagagtgctgggtcgtggcatggatcagtggcctgttcctcccgtcttgagtgtactgggtcatggcttggatccgtggcccattcctctcaccctgagcatgctgagttgtggtgtggatcggtggcccgttccttccgtcttcagcacactgggtcgtagcgtggatcagcggccccttccttccctcctgagtgctctgggtcatgtggatccctctggtcctttcttcctgctgctgagtgctgtggagcccatcccacgggtctcctccgctggcttctccagtggtcagtggaaggtaacagcgctgatgaaggtgtatatttaactacctttttttcttattgggtttttaaaagttgcaaagttatcttttaaaggtttttgtttctttgtttttgtgagatgaaggtaaaaatcaggcttaaagtgctccctgtgtttcctaagatgtgcctgagtggggagcctcggccaggcgcaccccacagggatgcagggccacaggtgggagatgtgcttttctcctgtgactctgcagtaggctctggagggcaggtctctgccctgggtggcacatggactctcctgatgctgagaggctcgggcgctgcaccccgccctgctgatgctgagatgcttgggtgctccaccccgccctgctgatgctgagatgctccgttgctgcaccctgccctgctgatgctgagatgctcgggcgctccaccctgccctgctgatgctcagagcctcgggcgctccaccccgccctgctgacgctcagagcctggggcgttccaccctgccctgctgatgctcagagcctcgggcgctgcaccccgccctgctgatgctcagagcctcgggcgctgcaccccgccctgctgatgctcagagcctcgggtgttccaccctgccctgctgatgctcagagcctcgggcgctccaccccgccctgctgatgctgagatgctcgggcgctccaccccgccctgctgatgctcagagcctcaggcgctgcacccaccctgctgagtctgagaggcttgggcaaggtttcagcctcagacacgctggtgggaagtagcacgcttcctcgctcaggccccagcgccctccttggcctcagcttgtggagctgttagaggagcaagtgctctgtgcagctggagttccgaggcatggtccccagaacggccagtgtgtttcgggttcagcatcagcacaggagcccttccgtgctgccacgtgacccctggagctcgttgatccactacctgtgtgttcaggacttctctcctgaacgctgtatatgttgtttggagaaacaattacagacatcagattttcttttctgttgtaagaagagatgatgcgtagacatttaaattaaatcatttgttaatatcctatactgacaacctggcagcgtttgcaggagtgacactgtggacatgctgtgccccacacgctgcggccccacttacgctgctggagacggtcccccaccccgggcctcatcctcagcgaggacccctgcctgacacacacggagctggcagtcagcaccccaccccgcatggagctgcgcagccctgtgtccccacacacggagctggcagtcagcaccccaccccacatggagctgcgcagccctgtgtccccacacacggagctggcagtcagcaccccaccccgcaccttgtgtccccgcacctcggccatatttcagttaactaccagattcatcagtgggccggggatgtgggaagcaagatgattccctggtaggctccacggggtctcagtgggaaatgcaggtggagggacacagcagccctggaagtcccgcggcgctgccacggggagggctggaccacccactgcggtgcctctccctcagcaggcacgccctcgtcggcctggttagaaaatcatcttttggcctcactgtgccagcctagtcagctctcagctccctgagtcagggaaaggggtggaaaagtggacagtccagcagcgcatgttctagaaccctctgcagatgcagccaggccagcgagcacacgcaggaggcagacggacaccttcttgggtgttgagtggctgctcctgcctcagggctgcagatgcaggctcacagccccctggtgagtcccggagtggctgcatccaatctccagatcctctgtcttcccataaaggagactgaacccgtggtggtttctagatcaagatttttctctgagtaactcagggttattataacatgcaacctatggtcactttgaaaaagctaatgaaaaagcacttggatttaactttgtgtcactcaattttggtataaaacaacaacaacagaaaaccaagaacaccaaatttaccgtaaccacagtaaaatacgcatttgaatattccggtaggagggcaggcatcccccaggtcctgatagatccactccagcagtcaggcctgtgcttggggacgcgtgggcgggttgaggattgagacggtctctgatgatgctaaagaagtgtttcctttctctttcccctttcccccaacagctgcggataaccgtctggtccttgtgcacaaaatccgtgtcttacatcaaatacccgaaagcttgtctgcagggtgagtcgatcggagccacgcaaaagccatttctttgcagctgtttgacttttttgtcacgggaagtttcaaagaggcagaagtagcttcagcagcatatcaacacatagccaacccccttgggctcttttaaaggagaccttaggtctcctcatacttcatctgtaaatatttccatatgtgtctcaaaaaggcaagagtccccttttaaaacatgaccacagtgactcaagacaacgtggcctaagacatggaagaacggtggcgttgatgccggtccccttgaccttggcggcagagctgggcgttcagaccccttccagccgatgcctgttgagaggcccacctcaggcatccagttctgagctttctctcttctgtggactaaatatgatgtctgcgaaagggggaggtcttgcctgtcatctctgggcatcccagagggatatgtgaccagtgtcccttccttgtggaagcctgtgagccatctggaagaaagggggccggcgcagggtggcccactgccagcgcgtctaactggaccgcgcagagctagtgggaggtgccactcctgagtcctggacgacaccgcactgtctttttgcggaatgaaacatacacagttggaatgatgctttcatttttctctgttgctgccagcggtttggaaattgcatttgaatgcaatgcagtcacactcttattcttatagttggaatattagcgtccctccagatgagatacatatctcttgataggagtggacaccgtggtttcgtcatttgaggtattcttttcactcaggaatgatgaattctggcctagggctttttaagctaatacagaactggaaaataagagtttaacattttcaactaaagtttcttccacattttcatcttattattacttttttgagacagggtcttgctctgtcacccaggctggagtgcagtgacacgatcacagctcagtgcagcctcgacctcctgggcttaagtgatcctctcacctcagcctcctgagtagctgggactacaggcacacaccatcacagccggctgatattttgtagagatggggtttcaccaagttgtctgggctcctttcaaattcctaggctcaagcgattcacctgcttcagcctcccaaagtgccgggattacaggcgtgagccaccatgcctggccacacattttcatcttcagtttacttcttccaaataattaaattcaaaggtaaaataaatatggtaggaaccagctgccttttgcctagctggagatagccctgggtgtcccgcagcttagacatggggggactcgaaggcctgtgtttctcactcatatttgtgattaatcagaggtcacatcacttggtatttttgacagagccttgcaaaccggctcctgtgtgtgtacagcttcccccagcacacactgcgttgtgagggtgaaggcaccgtgccttgggtttctcctctgcgctagaatgttcttgtctgtctgaggagcagtgctcggcattgtgccatttcccttcttttcacctctgctttcttttcctcctccttccttgtgaattactactgtggtttctcttgcttgattttgtggccgcttgatttcagtgcatttgcctcttcccacaggaatcaccttcaccagggacggccgctacatggcgctggcagaacggcgcgactgcaaagattacgtgagcatcttcgtctgcagtgattggcagctcctgcgggtaaggcgtcccccagactggaataaagttgtcctgctgactgtggagggatcgagtgaagtagatccttccagattccaggggatctttggtaggatatggagcaaggtgatgttgtattaacaaagggcaggtgcgctgcgaatcacttccagtggcacccggttgtgtgttcaggagacttggtgaaaaggacgcgtgcccaggtgttttaaaagtgaatatgctgtcacatttgagcccttttccttgtcctgtgtgatggtgtttgcgcatgtgtgtgccacgtcgggagttggggggacgtggaagttcaccttgactgagggcctgcattaggcagggcgctctgccaggtgcccaggcacagagggcactgcccccacagcagacatatgaacacgattccagcacaatccatcattaaggtagagcaacctctggaatgctgtcagagtgcagaacccgggatccaggcagtgctctggaagggacttgtctgagctgggtctaaaggagtgagcctggccaggcaaggaaaggtgagggggagagtgtgcacatgagtgcaggtgcggggagaccaagccaccttcgggggctggccaggcaaggaaaggtgagggggagagtgtgcacacgagtgcaggcgcggggagaccaagccgccctcgggggctggccaggcaaggaaaggtgagggggagagtgtgcacacgagtgcaggcgcggggagaccaagccgcccttgggggctggccaggcactgacgctggcatctgtggcggtgcagccccagcagcagctctacagggatttcatgcatttccaggatagagctgttgagattttgtcaatttctatataaggaaattaagtgcgttaagaacatcagtcataaatgcaattatttatttacgaaaatattttccaaatttaatgtttcccccccttcctttggaagcattttgatacggacacccaggatctcacagggattgagtgggccccaaacggctgtgtgctggcagtgtgggacacctgcttggaggtatgaagatgaccaggtgacatgtttgctttcgagagcttttcccggaatttcatgctttctgaatgctgcaatatagtttgctttatcttatttcagataacaagttgagctttgttttaaaagacagggtcttgctctgtcacccaggctggagtgcagtggtgtgatcacggcccactgcagcctcgacctcctggactcaagtgatcctcctacctcagcctcccaagtagctgggaccacaagcatgcaccaccacgcccggctaatttttgtattctttgtagagatggggtttcaccgtgttgcccagactgctcttgaacttctgtgttcaagtgatctgccagcttcggcctcccaaagtgctgggattacaggcgtgagccacagtgcccggccgagttgaacttttatcagcgagccattaggttgacctcccaatttcccctttttcctcatgctctcactgcctctccgtgactatctcagtactagctggcagttccagcttttccacggccacgcggacagtcctgccagctttgtgctatctgcaccaatgtcactctgcagcacccgcgtctgtggtgtgttcctggcgacgggggtgtattcattattaactgaaatgtctcatctgcagtacaagattctgctgtactcattggatggccggttgttgtccacgtacagcgcttacgagtggtccctgggcatcaagtctgtggcctggagccccagcagtcagttcctggcagttgggagctatgatggaaaggtgggaaccagtgcagggcactcaggaagtccgaccgcccagcgggccgggccctgcctcacctgtgcatggtttccttcctcaggtgcgcatccttaatcacgtgacttggaaaatgatcacggagtttgggcatcctgcagccattaatgatcccaagatagtaagtctggaacacgtgtttctgcctgagcaggctgttggggagggcggtgggaaactgctccttcactttgctttcttgattgtggtttattatttaaccgggcacttccatcaccgtaaacttgctactgtgacatttcacgcgtctggccctgccctggcctttgttcttcctcctccacgctgcccaaagggtctgaagtgggccttgccctggcctgtcctctgccgctgccaccccagtccagttttctcctttgtggtctgtctggatgcagatccctgtgctgtacagagtggccagggtctcctgcccccaaacactgggcatctgctgtcgtgccaggcactgtgctctgggccgcagccagagaagaagagacagacccctgcacttgcagagtgcggctggagagacagggaaggggctggagagctgggtgtctccacggggtggcagggagtggccaccagcgagggatcgcaggcacagatgctgcaccagtgatcagggactcggggagggagcgttccaggccaagggaacaggaaatgcaaaggccccgaggagtgggccacgcatccatgtgtgcagcgggcactgcgctgggagccagagacaaatgctgccctcctggtgttgacctgtaaccgacagagctgagtgaaaggcggcttggattcgatgaagaggccagtgagtggctggggctgcgcatgccagatcagccactgccggggatgcatcctggaggggtgtttaagtgaagaccaggaggaggagctgcgcggtgtctgggcagaaagggtgtttcaggcagaggggcctcctgacgtggccagtggcttgtgtggctggaggccagggtgtctggacaggtgagcccgagcagggctggccacacaggtcctgaaggtttgcctgtgattcctgcattctccaaatcccttcgccacacagcagcagagcatgctttaaaggcatgaaccaggccaggtgactcccttacctggggctcttcaggggcgtaggggctgcccgccttccactgagcctgggccgctgtcctttgcttgccaggctctcgggcacagatgcccccacctcgggtctgtgtctgctgctcccggggcctctgctgtgttggtgcaagtgttctctgtatcctcttatcacggagggaaggctgagcccactcccttgctggatgatggtggctgcagtggtctcccgtctgtaatcacagggcacacatttgctgcctgtgtctcaaagggtgggctcctcagcaggacacgagagagggtggctgggtgatctcagctctcgggactgcggggttcgtgcttccgtctctcccggtggcagcaggaccagctgtcatggcaggcacacctgttgaccatgcgctgtcatgggcatttgccacgtccatccttggcttccgggtccctgtcctgagccgggcgtggccaccttgtgtctttcaggtggtgtataaggaggccgagaagagcccacagctgggactgggctgcctctccttcccgccgccccgggccggggccggccctctcccgagctcagagagtaaatgtaagtagcacatgtgatgtcatttgttttccttttttaatgcaagattcctcgttgtccagataaacttcggatgttccttatgtttaaaaaaaaaaactttttttcccttccatgcttccagccagtgtgcccctcagcctgttggctggaggttgactagggtctgaattgcgcagtgggccgtgaaggtgcggcttctgttactgaatatgagccctggagacccggatctgacagcgggagctccgaggggatgtttttccggagctccgagaatgatgtttttcaaagaaacacttttccgtgggtggcgctcagagtgggtctggaacccggcggtgctcccgagcgctgtctccggcttgaggcccggttctgatgcagcctcttgactctgggcatttcctatgtgtctgtttcctcatcgctgaaatgggcatagtaaaagcacccatcccacaggggcatgagggcatctgaaggttgcaggtagaggaagcggtgccccaagtgccgacgggcccaccccggggctcggtgtgtgctcaggatgggggtcaggacaggtgcagcctggcacacaggaggcgctccccaggagcccaggggcgtccttgttccacagcccctgcttctgctgtgatgatggttcttccacagccggaaagtctaagggtactgacgctgaggtggaagggattgtgaactcagatgctggtagtcgacagaaaagtgctgaggagttggagctgctgagattgcagctaggacctgtccatgtccctctccagtcagcccctggcacgtggggtccaccccccacctagtcgggcctggcaggtgatggtgccactctcatccggggcacttgcgggcaggcccctcgctgcggctgccaggctttgcttagctcttcctgcccttgggccgcactttcctcccctttgaacagaggagctgggttcgcgatgcccgatgcaggcacagcagcctctcagccgcgtgcgtacttcccgaaagggtgacaatggcgtgttcttcctgttggtgttctagatgagatcgcctctgtcccagtctccttacagacactgaaacctgttaccgacagagcaaacccgaaaatgggcataggaatgctggcatttagtcctgacagctacttcctggcgacaaggaacggtcagttgtgctgacgcaggtgctgtctcaccctttactgtgtcctgtggccccaggtgtagaagcgactggaaaagaaaatgaagttgggggtttccgacacgtcacctcctttcacgtttctgcagtagccgaggtgctcacagaagctccttggaggcgctgggcctttgtcctgcagcagccgcaggtccgcgcatgagtcaggaggcctccaccctgctcacttgctccactctgctgagtagaaagcagggctgggcacactgcagcccgccttttgttaagggtttctccatttgtaaagggctaaaaaaaaaaaaaaaacaccaaagaaagaaacacatttgggccccttacagcccgtgatggccaccctggccctggccaggagccctacagggcccaactgaacccagactgcagggtgcagccgccccaccccaccggttcttcccctcccctcctgtggtgtcaacagcagagcagaggggcagggccaacacagaggagccaggcagggatgacaccggtgccctgtccttccttcctcagacaacattcccaatgccgtctgggtctgggacattcagaagctgaggctgttcgcggtgctcgagcagctgtccccagtgcgcgcgtttcagtgggacccgcagcagccgcggctggccatctgcacgggaggcagcaggctctacctgtggtccccagcgggctgcatgtcggtgcaggtgcctggggaaggtaagcacatccccgaggccacggacgtggcagccttgctgtgtgcagtctggcatcacacgggctgaactgttgaagcccagctgctgtctccggcagcactccagggaaactgccctcagagtctttgcgtaaacctcaagacggacacacaggggacccgctgccctcagctttatgggctagaagcagcctctccctgacttgaggcagctctggggctccaccctggctctgcctatgtgggtcactccaggcactgtgctggcctgcaggacaagggggtgcccatcccccagaatctgggcctcccctgctgtaatcctctggccacctgtctctctctaggcgactttgcagtgctctctctgtgctggcatttaagcggagactcgatggccctcctcagcaaggatcacttctgcctctgcttcctggagacagaggcagtggtcggcacagcctgcagacagctgggcggccacacgtagcagcggtgcactaacgtgtgcagaaacagggctactctgtgtttccagtgtgggaaaaaacacagcttcaccaggaggttctccactgtggtggtctggattcagtgattgattctatttttctatagcaaagcatttttgtaaatatgtatggtataaaactgtagttttattatttaaaataaatacttgctgatttatacaactacttctcttcttgaaacgtttagtcacaggcaggttttctaggttcgaggttctggtcagtcttagggaagatggatagccctacagtaactgagccccgggccactagctcccgccagacacccggcctgctctgggttctctgtttggatgtgttaggacaacgagggggcatctctgtggggcacatccagcctccccacagtgggtctgaagtgtgggcagggagccctggcacctgagagcagcgccacccccaggtccagtcccctcgggatgggagctgcaggtccccgaccagctgagtcagttcctcaggaagtctcatgtctactctggtttcggaaccagtgtgcgtgggtctccaccctggctatttgcaggaagtgcctagaatcacccagaaccattccatcaatctctccagctggtgctcggcaggcagccagacttggttggcaccacaggtggggggatc

SEQ ID NO: 11 - protein, 661 aa, Homo sapiens [sequence omitted]

SEQ ID NO: 12 - protein, 100 aa, Homo sapiens
  N-terminal segment of human delta-N p73 protein from Figure 4
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLK

SEQ ID NO: 13 - protein, 100 aa, Mus musculus
  N-terminal segment of mouse delta-N p73 protein from Figure 4
MLYVGDPMRHLATAQFNLLSSAMDQMGSRAAPASPYTPEHAASVPTHSPYAQPSSTFDTMSPAPVIPSNTDYPGPHHFEVTFQQSSTAKSATWTYSPLLK

SEQ ID NO: 14 - protein, 100 aa, Artificial sequence
  Variant (8, 22, 27, 32): Xaa indicates a non-conserved residue
MLYVGDPXRHLATAQFNLLSSXMDQMXSRAAXASPYTPEHAASVPTHSPYAQPSSTFDTMSPAPVIPSNTDYPGPHHFEVTFQQSSTAKSATWTYSPLLK

SEQ ID NO: 15 - DNA, 643 bp, Homo sapiens
gccctcatgcctgggaacagaggctgctttacggggtgagggcctggggccccccgagccttccccaggcaggcagcatctcggaaggagccctggtgggtttaattatggagccggcgctgaccggcgtccccgccctccccacgcagcctccttggtgcggtccaacacatcaccgggcaagctgaggcctgccccggacttggatgaatactcatgaggaataaaggggtgggccgcgggttttgttgttggattcagccagttgacagaactaagggagatgggaaaagcgaaaatgccaacaaacggcccgcatgttccccagcatcctcggctcctgcctcactagctgcggagcctctcccgctcggtccacgctgccgggcggccacgaccgtgacccttcccctcgggccgcccagatccatgcctcgtcccacgggacaccagttccctggcgtgtgcagaccccccggcgcctaccatgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaa

SEQ ID NO: 16 - protein, 52 aa, Homo sapiens
MLYVGDPARHLATAQFNLLSSTMDQMSSRAASASPYTPEHAASVPTHSPYAQ

SEQ ID NO: 17 - DNA, 20 bp, Artificial sequence
  forward primer for cloning 7S
gctactcgggaggctgagac

SEQ ID NO: 18 - DNA, 20 bp, Artificial sequence
  reverse primer for cloning 7S
aggcgcgatcccactactga

SEQ ID NO: 19 - DNA, 21 bp, Artificial sequence
  forward primer for cloning TA p73
acgcagcgaaaccggggcccg

SEQ ID NO: 20 - DNA, 21 bp, Artificial sequence
  reverse primer for cloning TA p73
gccgcgcggctgctcatctgg

SEQ ID NO: 21 - DNA, 20 bp, Artificial sequence
  forward primer for cloning delta-N p73
cccggacttggatgaatact

SEQ ID NO: 22 - DNA, 21 bp, Artificial sequence
  reverse primer for cloning delta-N p73
gccgcgcggctgctcatctgg

SEQ ID NO: 23 - DNA, 20 bp, Artificial sequence
  primer
tttccacaccctaactgaca

SEQ ID NO: 24 - DNA, 20 bp, Artificial sequence
  forward primer for cloning 7S RNA
accaccaggttgcctaagga

SEQ ID NO: 25 - DNA, 20 bp, Artificial sequence
  reverse primer for cloning 7S RNA
cacgggagttttgacctgct

SEQ ID NO: 26 - DNA, 20 bp, Artificial sequence
  forward primer for cloning TA p73
gcaccacgtttgagcacctc

SEQ ID NO: 27 - DNA, 19 bp, Artificial sequence
  reverse primer for cloning TA p73
tccgcccaccacctcatta

SEQ ID NO: 28 - DNA, 20 bp, Artificial sequence
  forward primer for cloning delta-N p73
ggagatgggaaaagcgaaat

SEQ ID NO: 29 - DNA, 18 bp, Artificial sequence
  reverse primer for cloning delta-N p73
gtggaccgagcgggagag

SEQ ID NO: 30 - DNA, 22 bp, Artificial sequence
  probe for 7S RNA
tgaaccggcccaggtcggaaac

SEQ ID NO: 31 - DNA, 23 bp, Artificial sequence
  probe for TA p73
tccgaccttccccagtcaagccg

SEQ ID NO: 32 - DNA, 20 bp, Artificial sequence
  probe for delta-N p73
caaacggcccgcatgttccc

SEQ ID NO: 33 - DNA, 20 bp, Artificial sequence
  primer designed to amplify C-terminal splice variants
atgctgtacgtcggtgaccc

SEQ ID NO: 34 - DNA, 19 bp, Artificial sequence
  primer designed to amplify C-terminal splice variants
tcagtggatctcggcctcc

SEQ ID NO: 35 - DNA, 19 bp, Artificial sequence
  forward primer designed to amplify 5-prime upstream region of delta-N p73
gctgggccttgggaacgtt

SEQ ID NO: 36 - DNA, 19 bp, Artificial sequence
  reverse primer designed to amplify 5-prime upstream region of delta-N p73
ggcagcgtggaccgagcgg

SEQ ID NO: 37 - DNA, 30 bp, Artificial sequence
  forward primer designed for exon 2 of TA p73
aagatggcccagtccaccgccacctcccct

SEQ ID NO: 38 - DNA, 20 bp, Artificial sequence
  forward primer designed for exon 3-prime of delta-N p73
atgctgtacgtcggtgaccc

SEQ ID NO: 39 - DNA, 19 bp, Artificial sequence
  common reverse primer designed for exon 14
tcagtggatctcggcctcc